(19) United States
(12) Patent Application Publication
(10) Pub. No.: US 2024/0025928 A1
(43) Pub. Date: Jan. 25, 2024

(54) CARDIOPROTECTIVE LIPIDS AND METHODS OF USE

(71) Applicant: Signpath Pharma, Inc., Sandy, UT (US)

(72) Inventor: Daniel Emil Levy, San Mateo, CA (US)

(21) Appl. No.: 18/457,037

(22) Filed: Aug. 28, 2023

Related U.S. Application Data
(63) Continuation-in-part of application No. 17/950,898, filed on Sep. 22, 2022.
Application No. 17/950,898 is a continuation-in-part of application No. 17/520,287, filed on Nov. 5, 2021, now Pat. No. 11,746,119.
Application No. 17/520,287 is a continuation of application No. 17/191,214, filed on Mar. 3, 2021, now Pat. No. 11,643,424.
Application No. 17/191,214 is a continuation of application No. 16/452,858, filed on Jun. 26, 2019, now Pat. No. 10,975,111.
(60) Provisional application No. 62/690,196, filed on Jun. 26, 2018.

(51) Int. Cl. C07F 9/09; A61P 9/00; C07F 9/655

(52) U.S. Cl. CPC C07F 9/09; A61P 9/00; C07F 9/65515; A61K 45/06

(57) ABSTRACT

The present invention includes a method of reducing or eliminating a cardiotoxic or cardiopathic effect of one or more active agents comprising: administering to a subject in need thereof an effective amount of one or more lipids that reduce or eliminate the cardiotoxic effect of the one or more active agents, wherein the lipid has formula:

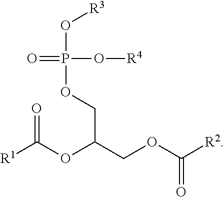

R¹ and R² is a C₁-C₂₀ branched or unbranched hydrocarbon possessing 0-10 double bonds, 0-10 triple bonds or a combination of 0-10 double and triple bonds;
R³ is

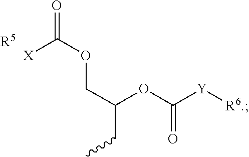

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, N(R⁷)₂ and COOH;
R⁶ is a C₁-C₁₀ branched or unbranched hydrocarbon optionally substituted with one or more groups selected from OH, N(R⁸)₂ and COOH;
Each R⁷ is independently H or a C₁-C₆ branched or unbranched alkyl group;
Each R⁸ is independently H or a C₁-C₆ branched or unbranched alkyl group;
X is a direct linkage;
Y is a direct linkage; and,

Each stereogenic center is independently R, S or racemic.

### CROSS-REFERENCE TO RELATED APPLICATIONS

This application is a continuation-in-part of and claims priority to U.S. patent application Ser. No. 17/950,898, filed Sep. 9, 2022, which is a continuation-in-part application of and claims priority to U.S. application Ser. No. 17/520,287, filed Nov. 5, 2021, now U.S. Pat. No. ______, issued on ______, which is a continuation application of and claims priority to U.S. patent application Ser. No. 17/191,214, filed on Mar. 3, 2021, now U.S. Pat. No. 11,643,424 issued on May 9, 2023, which is continuation application of and claims priority to U.S. patent application Ser. No. 16/452,858 filed on Jun. 26, 2019, now U.S. Pat. No. 10,975,111 issued on Apr. 13, 2021, which claims priority to U.S. Provisional Patent Application Ser. No. 62/690,196 filed Jun. 26, 2018, the contents of each of which are incorporated by reference herein in their entirety.

### STATEMENT OF FEDERALLY FUNDED RESEARCH

None.

### TECHNICAL FIELD OF THE INVENTION

The present invention relates in general to the field of novel lipids to reduce or eliminate cardiopathies, such as QT prolongation, cardiac muscle damage, or AV block, that are drug-induced or caused by a disease or condition.

### BACKGROUND OF THE INVENTION

Without limiting the scope of the invention, its background is described in connection with drug-induced QT prolongation and other cardiopathies and cardio-toxicities.

There are numerous pharmaceutical agents designed for the treatment of various diseases which are commonly prescribed, despite being known or suspecting of having adverse effects on the patient's heart. In addition to cardiac arrhythmias, including QT prolongation, supraventricular tachycardias (SVT), and atrial fibrillation (AF), a number of other cardiac toxicities can occur, including cardiac muscle damage, cardiomyopathy, congestive heart failure, and left ventricular hypertrophy (LVH) as a side effect of pharmaceutical agents.

The cardiotoxicity of those pharmaceutical agents can lead to significant complications that can affect patients being treated for various diseases, such as proliferative malignancies. The severity of such toxicity depends on many factors such as the immediate and cumulative dose, the method of administration, the presence of any underlying cardiac condition, and various congenital or acquired cardiac risk factors unique to a particular patient. Moreover, toxicity can be affected by current or previous treatment with other pharmaceutical agents. Cardiotoxic effects can occur immediately during administration of the drug, or they may not manifest themselves until months or years after the patient has been treated.

High-dose chemotherapy remains the therapy of choice for aggressive malignancies. Countless clinical studies have demonstrated that high-dose chemotherapy can significantly prolong patient survival; however, its use and effectiveness are limited by significant side effects, in particular cardiotoxicity. In mid-to-late phase cardiac toxicity, heart failure can appear many years after chemotherapy has ended. Treatment with chemotherapeutic agents is known to result in pericardial and endomyocardial fibrosis, heart failure, myocarditis, or pericarditis. Chemotherapy has also been associated with hemorrhagic myocardial necrosis and cardiomyopathy.

In addition, antineoplastic monoclonal antibodies are also linked to cardiotoxicity. Infusion-related cardiotoxic effects, such as left ventricular dysfunction, congestive heart failure, and other cardiac dysfunction can occur. The risk of such complications increases if the patient has preexisting cardiac disease, older age, prior cardiotoxic therapy, or radiation to the chest.

Tyrosine Kinase inhibitors (TKIs) have well known cardiotoxic effects. The antracyclins, trastuzumab, imatinib mesylate, dasatinib, nilotinib, sunitinib, sorafenib, vandetanib, and lapatinib have all been associated with a range of mechanical and electrical dysfunctions.

Among the toxic effects associated with TKIs are QT prolongation, sudden cardiac death (both considered rhythmic dysfunctions), as well as contractility issues such as reduction in left ventricular ejection fraction (LVEF), congestive heart failure (CHF), acute coronary disease, hypertension, and myocardial infarction (MI). Given the therapeutic potential of drugs such as the tyrosine kinase inhibitors, various strategies have been used to attempt to mitigate the cardiotoxicity of cancer treatment. The primary method for preventing cardiac toxicity is to limit the dose of cardiotoxic drugs. There is also some evidence that the method of drug administration may affect the risk of cardiac toxicity. Rapid administration of cardio toxic agents results in high blood levels, which may cause more heart damage than the same amount of drug given over a longer period of time. Giving smaller doses of drug more frequently can also decrease the toxicity compared to large doses of drugs at longer intervals.

The risk of cardiac toxicity from certain chemotherapy agents has been reduced by encapsulating these drugs in a liposome. For example, studies indicate that cardiotoxicity is considerably lower with liposomal doxorubicin formulations than with conventional doxorubicin.

Dexrazoxane is an aminopolycarboxylic acid that has been shown to prevent or reduce the severity of heart damage caused by doxorubicin. Dexrazoxane is thought to protect the heart muscle by blocking the formation of oxygen free radicals. One of the ways that radiation and chemotherapy drugs damage cells is by forming free radicals. Free radicals are unstable molecules that are formed during many normal cellular processes that involve oxygen, such as burning fuel for energy. They are also formed from exposure to elements in the environment, like tobacco smoke, radiation and chemotherapy drugs.

However, a need remains for new composition and methods for reducing cardiopathies, whether drug-induced, or as a result of a disease or condition.

### SUMMARY OF THE INVENTION

As embodied and broadly described herein, an aspect of the present disclosure relates to a compound of Formula I,

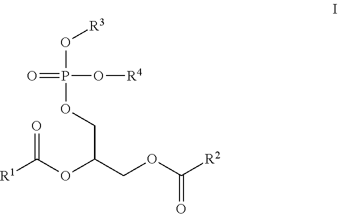

or a pharmaceutically acceptable salt thereof
wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   R³ is

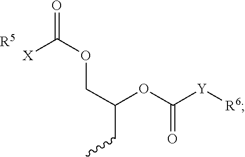

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
Each R⁷ is independently H or a C₁-C₆ branched or unbranched alkyl group;
Each R⁸ is independently H or a C₁-C₆ branched or unbranched alkyl group;
X is a direct linkage, O or NH;
Y is a direct linkage, O or NH; and,
Each stereogenic center is independently R, S or racemic.

In one aspect, the compound has Formula IA,

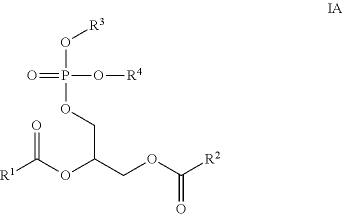

or a pharmaceutically acceptable salt thereof
wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   R³ is

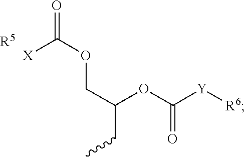

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, N(R⁷)₂ and COOH;
R⁶ is a C₁-C₁₀ branched or unbranched hydrocarbon optionally substituted with one or more groups selected from OH, N(R⁸)₂ and COOH;
Each R⁷ is independently H or a C₁-C₆ branched or unbranched alkyl group;
Each R⁸ is independently H or a C₁-C₆ branched or unbranched alkyl group;
X is a direct linkage;
Y is a direct linkage; and,
Each stereogenic center is independently R, S or racemic.

In another aspect, R⁴ is H, Li, Na, K, Mg, Ca, Zn, Cs, ammonium or tetraalkylammonium.

In another aspect, the compound of Formula I is selected from at least one of:

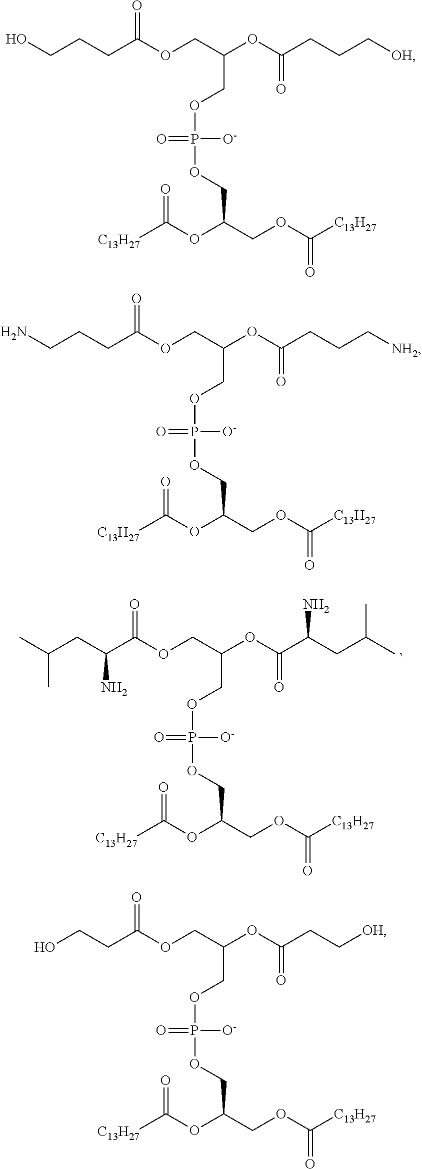

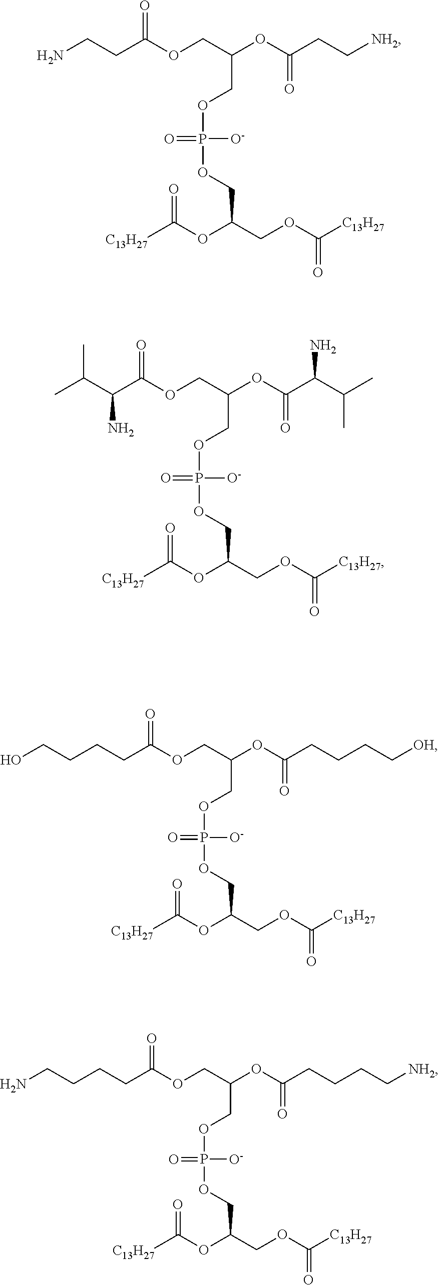

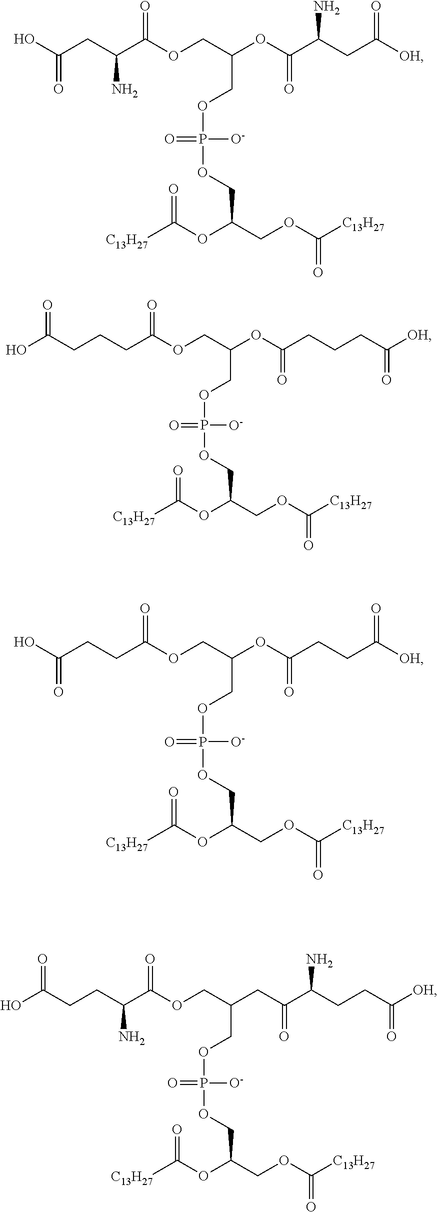

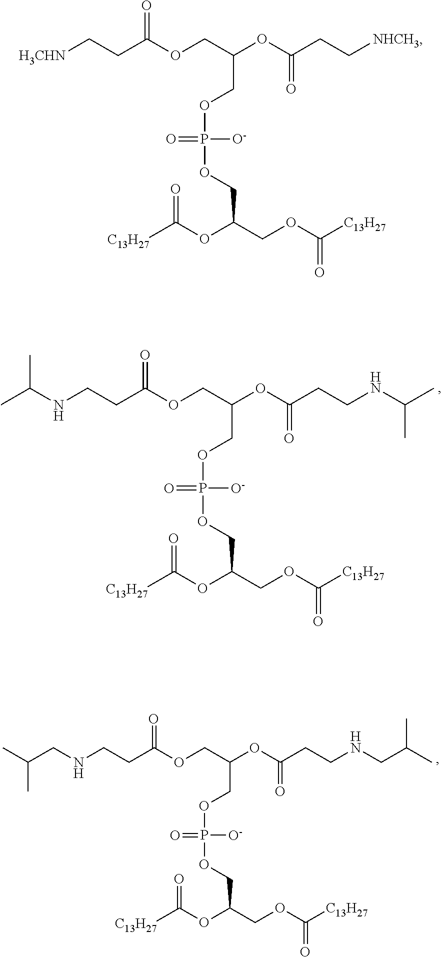

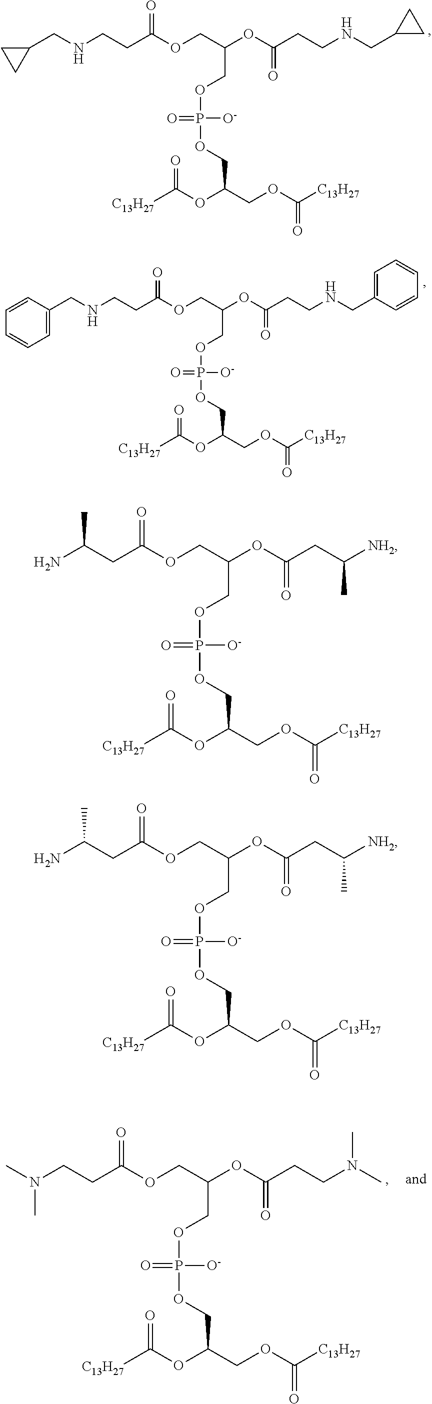

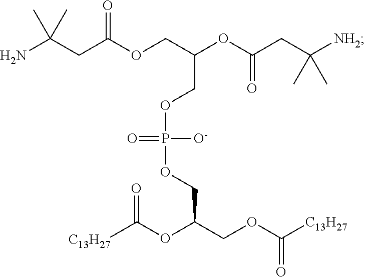

oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt. In another aspect, the compound is a single entity, a solvate, a hydrate, a crystal, an amorphous solid, a liquid or an oil. In another aspect, the compound is selected from one or more compounds of formula 31 to 51.

In another aspect, the compound of Formula I is

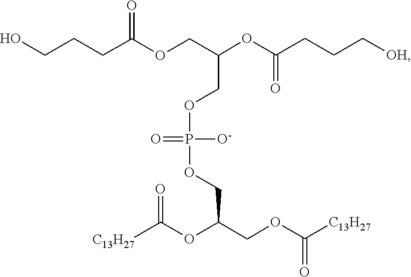

wherein the oxygen anion O⁻ is optionally paired with H, Li, Na, K, Mg, Ca, Zn, Cs, ammonium or tetraalkylammonium.

In another aspect, the compound of Formula I is

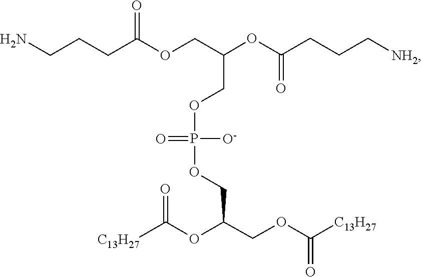

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

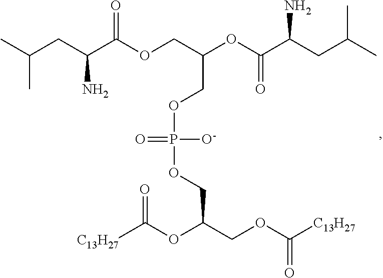

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

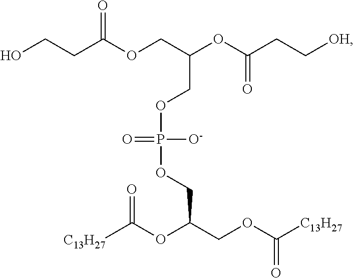

wherein the oxygen anion O⁻ is optionally paired with H, Li, Na, K, Mg, Ca, Zn, Cs, ammonium or tetraalkylammonium.

In another aspect, the compound of Formula I is

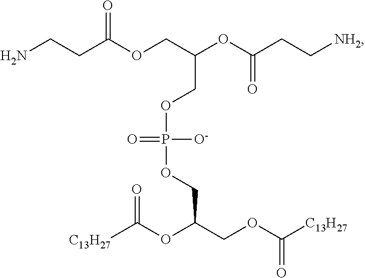

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

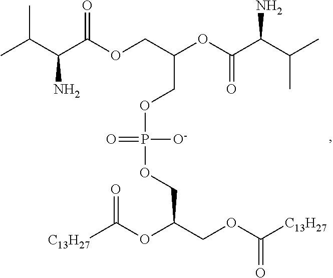

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

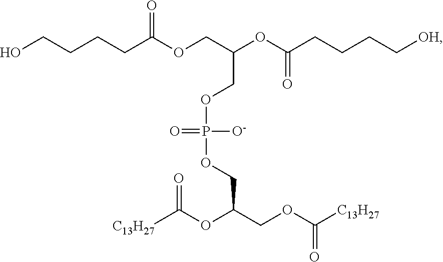

wherein the oxygen anion O⁻ is optionally paired with H, Li, Na, K, Mg, Ca, Zn, Cs, ammonium or tetraalkylammonium.

In another aspect, the compound of Formula I is

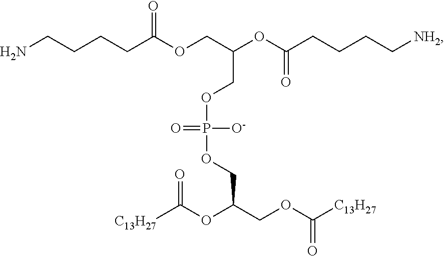

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

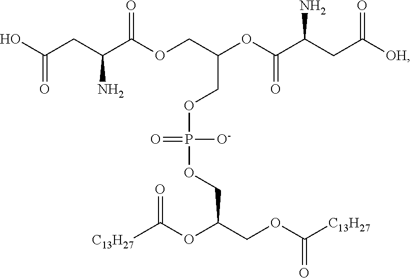

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

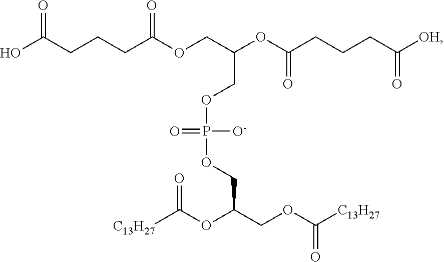

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

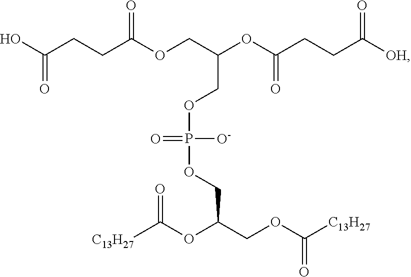

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

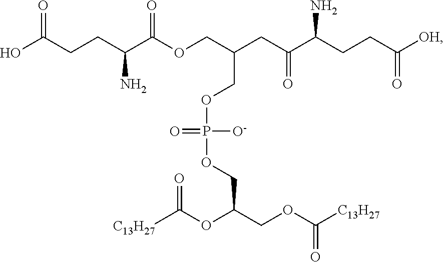

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

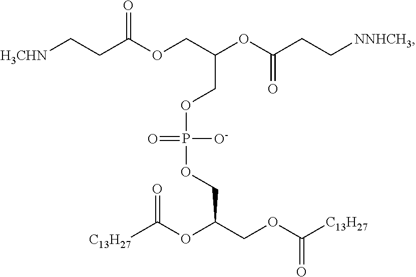

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

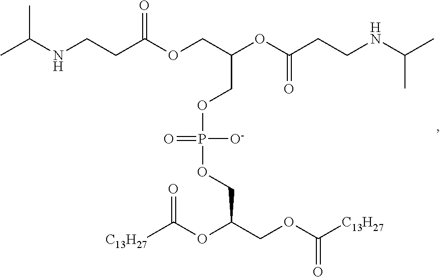

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

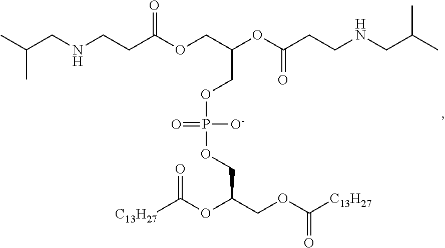

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

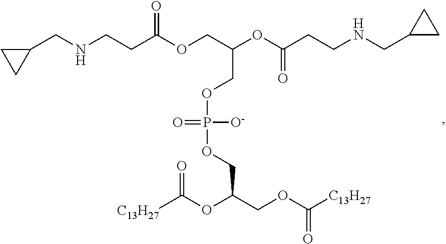

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

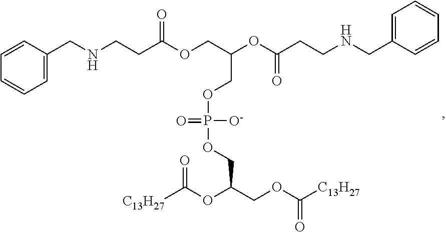

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

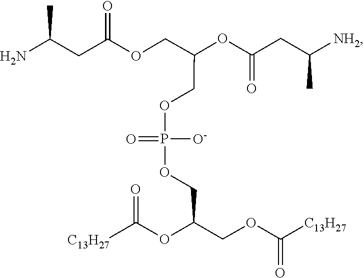

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

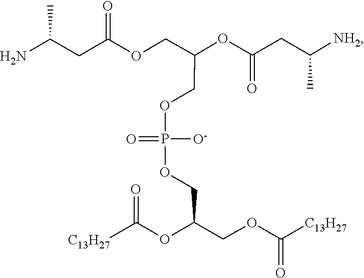

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

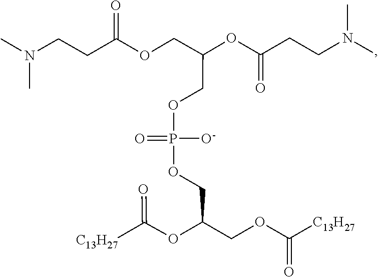

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

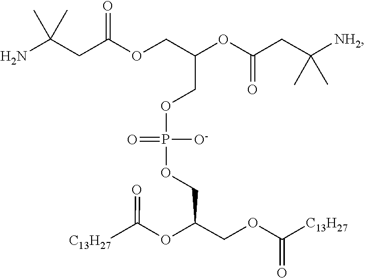

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

As embodied and broadly described herein, an aspect of the present disclosure relates a method of preparing a compound of Formula I

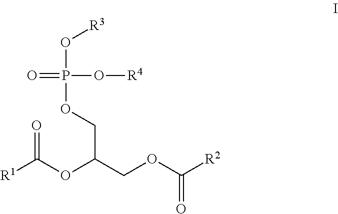

or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

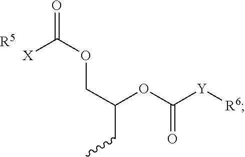

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
Each R⁷ is independently H or a C₁-C₆ branched or unbranched alkyl group;
Each R⁸ is independently H or a C₁-C₆ branched or unbranched alkyl group;
X is a direct linkage, O or NH;
Y is a direct linkage, O or NH; and,
each stereogenic center is independently R, S or racemic;
   comprising the steps of:
converting the hydroxyl groups of a compound of Formula II to esters, carbonates, or carbamates

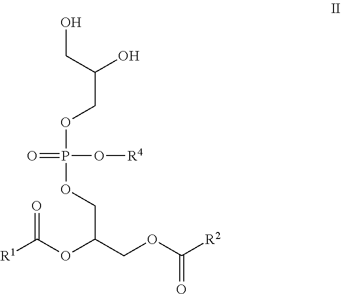

wherein, all substitutions are defined as above;
optionally converting a phosphorus-bound OH group to O—R⁴, wherein R⁴ is not H; and,
optionally removing one or more protecting groups; or
   comprising the steps of:
linking a compound of Formula III with a compound of Formula IV through creation of a phosphate diester bridge

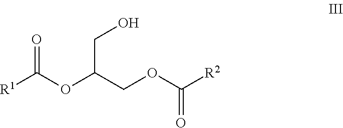

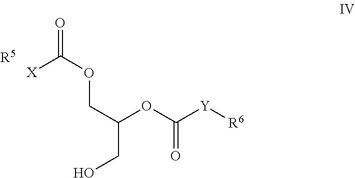

wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   Each R⁷ is independently H or a C₁-C₆ branched or unbranched alkyl group;
   Each R⁸ is independently H or a C₁-C₆ branched or unbranched alkyl group;
   R⁹ is a C₁-C₁₀ branched or unbranched hydrocarbon substituted with one or more groups independently selected from OR¹, N(R⁷)R¹², N(R⁷)₂, SR¹, CN, COOR¹⁴, CONH₂, Cl, Br and I;
   R¹⁰ is a C₁-C₁₀ branched or unbranched hydrocarbon substituted with one or more groups independently selected from OR¹¹, N(R⁸)R¹², N(R⁸)₂, SR¹, CN, COOR¹⁴, CONH₂, Cl, Br and I;
   each R¹¹ is independently H, Ac, Me, tert-Butyl, Benzyl, Trityl, Benzoyl, para-nitrobenzoyl, MOM, BOM or Si comprising the core of a silyl ether;
   each R¹² is independently H, Me, Boc, Cbz, Fmoc, Benzyl, 4-Methoxybenzyl, tert-Butyl, or Trityl;
   each R¹³ is independently H, Ac, Benzoyl, para-nitrobenzoyl or Trityl;
   each R¹⁴ is independently H, C₁-C₆ branched or unbranched alkyl, Benzyl or 4-Methoxybenzyl;
   X is a direct linkage, O or NH;
   Y is a direct linkage, O or NH; and,
   each stereogenic center is independently R, S or racemic;
optionally converting a phosphorus-bound OH group to O—R⁴, wherein R⁴ is not H; and,
optionally converting each OR¹¹, N(R⁷)R¹², N(R⁸)R¹², SR¹³ or COOR¹⁴ to OH, NHR⁷, NHR⁸, SH or COOH, respectively.

In one aspect, the method is used for preparing a compound of Formula IA

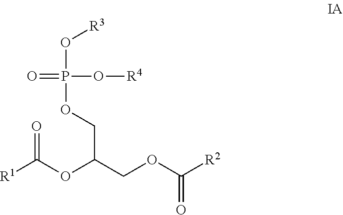

or a pharmaceutically acceptable salt thereof
wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   R³ is

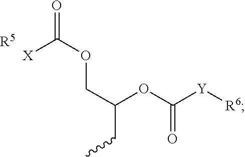

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, N(R⁷)₂ and COOH;
R⁶ is a C₁-C₁₀ branched or unbranched hydrocarbon optionally substituted with one or more groups selected from OH, N(R⁸)₂ and COOH;
Each R⁷ is independently H or a C₁-C₆ branched or unbranched alkyl group;
Each R⁸ is independently H or a C₁-C₆ branched or unbranched alkyl group;
X is a direct linkage;
Y is a direct linkage; and,
each stereogenic center is independently R, S or racemic;
   comprising the steps of:
converting the hydroxyl groups of a compound of Formula II to esters, carbonates, or carbamates

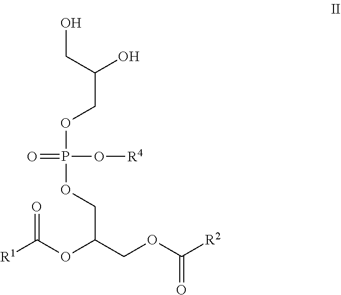

wherein, all substitutions are defined as above;
optionally converting a phosphorus-bound OH group to O—R⁴, wherein R⁴ is not H; and,
optionally removing one or more protecting groups; or
   comprising the steps of:
linking a compound of Formula III with a compound of Formula IV through creation of a phosphate diester bridge

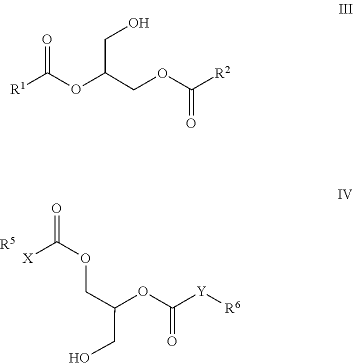

wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   each R⁷ is independently H or a C₁-C₆ branched or unbranched alkyl group;
   each R⁸ is independently H or a C₁-C₆ branched or unbranched alkyl group;
   R⁹ is a C₁-C₁₀ branched or unbranched hydrocarbon substituted with one or more groups independently selected from OR¹¹, N(R⁷)R¹², N(R⁷)₂, and COOR¹⁴;
   R¹⁰ is a C₁-C₁₀ branched or unbranched hydrocarbon substituted with one or more groups independently selected from OR¹¹, N(R⁸)R¹², N(R⁸)₂, and COOR¹⁴;
   each R¹¹ is independently H, Ac, Me, tert-Butyl, Benzyl, Trityl, Benzoyl, para-nitrobenzoyl, MOM, BOM or Si comprising the core of a silyl ether;
   each R¹² is independently H, Me, Boc, Cbz, Fmoc, Benzyl, 4-Methoxybenzyl, tert-Butyl, or Trityl;
   each R¹⁴ is independently H, C₁-C₆ branched or unbranched alkyl, Benzyl or 4-Methoxybenzyl;
   X is a direct linkage, O or NH;
   Y is a direct linkage, O or NH; and,
   Each stereogenic center is independently R, S or racemic;
optionally converting a phosphorus-bound OH group to O—R⁴, wherein R⁴ is not H; and,
optionally converting each OR¹¹, N(R⁷)R¹², N(R⁸)R¹², or COOR¹⁴ to OH, NHR⁷, NHR⁸ or COOH, respectively.

In another aspect, the method comprises the steps of:
converting the hydroxyl groups of a compound of Formula TT to esters, carbonates, or carbamates

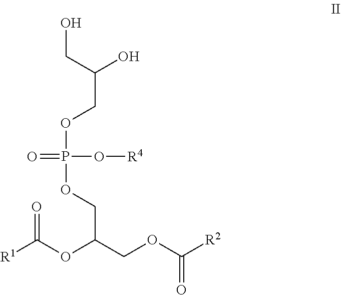

wherein, all substitutions are defined as above;
optionally converting a phosphorus-bound OH group to O—R⁴, wherein R⁴ is not H; and,
optionally removing one or more protecting groups; or
   comprising the steps of:
linking a compound of Formula III with a compound of Formula IV through creation of a phosphate diester bridge

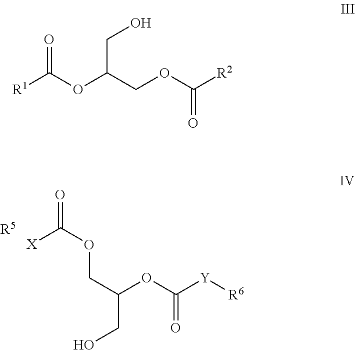

wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   Each R⁷ is independently H or a C₁-C₆ branched or unbranched alkyl group;
   Each R⁸ is independently H or a C₁-C₆ branched or unbranched alkyl group;
   R⁹ is a C₁-C₁₀ branched or unbranched hydrocarbon substituted with one or more groups independently selected from OR¹¹, N(R⁷)R¹², N(R⁷)₂, SR¹³, CN, COOR¹⁴, CONH₂, Cl, Br and I;
   R¹⁰ is a C₁-C₁₀ branched or unbranched hydrocarbon substituted with one or more groups independently selected from OR¹¹, N(R⁸)R¹², N(R⁸)₂, SR¹³, CN, COOR¹⁴, CONH₂, Cl, Br and I;
   each R¹¹ is independently H, Ac, Me, tert-Butyl, Benzyl, Trityl, Benzoyl, para-nitrobenzoyl, MOM, BOM or Si comprising the core of a silyl ether;
   each R¹² is independently H, Me, Boc, Cbz, Fmoc, Benzyl, 4-Methoxybenzyl, tert-Butyl, or Trityl; each R¹³ is independently H, Ac, Benzoyl, para-nitrobenzoyl or Trityl;
   each R¹⁴ is independently H, C₁-C₆ branched or unbranched alkyl, Benzyl or 4-Methoxybenzyl;
   X is a direct linkage, O or NH;
   Y is a direct linkage, O or NH; and,
   each stereogenic center is independently R, S or racemic;
optionally converting a phosphorus-bound OH group to O—R⁴, wherein R⁴ is not H; and,
optionally converting each OR¹¹, N(R⁷)R¹², N(R⁸)R¹², SR¹³ or COOR¹⁴ to OH, NHR⁷, NHR⁸, SH or COOH, respectively. In another aspect, R⁴ is H, Li, Na, K, Mg, Ca, Zn, Cs, ammonium or tetraalkylammonium.

In another aspect, the compound of Formula I is selected from at least one of:

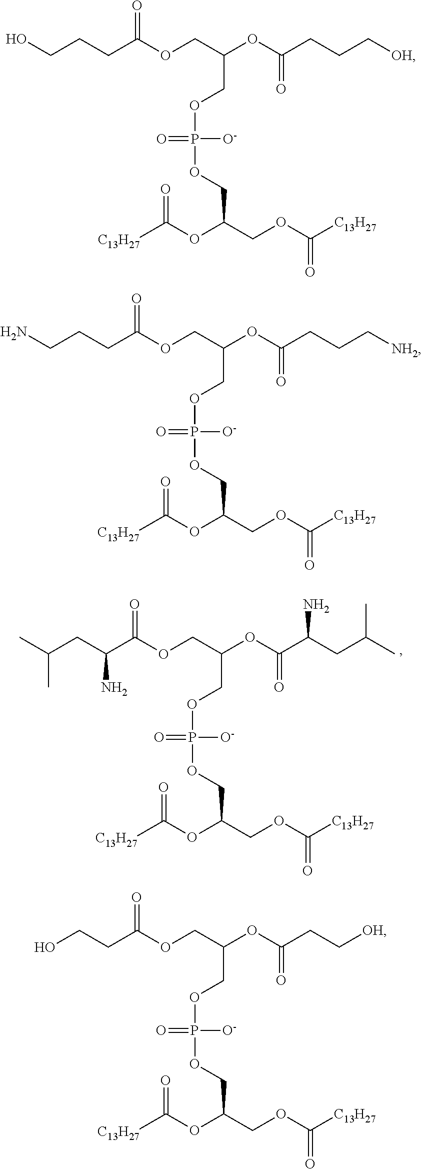

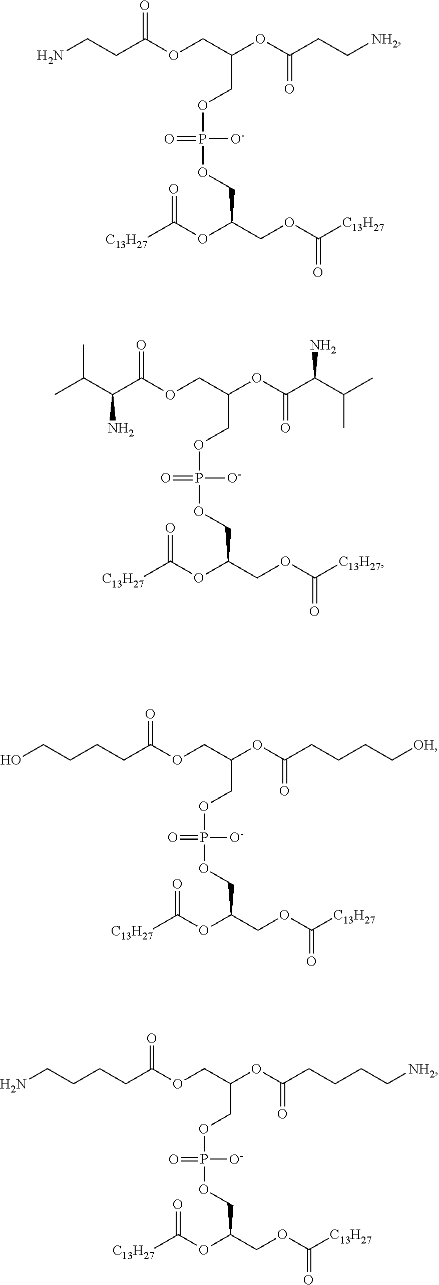

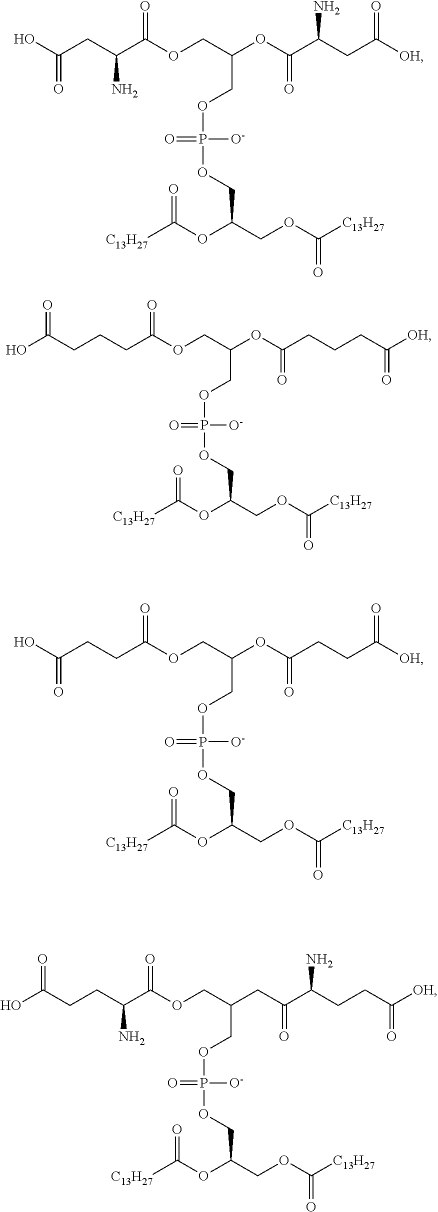

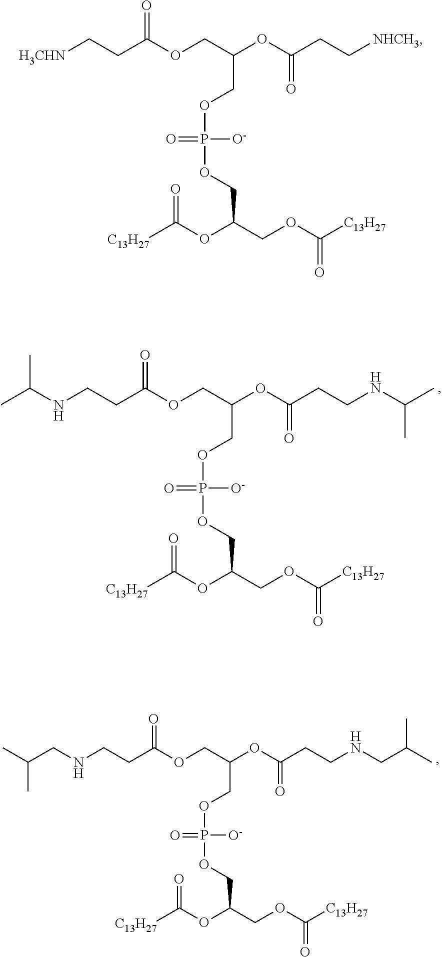

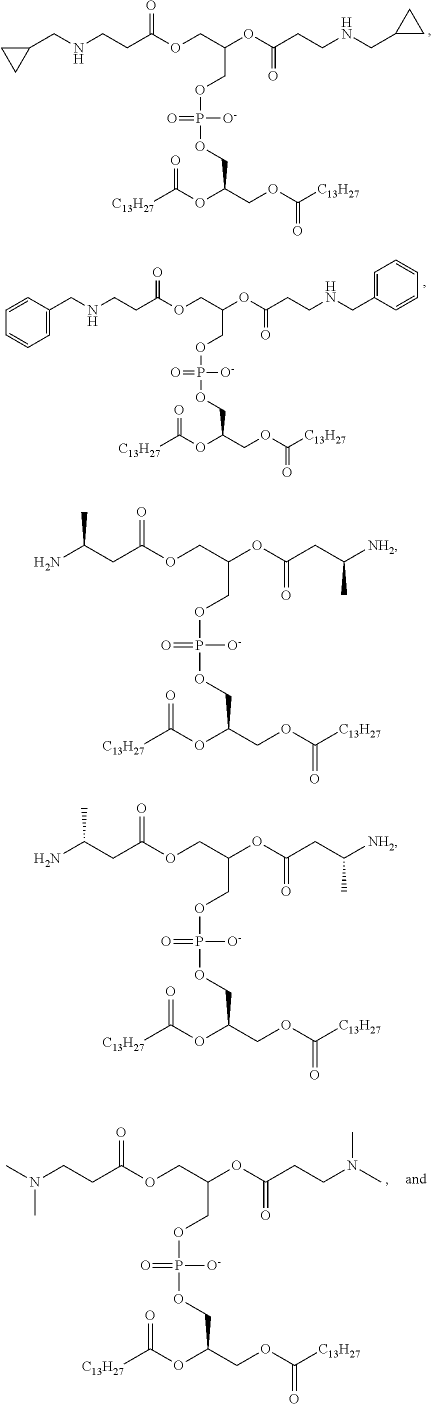

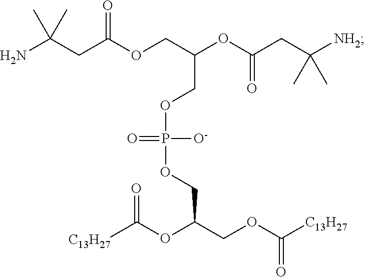

wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt. In another aspect, the method produces compounds that individually exist as a single entity, a solvate, a hydrate, a crystal, an amorphous solid, a liquid, or an oil. In another aspect, the compound is selected from the at least one of the compounds 31 to 51.

In another aspect, the compound of Formula I is

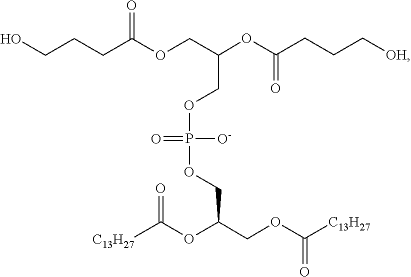

wherein the oxygen anion O⁻ is optionally paired with H, Li, Na, K, Mg, Ca, Zn, Cs, ammonium or tetraalkylammonium.

In another aspect, the compound of Formula I is

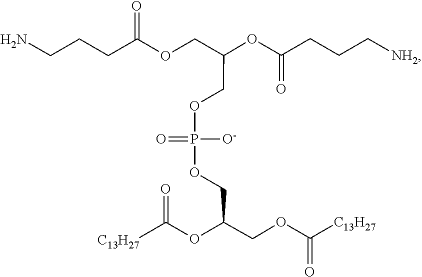

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

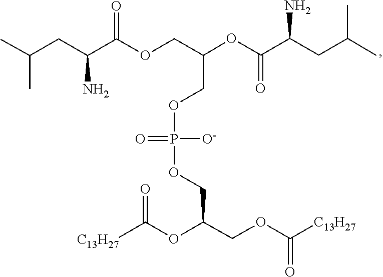

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

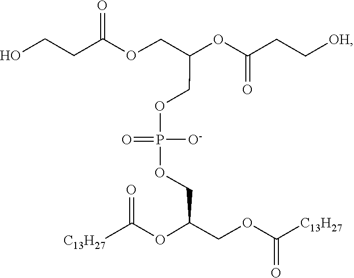

wherein the oxygen anion O⁻ is optionally paired with H, Li, Na, K, Mg, Ca, Zn, Cs, ammonium or tetraalkylammonium.

In another aspect, the compound of Formula I is

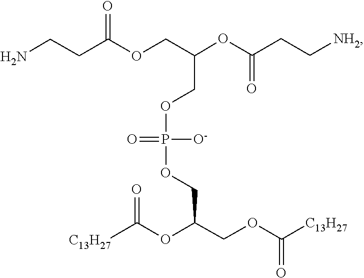

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

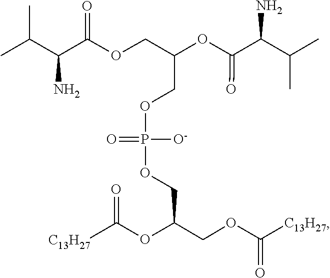

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

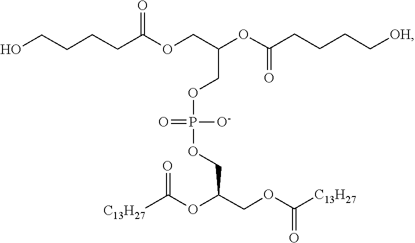

wherein the oxygen anion O⁻ is optionally paired with H, Li, Na, K, Mg, Ca, Zn, Cs, ammonium or tetraalkylammonium.

In another aspect, the compound of Formula I is

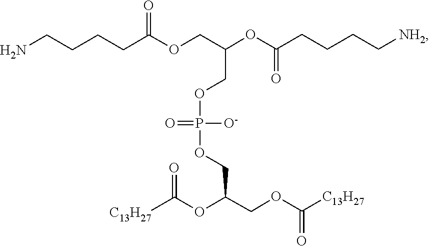

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

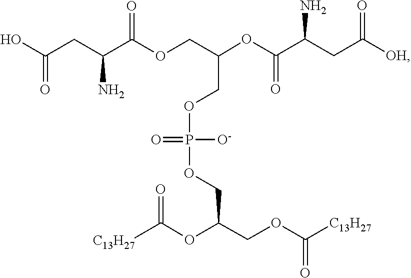

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

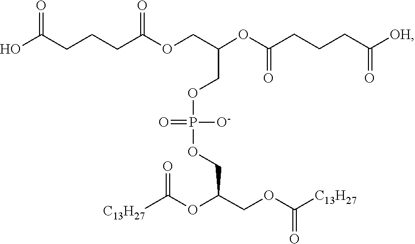

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

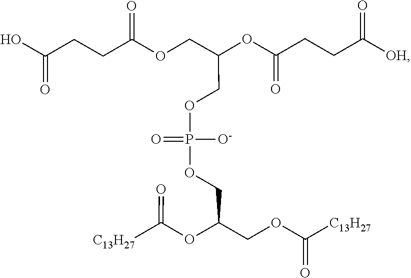

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

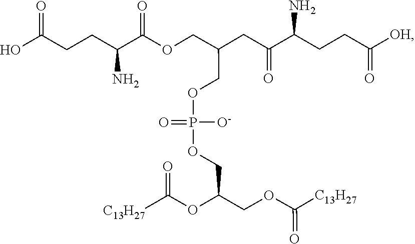

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

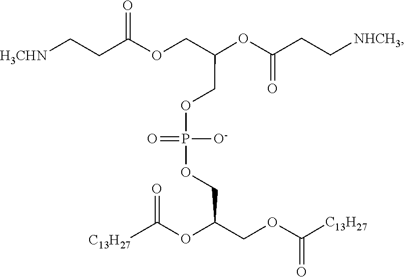

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

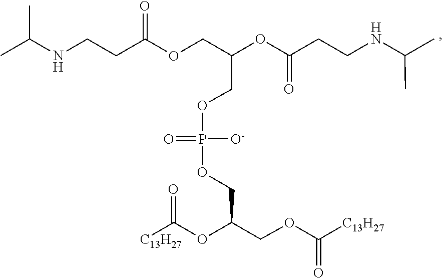

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

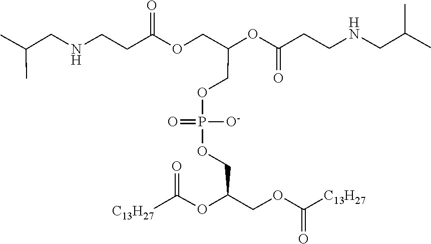

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

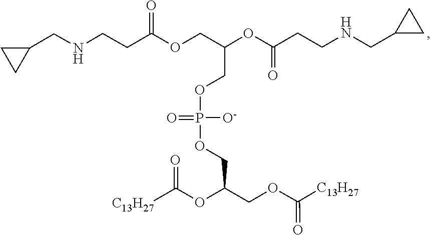

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

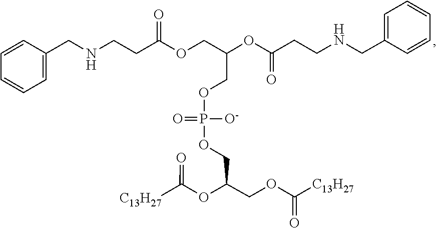

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

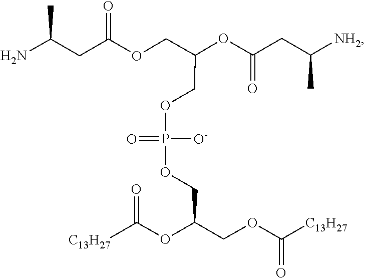

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

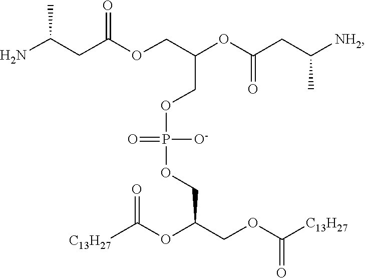

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

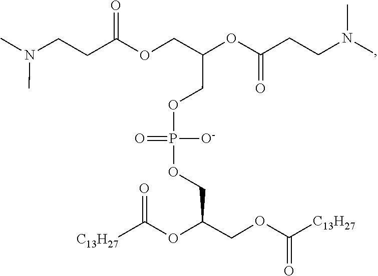

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

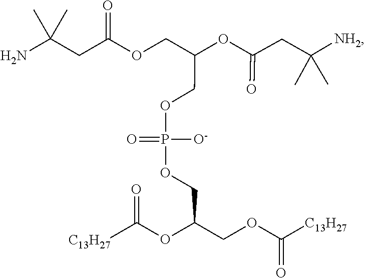

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

As embodied and broadly described herein, an aspect of the present disclosure relates a pharmaceutical composition comprising a compound of Formula I and a pharmaceutically acceptable diluent or carrier comprising:

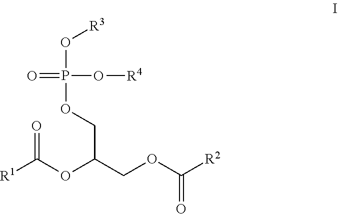

or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

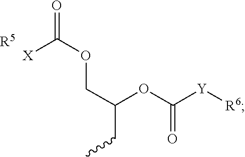

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, Ome, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, Oac, Ome, NHAc, N(R⁸)₂, SH, CN, COOH, CONH₂, Cl, Br and I;
Each R⁷ is independently H or a C₁-C₆ branched or unbranched alkyl group;
Each R⁸ is independently H or a C₁-C₆ branched or unbranched alkyl group; X is a direct linkage, O or NH;
Y is a direct linkage, O or NH; and,
each stereogenic center is independently R, S or racemic.

In one aspect, R⁴ of the compound of Formula I is H, Li, Na, K, Mg, Ca, Zn, Cs, ammonium or tetraalkylammonium.

In another aspect, the compound of Formula I is selected from at least one of:

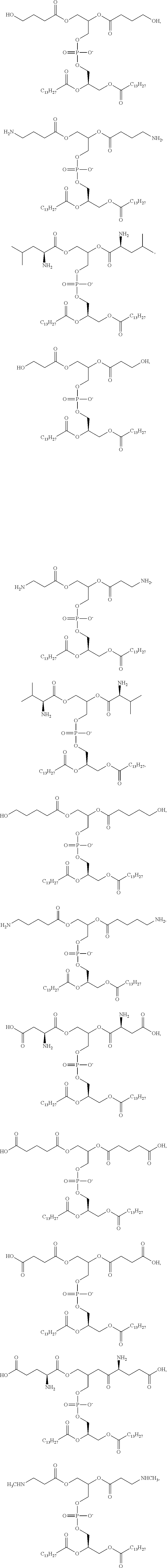

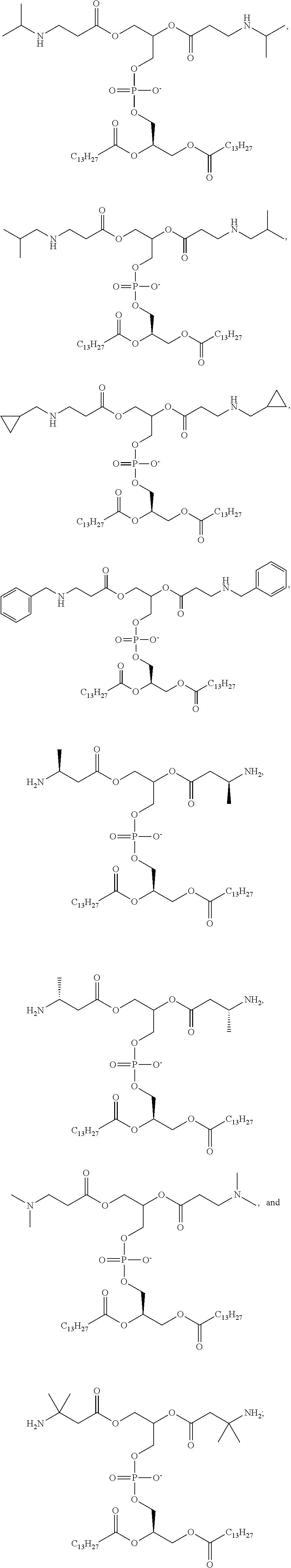

wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt. In another aspect, the compound of Formula I exists as a single entity, a solvate, a hydrate, a crystal, an amorphous solid, a liquid, or an oil. In another aspect, the pharmaceutical composition further comprises one or more agents that induce a cardiopathy as a side effect. In another aspect, the one or more agents that induces a cardiopathy as a side effect is selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone. In another aspect, the pharmaceutical composition further comprises one or more excipients, binders, anti-adherents, coatings, disintegrants, fillers, flavors, dyes, colors, glidants, lubricants, preservatives, sorbents, sweeteners, derivatives thereof, or combinations thereof. In another aspect, the binder is selected from the group consisting of hydroxypropylmethylcellulose, ethyl cellulose, povidone, acrylic and methacrylic acid co-polymers, pharmaceutical glaze, gums, and milk derivatives. In another aspect, the pharmaceutical composition comprises a compound of Formula I in an amount per unit dose of between about 1 mg and about 1 gram per unit dose. In another aspect, the pharmaceutical composition is a formulation for oral, sublingual, transdermal, suppository, intrathecal, enteral, parenteral, intravenous, intraperitoneal, cutaneous, subcutaneous, topical, pulmonary, rectal, vaginal, or intramuscular administration. In another aspect, the formulation for oral administration is a tablet, capsule, caplet, pill, powder, troche, lozenge, slurry, liquid solution, suspension, emulsion, elixir or oral thin film (OTF). In another aspect, the formulation is a solid form, a solution, a suspension, or a soft gel form. In another aspect, the compound is selected from one or more compounds of formula 31 to 51.

As embodied and broadly described herein, an aspect of the present disclosure relates to a method of reducing or eliminating one or more of a cardiac channelopathy, cardiac muscle damage, or a condition resulting from the irregularity or alteration in the cardiac pattern, in a human or animal subject, comprising the step of administering to the human or animal subject one or more of a compound of Formula I

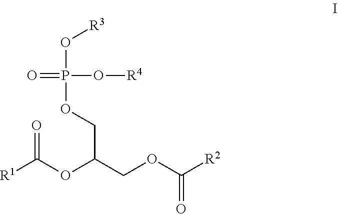

or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

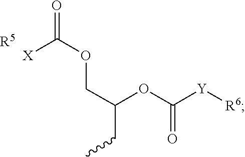

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, Oac, Ome, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, Oac, Ome, NHAc, N(R⁸)₂, SH, CN, COOH, CONH₂, Cl, Br and I;
Each R⁷ is independently H or a C₁-C₆ branched or unbranched alkyl group;
Each R⁸ is independently H or a C₁-C₆ branched or unbranched alkyl group;
X is a direct linkage, O or NH;
Y is a direct linkage, O or NH; and,
each stereogenic center is independently R, S or racemic.

In one aspect, the R⁴ of the compound of Formula I is H, Li, Na, K, Mg, Ca, Zn, Cs, ammonium or tetraalkylammonium. In another aspect, the compound of Formula I exists as a single entity, a solvate, a hydrate, a crystal, an amorphous solid, a liquid or an oil. In another aspect, the compound of Formula I reduces or eliminates one or more of a cardiac channelopathy or a condition resulting from the irregularity or alteration in the cardiac pattern caused by the active agent used to treat a disease. In another aspect, the compound of Formula I is administered in an amount per unit dose of between about 1 mg and about 1 gram. In another aspect, the compound of Formula I is formulated for oral, sublingual, transdermal, suppository, intrathecal, enteral, parenteral, intravenous, intraperitoneal, cutaneous, subcutaneous, topical, pulmonary, rectal, vaginal, or intramuscular administration. In another aspect, the compound of Formula I is formulated for oral administration as a tablet, capsule, caplet, pill, powder, troche, lozenge, slurry, liquid solution, suspension, emulsion, elixir or oral thin film (OTF). In another aspect, the compound of Formula I is formulated as a solid form, a solution, a suspension, or a soft gel form. In another aspect, the solid form further comprises one or more excipients, binders, anti-adherents, coatings, disintegrants, fillers, flavors, dyes, colors, glidants, lubricants, preservatives, sorbents, sweeteners, derivatives thereof, or combinations thereof. In another aspect, the compound of Formula I is co-administered with one or more agents that induce a cardiopathy as a side effect. In another aspect, the one or more active agent that induce a cardiopathy as a side effect are selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone. In another aspect, the compound of Formula I reduces or eliminates cardiopathies, such as QT prolongation, cardiac muscle damage, or AV block, that are drug-induced or caused by a disease or condition. In another aspect, the compound is selected from one or more compounds of formula 31 to 51.

In another aspect, the compound of Formula I is selected from at least one of:

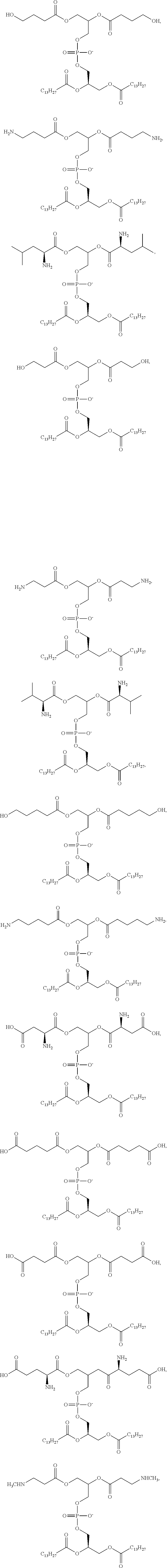

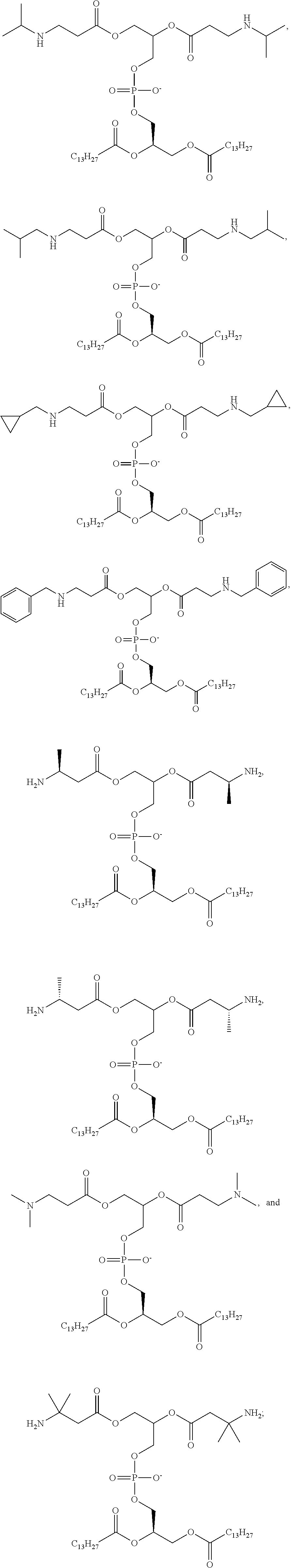

wherein any oxygen anion O⁻ of any compound is paired with H, Li, Na, K, Mg, Ca, Zn, Cs, ammonium or tetraalkylammonium; and wherein, any NH-2 or COOH of any compound is optionally in the form of a pharmaceutically acceptable salt.

In another aspect, the compound of Formula I is

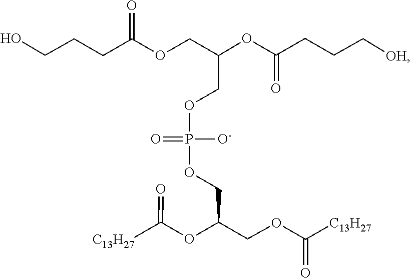

wherein the oxygen anion O⁻ is optionally paired with H, Li, Na, K, Mg, Ca, Zn, Cs, ammonium or tetraalkylammonium.

In another aspect, the compound of Formula I is

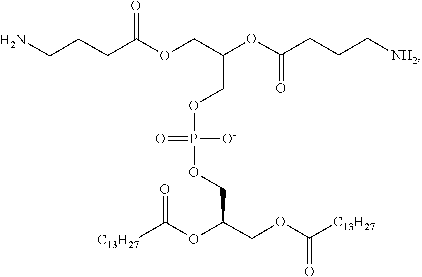

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

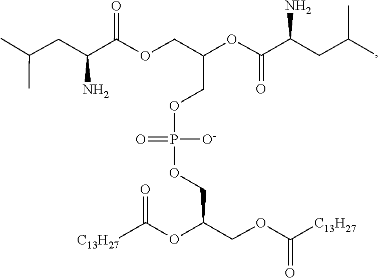

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

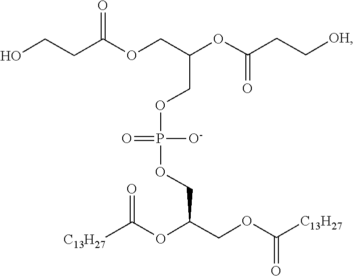

wherein the oxygen anion O⁻ is optionally paired with H, Li, Na, K, Mg, Ca, Zn, Cs, ammonium or tetraalkylammonium.

In another aspect, the compound of Formula I is

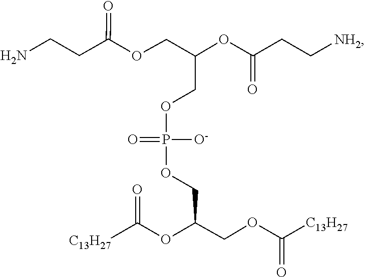

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

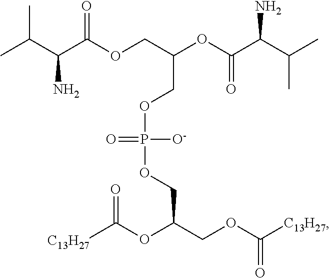

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

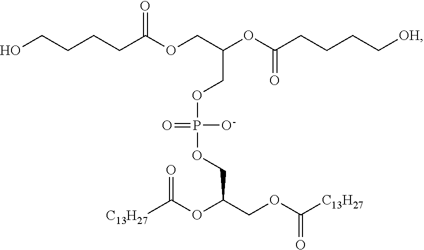

wherein the oxygen anion O⁻ is optionally paired with H, Li, Na, K, Mg, Ca, Zn, Cs, ammonium or tetraalkylammonium.

In another aspect, the compound of Formula I is

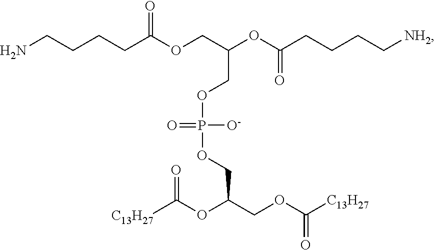

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

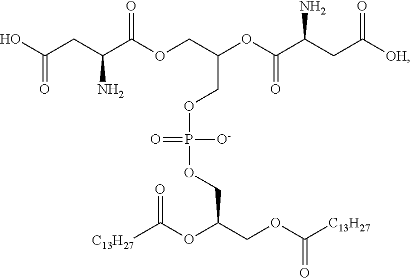

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

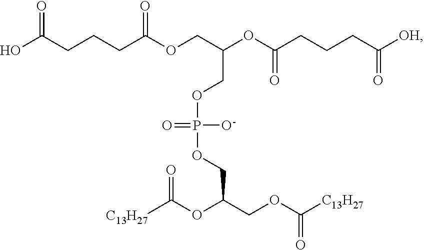

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

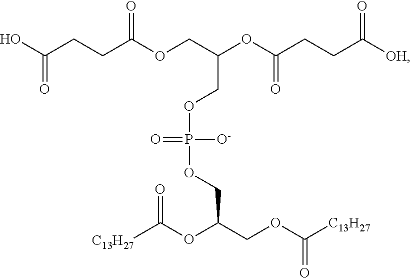

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

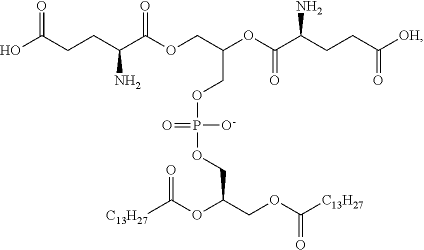

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

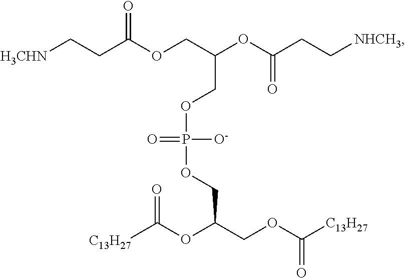

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

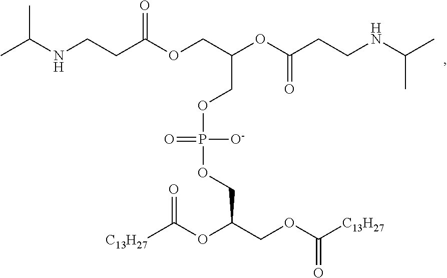

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

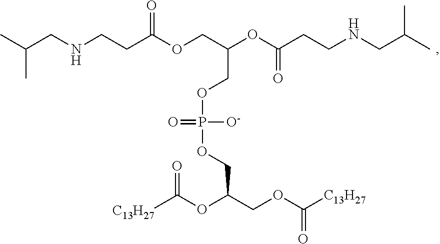

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

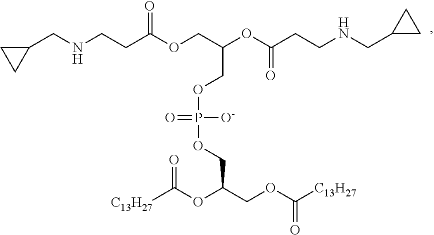

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

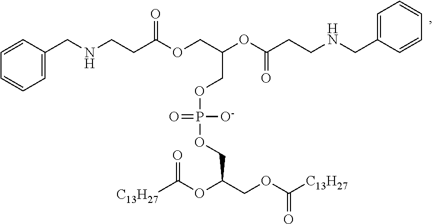

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

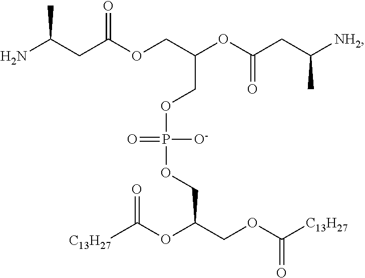

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

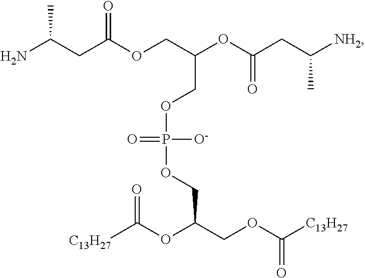

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

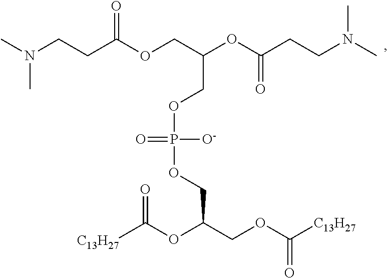

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

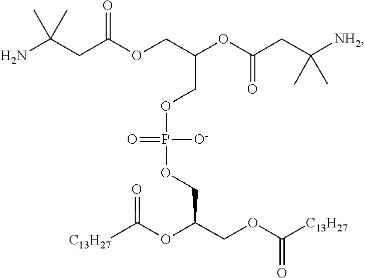

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

As embodied and broadly described herein, an aspect of the present disclosure relates to a method of reducing or eliminating a cardiotoxic or cardiopathic effect of one or more active agents comprising: administering to a subject in need of treatment for a disease or disorder one or more one or more active agents that are cardiotoxic; and providing a combination therapy with an effective amount of one or more lipids that reduce or eliminate the cardiotoxic effect of the one or more active agents, wherein the lipid has formula:

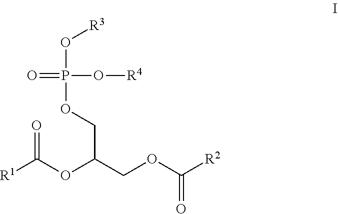

or a pharmaceutically acceptable salt thereof wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

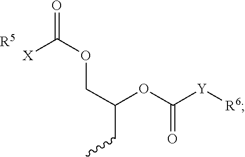

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, Oac, Ome, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, Oac, Ome, NHAc, N(R⁸)₂, SH, CN, COOH, CONH₂, Cl, Br and I;
Each R⁷ is independently H or a C₁-C₆ branched or unbranched alkyl group;
Each R⁸ is independently H or a C₁-C₆ branched or unbranched alkyl group;
X is a direct linkage, O or NH;
Y is a direct linkage, O or NH; and,
each stereogenic center is independently R, S or racemic, and
   wherein the reduction in cardiotoxicity is at least 25, 30, 40, 50, 60, 70, 75, 80, 90, 95, or 100% when compared to a treatment without the lipid, and optionally, wherein R⁴ is H, Li, Na, K, Mg, Ca, Zn, Cs, ammonium or tetraalkylammonium. In one aspect, the cardiotoxicity or cardiopathicity is selected from at least one of: minimal left ventricular dilation, contractile dysfunction, moderate valve regurgitation, a decline in left ventricular ejection fraction (LVEF), cardiac hypertrophy, reduced cardiac contractility, reduced cardiac output, pressure and volume overload hypertrophy, myocardial dysfunction, cardiac remodeling, post-myocardial infarction heart failure, or cardiopathy. In another aspect, the one or more active agents is selected from doxorubicin, trastuzumab, or both. In another aspect, the one or more active agents and the lipid are administered concurrently. In another aspect, the one or more active agents and the lipid are formulated for oral, sublingual, transdermal, suppository, intrathecal, enteral, parenteral, intravenous, intraperitoneal, cutaneous, subcutaneous, topical, pulmonary, rectal, vaginal, or intramuscular administration. In another aspect, the one or more lipids, the one or more active agents, or both, are infused over 3 hours.

In another aspect, the compound of Formula I is selected from at least one of:

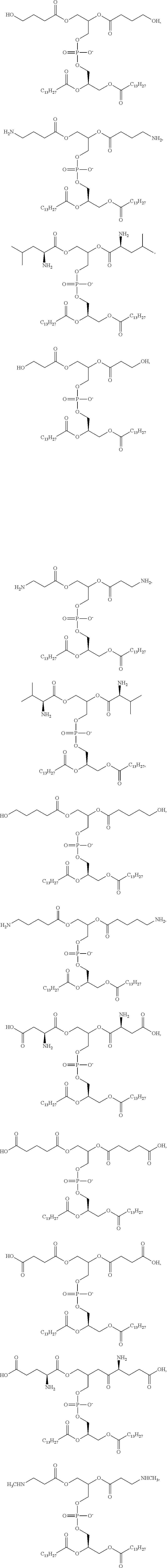

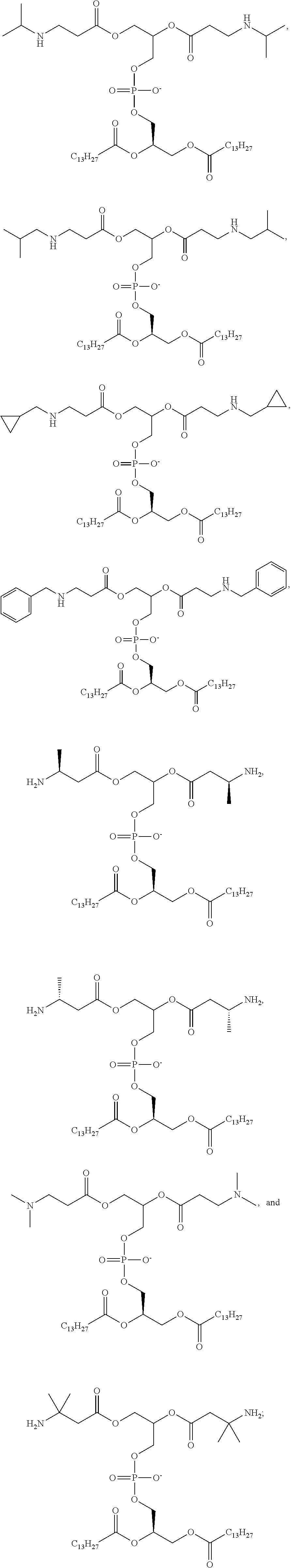

wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt.

In another aspect, the compound of Formula I is

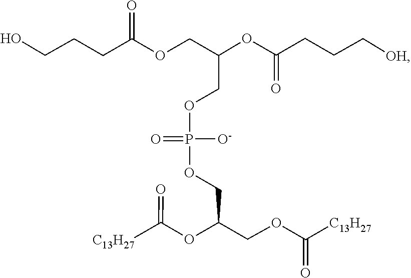

wherein the oxygen anion O⁻ is optionally paired with H, Li, Na, K, Mg, Ca, Zn, Cs, ammonium or tetraalkylammonium.

In another aspect, the compound of Formula I is

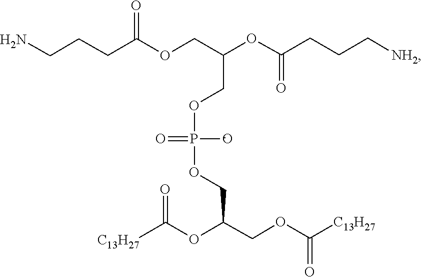

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

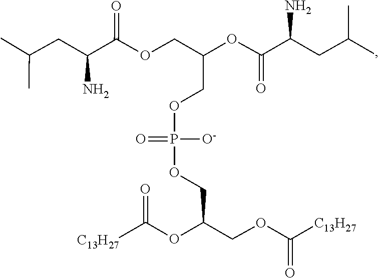

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

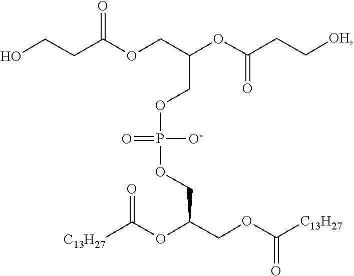

wherein the oxygen anion O⁻ is optionally paired with H, Li, Na, K, Mg, Ca, Zn, Cs, ammonium or tetraalkylammonium.

In another aspect, the compound of Formula I is

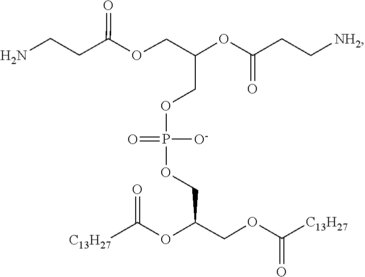

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

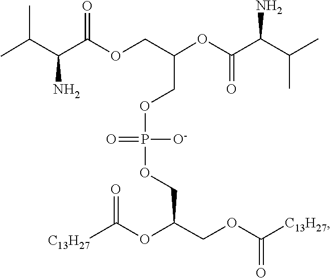

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

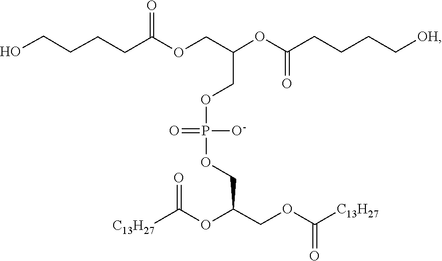

wherein the oxygen anion O⁻ is optionally paired with H, Li, Na, K, Mg, Ca, Zn, Cs, ammonium or tetraalkylammonium.

In another aspect, the compound of Formula I is

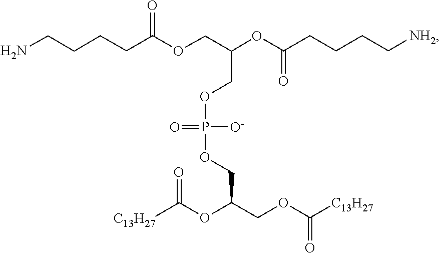

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

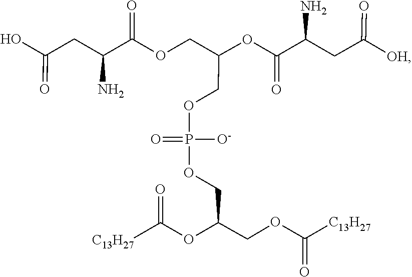

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

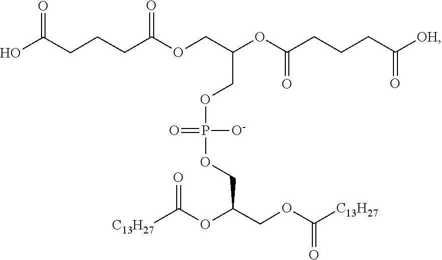

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

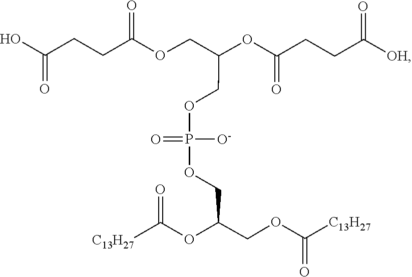

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

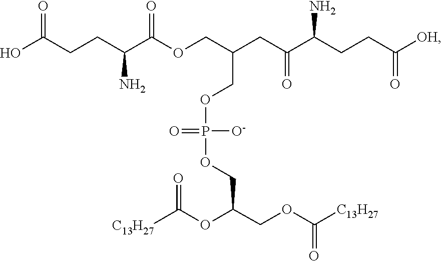

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

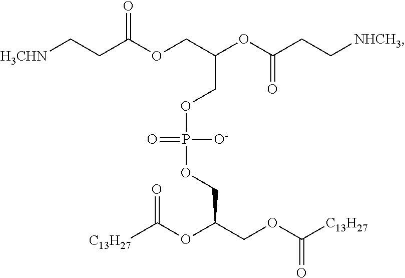

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

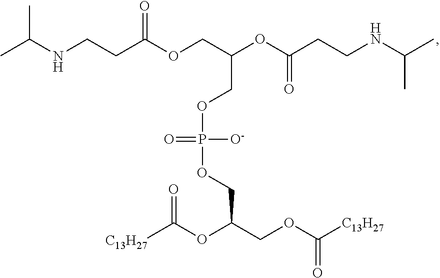

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

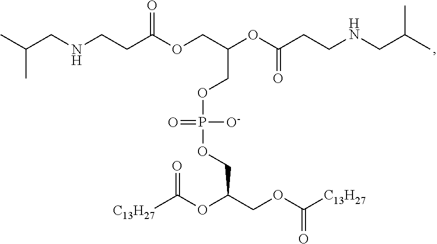

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

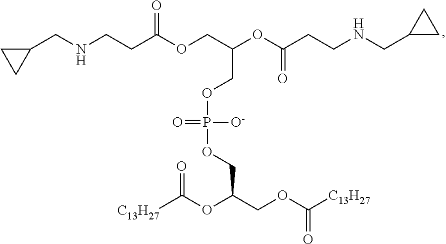

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

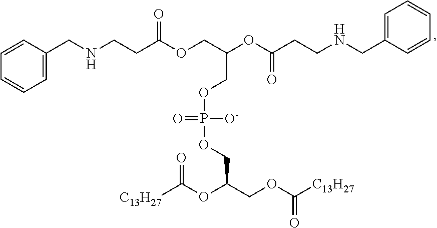

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

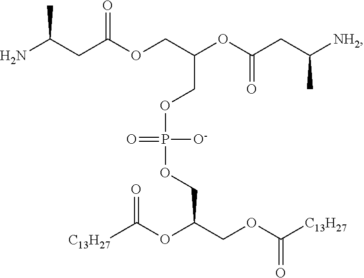

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

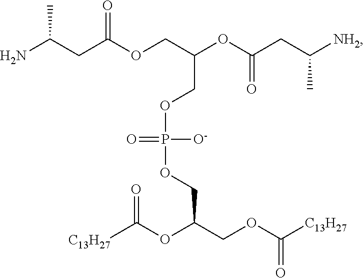

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

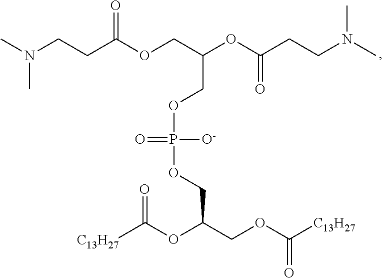

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

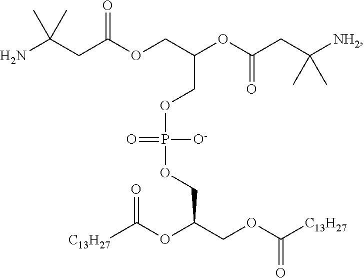

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the one or more agents that induces a cardiotoxic or cardiopathic effect is selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone. In another aspect, a pharmaceutical composition comprising the one or more lipids further comprises one or more excipients, binders, anti-adherents, coatings, disintegrants, fillers, flavors, dyes, colors, glidants, lubricants, preservatives, sorbents, sweeteners, derivatives thereof, or combinations thereof. In another aspect, the pharmaceutical composition comprises a compound of Formula I in an amount per unit dose of about 0.1, 0.5, 1, 2, 3, 4, 5, 6, 7, 8, 9, 10, 15, 20, 24, 30, 40, 50, 60, 75, 80, 90, 100, 125, 150, 175, 200, 225, 250, 275, 300, 325, 350, 375, 400, 450, 500, 550, 600, 650, 700, 750, 800, 850, 900, 950, 1,000, 2,000, 3,000, 4,000, 5,000, 6,000, 7,000, 8,000, 9,000, or 10,000 milligrams per unit dose. In another aspect, the pharmaceutical composition is a formulation for oral, sublingual, transdermal, suppository, intrathecal, enteral, parenteral, intravenous, intraperitoneal, cutaneous, subcutaneous, topical, pulmonary, rectal, vaginal, or intramuscular administration. In another aspect, the composition is formulated for oral administration is a tablet, capsule, caplet, pill, powder, troche, lozenge, slurry, liquid solution, suspension, emulsion, elixir or oral thin film (OTF). In another aspect, the formulation is a solid form, a solution, a suspension, or a soft gel form. In another aspect, the compound of Formula I is selected from at least one of:

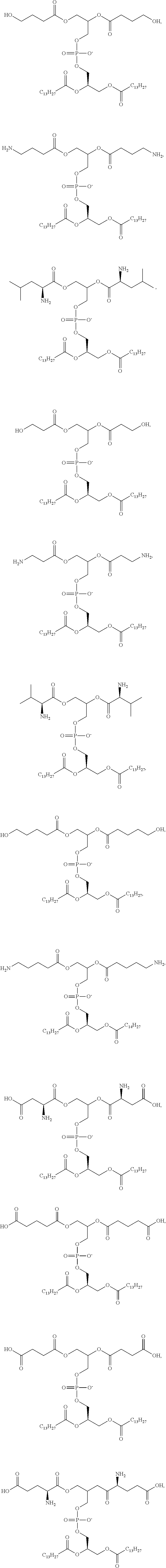

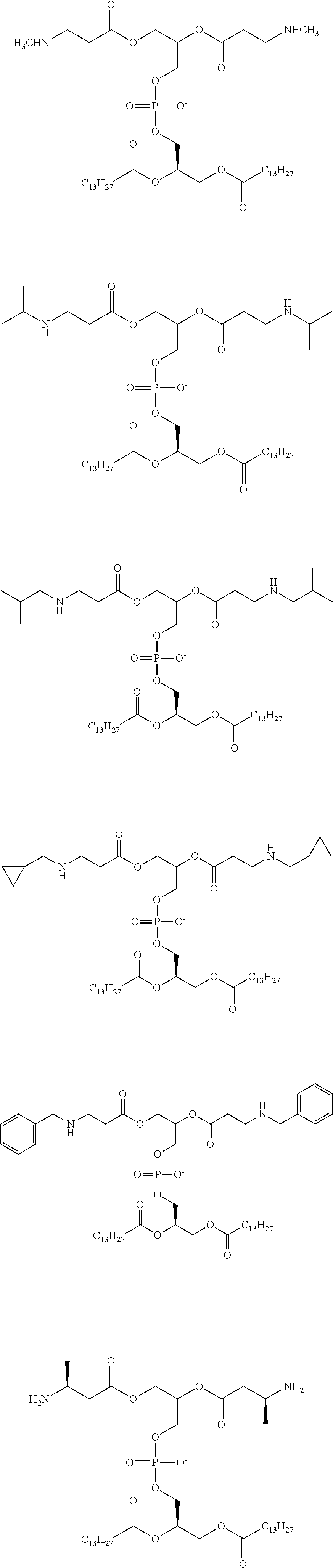

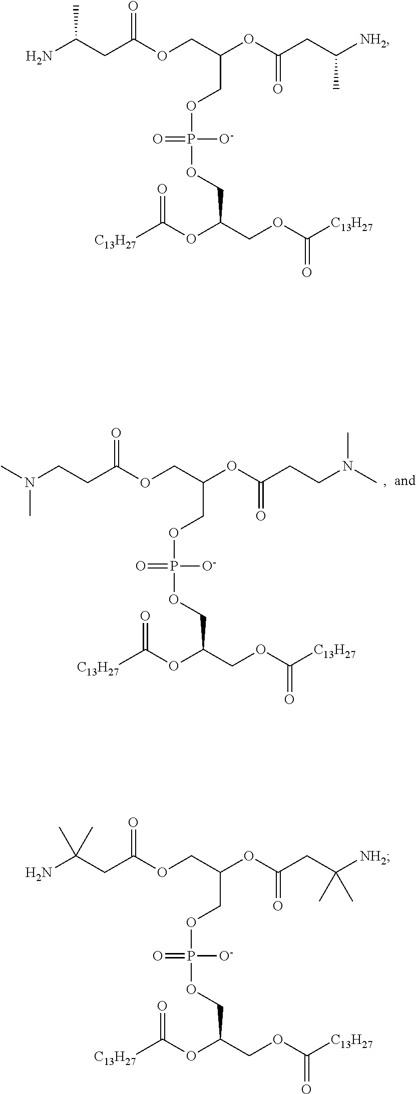

wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt.

In another aspect, the compound of Formula I is

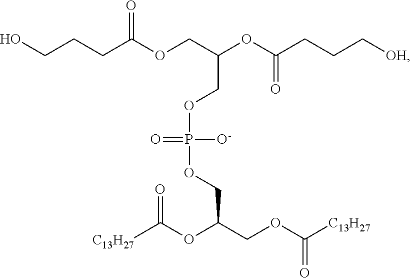

wherein the oxygen anion O⁻ is optionally paired with H, Li, Na, K, Mg, Ca, Zn, Cs, ammonium or tetraalkylammonium.

In another aspect, the compound of Formula I is

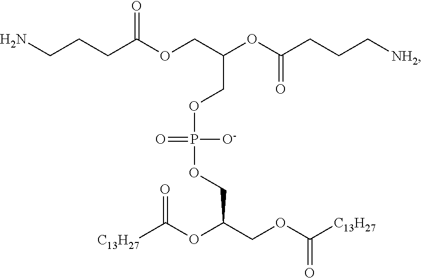

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

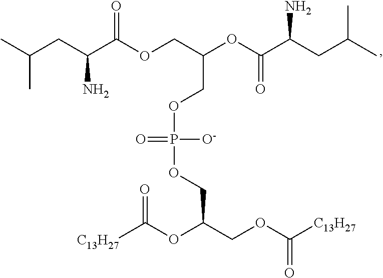

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

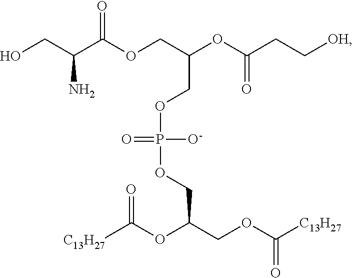

wherein the oxygen anion O⁻ is optionally paired with H, Li, Na, K, Mg, Ca, Zn, Cs, ammonium or tetraalkylammonium.

In another aspect, the compound of Formula I is

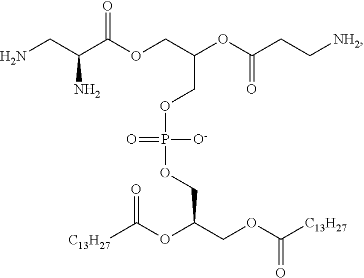

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

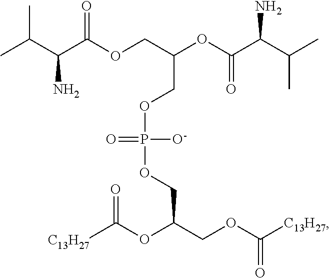

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

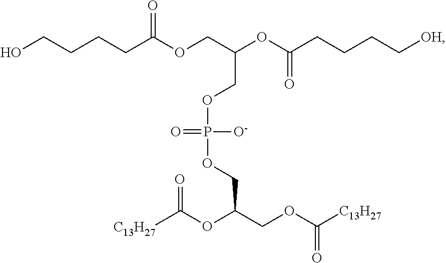

wherein the oxygen anion O⁻ is optionally paired with H, Li, Na, K, Mg, Ca, Zn, Cs, ammonium or tetraalkylammonium.

In another aspect, the compound of Formula I is

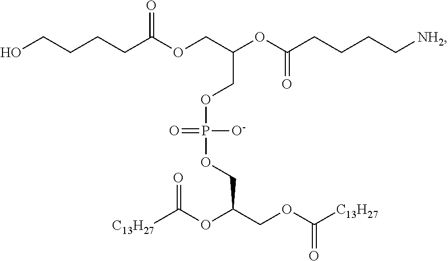

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

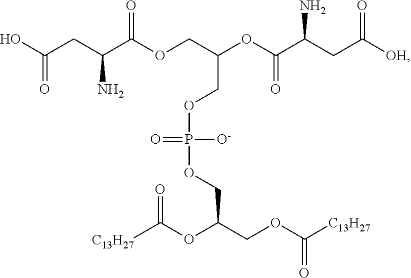

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

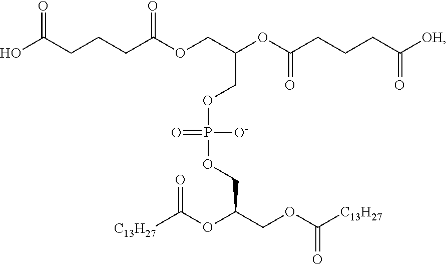

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

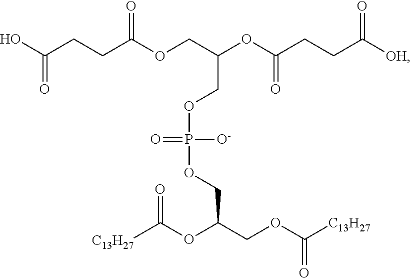

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

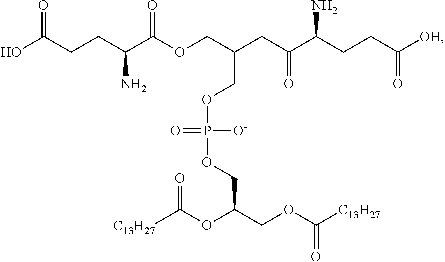

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

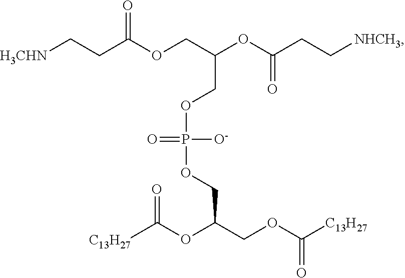

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

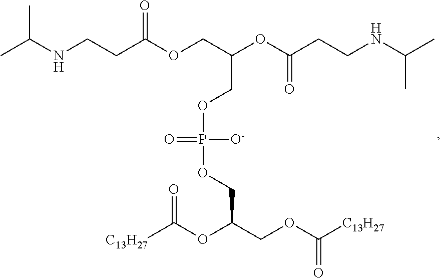

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

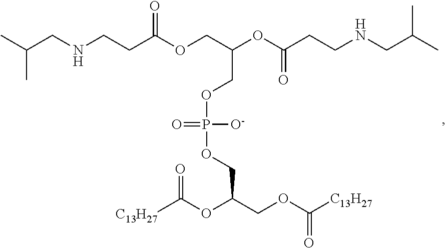

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

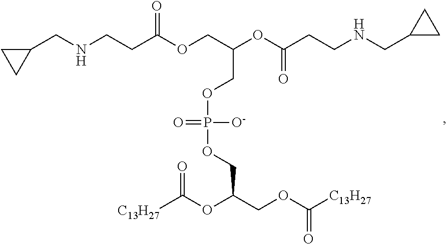

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

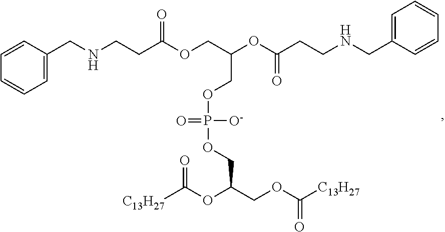

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

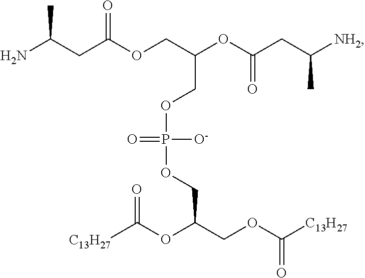

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

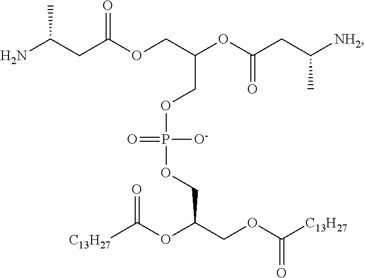

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

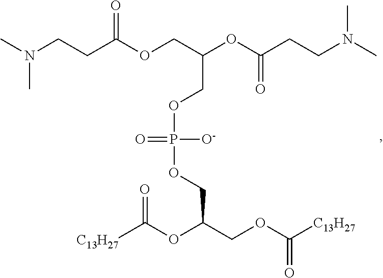

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

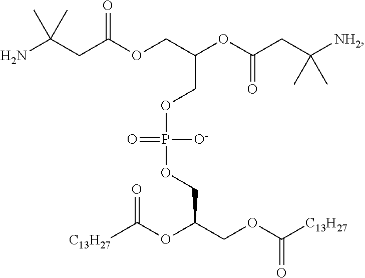

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

As embodied and broadly described herein, an aspect of the present disclosure relates to a method of reducing or eliminating a cardiotoxic effect of one or more antiproliferative agents comprising: administering to a subject in need of treatment for a proliferative disorder one or more antiproliferative agents that are cardiotoxic; and providing a combination therapy with an effective amount of one or more lipids that reduce or eliminate the cardiotoxic effect of one or more antiproliferative agents, wherein the lipid has formula:

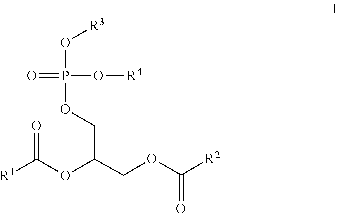

or a pharmaceutically acceptable salt thereof wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   R³ is

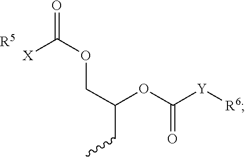

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, Oac, Ome, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, Oac, Ome, NHAc, N(R⁸)₂, SH, CN, COOH, CONH₂, Cl, Br and I;
Each R⁷ is independently H or a C₁-C₆ branched or unbranched alkyl group;
Each R⁸ is independently H or a C₁-C₆ branched or unbranched alkyl group;
X is a direct linkage, O or NH;
Y is a direct linkage, O or NH; and,
Each stereogenic center is independently R or S or racemic, and optionally, wherein
R⁴ is H, Li, Na, K, Mg, Ca, Zn, Cs, ammonium or tetraalkylammonium; and
   wherein the reduction in cardiotoxicity is at least 25, 30, 40, 50, 60, 70, 75, 80, 90, 95, or 100% when compared to a treatment without the lipid. In one aspect, the cardiotoxicity is selected from at least one of: minimal left ventricular dilation, contractile dysfunction, moderate valve regurgitation, a decline in left ventricular ejection fraction (LVEF), cardiac hypertrophy, reduced cardiac contractility, reduced cardiac output, pressure and volume overload hypertrophy, myocardial dysfunction, cardiac remodeling, post-myocardial infarction heart failure, or cardiopathy. In another aspect, the one or more antiproliferative agents is selected from doxorubicin, trastuzumab, or both. In another aspect, the one or more antiproliferative agents and the lipid are administered concurrently. In another aspect, the one or more antiproliferative agents and the lipid administered orally, or intravenously. In another aspect, the one or more lipids, the one or more antiproliferative agents, or both, are infused over 3 hours. In another aspect, the one or more antiproliferative agents that induce a cardiopathy as a side effect are selected from at least one of: Bosutinib, Crizotinib, Dabrafenib, Dasatinib, Doxorubicin Lapatinib, Nilotinib, Sorafenib, Sunitinib, Vandetanib, or Vemurafenib.

In another aspect, the compound of Formula I is selected from at least one of:

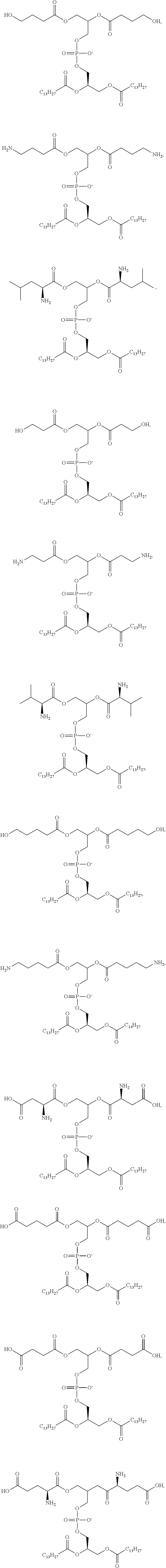

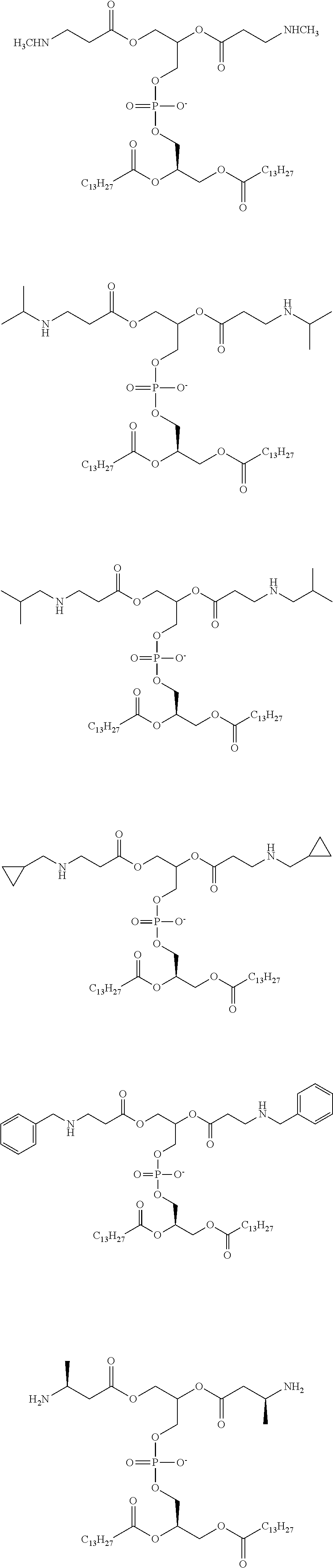

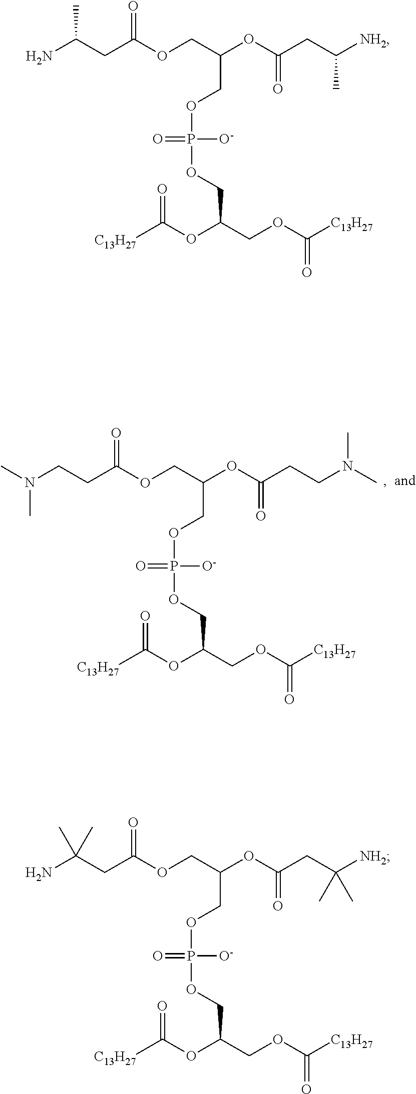

wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt.

In another aspect, the compound of Formula I is

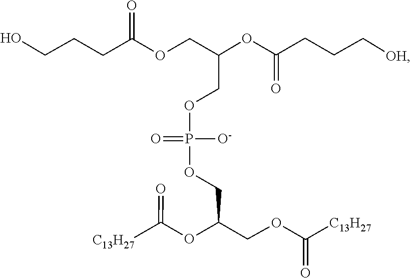

wherein the oxygen anion O⁻ is optionally paired with H, Li, Na, K, Mg, Ca, Zn, Cs, ammonium or tetraalkylammonium.

In another aspect, the compound of Formula I is

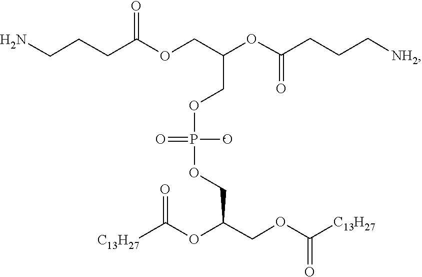

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

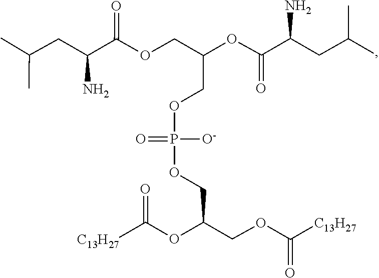

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

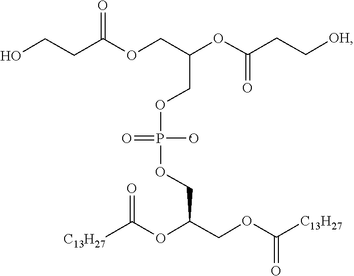

wherein the oxygen anion O⁻ is optionally paired with H, Li, Na, K, Mg, Ca, Zn, Cs, ammonium or tetraalkylammonium.

In another aspect, the compound of Formula I is

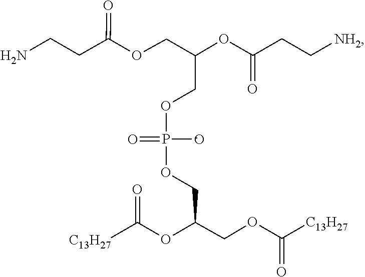

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

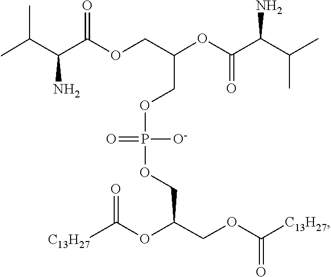

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

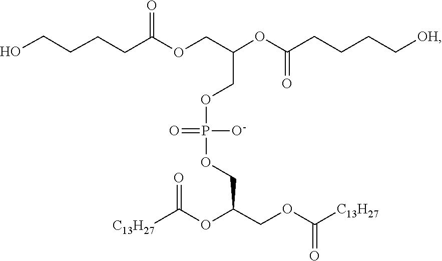

wherein the oxygen anion O⁻ is optionally paired with H, Li, Na, K, Mg, Ca, Zn, Cs, ammonium or tetraalkylammonium.

In another aspect, the compound of Formula I is

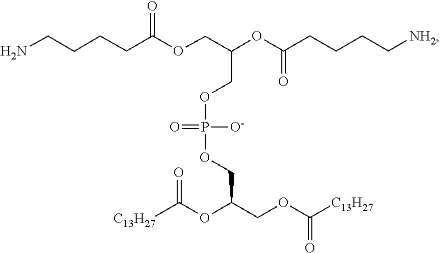

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

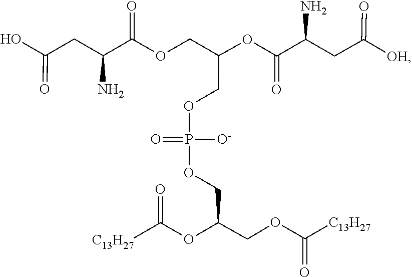

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

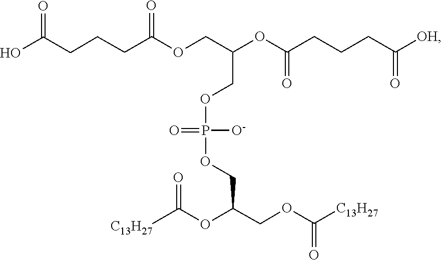

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

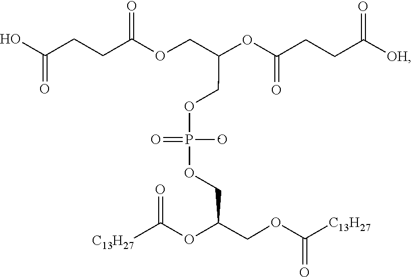

wherein the oxygen anion O⁻ is optionally paired with H, Li, Na, K, Mg, Ca, Zn, Cs, ammonium or tetraalkylammonium; and wherein any COOH is optionally in the form of a pharmaceutically acceptable salt.

In another aspect, the compound of Formula I is

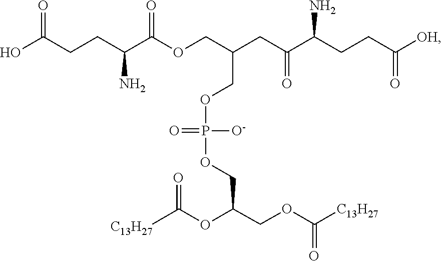

wherein the oxygen anion O⁻ is optionally paired with H, Li, Na, K, Mg, Ca, Zn, Cs, ammonium or tetraalkylammonium; and wherein any NH₂ or COOH is optionally in the form of a pharmaceutically acceptable salt.

In another aspect, the compound of Formula I is

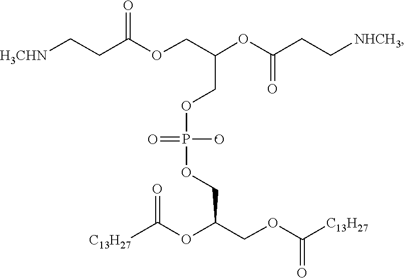

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

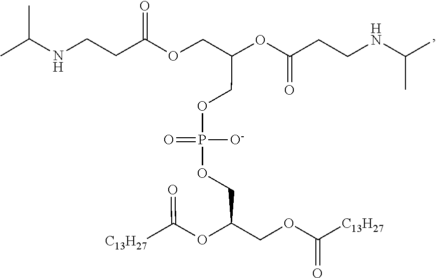

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

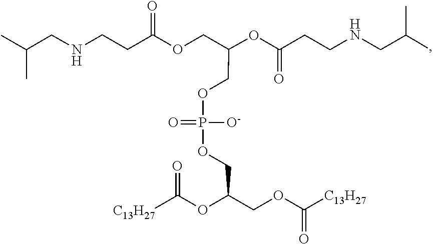

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

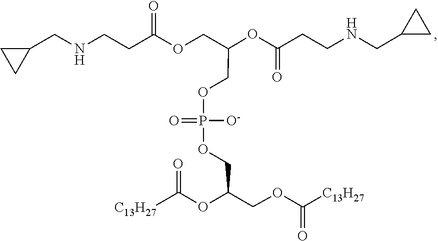

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

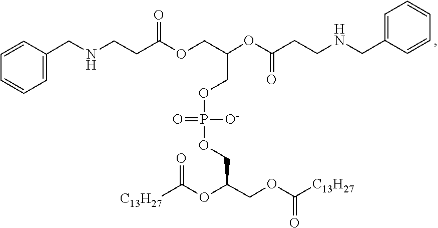

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

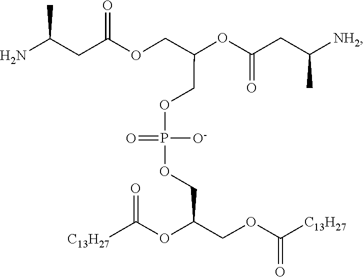

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

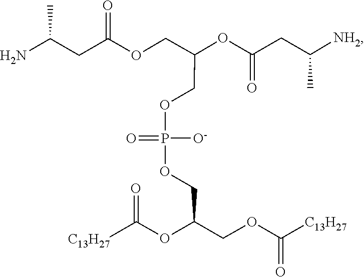

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

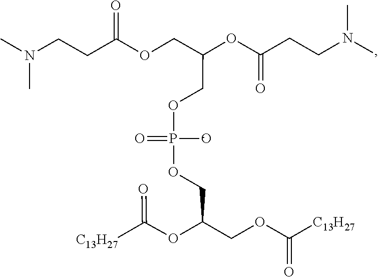

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

In another aspect, the compound of Formula I is

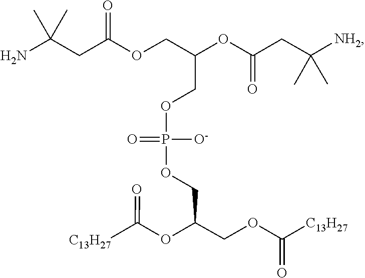

wherein the oxygen anion O⁻ is optionally paired with H, Li, Na, K, Mg, Ca, Zn, Cs, ammonium or tetraalkylammonium; and wherein any nitrogen atom is optionally in the form of a pharmaceutically acceptable salt.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the effect of an oral single dose of Moxifloxacin (20 mg/kg) on QTc interval of guinea pigs compared to the same oral single dose of Moxifloxacin administrated concomitantly with an oral single dose of Compound 31.

FIG. 2 is a graph that shows the effect of an oral single dose of Moxifloxacin (20 mg/kg) on QTc interval of guinea pigs compared to the same oral single dose of Moxifloxacin administrated concomitantly with an oral single dose of Compound 36.

FIG. 3 is a graph that shows the effect of an oral single dose of Moxifloxacin (20 mg/kg) on QTc interval of guinea pigs compared to the same oral single dose of Moxifloxacin administrated concomitantly with an oral single dose of Compound 34.

FIG. 4 is a graph that shows the effect of an oral single dose of Moxifloxacin (20 mg/kg) on QTc interval of guinea pigs compared to the same oral single dose of Moxifloxacin administrated concomitantly with an oral single dose of Compound 32.

FIG. 5 is a graph that shows the effect of an oral single dose of Moxifloxacin (20 mg/kg) on QTc interval of guinea pigs compared to the same oral single dose of Moxifloxacin administrated concomitantly with an oral single dose of Compound 35.

FIG. 6A and FIG. 6B are composite graphs that shows the effect of an oral single dose of Moxifloxacin (20 mg/kg) on QTc interval of guinea pigs compared to the same oral single dose of Moxifloxacin administrated concomitantly with an oral single dose of Compound 40, Compound 41, Compound 32, Compound 34, Compound 35, Compound 36, Compound 37, Compound 38, Compound 39, Compound 40, Compound 41, Compound 44, Compound 45, Compound 47 and Compound 51.

FIG. 7 is a depiction of example chemical structures that are embodiments of the present invention.

FIG. 8 shows the 9-week study outline for determining the cardioprotective effect of the lipids.

FIG. 9 shows M-Mode echography images comparing the effects of a sham treatment, treatment with doxorubicin and Trastuzumab, and doxorubicin and Trastuzumab+ lipid Compound 35.

FIG. 10 shows left ventricular pressure recordings comparing a sham treatment, treatment with doxorubicin and Trastuzumab, and doxorubicin and Trastuzumab+ lipid Compound 35 at two concentrations, 10 mg·kg and 50 mg/kg.

FIGS. 11A to 11H are graphs that show the systolic left ventricular pressure (FIG. 11A), heart rate (FIG. 11B), stroke volume (FIG. 11C), left ventricular ejection fraction (FIG. 11D), percent fractional shortening (FIG. 11E), N-terminal (NT)-pro hormone BNP (NT-proBNP) at day 49 (FIG. 11F), anterior wall thickness end-diastole (AWT-ED) (mm) (FIG. 11G), anterior wall thickness end-systole (AWT-ES) mm (FIG. 11H), and left ventricular mass (echo) (FIG. 11I).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

Compounds of the present invention include those described generally above, and are further illustrated by the classes, subclasses, and species disclosed herein. As used herein, the following definitions shall apply unless otherwise indicated. In at least some embodiments, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

To facilitate the understanding of this invention, a number of terms are defined below. Terms defined herein have meanings as commonly understood by a person of ordinary skill in the areas relevant to the present invention.

Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims. Specifically, the use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the measuring device, the method being employed to determine the value, or the variation that exists among the study subjects.

General description of the compounds in at least some embodiments of the invention. At least one embodiment of the present invention provides a structure of Formula I:

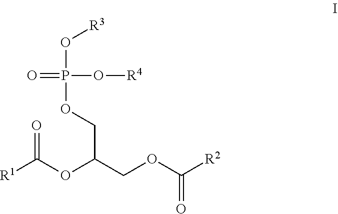

or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

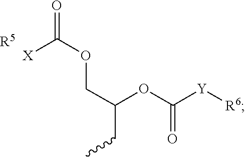

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic.

Compounds and definitions:

An “alkyl” group refers, in one embodiment, to a saturated aliphatic hydrocarbon, including straight-chain, branched-chain and cyclic alkyl groups. In one embodiment, the alkyl group has 1-20 carbons. In another embodiment, the alkyl group has 1-15 carbons. In another embodiment, the alkyl group has 1-10 carbons. In another embodiment, the alkyl group has 11-20 carbons. In another embodiment, the alkyl group has 5-15 carbons. In yet still another embodiment, the alkyl group has 1-5 carbons. The alkyl group may be unsubstituted or substituted by one or more groups selected from halogen, hydroxy, alkoxy, carboxylic acid, aldehyde, carbonyl, amido, cyano, alkylamido, dialkylamido, nitro, amino, alkylamino, dialkylamino, carboxyl, thio and thioalkyl.

An “alkenyl” group refers, in one embodiment, to an unsaturated hydrocarbon, including straight chain, branched chain and cyclic groups having one or more double bonds. The alkenyl group may have one double bond, two double bonds, three double bonds, etc. In another embodiment, the alkenyl group has 2-20 carbons. In another embodiment, the alkenyl group has 11-20 carbons. In another embodiment, the alkenyl group has 5-15 carbons. In another embodiment, the alkenyl group has 2-5 carbons. In another embodiment, the alkenyl group has 2-10 carbons. In another embodiment the alkenyl group is ethenyl (—CH═CH2) Examples of alkenyl groups that may be included are ethenyl, propenyl, butenyl, cyclohexenyl, etc. The alkenyl group may be unsubstituted or substituted by a halogen, hydroxy, alkoxy carbonyl, amido, alkylamido, dialkylamido, nitro, cyano, amino, alkylamino, dialkylamino, carboxyl, thio and/or thioalkyl.

An “alkynyl” group refers, in one embodiment, to an unsaturated hydrocarbon, including straight chain, branched chain and cyclic groups having one or more triple bonds. The alkynyl group may have one triple bond, two triple bonds, three triple bonds, etc. In another embodiment, the alkynyl group has 2-20 carbons. In another embodiment, the alkynyl group has 11-20 carbons. In another embodiment, the alkynyl group has 5-15 carbons. In another embodiment, the alkynyl group has 2-15 carbons. In another embodiment, the alkynyl group has 2-10 carbons. In another embodiment the alkynyl group is ethynyl. Examples of alkenyl groups are ethynyl, propynyl, butynyl, cyclohexynyl, etc. The alkynyl group may be unsubstituted or substituted by a halogen, hydroxy, alkoxy carbonyl, cyano, amido, alkylamido, dialkylamido, nitro, amino, alkylamino, dialkylamino, carboxyl, thio and/or thioalkyl.

In one embodiment, the term “halogen” refers, in one embodiment to F, in another embodiment to Cl, in another embodiment to Br, and in another embodiment to I.

A “pharmaceutically acceptable cation” refers in one embodiment to those organic cations or inorganic cations that are pharmaceutically acceptable for use in a mammal and are well known in the art. For example, inorganic cations or organic cations include but are not limited to lithium, sodium, potassium, magnesium, calcium, barium, zinc, aluminum, cesium, and amine cations. Amine cations include but are not limited to cations derived from ammonia, triethylamine, tromethamine (TRIS), triethanolamine, ethylenediamine, glucamine, N-methylglucamine, glycine, lysine, ornithine, arginine, ethanolamine, choline and the like. In one embodiment, the amine cations are cations wherein X+ is of the formula YH+ wherein Y is ammonia, triethylamine, trimethylamine (TRIS), triethanolamine, ethylenediamine, glucamine, N-methylglucamine, glycine, lysine, ornithine, arginine, ethanolamine, choline and the like. In one embodiment suitable cationic organic or inorganic salts that can be used include cationic moieties that can form an ionic association with the O moieties on the compound and not significantly adversely affecting the desired properties of the compound for purposes of the present invention, e.g., increased solubility, stability and the like. It will be appreciated by those skilled in the art that a compound of Formula I wherein R⁴ is an organic cation or inorganic cation can be converted to a compound of formula I comprising one or more different organic or inorganic cation.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

As used herein, the term “in vivo” refers to being inside the body. The term “in vitro” used as used in the present application is to be understood as indicating an operation carried out in a non-living system.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes (i) inhibiting the disease in an animal that is experiencing or displaying the pathology or symptomatology of the diseased (i.e., arresting further development of the pathology and/or symptomatology); or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, the terms “effective amount” or “therapeutically effective amount” described herein means the amount of the subject compound that will elicit the biological or medical response of a tissue, system, animal or human that is being sought by the researcher, veterinarian, medical doctor or other clinician.

As used herein, the terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

As used herein the term “intravenous administration” includes injection and other modes of intravenous administration.

As used herein, the term “pharmaceutically acceptable” as used herein to describe a carrier, diluent or excipient must be compatible with the other ingredients of the formulation and not deleterious to the recipient thereof.

Description of exemplary embodiments. In one embodiment, the present invention relates to a compound of Formula I:

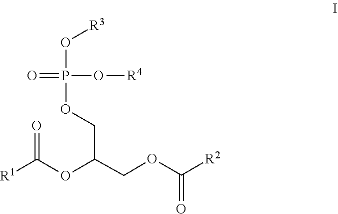

or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

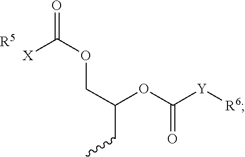

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
Each R⁷ is independently H or a C₁-C₆ branched or unbranched alkyl group;
Each R⁸ is independently H or a C₁-C₆ branched or unbranched alkyl group;
X is a direct linkage, O or NH;
Y is a direct linkage, O or NH; and,
Each stereogenic center is independently R, S or racemic.

In one embodiment, the present invention relates to a compound of Formula IA:

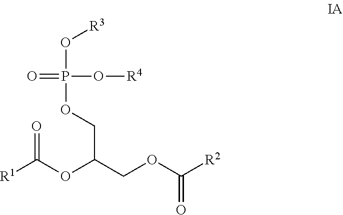

or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

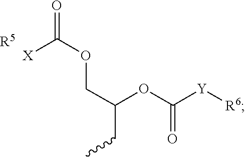

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, N(R⁷)₂ and COOH;
R⁶ is a C₁-C₁₀ branched or unbranched hydrocarbon optionally substituted with one or more groups selected from OH, N(R⁸)₂ and COOH;
Each R⁷ is independently H or a C₁-C₆ branched or unbranched alkyl group;
Each R⁸ is independently H or a C₁-C₆ branched or unbranched alkyl group;
X is a direct linkage;
Y is a direct linkage; and,
Each stereogenic center is independently R, S or racemic.

In one embodiment, the present invention relates to a method of preparing a compound of Formula I

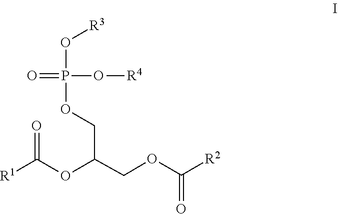

or a pharmaceutically acceptable salt thereof
wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   R³ is

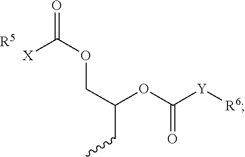

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic;
   comprising the steps of:
Converting the hydroxyl groups of a compound of Formula II to esters, carbonates, or carbamates

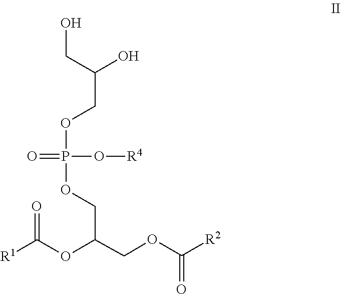

wherein, all substitutions are defined as above;
optionally converting a phosphorus-bound OH group to O—R⁴, wherein R⁴ is not H; and,
optionally removing one or more protecting groups; or
   comprising the steps of:
linking a compound of Formula III with a compound of Formula IV through creation of a phosphate diester bridge

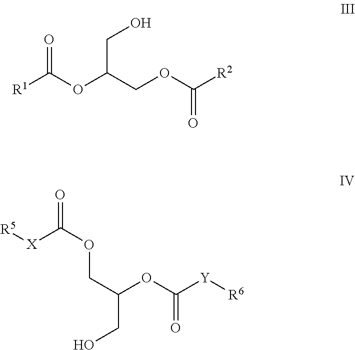

wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   Each R⁷ is independently H or a C₁-C₆ branched or unbranched alkyl group;
   Each R⁸ is independently H or a C₁-C₆ branched or unbranched alkyl group;
   R⁹ is a C₁-C₁₀ branched or unbranched hydrocarbon substituted with one or more groups independently selected from OR¹, N(R⁷)R¹², N(R⁷)₂, SR¹, CN, COOR¹⁴, CONH₂, Cl, Br and I;
   R¹⁰ is a C₁-C₁₀ branched or unbranched hydrocarbon substituted with one or more groups independently selected from OR¹¹, N(R⁸)R¹², N(R⁸)₂, SR¹, CN, COOR¹⁴, CONH₂, Cl, Br and I;
   each R¹¹ is independently H, Ac, Me, tert-Butyl, Benzyl, Trityl, Benzoyl, para-nitrobenzoyl, MOM, BOM or Si comprising the core of a silyl ether;
   each R¹² is independently H, Me, Boc, Cbz, Fmoc, Benzyl, 4-Methoxybenzyl, tert-Butyl, or Trityl;
   each R¹³ is independently H, Ac, Benzoyl, para-nitrobenzoyl or Trityl;
   each R¹⁴ is independently H, C₁-C₆ branched or unbranched alkyl, Benzyl or 4-Methoxybenzyl;
   X is a direct linkage, O or NH;
   Y is a direct linkage, O or NH; and,
   Each stereogenic center is independently R, S or racemic;
optionally converting a phosphorus-bound OH group to O—R⁴, wherein R⁴ is not H; and,
optionally converting each OR¹¹, N(R⁷)R¹², N(R⁸)R¹², SR¹³ or COOR¹⁴ to OH, NHR⁷, NHR⁸, SH or COOH, respectively.

In another embodiment, the present invention relates to a method of preparing a compound of Formula IA

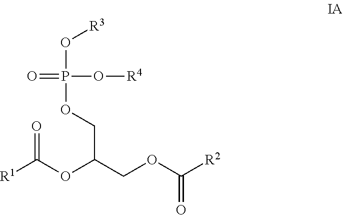

or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is

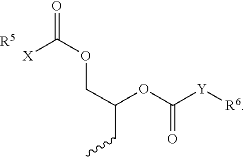

R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, N(R⁷)₂ and COOH;
R⁶ is a C₁-C₁₀ branched or unbranched hydrocarbon optionally substituted with one or more groups selected from OH, N(R⁸)₂ and COOH;
each R⁷ is independently H or a C₁-C₆ branched or unbranched alkyl group;
each R⁸ is independently H or a C₁-C₆ branched or unbranched alkyl group;
X is a direct linkage;
Y is a direct linkage; and,
each stereogenic center is independently R, S or racemic;
   comprising the steps of:
converting the hydroxyl groups of a compound of Formula II to esters, carbonates, or carbamates

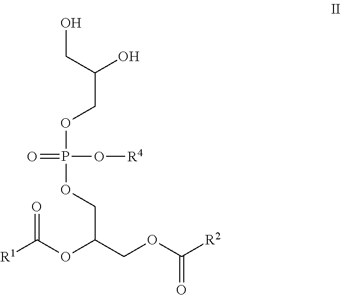

wherein, all substitutions are defined as above;
optionally converting a phosphorus-bound OH group to O—R⁴, wherein R⁴ is not H; and,
optionally removing one or more protecting groups; or
   comprising the steps of:
linking a compound of Formula III with a compound of Formula IV through creation of a phosphate diester bridge

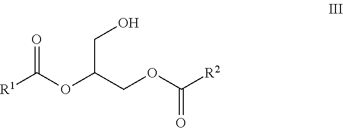

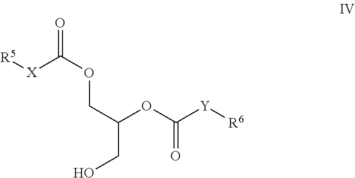

wherein,
   R¹ is a C₁-C₂₀ branched or unbranched hydrocarbon possessing 0-10 double bonds, 0-10 triple bonds or a combination of 0-10 double and triple bonds;
   R² is a C₁-C₂₀ branched or unbranched hydrocarbon possessing 0-10 double bonds, 0-10 triple bonds or a combination of 0-10 double and triple bonds;
   each R⁷ is independently H or a C₁-C₆ branched or unbranched alkyl group;
   each R⁸ is independently H or a C₁-C₆ branched or unbranched alkyl group;
   R⁹ is a C₁-C₁₀ branched or unbranched hydrocarbon substituted with one or more groups independently selected from OR¹¹, N(R⁷)R¹², N(R⁷)₂, and COOR¹⁴;
   R¹⁰ is a C₁-C₁₀ branched or unbranched hydrocarbon substituted with one or more groups independently selected from OR¹¹, N(R⁸)R¹², N(R⁸)₂, and COOR¹⁴;
   each R¹¹ is independently H, Ac, Me, tert-Butyl, Benzyl, Trityl, Benzoyl, para-nitrobenzoyl, MOM, BOM or Si comprising the core of a silyl ether;
   each R¹² is independently H, Me, Boc, Cbz, Fmoc, Benzyl, 4-Methoxybenzyl, tert-Butyl, or Trityl;
   each R¹⁴ is independently H, C₁-C₆ branched or unbranched alkyl, Benzyl or 4-Methoxybenzyl;
   X is a direct linkage, O or NH;
   Y is a direct linkage, O or NH; and,
   each stereogenic center is independently R, S or racemic;
optionally converting a phosphorus-bound OH group to O—R⁴, wherein R⁴ is not H; and,
optionally converting each OR¹¹, N(R⁷)R¹², N(R⁸)R¹², or COOR¹⁴ to OH, NHR⁷, NHR⁸ or COOH, respectively.

As defined generally above, R¹ is a C₁-C₂₀ branched or unbranched hydrocarbon possessing 0-10 double bonds, 0-10 triple bonds or a combination of 0-10 double and triple bonds. In some embodiments, R¹ is a C₁-C₁₅ branched or unbranched hydrocarbon possessing 0-7 double bonds, 0-7 triple bonds or a combination of 0-7 double and triple bonds. In some embodiments, R¹ is a C₁-C₁₀ branched or unbranched hydrocarbon possessing 0-5 double bonds, 0-5 triple bonds or a combination of 0-5 double and triple bonds. In some embodiments, R¹ is a C₁₁-C₂₀ branched or unbranched hydrocarbon possessing 0-5 double bonds, 0-5 triple bonds or a combination of 0-5 double and triple bonds. In some embodiments, R¹ is a C₅-C₁₅ branched or unbranched hydrocarbon possessing 0-5 double bonds, 0-5 triple bonds or a combination of 0-5 double and triple bonds. In some embodiments, R¹ is a C₁-C₅ branched or unbranched hydrocarbon possessing 0-2 double bonds, 0-2 triple bonds or a combination of 0-2 double and triple bonds. In some embodiments, R¹ is a C₁₀-C₁₅ branched or unbranched hydrocarbon.

As defined generally above, R² is a C₁-C₂₀ branched or unbranched hydrocarbon possessing 0-10 double bonds, 0-10 triple bonds or a combination of 0-10 double and triple bonds. In some embodiments, R² is a C₁-C₁₅ branched or unbranched hydrocarbon possessing 0-7 double bonds, 0-7 triple bonds or a combination of 0-7 double and triple bonds. In some embodiments, R² is a C₁-C₁₀ branched or unbranched hydrocarbon possessing 0-5 double bonds, 0-5 triple bonds or a combination of 0-5 double and triple bonds. In some embodiments, R² is a C₁₁-C₂₀ branched or unbranched hydrocarbon possessing 0-5 double bonds, 0-5 triple bonds or a combination of 0-5 double and triple bonds. In some embodiments, R² is a C₅-C₁₅ branched or unbranched hydrocarbon possessing 0-5 double bonds, 0-5 triple bonds or a combination of 0-5 double and triple bonds. In some embodiments, R² is a C₁-C₅ branched or unbranched hydrocarbon possessing 0-2 double bonds, 0-2 triple bonds or a combination of 0-2 double and triple bonds. In some embodiments, R² is a C₁₀-C₁₅ branched or unbranched hydrocarbon.

As defined generally above, both R¹ and R² have the same definition. In some embodiments, R¹ and R² are the same. In some embodiments, R¹ and R² are different.

As defined generally above, R³ is

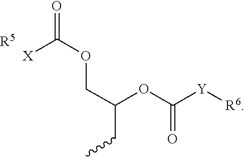

As defined generally above, R⁴ is H or a pharmaceutically acceptable cation, wherein incorporation of said pharmaceutically acceptable cation results in a salt, e.g., monomeric salt, a dimeric salt, a trimeric salt, or even a multimeric salt. In preferred embodiments, R⁴ is H, Li, Na, K, Mg, Ca, Zn, Cs, ammonium and tetraalkylammonium. In some embodiments, R⁴ is H. In some embodiments, R⁴ is Li. In some embodiments, R⁴ is Na. In some embodiments, R⁴ is K. In some embodiments, R⁴ is Mg. In some embodiments, R⁴ is Ca. In some embodiments, R⁴ is Zn. In some embodiments, R⁴ is Cs. In some embodiments, R⁴ is ammonium. In some embodiments, R⁴ is tetraalkylammonium.

As defined generally above, R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I.

As defined generally above, R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I.

As defined generally above, both R⁵ and R⁶ have the same definition. In some embodiments, R⁵ and R⁶ are the same. In some embodiments, R⁵ and R⁶ are different.

As defined generally above, each R⁷ is independently H or a C₁-C₆ branched or unbranched alkyl group. In some embodiments, each R⁷ is H. In some embodiments, each R⁷ is a C₁-C₆ branched or unbranched alkyl group. In some embodiments, at least one of R⁷ is H. In some embodiments, at least one of R⁷ is a C₁-C₆ branched or unbranched alkyl group.

As defined generally above, each R⁸ is independently H or a C₁-C₆ branched or unbranched alkyl group. In some embodiments, each R⁸ is H. In some embodiments, each R⁸ is a C₁-C₆ branched or unbranched alkyl group. In some embodiments, at least one of R⁷ is H. In some embodiments, at least one of R⁷ is a C₁-C₆ branched or unbranched alkyl group.

As defined generally above, R⁷ and R⁸ are similar. In some embodiments, R⁷ and R⁸ are the same. In some embodiments, R⁷ and R⁸ are different.

As defined generally above, R⁹ is a C₁-C₁₀ branched or unbranched hydrocarbon substituted with one or more groups independently selected from OR¹¹, N(R⁷)R¹², N(R⁷)₂, SR¹³, CN, COOR¹⁴, CONH₂, Cl, Br and I.

As defined generally above, R¹⁰ is a C₁-C₁₀ branched or unbranched hydrocarbon substituted with one or more groups independently selected from OR¹¹, N(R⁸)R¹², N(R⁸)₂, SR¹³, CN, COOR¹⁴, CONH₂, Cl, Br and I.

As defined generally above, each R¹¹ is independently H, Ac, Me, tert-Butyl, Benzyl, Trityl, Benzoyl, para-nitrobenzoyl, MOM, BOM or Si comprising the core of a silyl ether. In some embodiments, each R¹¹ is H. In some embodiments, each R¹¹ is Ac. In some embodiments, each R¹¹ is Me. In some embodiments, each R¹¹ is tert-Butyl. In some embodiments, each R¹¹ is Benzyl. In some embodiments, each R¹¹ is Trityl. In some embodiments, each R¹¹ is Benzoyl. In some embodiments, each R¹¹ is para-nitrobenzoyl. In some embodiments, each R¹¹ is MOM. In some embodiments, each R¹¹ is BOM. In some embodiments, each R¹¹ is Si comprising the core of a silyl ether. In some embodiments, each R¹¹ is the same. In some embodiments, each R¹¹ is different.

As defined generally above, each R¹² is independently H, Me, Boc, Cbz, Fmoc, Benzyl, 4-Methoxybenzyl, tert-Butyl, or Trityl. In some embodiments, each R¹² is H. In some embodiments, each R¹² is Me. In some embodiments, each R¹² is Boc. In some embodiments, each R¹² is Cbz. In some embodiments, each R¹² is Fmoc. In some embodiments, each R¹² is Benzyl. In some embodiments, each R¹² is 4-Methoxybenzyl. In some embodiments, each R¹² is tert-Butyl. In some embodiments, each R¹² is Trityl. In some embodiments, each R¹² is the same. In some embodiments, each R¹² is different.

As defined generally above, each R¹³ is independently H, Ac, Benzoyl, para-nitrobenzoyl or Trityl. In some embodiments, each R¹³ is H. In some embodiments, each R¹³ is Ac. In some embodiments, each R¹³ is Benzoyl. In some embodiments, each R¹³ is para-nitrobenzoyl. In some embodiments, each R¹³ is Trityl. In some embodiments, each R¹³ is the same. In some embodiments, each R¹³ is different.

As defined generally above, each R¹⁴ is independently H, C₁-C₆ branched or unbranched alkyl, Benzyl or 4-Methoxybenzyl. In some embodiments, each R¹⁴ is H. In some embodiments, each R¹⁴ is C₁-C₆ branched or unbranched alkyl. In some embodiments, each R¹⁴ is Benzyl. In some embodiments, each R¹⁴ is 4-Methoxybenzyl. In some embodiments, each R¹⁴ is the same. In some embodiments, each R¹⁴ is different.

As defined generally above, X is a direct linkage, O or NH. In some embodiments, X is a direct linkage. In some embodiments, X is O. In some embodiments, X is NH.

As defined generally above, Y is a direct linkage, O or NH. In some embodiments, Y is a direct linkage. In some embodiments, Y is O. In some embodiments, Y is NH.

As defined generally above, both X and Y have the same definition. In some embodiments, X and Y are the same. In some embodiments, X and Y are different.

As defined generally above, each stereogenic center is independently R, S or racemic.

In different embodiments, the present invention has a structure of Compounds 1-51.

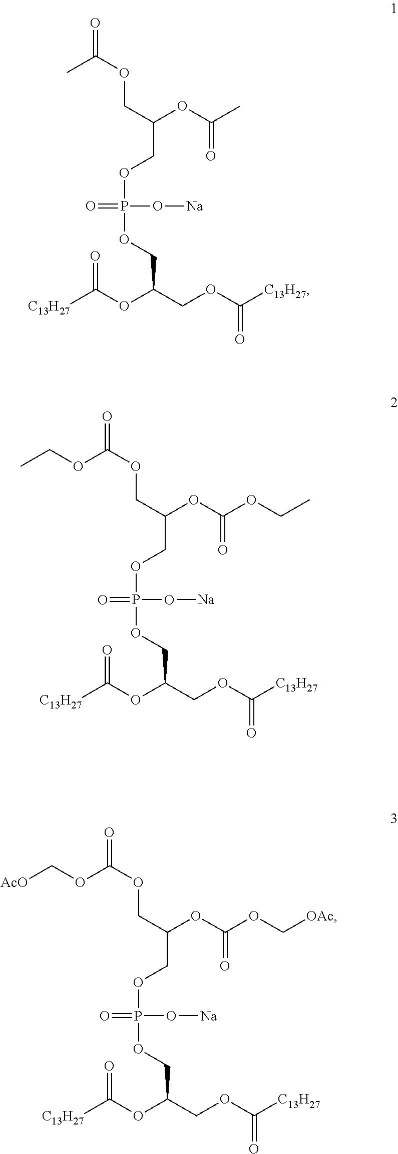

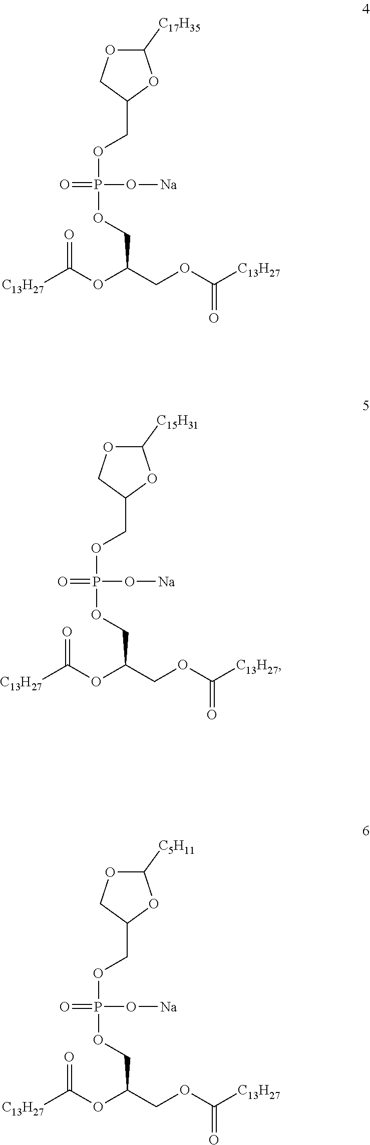

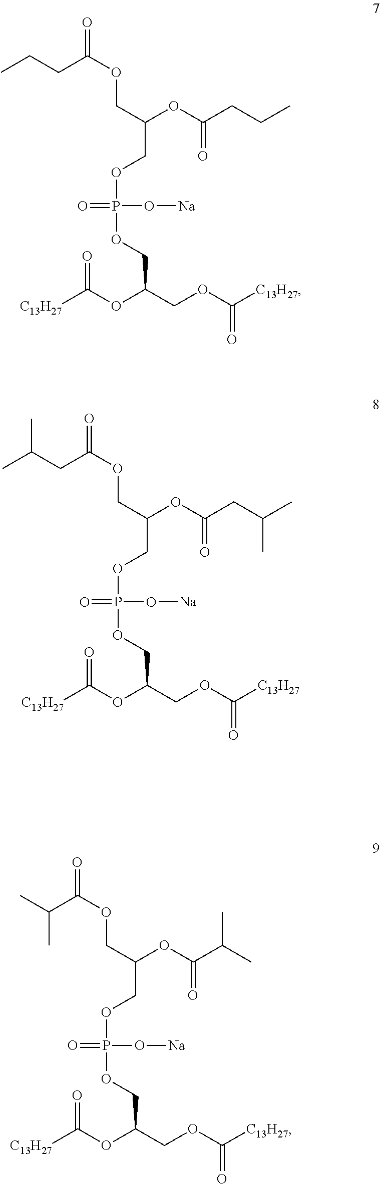

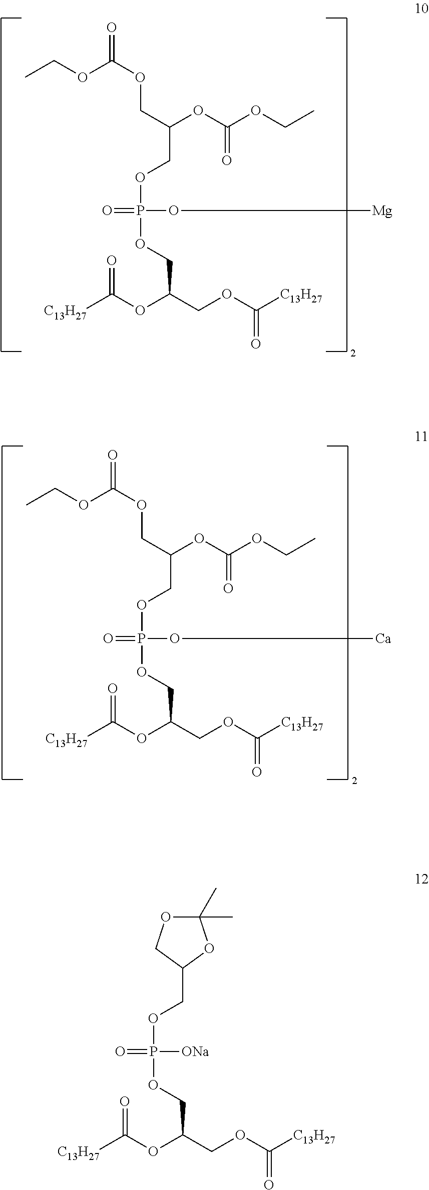

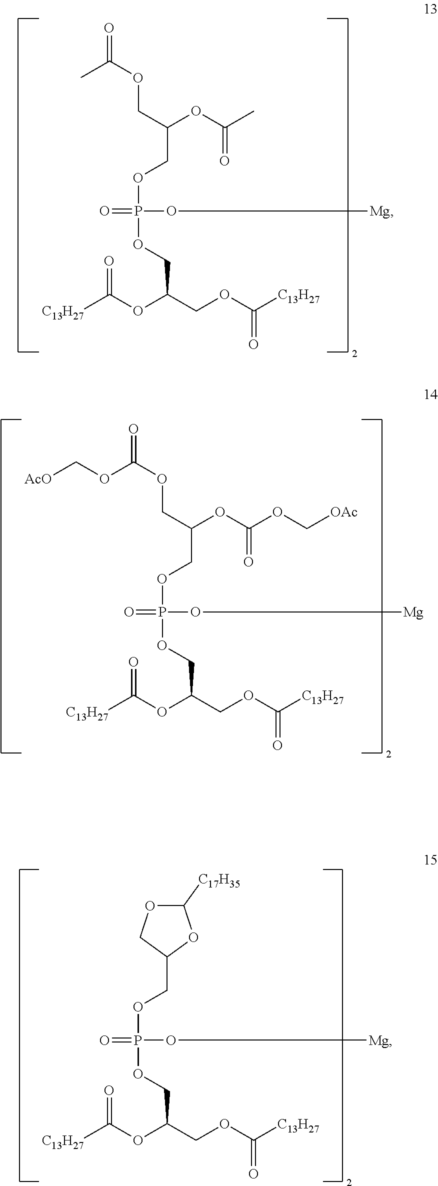

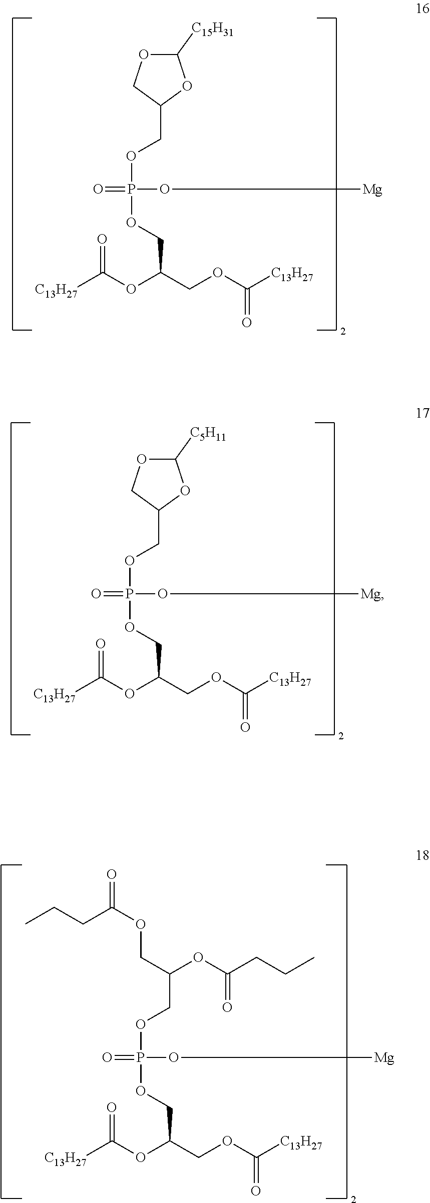

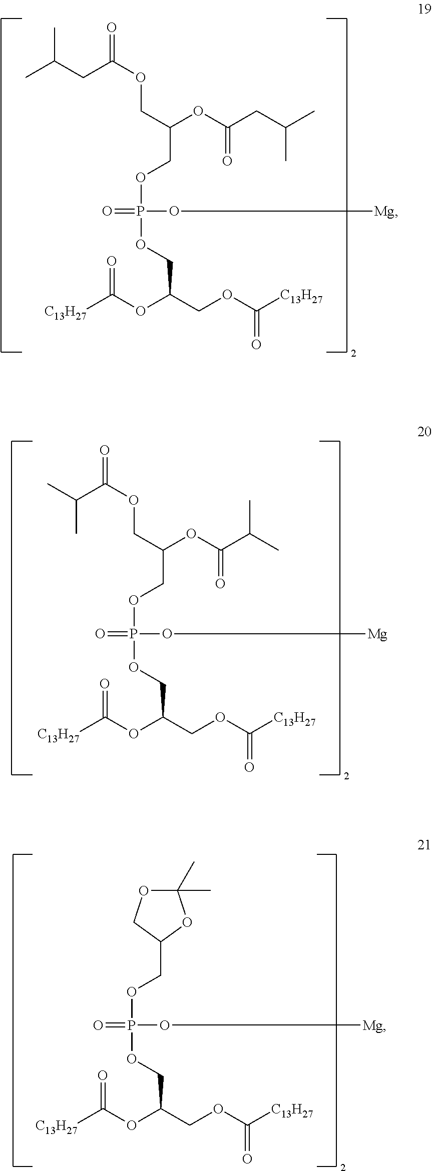

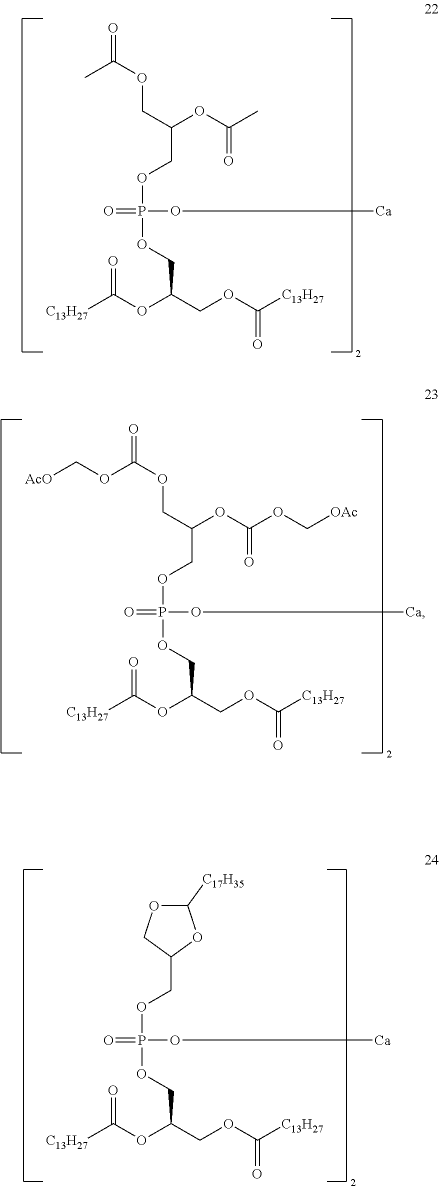

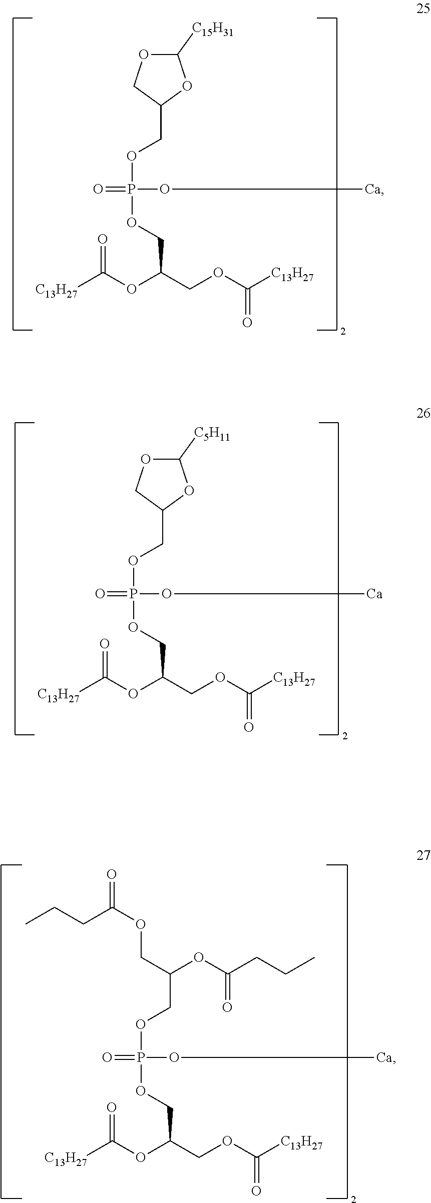

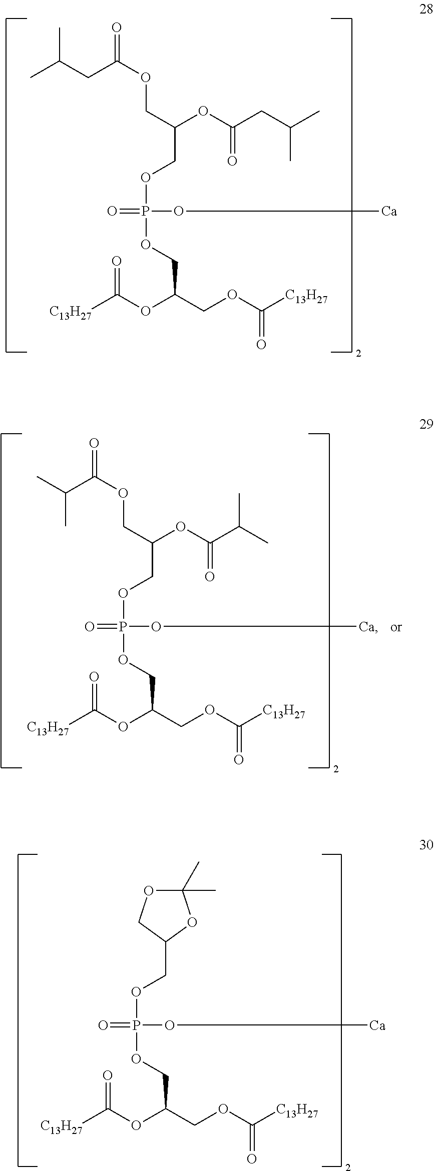

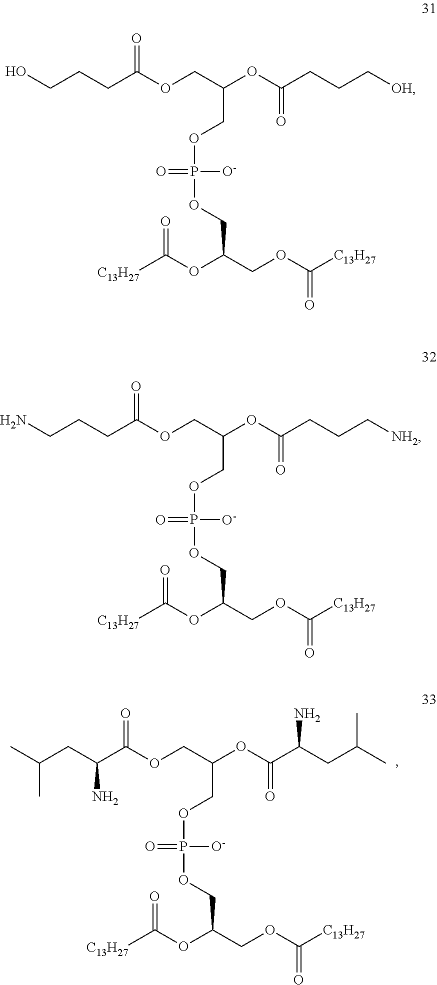

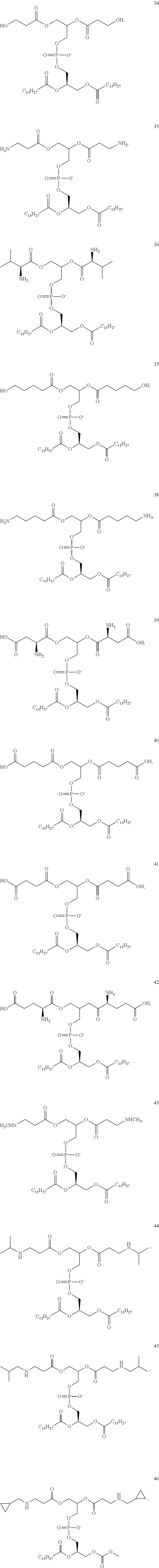

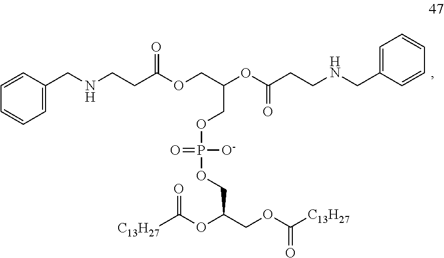

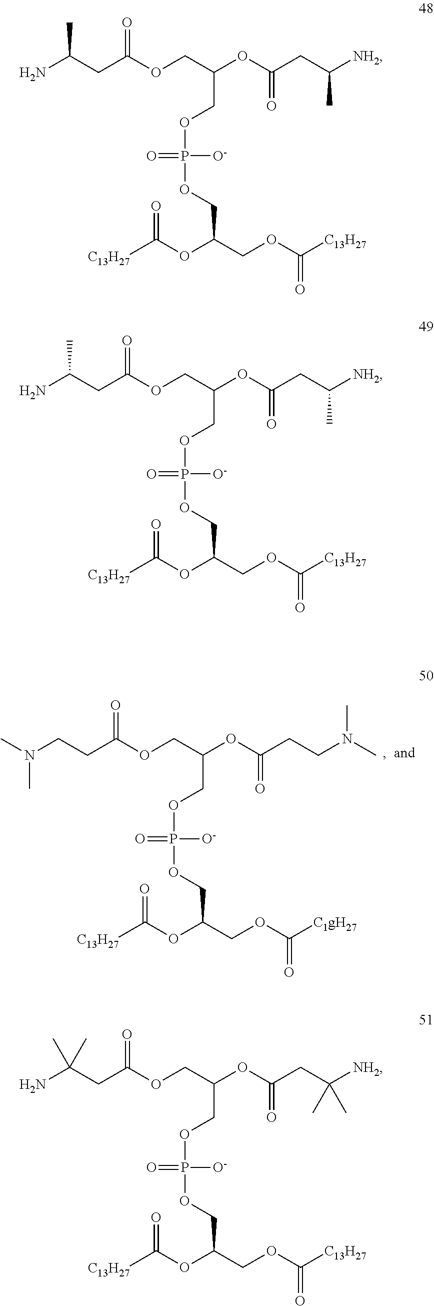

wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt.

One embodiment of this invention relates to pharmaceutical compositions comprising a compound of Formula I and a pharmaceutically acceptable diluent or carrier. In one embodiment, said pharmaceutical compositions comprise a compound of Formula I in an amount per unit dose of between about 1 mg and about 1 gram. In some embodiments, the amount per unit dose is between about 1 mg and about 500 mg. In some embodiments, the amount per unit dose is between about 500 mg and about 1 gram. In some embodiments, the amount per unit dose is between about 250 mg and about 750 mg. In some embodiments, the amount per unit dose is between about 50 mg and about 450 mg. In some embodiments, the amount per unit dose is between about 100 mg and about 300 mg.

Another embodiment of this invention relates to pharmaceutical compositions comprising a compound of Formula IA and a pharmaceutically acceptable diluent or carrier. In one embodiment, said pharmaceutical compositions comprise a compound of Formula IA in an amount per unit dose of between about 1 mg and about 1 gram. In some embodiments, the amount per unit dose is between about 1 mg and about 500 mg. In some embodiments, the amount per unit dose is between about 500 mg and about 1 gram. In some embodiments, the amount per unit dose is between about 250 mg and about 750 mg. In some embodiments, the amount per unit dose is between about 50 mg and about 450 mg. In some embodiments, the amount per unit dose is between about 100 mg and about 300 mg.

In some embodiments, said pharmaceutical compositions additionally comprise one or more agents that induce a cardiopathy as a side effect, and wherein the compound of Formula I or the compound of Formula IA reduces or eliminates the cardiopathy. In some embodiments, the one or more agents that induce a cardiopathy as a side effect are selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone. One of ordinary skill in the art will recognize that additional agents that induce a cardiopathy exist and may benefit from inclusion in formulations of the present invention.

In some embodiments, the present invention includes a composition comprising an active agent that causes a cardiopathy and a compound of Formula I or a compound of Formula IA represented by one or more compounds of Formula I or Formula IA, for example, Compounds 1 to 42, as set forth above.

One embodiment of this invention provides a pharmaceutical composition comprising a structure of Formula I or as structure of Formula IA, for example, Compounds 1 to 42, formulated for oral, sublingual, transdermal, suppository, intrathecal, enteral, parenteral, intravenous, intraperitoneal, cutaneous, subcutaneous, topical, pulmonary, rectal, vaginal, or intramuscular administration, as set forth above. In some embodiments, the composition formulated for oral administration is a tablet, capsule, caplet, pill, powder, troche, lozenge, slurry, liquid solution, suspension, emulsion, elixir or oral thin film (OTF). In some embodiments, the composition is a solid form, a solution, a suspension, or a soft gel form.

One embodiment of this invention provides pharmaceutical compositions comprising an active agent that causes a cardiopathy as a side effect and a compound of Formula I or a compound of Formula IA, for example Compounds 1 to 42, as set forth above.

One embodiment of this invention provides a method of reducing or eliminating one or more of a cardiac channelopathy, cardiac muscle damage, or a condition resulting from the irregularity or alteration in the cardiac pattern, in a human or animal subject caused by an active agent used to treat a disease, comprising the steps of: administering to the human or animal subject a pharmaceutical composition comprising a compound of Formula I or a compound of Formula IA, for example, Compounds 1 to 42, as set forth above.

In some embodiments, said pharmaceutical compositions additionally comprise one or more excipients, binders, anti-adherents, coatings, disintegrants, fillers, flavors, dyes, colors, glidants, lubricants, preservatives, sorbents, sweeteners, derivatives thereof, or combinations thereof. In some embodiments, the binder is selected from the group consisting of hydroxypropylmethylcellulose, ethyl cellulose, povidone, acrylic and methacrylic acid co-polymers, pharmaceutical glaze, gums, and milk derivatives.

In one embodiment, said pharmaceutical compositions comprise a compound of Formula I or a compound of Formula IA in an amount per unit dose of between about 1 mg and about 1 gram. In some embodiments, the amount per unit dose is between about 1 mg and about 500 mg. In some embodiments, the amount per unit dose is between about 500 mg and about 1 gram. In some embodiments, the amount per unit dose is between about 250 mg and about 750 mg. In some embodiments, the amount per unit dose is between about 50 mg and about 450 mg. In some embodiments, the amount per unit dose is between about 100 mg and about 300 mg.

In some embodiments, said pharmaceutical compositions additionally comprise one or more excipients, binders, anti-adherents, coatings, disintegrants, fillers, flavors, dyes, colors, glidants, lubricants, preservatives, sorbents, sweeteners, derivatives thereof, or combinations thereof. In some embodiments, the binder is selected from the group consisting of hydroxypropylmethylcellulose, ethyl cellulose, povidone, acrylic and methacrylic acid co-polymers, pharmaceutical glaze, gums, and milk derivatives.

In one embodiment, the present invention includes a composition, a pharmaceutical composition, and a method in which the active agent that causes a cardiopathy as a side effect is selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone. One of ordinary skill in the art will recognize that additional agents that induce a cardiopathy exist and may benefit from inclusion in formulations of the present invention.

In some embodiments, said pharmaceutical compositions are formulated for oral, sublingual, transdermal, suppository, intrathecal, enteral, parenteral, intravenous, intraperitoneal, cutaneous, subcutaneous, topical, pulmonary, rectal, vaginal, or intramuscular administration. In some embodiments, said pharmaceutical composition formulated for oral administration is a tablet, capsule, caplet, pill, powder, troche, lozenge, slurry, liquid solution, suspension, emulsion, elixir or oral thin film (OTF). In some embodiments, the composition is a solid form, a solution, a suspension, or a soft gel form. In some embodiments, the solid form further comprises one or more excipients, binders, anti-adherents, coatings, disintegrants, fillers, flavors, dyes, colors, glidants, lubricants, preservatives, sorbents, sweeteners, derivatives thereof, or combinations thereof. In some embodiments, the binder is selected from the group consisting of hydroxypropylmethylcellulose, ethyl cellulose, povidone, acrylic and methacrylic acid co-polymers, pharmaceutical glaze, gums, and milk derivatives.

In one embodiment, said method provides pharmaceutical compositions that comprise a compound of Formula I or a compound of Formula IA in an amount per unit dose of between about 1 mg and about 1 gram. In some embodiments, the amount per unit dose is between about 1 mg and about 500 mg. In some embodiments, the amount per unit dose is between about 500 mg and about 1 gram. In some embodiments, the amount per unit dose is between about 250 mg and about 750 mg. In some embodiments, the amount per unit dose is between about 50 mg and about 450 mg. In some embodiments, the amount per unit dose is between about 100 mg and about 300 mg.

In one embodiment, said method provides a pharmaceutical composition formulated for oral, sublingual, transdermal, suppository, intrathecal, enteral, parenteral, intravenous, intraperitoneal, cutaneous, subcutaneous, topical, pulmonary, rectal, vaginal, or intramuscular administration. In some embodiments, said pharmaceutical composition formulated for oral administration is a tablet, capsule, caplet, pill, powder, troche, lozenge, slurry, liquid solution, suspension, emulsion, elixir or oral thin film (OTF). In some embodiments, the composition is a solid form, a solution, a suspension, or a soft gel form. In some embodiments, the solid form further comprises one or more excipients, binders, anti-adherents, coatings, disintegrants, fillers, flavors, dyes, colors, glidants, lubricants, preservatives, sorbents, sweeteners, derivatives thereof, or combinations thereof. In some embodiments, the binder is selected from the group consisting of hydroxypropylmethylcellulose, ethyl cellulose, povidone, acrylic and methacrylic acid co-polymers, pharmaceutical glaze, gums, and milk derivatives.

In one embodiment, said method provides pharmaceutical compositions. One embodiment of this invention provides administration of a compound of Formula I or a compound of Formula IA, wherein said compound is a lipid that reduces or eliminates cardiopathies, such as QT prolongation, cardiac muscle damage, or AV block, that are drug-induced or caused by a disease or condition.

The single most common cause of the withdrawal or restriction of the use of marketed drugs has been QT-interval prolongation associated with polymorphic ventricular tachycardia, or torsade de pointes, a condition that can be fatal.

5-HT3 antagonists block serotonin binding. Aloxi (or palonasitron HCL) is an antiemetic for chemotherapy induced nausea and vomiting, a 5-HT 3 antagonist, blocks serotonin binding to 5-HT3. In a study there was no significant difference in the QTc intervals during the perioperative period, whether 0.075 mg of palonosetron is administered before or after sevoflurane anesthesia. Palonosetron may be safe in terms of QTc intervals during sevoflurane anesthesia.

5-HT4 receptor agonist. Cisapride is a gastroprokinetic agent, a drug that increases motility in the upper gastrointestinal tract. It acts directly as a serotonin 5-HT4 receptor agonist and indirectly as a parasympathomimetic. Cisapride dose-dependently prolongs the QT interval. Neither torsade de pointe nor ventricular tachycardia were noted when monitoring 33 patients during a higher dose stage.

Histamine Antagonist. Antihistamines used in the treatment of allergy act by competing with histamine for H1-receptor sites on effector cells. They thereby prevent, but do not reverse, responses mediated by histamine alone.

Pain and Premenstrual Symptom Relief H1 antagonists are most useful in acute exudative types of allergy that present with symptoms of rhinitis, urticaria, and conjunctivitis. Their effect, however, is purely palliative and confined to the suppression of symptoms attributable to the histamine-antibody reaction.

Pyrilamine is a diuretic first-generation histamine H1 antagonist. There is a case of an adolescent with prolonged QT interval after an overdose of pyrilamine. Reports of deaths resulting from ventricular tachyarrhythmias have been made.

Terfenidine is an antihistamine, used to treat allergies, hives (urticaria), and other allergic inflammatory conditions. The brand name Seldane is discontinued in the U.S. Rare reports of severe cardiovascular adverse effects have been received which include ventricular tachyarrhythmias (torsades de pointes, ventricular tachycardia, ventricular fibrillation, and cardiac arrest), hypotension, palpitations, or syncope.

Loratidine is a first-line antihistamine is a second-generation peripheral histamine H1-receptor blocker. In structure, it is closely related to tricyclic antidepressants, such as imipramine, and is distantly related to the atypical antipsychotic quetiapine. Some antihistamines, such as mizolastine and ebastine, can prolong the QT interval and provoke severe cardiac arrhythmias. As of mid 2009 very few clinical data had been published on the risk of QT prolongation with loratadine. Very rare reported cases of torsades de pointes linked to loratadine mainly appear to involve drug interactions, especially with amiodarone and enzyme inhibitors. There are no reports of QT prolongation attributed to desloratadine, the main metabolite of loratadine. Patients who have risk factors for torsades de pointes or who are taking certain enzyme inhibitors should avoid using loratadine.

Astemizole is a long-acting and highly selective H1 antagonist, acting on histamine H-1 receptor and H-3 receptors. It has antipruritic, and anticholinergic effects. It is also afunctional inhibitor of acid sphingomyelinase. An overdose of astemizole predisposes the myocardium to ventricular dysrhythmias, including torsades de pointes. However, dysrhythmias developed only in patients with corrected QT intervals greater than 500 ms.

Calcium channel blocker. Prenylamine is a calcium channel blocker of the amphetamine chemical class that is used as a vasodilator in the treatment of angina pectoris. Resting ECGs were recorded in 29 patients with angina pectoris before, during and after treatment with prenylamine 180 mg daily. The QT interval became significantly prolonged after one week of treatment. The prolongation persisted as long as therapy was continued, which was up to 6 months. After withdrawal of treatment the QT interval returned to normal within 2 weeks.

Lidoflazine is a piperazine calcium channel blocker is a coronary vasodilator with some antiarrhythmic action. As a tricyclic antihistamine, it acts as a selective inverse agonist of peripheral histamine H1-receptors. It carries a significant risk of QT interval prolongation and ventricular arrhythmia. Lidoflazine inhibits potently HERG current (I(HERG)) recorded from HEK 293 cells stably expressing wild-type HERG (IC(50) of approximately 16 nM). It is approximately 13-fold more potent against HERG than verapamil under similar conditions in preferentially inhibiting activated/open HERG channels. Lidoflazine produces high affinity blockade of the alpha subunit of the HERG channel by binding to aromatic amino acid residues within the channel pore and, second, that this is likely to represent the molecular mechanism of QT interval prolongation by this drug.

Bepridil is an antihypertensive drug which disrupts the movement of calcium (Ca²⁺) through calcium channels. While it prolongs the QT interval. Bepridil prolongs the QT and refractoriness and a linear correlation could be demonstrated between the percent change in QTc and refractory period prolongation. Bepridil in one patient reduced by one the number of stimuli required to induce VT, but no spontaneous arrhythmias were noted, It possesses antiarrhythmic properties with a minimal proarrhythmic effect.

Antimalarials. Chloroquine-Chlorpheniramine (chloroquine plus chloropheniramine) is a histamine H1 receptor blocker that reverses chloroquine insensitivity in Plasmodium falciparum in vitro, Chloroquine/chloropheniramine produces a higher cure rate than chloroquine alone. Short QT Syndrome (SQTS) is a sporadic or autosomal dominant disorder characterized by markedly accelerated cardiac repolarization, ventricular arrhythmias and sudden cardiac death. To date, mutations in 5 different ion channel genes (KCNH2, KCNQ1, KCNJ2, CACNA1C and CACNB2) have been identified to cause SQTS. The risk of ventricular arrhythmias and sudden death is remarkably high in SQTS with cardiac arrest reported as a presenting symptom in 31% of SQTS subjects. Chloroquine Blocks a Mutant Kir2.1 Channel Responsible for Short QT Syndrome and Normalizes Repolarization Properties in silico.

Halofantrine is an antimalarial agent with a substituted phenanthrene, and is related to the antimalarial drugs quinine and lumefantrine. It can be associated with cardiotoxicity. The most dangerous side effect is cardiac arrhythmias: halofantrine causes significant QT prolongation, and this effect is seen even at standard doses. The drug should therefore not be given to patients with cardiac conduction defects and should not be combined with mefloquine. The mechanism of action of halofantrine is unknown.

Quinidine is an antimalarial that acts as a class I antiarrhythmic agent (Ia) in the heart. It is a stereoisomer of quinine, this alkaloid dampens the excitability of cardiac and skeletal muscles by blocking sodium and potassium currents across cellular membranes. It prolongs cellular action potential, and decreases automaticity. Quinidine also blocks muscarinic and alpha-adrenergic neurotransmission. Quinidine causes greater QT prolongation in women than in men at equivalent serum concentrations. This difference may contribute to the greater incidence of drug-induced torsades de pointes observed in women taking quinidine and has implications for other cardiac and noncardiac drugs that prolong the QTc interval.

Antipsychotics. First-generation antipsychotics, known as typical antipsychotics, were discovered in the 1950s. Most second-generation drugs, known as atypical antipsychotics, have been developed more recently, although the first atypical antipsychotic, clozapine, was discovered in the 1960s and introduced clinically in the 1970s. Both generations of medication tend to block receptors in the brain's dopamine pathways, but atypicals tend to act on serotonin receptors as well. Both generations of medication tend to block receptors in the brain's dopamine pathways, but atypicals tend to act on serotonin receptors as well. QTc interval prolongation can occur as a result of treatment with both conventional and novel antipsychotic medications and is of clinical concern because of its association with the potentially fatal ventricular arrhythmia, torsade de pointes.

Pimozide is an antipsychotic drug of the diphenylbutylpiperidine class, Can induce prolongation of the QT interval. Pimozide is contraindicated in individuals with either acquired, congenital or a family history of QT interval prolongation. Its use is advised against in individuals with people with either a personal or a family history of arrhythmias or torsades de pointe acts as an antagonist of the D2, D3, and D4 receptors and the 5-HT7 receptor. It is also a hERG blocker.

Sertindole is an antipsychotic medication. Like other atypical antipsychotics, it has activity at dopamine and serotonin receptors in the brain. Abbott Labs first applied for U.S. Food and Drug Administration (FDA) approval for sertindole in 1996, but withdrew this application in 1998 following concerns over the increased risk of sudden death from QTc prolongation. In a trial of 2000 patients on taking sertindole, 27 patients died unexpectedly, including 13 sudden deaths. The drug has not been approved by the FDA for use in the USA. In Europe, Sertindole was approved and marketed in 19 countries from 1996, but its marketing authorization was suspended by the European Medicines Agency in 1998 and the drug was withdrawn from the market. In 2002, based on new data, the EMA's CHMP suggested that Sertindole could be reintroduced for restricted use in clinical trials, with strong safeguards including extensive contraindications and warnings for patients at risk of cardiac dysrhythmias, a recommended reduction in maximum dose from 24 mg to 20 mg in all but exceptional cases, and extensive ECG monitoring requirement before and during treatment

Chlorpromazine, marketed as Thorazine and Largactil, is an antipsychotic medication in the typical antipsychotic class. Its mechanism of action is not entirely clear but believed to be related to its ability as a dopamine antagonist. It also has anti-serotonergic and anti-histaminergic properties. Chlorpromazine is a very effective antagonist of D2 dopamine receptors and similar receptors, such as D3 and D5. Unlike most other drugs of this genre, it also has a high affinity for D1 receptors. Electrocardiogram QT corrected interval prolonged is reported only by a few people who take Thorazine. In a study of 2,633 people who have side effects while taking Thorazine from FDA and social media, 5 have electrocardiogram QT corrected interval prolonged.

Thioridazine is a piperidine typical antipsychotic drug belonging to the phenothiazine drug branded product was withdrawn worldwide in 2005 because it caused severe cardiac arrhythmias, however, generic versions are available in the US. The drug was voluntarily discontinued by its manufacturer, Novartis, worldwide because it caused severe cardiac arrhythmias. Thioridazine prolongs the QTc interval in a dose-dependent manner. The ratio of 5-HT2A to D2 receptor binding is believed to dictate whether or not most antipsychotics are atypical or typical. In thioridazine's case its ratio of 5-HT2A to D2 receptor binding is below the level that's believed to be required for atypicality despite its relatively low extrapyramidal side effect liability in practice.

Haldol, Haloperidol. A typical antipsychotic medication QT interval prolongation is meperidine. It is on the WHO Model List of Essential Medicines, It is the most commonly used typical antipsychotic, Special cautions: patients at special risk for the development of QT prolongation (hypokalemia, concomitant use of other drugs causing QT Amiodarone: Q-Tc interval prolongation (potentially dangerous change in heart rhythm prolongation).

Mesoridazone is a piperidine neuroleptic drug belonging to the class of drugs called phenothiazines, used in the treatment of schizophrenia. It is a metabolite of thioridazine. Mesoridazine was withdrawn from the United States market in 2004 due to dangerous side effects, namely irregular heartbeat and QT-prolongation of the electrocardiogram.

Selective serotonin reuptake inhibitors. Celexa (citalopram) is an antidepressant in a group of drugs called selective serotonin reuptake inhibitors (SSRIs). Its chemical structure a racemic bicyclic phthalane derivative designated (±)-1-(3-dimethylaminopropyl)-1-(4-fluorophenyl)-1,3-dihydroisobenzofuran-5-carbonitrile, is unrelated to that of other SSRIs, or other available antidepressant agents. Citalopram may cause a condition that affects the heart rhythm (QT prolongation).

Antibiotics. Moxifloxacin is a fourth-generation synthetic fluoroquinolone antibacterial agent. It functions by inhibiting DNA gyrase, a type II topoisomerase, and topoisomerase IV (enzymes necessary to separate bacterial DNA thereby inhibiting cell replication) may cause torsade de pointes. Coadministration of moxifloxacin with other drugs that also prolong the QT interval or induce bradycardia (e.g., beta-blockers, amiodarone) should be avoided. Careful consideration should be given in the use of moxifloxacin in patients with cardiovascular disease, including those with conduction abnormalities. Drugs that prolong the QT interval may have an additive effect on QT prolongation and lead to increased risk of ventricular arrhythmias.

Pentamadine is an antimicrobial medication given to prevent and treat pneumocystis pneumonia. The exact mechanism of its anti-protozoal action is unknown (though it may involve reactions with ubiquitin and mitochondrial function. Severe or fatal arrhythmias and heart failure are quite frequent. the aromatic diamidine pentamidine acts via inhibition of hERG channel trafficking. Pentamidine has no acute effects on currents produced by hERG, KvLQT1/mink, Kv4.3, or SCNA5. After overnight exposure, however, pentamidine reduces hERG currents and inhibited trafficking and maturation of hERG with IC50 values of 5 to 8 μM similar to therapeutic concentrations.

Clarithromycin is an antibiotic made from erythromycin is chemically known as 6-O-methylerythromycin. It is in the macrolide class and works by stopping the making of protein by some bacteria. It causes QT prolongation or ventricular cardiac arrhythmias, including torsade de pointes.

Erythromycin is an antibiotic with common side effects that include serious side effects arrhythmia with prolonged QT intervals including torsades de pointes.

Grepafloxacin is an oral broad-spectrum fluoroquinolone antibacterial agent used to treat bacterial infections. Grepafloxacin was withdrawn worldwide from markets in 1999, owing to its side effect of lengthening the QT interval on the electrocardiogram, leading to cardiac events and sudden death.

Sparfloxacin is a fluoroquinolone broad-spectrum antibiotic used in the treatment of bacterial infections. It has a controversial safety profile. The use of sparfloxacin is contraindicated in patients with known QTc prolongation and in patients treated concomitantly with class IA or III antiarrhythmic drugs. In a study, the maximum plasma concentration (Cmax) after the 1200- and 1600-mg doses was lower than would be expected for a linear dose relationship. This was also the case with the mean increase and mean maximum increase in QTc interval. Increases in the QTc interval correlated well with Cₘₐₓ but not with AUCo-infinity.

Curcumin (diferuloylmethane) is a bright yellow chemical produced by some plants. It is the principal curcuminoid of turmeric (Curcuma longa) and exerts antioxidant, anti-inflammatory, antiviral, antibacterial, antifungal, and anti-tumor activities. In whole-cell patch-clamp experiments, curcumin inhibited hERG K+ currents in HEK293 cells stably expressing hERG channels in a dose-dependent manner, with IC50 value of 5.55 μM. The deactivation, inactivation and the recovery time from inactivation of hERG channels were significantly changed by acute treatment of 10 μM curcumin.

Antiarrhythmics. Antiarrhythmics are used to suppress abnormal rhythms of the heart (cardiac arrhythmias), such as atrial fibrillation, ventricular tachycardia, and ventricular fibrillation. Procainamide is an antiarrhythmic class used for the treatment of cardiac arrhythmias. It is classified by the Vaughan Williams classification system as class Ia, and is used for both supraventricular and ventricular arrhythmias. It was also detected that the antiarrhythmic drug procainamide interferes with pacemakers. Because a toxic level of procainamide leads to decrease in ventricular conduction velocity and increase of the ventricular refractory period. This results in a disturbance in the artificial membrane potential and leads to a supraventricular tachycardia, which induces failure of the pacemaker and death. It induces rapid block of the batrachotoxin (BTX)-activated sodium channels of the heart muscle and acts as antagonist to long gating closures Procainamide belongs to the aminobenzamides, which has similar cardiac effects as quinidine it has the same toxicity profile as quinidine.

Propafenone is a class 1C anti-arrhythmic medication, which treats illnesses associated with rapid heartbeats such as atrial and ventricular arrhythmias and works by slowing the influx of sodium ions into the cardiac muscle cells, causing a decrease in excitability of the cells. Propafenone is more selective for cells with a high rate, but also blocks normal cells more than class Ia or Ib. Propafenone differs from the prototypical class Ic antiarrhythmic in that it has additional activity as a beta-adrenergic blocker, which can cause bradycardia.

Methanesulphonanilide (E-4031) is an experimental class III antiarrhythmic drug that blocks potassium channels of class III antiarrhythmic drug. E-4031 acts on a specific class of voltage-gated potassium channels mainly found in the heart, the hERG channels. hERG channels (Kv11.1) mediate the IKr current, which repolarizes the myocardial cells. The hERG channel is encoded by ether-a-go-go related gene (hERG). E-4031 blocks hERG-type potassium channels by binding to the open channels. Its structural target within the hERG-channel is unclear, but some other methanesulfonanilide class III antiarrhythmic drugs are known to bind to the S6 domain or C-terminal of the hERG-channel. As E-4031 can prolong the QT-interval, it can cause lethal arrhythmias. So far, one clinical trial has been conducted to test the effect of E-4031 on prolongation of the QT-interval.

Amiodarone is a class III antiarrhythmic for ventricular fibrillation or tachycardia, prolongs phase 3 of the cardiac action potential. Amiodarone is an antiarrhythmic agent known to cause prolongation of action potential duration, which is reflected in the electrocardiogram as a prolongation of the QT. Amiodarone has multiple effects on myocardial depolarization and repolarization that make it an extremely effective antiarrhythmic drug. Its primary effect is to block the potassium channels, but it can also block sodium and calcium channels and the beta and alpha adrenergic receptors. Amiodarone significantly prolongs the QT interval and the QTc value.

Dronedarone is a benzofuran derivative related to amiodarone, is a drug used mainly for cardiac arrhythmias (approved by the FDA in 2009). It is a “multichannel blocker”, however, it is unclear which channel(s) play a pivotal role in its success. Dronedarone's actions at the cellular level are controversial with most studies suggesting an inhibition in multiple outward potassium currents including rapid delayed rectifier, slow delayed rectifier and ACh-activated inward rectifier. It is also believed to reduce inward rapid Na current and L-type Ca channels. The reduction in K current in some studies was shown to be due to the inhibition of K-ACh channel or associated GTP-binding proteins. A reduction of K+ current by 69% led to increased AP duration and increased effective refractory periods, Displays amiodarone-like class III antiarrhythmic activity in vitro and in clinical trials. The drug also appears to exhibit activity in each of the 4 Vaughan-Williams antiarrhythmic classes. Contraindicated in Concomitant use of drugs or herbal products that prolong the QT interval and may induce Torsade de Pointes QTc Bazett interval≥500 ms, or use with drugs or herbal supplements that prolong QT interval or increase risk of torsades de points (Class I or III antiarrhythmic agents, phenothiazines, tricyclic antidepressants, certain oral macrolides, ephedra).

Disopyramide is an antiarrhythmic medication used in the treatment of ventricular tachycardia. It is a sodium channel blocker and therefore classified as a Class 1a anti-arrhythmic agent. Disopyramide's Class 1a activity is similar to that of quinidine in that it targets sodium channels to inhibit conduction. Disopyramide depresses the increase in sodium permeability of the cardiac Myocyte during Phase 0 of the cardiac action potential, in turn decreasing the inward sodium current. This results in an increased threshold for excitation and a decreased upstroke velocity Disopyramide prolongs the PR interval by lengthening both the QRS and P wave duration. Concern about disopyramide has been the hypothetical potential for inducing sudden death from its type 1 anti-arrhythmic effects.

Dofetilide is a class III antiarrhythmic agent. Due to the pro-arrhythmic potential of dofetilide, it is only available by prescription from physicians who have undergone specific training in the risks of treatment with dofetilide. In addition, it is only available by mail order or through specially trained local pharmacies Dofetilide works by selectively blocking the rapid component of the delayed rectifier outward potassium current. There is a dose-dependent increase in the QT interval and the corrected QT interval (QTc). Because of this, many practitioners will initiate dofetilide therapy only on individuals under telemetry monitoring or if serial EKG measurements of QT and QTc can be performed.

Sotalol is a non-selective competitive beta-adrenergic receptor blocker that also exhibits Class III antiarrhythmic properties. The U.S. Food and Drug Administration advises that sotalol only be used for serious arrhythmias, because its prolongation of the QT interval carries a small risk of life-threatening torsade de pointes. Sotalol also acts on potassium channels and causes a delay in relaxation of the ventricles. By blocking these potassium channels, sotalol inhibits efflux of K+ ions, which results in an increase in the time before another electrical signal can be generated in ventricular myocytes. This increase in the period before a new signal for contraction is generated.

Ibutilide is a Class III antiarrhythmic agent that is indicated for acute cardioconversion of atrial fibrillation and atrial flutter and prolongs action potential and refractory period of myocardial cells. Because of its Class III antiarrhythmic activity, there should not be concomitant administration of Class Ia and Class III agents. Unlike most other Class III antiarrhythmic drugs, ibutilide does not produce its prolongation of action potential via blockade of cardiac delayed rectifier of potassium current, nor does it have a sodium-blocking, antiadrenergic, and calcium blocking activity that other Class III agents possess. Thus, it is often referred as a “pure” Class III antiarrhythmic drug. Ibutilide, like other class III antiarrhythmic drugs, blocks delayed rectified potassium current. It does have action on the slow sodium channel and promotes the influx of sodium through these slow channels. Like other antiarrhythmics, ibutilide can lead to abnormal heart rhythms due to its ability to prolong the QT interval, which can lead to the potentially fatal abnormal heart rhythm known as torsades de pointes. The drug is contraindicated in patients that are likely to develop abnormal heart rhythms; persons that have had polymorphic ventricular tachycardia in the past, have a long QT interval, sick sinus syndrome, or a recent myocardial infarction, among others.

Dopamine receptor antagonists. A dopamine antagonist (antidopaminergic) is a type of drug that blocks dopamine receptors by receptor antagonism. Most antipsychotics are dopamine antagonists, and as such they have found use in treating schizophrenia, bipolar disorder, and stimulant psychosis. Several other dopamine antagonists are antiemetics used in the treatment of nausea and vomiting.

Droperidol is an antidopaminergic butyrophenone, used as an antiemetic and antipsychotic, and is a potent D2 (dopamine receptor) antagonist with some histamine and serotonin antagonist activity. There are concerns about QT prolongation and torsades de pointes. The evidence for this is disputed, with 9 reported cases of torsades in 30 years and all of those having received doses in excess of 5 mg. QT prolongation is a dose-related effect, and it appears that droperidol is not a significant risk in low doses, however, prolongation of QT interval leads to torsades de pointes.

Domperidone is a peripherally selective dopamine D2 receptor antagonist that is a drug useful in Parkinson's disease, caution is needed due to the cardiotoxic side effects of domperidone especially when given intravenously, in elderly people and in high doses (>30 mg per day). A clinical sign of domperidone's potential toxicity to the heart is the prolongation (lengthening) of the QT interval (a segment of the heart's electrical pattern). Domperidone use is associated with an increased risk of sudden cardiac death (by 70%) most likely through its prolonging effect of the cardiac QT interval and ventricular arrhythmias. The cause is thought to be blockade of hERG voltage-gated potassium channels. The risks are dose-dependent, and appear to be greatest with high/very high doses via intravenous administration and in the elderly, as well as with drugs that interact with domperidone and increase its circulating concentrations (namely CYP3A4 inhibitors). Conflicting reports exist, however. In neonates and infants, QT prolongation is controversial and uncertain.

Anticancer agents. Doxorubicin and anthracycline prolongation of QTc, increased QT dispersion and development of late potentials are indicative of doxorubicin-induced abnormal ventricular depolarization and repolarization. QT dispersion and late potentials are both known to be associated with increased risk of serious ventricular dysrhythmias and sudden death in various cardiac diseases.

Arsenic trioxide is an anti-leukemic can prolong the QTc interval. Cardiac Conduction Abnormalities: Before initiating therapy, perform a 12-lead ECG, assess serum electrolytes and creatinine, correct preexisting electrolyte abnormalities, and consider discontinuing drugs known to prolong QT interval. Arsenic trioxide can cause QT interval prolongation and complete atrioventricular block. QT prolongation can lead to a torsade de pointes-type ventricular arrhythmia, which can be fatal. The risk of torsade de pointes is related to the extent of QT prolongation, concomitant administration of QT prolonging drugs, a history of torsade de pointes, preexisting QT interval prolongation, congestive heart failure, administration of potassium-wasting diuretics, or other conditions that result in hypokalemia or hypomagnesemia. One patient (also receiving amphotericin B) had torsade de pointes during induction therapy for relapsed APL with arsenic trioxide. Arsenic trioxide (As₂O₃) used in the treatment of acute promyelocytic leukemia reduced hERG/IKr currents not by direct block, but by inhibiting the processing of hERG protein in the endoplasmic reticulum (ER) thereby decreasing surface expression of hERG.

Opioids. Levomethadyl is a levo isomer of α-methadyl acetatea synthetic opioid similar in structure to methadone. It has a long duration of action due to its active metabolites. In 2001, levacetylmethadol was removed from the European market due to reports of life-threatening ventricular rhythm disorders.

Methadone is an opioid used to treat pain and drug addiction. Serious risks include opioid abuse and heart arrhythmia may also occur including prolonged QT. The number of deaths in the United States involving methadone poisoning was 4,418 in 2011, which was 26% of total deaths from opioid poisoning.

Hypolipidemic agents. Lovostatin is a drug used for lowering cholesterol an inhibitor of 3-hydroxy-3-methylglutaryl-coenzyme A reductase (HMG-CoA reductase), an enzyme that catalyzes the conversion of HMG-CoA to mevalonate. Mevalonate is a required building block for cholesterol biosynthesis and lovastatin interferes with its production by acting as a reversible competitive inhibitor for HMG-CoA, which binds to the HMG-CoA reductase. QTc prolongation associated with antipsychotic medication occurs in a dose-dependent manner. The addition of lovastatin causes an increase in plasma quetiapine levels through competitive inhibition of the cytochrome P(450) (CYP) isoenzyme 3A4. This highlights the potential for a drug interaction between quetiapine and lovastatin leading to QTc prolongation during the management of dysipidemia in patients with schizophrenia.

Probucol is an anti-hyperlipidemic drug initially developed in the treatment of coronary artery disease. Probucol is associated with QT interval prolongation. Probucol aggravates long QT syndrome associated with a novel missense mutation M124T in the N-terminus of HERG.

Channelopathies. The human ether-a-go-go gene related cardiac tetrameric potassium channel, when mutated, can render patients sensitive to over 163 drugs, which inhibit ion conduction and deregulate action potentials. Prolongation of the action potential follows effects in the potassium channel. Ion channel active drugs may directly increase the QTc interval, and increase the risk of torsade de point and sudden cardiac death. Exacerbation of cardiomyocyte potassium channel sensitivity to drugs may also be associated with metabolic diseased states including diabetes or may be of idiopathic origin.

As used herein, the term “liposome” refers to a capsule wherein the wall or membrane thereof is formed of one or more of the novel lipids of the present invention. The lipids of the present invention can be used alone or in conjunction with other, known lipids. In one specific non-limiting example the novel lipids form, or are used in, liposomes that are empty liposomes and can be formulated from a single type of phospholipid or combinations of phospholipids. The empty liposomes can further includes one or more surface modifications, such as proteins, carbohydrates, glycolipids or glycoproteins, and even nucleic acids such as aptamers, thio-modified nucleic acids, protein nucleic acid mimics, protein mimics, stealthing agents, etc. In one embodiment, the novel liposome or novel liposome precursor comprising a novel lipid-monoglyceride-fatty acid eutectic, such as a eutectic that includes: lysophosphatidyl compound, a monoglyceride, and free fatty acid, and in certain aspects the ratios of the composition are 1:4:2, 1:3:3, 2:4:2, or 1:2:4 mole percent novel lipid:monoglyceride:free fatty acid. The composition may comprise a eutectic mixture comprising a novel lipid, a myristoyl monoglyceride, and a myristic acid. In one specific, non-limiting example the composition also comprises an active agent in or about the novel lipid liposome, which can be an empty liposome, and the composition has a ratio of phospholipids to active agent of 3:1, 1:1, 0.3:1, and 0.1:1.

Prior work from the some of the present inventors has demonstrated that formulation with a liposome containing 1,2-Dimyristoyl-sn-glycero-3-phosphorylcholine (DMPC), 1,2-Dimyristoyl-sn-glycero-3-phosphorylglycerol (DMPG), DMPC/DMPG, 1-Myristoyl-2-Hydroxy-sn-Glycero-3-Phosphocholine, 12-Mysteroyl-2-Hydroxy-sn-Glycero-3-[Phospho-rac-(glycerol)], 1-myristoyl-2-hydroxy-sn-glycero-3-phospho-(1′-rac-glycerol) (LysoPG), 12-Mysteroyl-2-Hydroxy-sn-Glycero-3-[Phospho-rac-(glycerol)], 1-myristoyl-2-hydroxy-sn-glycero-3-phospho-(1′-rac-glycerol) (LysoPG), or 1-myristoyl-2-hydroxy-sn-glycero-3-phosphocholine (LysoPC), lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine, prevented hERG channel inhibition by a variety of QT prolonging agents.

More than 20 QTc-prolonging drugs have had their QTc prolongation eliminated in various regulatory-validated preclinical models using the above lipids.

The novel lipids of the present invention may be manufactured in a native form, or in the form of a salt, hydrate, or solvate thereof salt. Salts further include, by way of example only, lithium, sodium, potassium, calcium, magnesium, ammonium, tetraalkylammonium, and the like.

### EXAMPLES

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

While certain reactions are represented in the claims below, it is to be understood that variations in final results are to be expected as the following examples are based on laboratory-scale reactions and not on manufacturing processes. Additionally, it is to be understood that in many cases the isolated product quantities are quantities isolated from multiple repeat-batches of said reactions and not from single reactions.

### Example 1. Effect of Multiple Lipids

The present invention demonstrates an enhanced effect in reducing, or eliminating, QT prolongation in a guinea pig model system. The guinea pig model system used herein is the closes model system to the functioning of the human heart and is well-accepted for testing of QT prolonging agents. Briefly, guinea pigs were instrumented with ECG leads, and administered increasing oral doses of moxifloxacin. Guinea pigs are the preferred species in Europe and Canada for QT prolongation testing, because they possess a complement of cardiac ion channels most similar to that of humans, and are exquisitely sensitive to proarrhythmic drugs. On the drug side, Moxifloxacin is the preferred QTc-prolonging positive control drug in Thorough QT (TQT) clinical studies because it causes a dose-dependent QTc prolongation in all species, and exhibits very linear pharmacokinetics, making it easy to dose and relatively safe at sub-toxic exposure levels.

Those guinea pigs administered only moxifloxacin exhibited severe (+10 ms) and life-threatening (+30 ms) QTc prolongation. In contrast, those animals that had received a concomitant dose of, as an example, 14:0 lyso PG, exhibited no, or very little, changes in QTc. There resulted a statistically significant right-shift in the QTc-dose response of Moxifloxacin, actually preventing the QTc prolongation from becoming dose-limiting.

AV block represents an interesting conundrum in cardiology: drug-induced AV block patients are not treated by pacemaker implantation, unlike patients suffering from disease-induced AV block. Yet, there is evidence that drug-induced AV block is irreversible after drug discontinuation in 56% of cases (Zeltser D, Justo D, Halkin A, et al. Drug-induced atrioventricular block: prognosis after discontinuation of the culprit drug. J Am Coll Cardiol. 2004; 44(1):105-108). Current practice is to immediately discontinue AV blocking drugs upon discovering the effect. This withdraws useful, efficient drugs from the pharmacopeia available to oncologists, while directly impacting drug adoption in the clinic.

The ionic channels involved in AV block and QTc prolongation are completely distinct: sodium (Na⁺) and calcium (Ca²⁺) channel inhibition are responsible for the onset of AV block, while delays in repolarization due to potassium (K⁺) inhibition lead to QTc prolongation. Yet, the hypothesized mechanism by which lipids rescues K⁺ currents could also benefit Na⁺ and Ca²⁺ currents.

To test this hypothesis, guinea pigs were instrumented (subcutaneous ECG leads) and exposed to increasing intravenous doses of Fingolimod and/or verapamil, without and with an oral dose of 14:0 lyso PG. ECG signals were recorded continuously for 2 hours post-dose for the AV blockers Fingolimod and verapamil. PR intervals were measured following the infusion of Fingolimod. Measurements of PR were stopped when the P wave disconnected from the QRS complexes, indicating 3rd degree AV block.

Guinea pigs exposed to an intravenous infusion of Fingolimod alone transitioned to 1st degree AV block as of a dose of 15 μg/kg, which rapidly progressed to a Mobitz Type-1, 2nd-degree AV block at 20 μg/kg, and finally progressed to 3rd degree AV block as of a dose of 23 μg/kg. The progression of the AV block was rapid and irreversible: stopping infusion did not prevent the onset of P-QRS dissociation.

The cohort of guinea pigs exposed to verapamil received an i.v. injection of 0.5 mg/kg, followed 60 minutes later by an intravenous infusion of Fingolimod. A 1st-degree AV block appeared at a dose of 7 μg/kg, changed to a Mobitz-Type-1 2nd degree AV block at 10 μg/kg, and transitioned to 3rd-degree dissociation between P waves and QRS complexes as of 45 μg/kg.

The third cohort of animals received an initial oral gavage of 1.0 mg/kg 14:0 lyso PG, followed 60 minutes later by an intravenous dose of 0.5 mg/kg verapamil. Sixty (60) minutes post-verapamil, Fingolimod was infused into the animals as described above. The animals exhibited modest changes in PR intervals up to a dose of 200 μ/kg, at which point a Is-degree AV block appeared. A Mobitz-Type-2 AV block appeared in 2 out of 6 animals with P-QRS dissociation observed at a dose of 51 μ/kg in those two animals, and at 300 g/kg in the rest of the animals in the cohort.

In human patients, Fingolimod is counter-indicated in patients presenting a history of Mobitz Type II second-degree or third-degree AV block or sick sinus syndrome. The drug has been shown to produce AV block from the first dose, and avoiding treatment with Fingolimod and AV blockers is recommended (Fingolimod (Fingolimod) Full Prescribing Information. Novartis: T2016-22, February 2016). Given the history of translatability of the guinea pig cardiovascular data to other species, including man, these results suggest that 14:0 lyso PG could alleviate the risk of AV block associated with Fingolimod use, thus enhancing the safety profile of the drug, and allowing the treatment of patients, which cannot otherwise receive Fingolimod due to AV block issues.

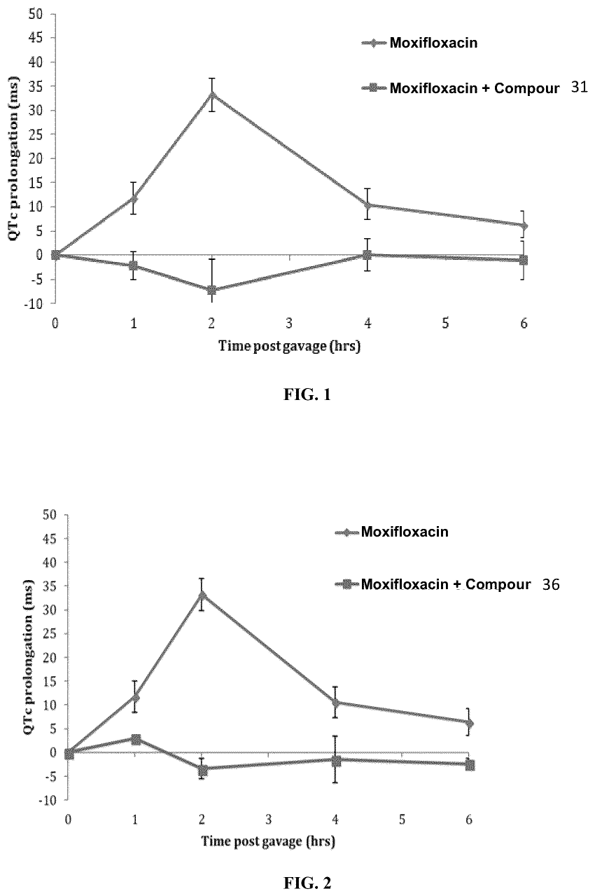

FIG. 1 is a graph that shows the effect of an oral single dose of Moxifloxacin (20 mg/kg) on QTc interval of guinea pigs compared to the same oral single dose of Moxifloxacin administrated concomitantly with an oral single dose of Compound 31.

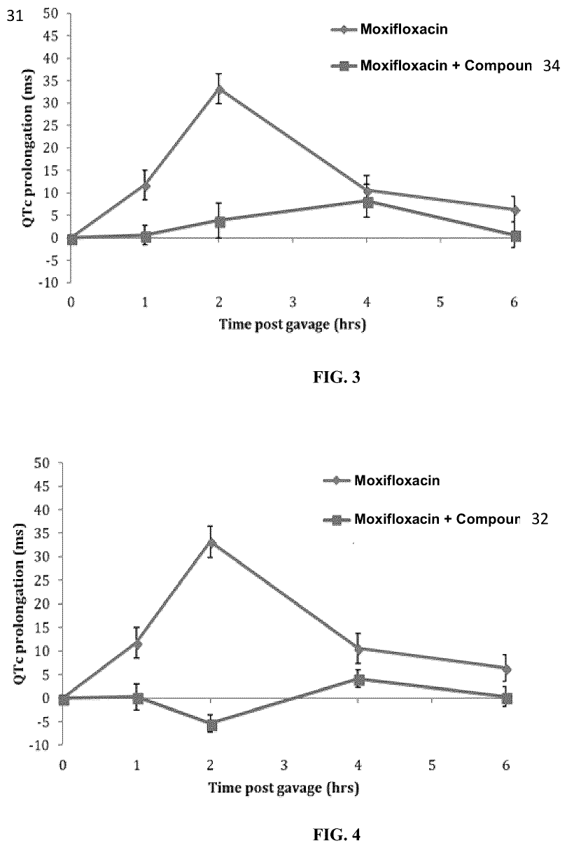

FIG. 2 is a graph that shows the effect of an oral single dose of Moxifloxacin (20 mg/kg) on QTc interval of guinea pigs compared to the same oral single dose of Moxifloxacin administrated concomitantly with an oral single dose of Compound 36.

FIG. 3 is a graph that shows the effect of an oral single dose of Moxifloxacin (20 mg/kg) on QTc interval of guinea pigs compared to the same oral single dose of Moxifloxacin administrated concomitantly with an oral single dose of Compound 4.

FIG. 4 is a graph that shows the effect of an oral single dose of Moxifloxacin (20 mg/kg) on QTc interval of guinea pigs compared to the same oral single dose of Moxifloxacin administrated concomitantly with an oral single dose of Compound 32.

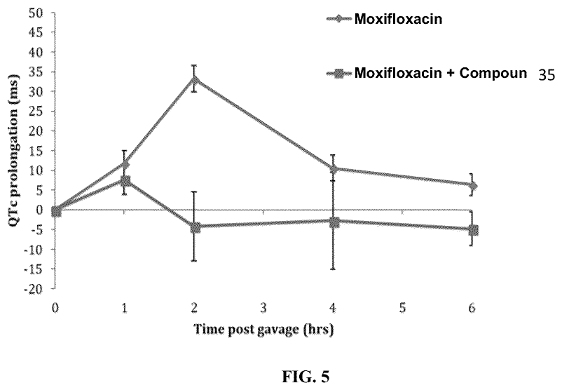

FIG. 5 is a graph that shows the effect of an oral single dose of Moxifloxacin (20 mg/kg) on QTc interval of guinea pigs compared to the same oral single dose of Moxifloxacin administrated concomitantly with an oral single dose of Compound 35.

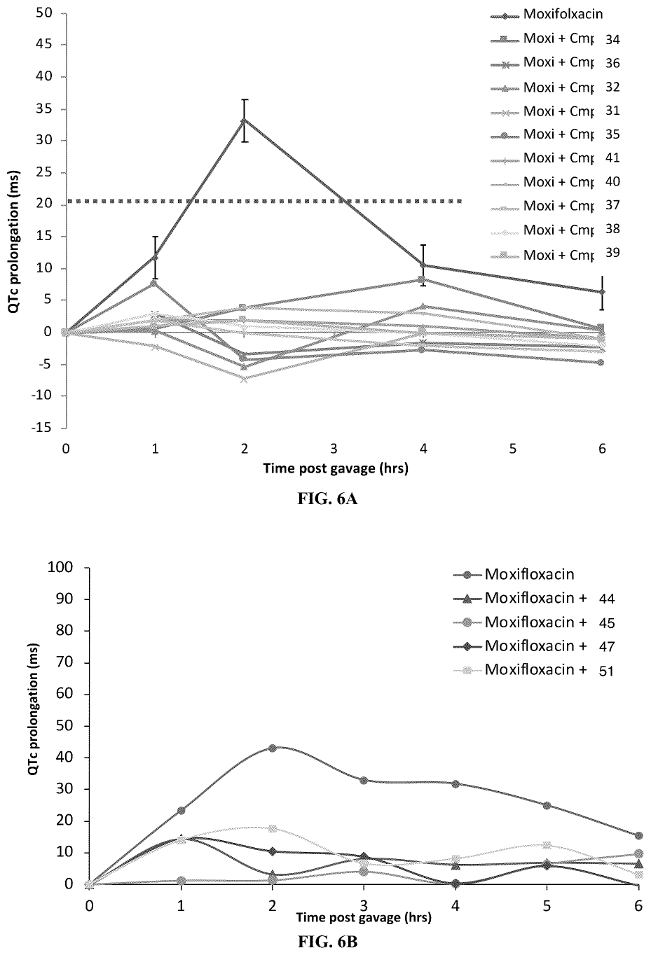

FIG. 6A and FIG. 6B are composite graphs that shows the effect of an oral single dose of Moxifloxacin (20 mg/kg) on QTc interval of guinea pigs compared to the same oral single dose of Moxifloxacin administrated concomitantly with an oral single dose of Compound 40, Compound 41, Compound 32, Compound 34, Compound 35, Compound 36, Compound 37, Compound 38, Compound 39, Compound 40, Compound 41, Compound 44, Compound 45, Compound 47 and Compound 51

### Example 2. Cardioprotection Against Cardiotoxic Agents

The highly active chemotherapeutic agent doxorubicin has been associated with acute but reversible cardiotoxic effects and a longer-term, dose-related cardiomyopathy. This cardiomyopathy is characterized by minimal left ventricular dilation and overall contractile dysfunction, often concurrent with moderate valve regurgitation (Keefe, 2001). More than one-half of all patients exposed to doxorubicin will present cardiac dysfunction within 10 to 20 years following chemotherapy, and 5% of them will develop overt HF (Cardinale, 2010). The incidence of cardiomyopathy in doxorubicin-treated patients is such that it is now used as an adjuvant, in combination with safer, albeit often less effective, therapies. One such therapy is HERCEPTIN® (Trastuzumab), a blockbuster humanized monoclonal antibody targeting the extracellular domain of HER2 in patients with breast cancer. However, Herceptin treatment is also hampered by cardiac toxicity, in this case a decline in left ventricular ejection fraction (LVEF) with a reported incidence up to 27% (Bouwer, 2020).

The combination of doxorubicin and herceptin has been shown in clinical studies to improve overall survival by 24-33% in breast cancer patients (Romond, 2005). Since doxorubicin-induced, and herceptin-induced cardiomyopathies are largely irreversible and cumulative, finding ways to minimize the cardiac side effects of the combination is a key strategy if these very potent oncology tools are to remain in clinical use.

Vitamin D has been reported to offer some protection against anthracyclines, as has the iron chelator, dexrazoxane (Lee, 2021). Although the mechanism of protection is not fully understood, it could involve prevention of oxidation within biological membranes. In this ongoing research project, rats and mice were used to evaluate the protection offered by an anti-inflammatory complex lipid (SPP05) which integrates preferentially into cardiomyocyte membranes.

All experimentation was conducted in accordance with the guidelines on laboratory animal use of the Canadian Council against Animal Cruelty (CCAC) and IPST's IACUC. IPST is AAALAC accredited.

Test system and treatment: Adult female C57/BL6 mice weighing 25 grams (n=10/group) at time of study start were administered 24 mg/kg DOX ip over 2 weeks. Following a 1-week break, 10 mg/kg HER ip were administered over 2 weeks. In parallel, an anti-inflammatory lipid (SPP05) was administered from Day −10 at 10 mg/kg/day for one group of animals and at 50 mg/kg for another, throughout the project.

Experimental endpoints. Body weights were measured weekly.

Cardiac echography was conducted on Day −10, on Days, 14 and 42, and 49 using a Vivid 9 instrument and linear 13 MHz probe. Blood draws were performed on Days −7, 0, 21, and 49. Invasive hemodynamics were measured by cannulating the left ventricle using a fluid-filled PE15 catheter, connected to a Millar pressure transducer.

Troponin I, NT-Pro-BNP, and caspase-3 activity were measured by Q-ELISA.

Histological examination of the heart and liver were performed following fixation in 10% NBF, histology and staining with H&E and Picro Sirius Red.

Doxorubicin combined with Herceptin was considered the standard of care for HER-2-expressing breast cancer patients until the incidence of drug-induced heart failure prompted a shift in clinical approaches. In this mouse model, Doxo+Here animals exhibited LV contractile dysfunction as evidenced by losses in ejection fraction, stroke volume, fractional shortening and pulse pressure. Furthermore, Doxo+Here animals also exhibited LV tissue loss overall and at end systole and end-diastole. Finally, the biomarker of HF NT-Pro-BNP levels were greater in Doxo+Here than in healthy (Sham) animals.

Treating the animals with compound 35, a complex anti-inflammatory lipid with known membrane altering properties partially prevented the damage caused by the oncology treatment in the female mice. Given the mechanism of toxicity for Doxo+Here, it is hypothesized that prevention of sphingomyelinases activation by reactive oxygen species is involved in the protection afforded by compound 35.

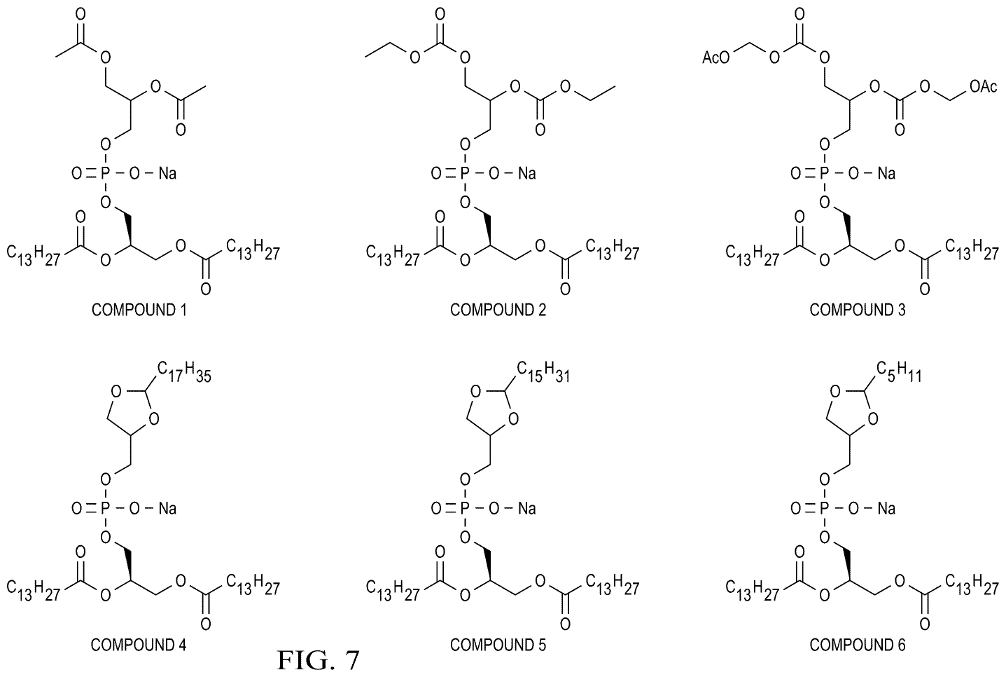

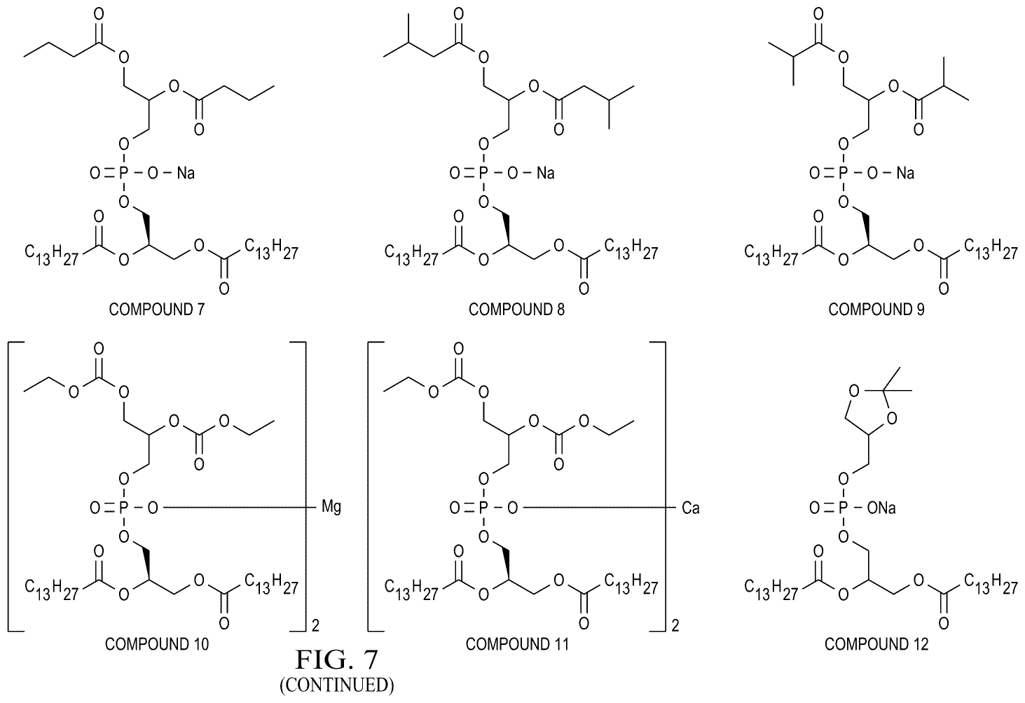

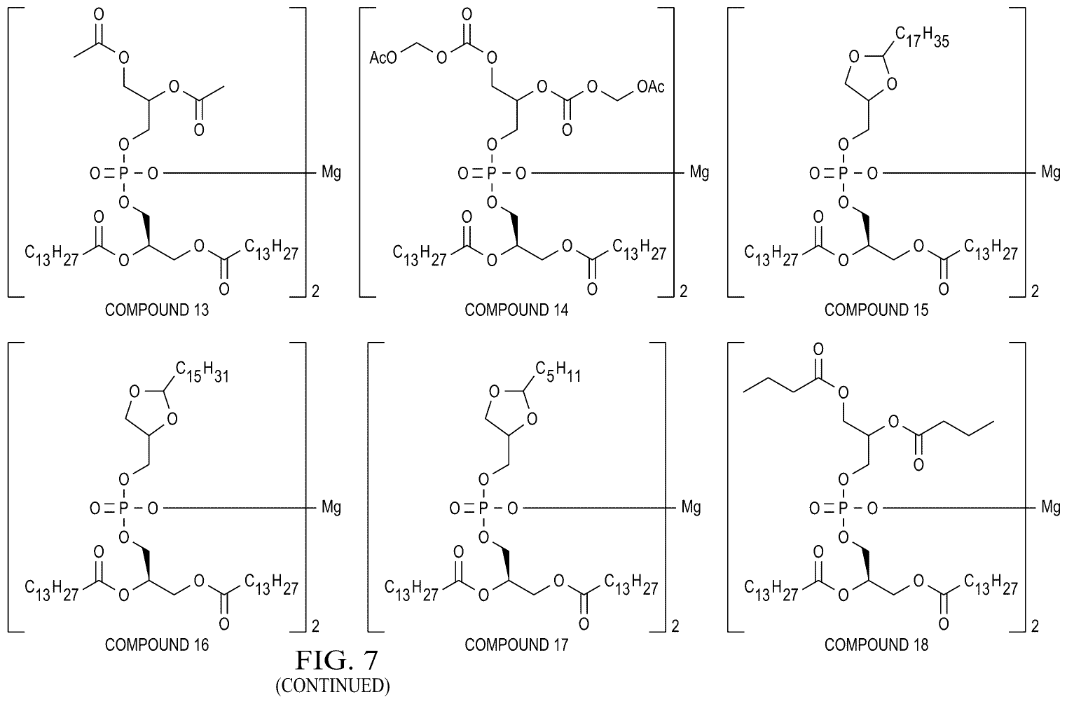

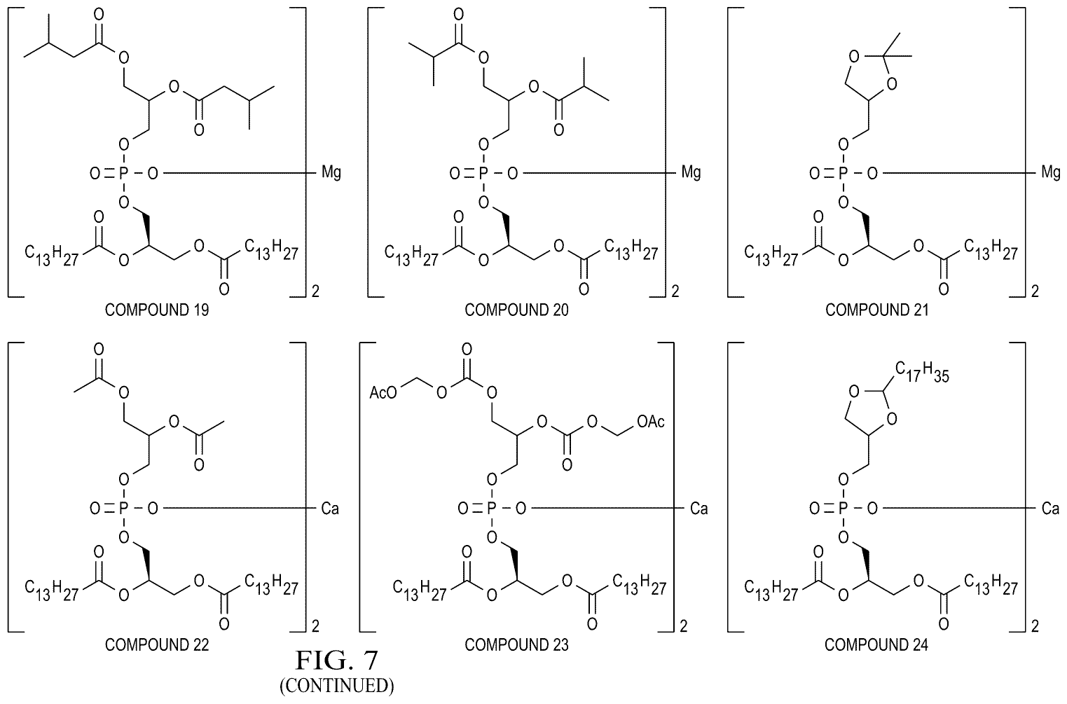

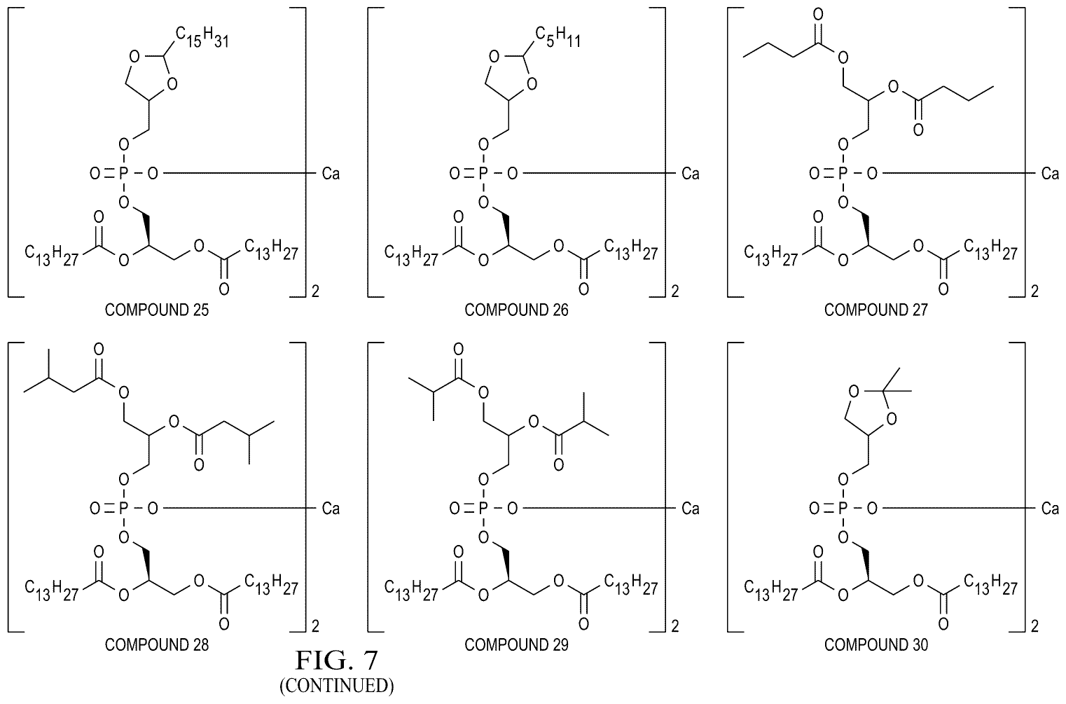

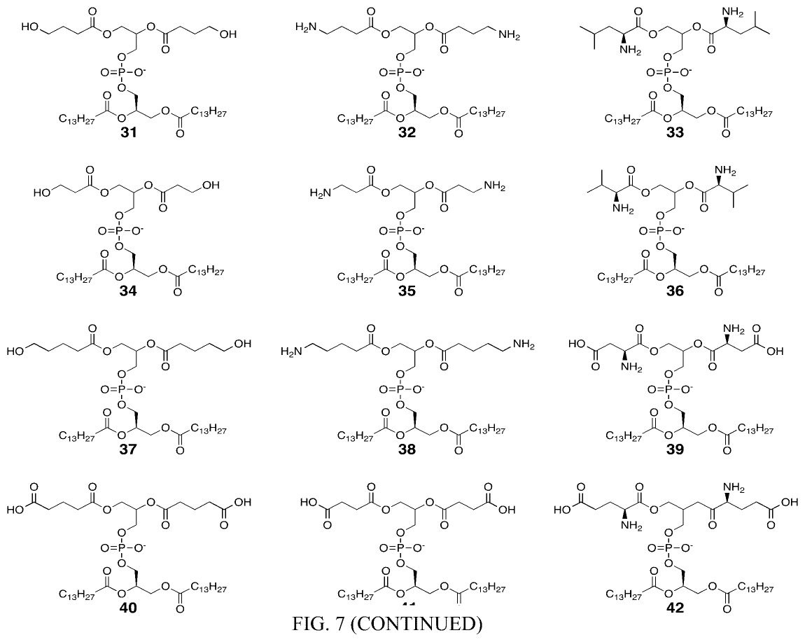

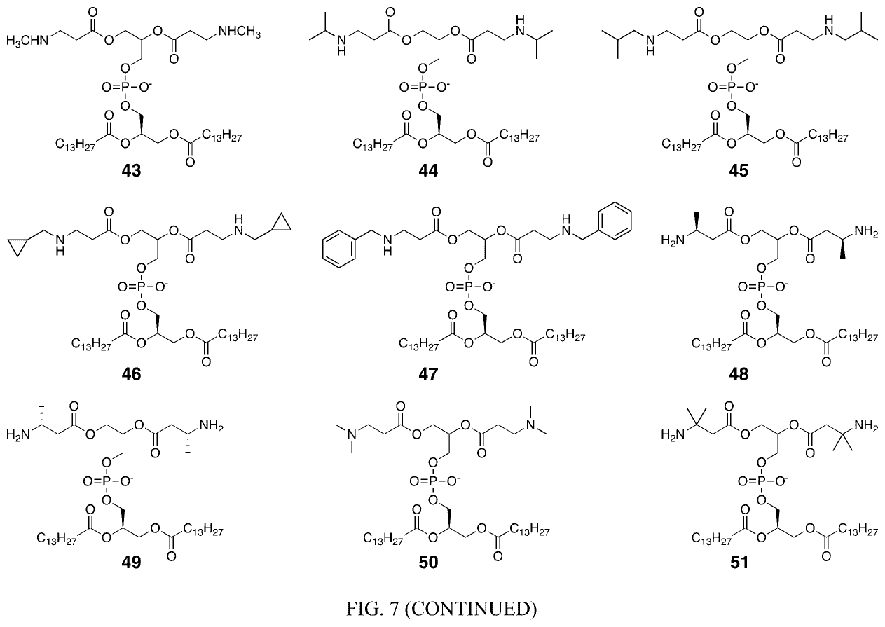

FIGS. 7A and 7C are depictions of example chemical structures which are embodiments of the present invention.

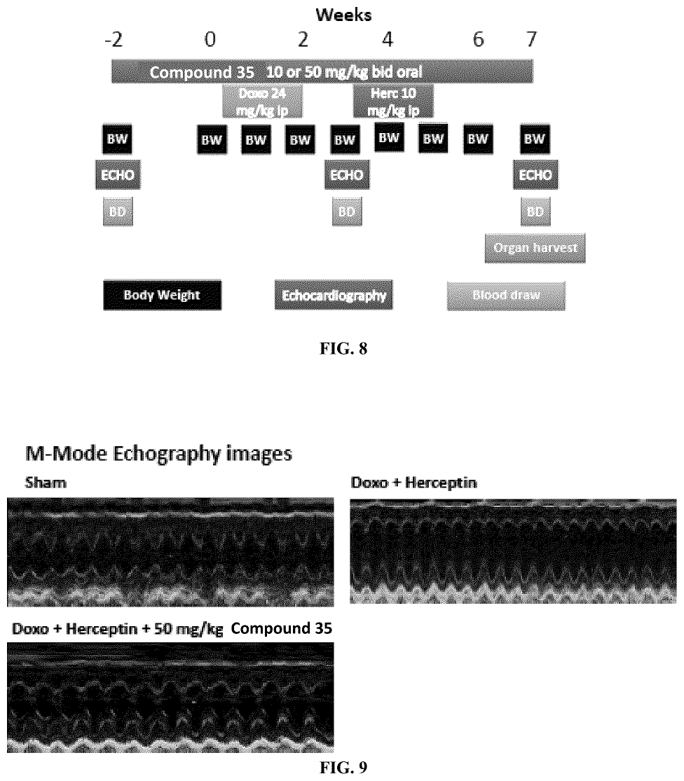

FIG. 8 shows the 9-week study outline for determining the cardioprotective effect of the lipids.

FIG. 9 shows M-Mode echography images comparing the effects of a sham treatment, treatment with doxorubicin and Trastuzumab, and doxorubicin and Trastuzumab+ lipid compound 35.

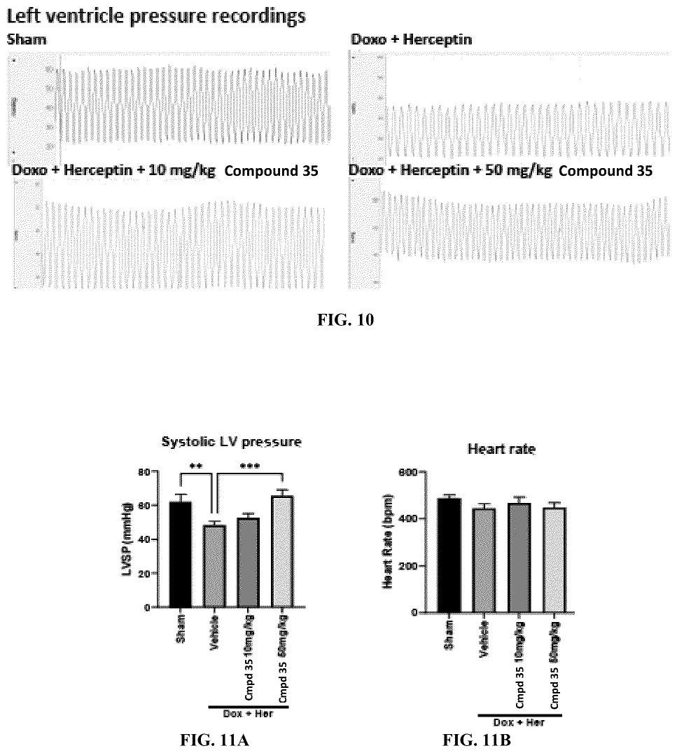

FIG. 10 shows left ventricular pressure recordings comparing a sham treatment, treatment with doxorubicin and Trastuzumab, and doxorubicin and Trastuzumab+ lipid compound 35 at two concentrations, 10 mg·kg and 50 mg/kg.

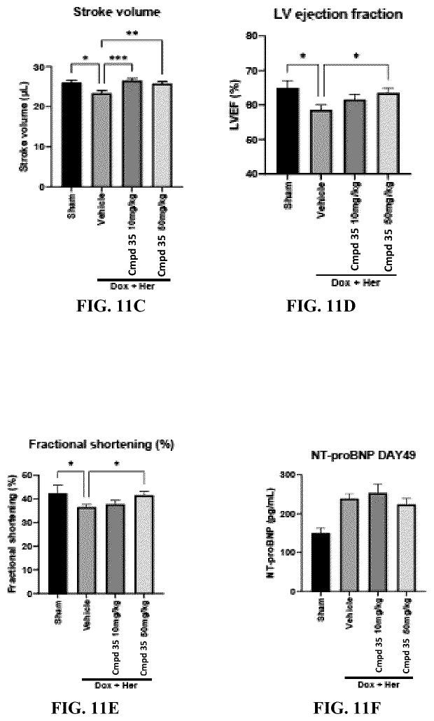

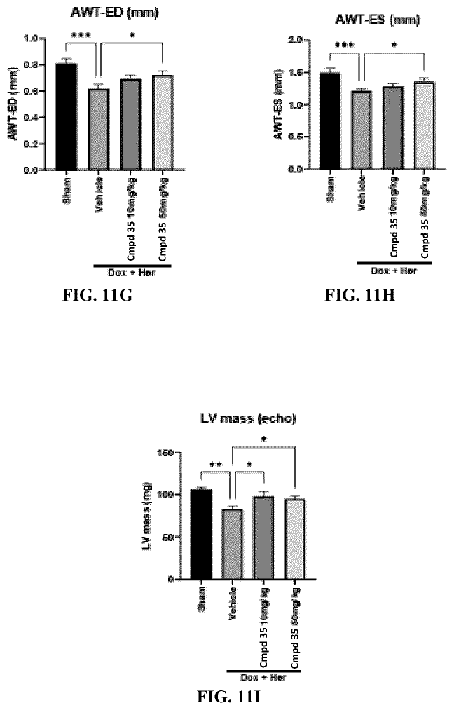

FIGS. 11A to 11H are graphs that show the systolic left ventricular pressure (FIG. 11A), heart rate (FIG. 11B), stroke volume (FIG. 11C), left ventricular ejection fraction (FIG. 11D), percent fractional shortening (FIG. 11E), N-terminal (NT)-pro hormone BNP (NT-proBNP) at day 49 (FIG. 11F), anterior wall thickness end-diastole (AWT-ED) (mm) (FIG. 11G), anterior wall thickness end-systole (AWT-ES) mm (FIG. 11H), and left ventricular mass (echo) (FIG. 11I).

In at least some embodiments of the present invention, compounds of Formula I or Formula IA are prepared according to the following schemes. For reference, all variable groups included in the following schemes relate to the corresponding variables defined generally above. One of ordinary skill in the art will recognize that alternative reagents and reactants can be used to generate the same target compounds and intermediates.

As illustrated in Scheme 1, compounds of Formula V are reacted with anhydrides followed by subsequent salt formation giving compounds of Formula VI. One of ordinary skill in the art will recognize that alternatives to anhydrides will produce similar results. Such alternatives include, but are not limited to, acid chlorides, acyl imidazoles, acyl succinimides and the like. Additionally, one of ordinary skill in the art will recognize that carboxylic acids in the presence of activating agents will produce similar results. Suitable activating agents include, but are not limited to, DCC, EDC, HBTU, BOP, PyBOP, carbonyl diimidazole, disuccinimidyl carbonate and the like. One of ordinary skill in the art will recognize that alternatives to DOWEX Na⁺ resin for salt formation are useful. Such alternatives include, but are not limited to, sodium bicarbonate, sodium carbonate and the like. One of ordinary skill in the art will recognize that the “Optional Deprotection” step is required when R⁵ and/or R⁶ contain a functional group bearing a protecting group. One of ordinary skill in the art will understand that protecting groups are chemical appendages that prevent reactivity with a given functional group until said protecting group is removed or cleaved. One of ordinary skill in the art will understand that protecting groups include, but are not limited to, Ac, tert-Butyl, Benzyl, Trityl, Benzoyl, para-nitrobenzyol, MOM, BOM, Si comprising the core of a silyl ether, Boc, Cbz, Fmoc, 4-Methoxybenzyl and an ester form of a carboxylic acid wherein said ester comprises a C₁-C₆ branched or unbranched alkyl group. One of ordinary skill in the art will recognize that compounds of Formula VI include compounds 1, 7, 8, 9, 31, 32, 33, 34, 35, 36, 37, 38, 39, 40, 41, 42, 43, 44, 45, 46, 47, 48, 49, 50 and 51.

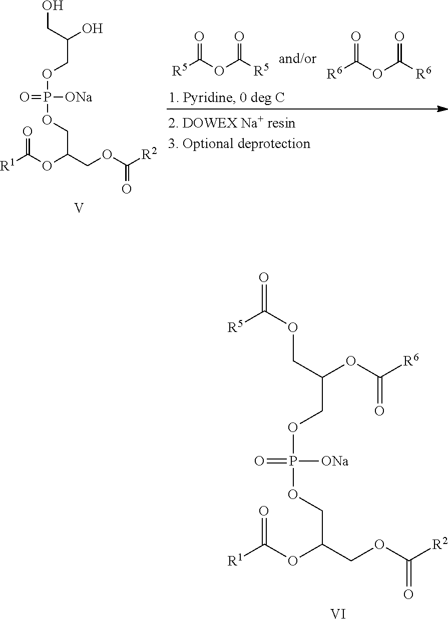

As illustrated in Scheme 2, compounds of Formula V are reacted with chloroformates followed by subsequent salt formation giving compounds of Formula VII. One of ordinary skill in the art will recognize that alternatives to chloroformates will produce similar results. Such alternatives include, but are not limited to, pyrocarbonates and the like. One of ordinary skill in the art will recognize that bases other than triethylamine are useful in carbonate formation reactions. Such bases include, but are not limited to, triisopropylamine, diisopropylethylamine, DBU, N-methylmorpholine, N-methylpyridine, N,N-dimethylpiperazine and the like. One of ordinary skill in the art will recognize that acyl transfer catalysts other than DMAP are useful in carbonate formation reactions. Such acyl transfer catalysts include, but are not limited to, pyridine, 2-methylpyridine and the like. Additionally, one of ordinary skill in the art will recognize that introduction of Lewis acid catalysts may facilitate carbonate formation. Such Lewis acid catalysts include, but are not limited to, zinc chloride, zinc acetate, zinc bromide, aluminum trichloride, titanium trichloride, titanium isopropoxide, boron trifluoride, tin chloride, alumina, silica gel and the like. One of ordinary skill in the art will recognize that alternatives to sodium bicarbonate for salt formation are useful. Such alternatives include, but are not limited to, DOWEX Na⁺ resin, sodium carbonate and the like. One of ordinary skill in the art will recognize that compounds of Formula VII include compounds 2 and 3.

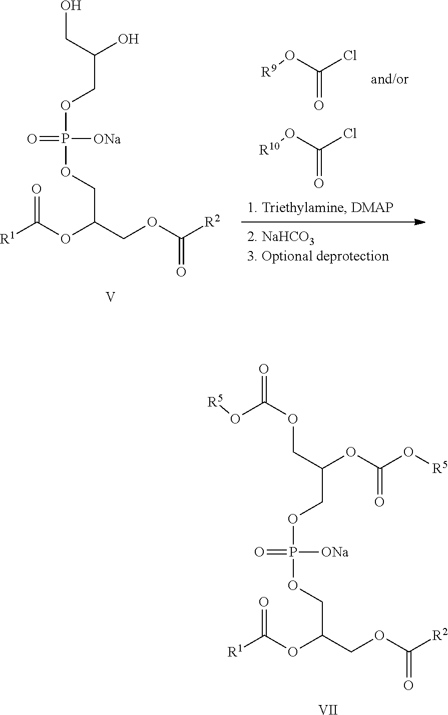

As illustrated in Scheme 3, compounds of Formula V are reacted with isocyanates followed by subsequent salt formation giving compounds of Formula VIII. One of ordinary skill in the art will recognize that alternatives to isocyanates will produce similar results. One of ordinary skill in the art will recognize that bases can facilitate carbamate formation. Such bases include, but are not limited to, triethylamine, triisopropylamine, diisopropylethylamine, DBU, N-methylmorpholine, N-methylpyridine, N,N-dimethylpiperazine and the like. One of ordinary skill in the art will recognize that acyl transfer catalysts can facilitate carbamate formation. Such acyl transfer catalysts include, but are not limited to, DMAP, pyridine, 2-methylpyridine and the like. Additionally, one of ordinary skill in the art will recognize that introduction of Lewis acid catalysts may facilitate carbamate formation. Such Lewis acid catalysts include, but are not limited to, zinc chloride, zinc acetate, zinc bromide, aluminum trichloride, titanium trichloride, titanium isopropoxide, boron trifluoride, tin chloride, alumina, silica gel and the like. One of ordinary skill in the art will recognize that alternatives to sodium bicarbonate for salt formation are useful. Such alternatives include, but are not limited to, DOWEX Na⁺ resin, sodium carbonate and the like.

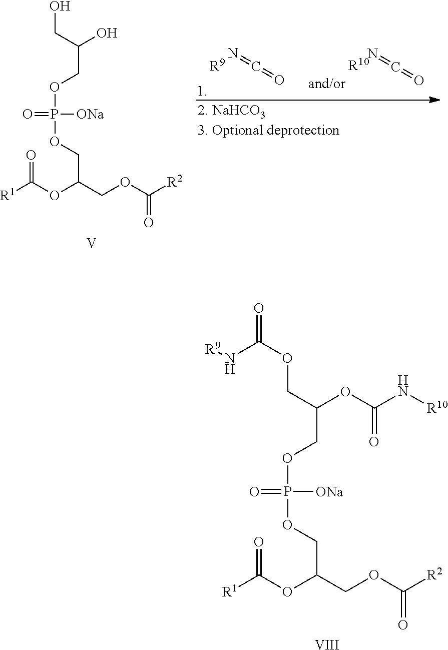

As illustrated in Scheme 4, compounds of Formula IX are reacted with carboxylic acids, acid halides, anhydrides, chloroformates or isocyanates followed by subsequent cleavage of the benzyl ether protecting group giving compounds of Formula X. Coupling of compounds of Formula X with compounds of Formula XI using Phospholipase D generates compounds of Formula XII. One of ordinary skill in the art will recognize that for preparation of compounds of Formula X alternatives to carboxylic acids, acid halides and anhydrides will produce similar results. Such alternatives include, but are not limited to, acyl imidazoles, acyl succinimides and the like. Additionally, one of ordinary skill in the art will recognize that, for preparation of compounds of Formula X, reaction with carboxylic acids requires activating agents. Suitable activating agents include, but are not limited to, DCC, EDC, HBTU, BOP, PyBOP, carbonyl diimidazole, disuccinimidyl carbonate and the like. One of ordinary skill in the art will recognize that, for preparation of compounds of Formula X, alternatives to chloroformates will produce similar results. Such alternatives include, but are not limited to, pyrocarbonates and the like. One of ordinary skill in the art will recognize that, for preparation of compounds of Formula X, alternatives to isocyanates will produce similar results. One of ordinary skill in the art will recognize that bases can facilitate ester, carbonate and carbamate formation. Such bases include, but are not limited to, triethylamine, triisopropylamine, diisopropylethylamine, DBU, N-methylmorpholine, N-methylpyridine, N,N-dimethylpiperazine and the like. One of ordinary skill in the art will recognize that acyl transfer catalysts can facilitate ester, carbonate and carbamate formation. Such acyl transfer catalysts include, but are not limited to, DMAP, pyridine, 2-methylpyridine and the like. Additionally, one of ordinary skill in the art will recognize that introduction of Lewis acid catalysts may facilitate ester, carbonate and carbamate formation. Such Lewis acid catalysts include, but are not limited to, zinc chloride, zinc acetate, zinc bromide, aluminum trichloride, titanium trichloride, titanium isopropoxide, boron trifluoride, tin chloride, alumina, silica gel and the like. One of ordinary skill in the art will recognize that alternatives to the benzyl ether protecting group, and associated reaction conditions for their cleavage, are useful. Various appropriate alcohol protecting groups are broadly described in “Green's Protective Groups in Organic Synthesis”. Such benzyl ether alternatives include, but are not limited to, trimethylsilyl ethers, tert-butyl dimethylsilyl ethers, triisopropylsilyl ethers, tert-butyl diphenylsilyl ethers, acetates, benzoates, 4-nitrobenzoates, tert-butyl ethers, 4-methoxybenzyl ethers and the like. One of ordinary skill in the art will recognize that alternative enzyme and alternate enzyme reaction conditions are useful in the enzymatic formation of phospho diesters. One of ordinary skill in the art will recognize that, for Schemes 1-4, R⁵ and R⁶ may be replaced with R⁷ and R⁸, respectively. Furthermore, one of ordinary skill in the art will understand that, when R⁵ and R⁶ are replaced with R⁷ and R⁸, cleavage of protecting groups may be required. One of ordinary skill in the art will recognize that suitable protecting groups associated with R⁷ and R⁸ are dependent upon the functional groups for which said protecting groups may be required. Such protecting groups may be selected from the group consisting of but not limited to Ac, C₁-C₆ branched or unbranched alkyl, tert-Butyl, Benzyl, 4-Methoxybenzyl, Trityl, Benzoyl, para-nitrobenzoyl, MOM, BOM, Boc, Cbz, Fmoc or Si comprising the core of a silyl ether. Various appropriate protecting groups are broadly described in “Green's Protective Groups in Organic Synthesis”. One of ordinary skill in the art will recognize that compounds of Formula XII include compounds 1, 2, 3, 7, 8, 9, 31, 32, 33, 34, 35, 36, 37, 38, 39, 40, 4142, 43, 44, 45, 46, 47, 48, 49, 50 and 51.

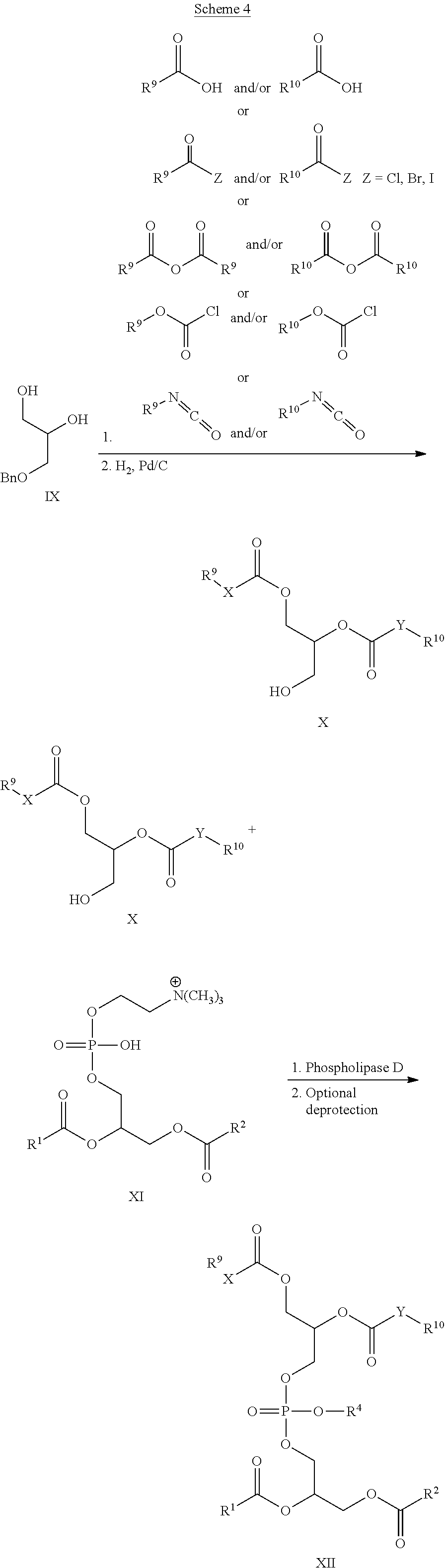

Scheme 5, Scheme 6, Scheme 7 and Scheme 8 collectively illustrate preparation of compounds of Formula XXVII. As illustrated in Scheme 7, a compound of Formula XIII is converted to a benzyl ether giving a compound of Formula XIV. The ketal of a compound of Formula XIV is then cleaved giving a compound of Formula XV. On reaction with one or more carboxylic acids and an appropriate activating reagent, a compound of Formula XV is converted to a compound of Formula XVI. Subsequent cleavage of the benzyl ether of a compound of Formula XVI gives a compound of Formula XVII. One of ordinary skill in the art will recognize that alternate reagents and reaction conditions are useful for formation of a benzyl ether. One of ordinary skill in the art will also recognize that alternatives to the benzyl ether protecting group, and associated reaction conditions for their formation, are useful. Various appropriate alcohol protecting groups are broadly described in “Green's Protective Groups in Organic Synthesis”. Such benzyl ether alternatives include, but are not limited to, trimethylsilyl ethers, tert-butyl dimethylsilyl ethers, triisopropylsilyl ethers, tert-butyl diphenylsilyl ethers, acetates, benzoates, 4-nitrobenzoates, tert-butyl ethers, 4-methoxybenzyl ethers and the like. Similarly, one of ordinary skill in the art will recognize that alternative reaction conditions are useful for the cleavage of acetals and ketals. Such conditions are generally described in Green's “PROTECTIVE GROUPS IN ORGANIC SYNTHESIS”, Wiley, Online Edition. One of ordinary skill in the art will recognize that carboxylic acids and associated activating agents are useful for the formation of esters. One of ordinary skill in the art will recognize that DCC is an appropriate activating agent for coupling of alcohols and carboxylic acids to form esters. One of ordinary skill in the art will recognize that alternate activating agents are also useful for the coupling of alcohols and carboxylic acids to form esters. Such alternate activating agents include, but are not limited to, EDC, HBTU, BOP, PyBOP, carbonyl diimidazole, disuccinimidyl carbonate and the like. One of ordinary skill in the art will further recognize that alternatives to carboxylic acids with activating agents are useful for the formation of esters from alcohols. Such alternatives include functional reagents that include, and are not limited to, anhydrides, acid chlorides, acyl imidazoles, acyl succinimides and the like. One of ordinary skill in the art will recognize that alternatives to the benzyl ether protecting group, and associated reaction conditions for their cleavage, are useful. Various appropriate alcohol protecting groups are broadly described in “Green's PROTECTIVE GROUPS IN ORGANIC SYNTHESIS”. Such benzyl ether alternatives include, but are not limited to, trimethylsilyl ethers, tert-butyl dimethylsilyl ethers, triisopropylsilyl ethers, tert-butyl diphenylsilyl ethers, acetates, benzoates, 4-nitrobenzoates, tert-butyl ethers, 4-methoxybenzyl ethers and the like.

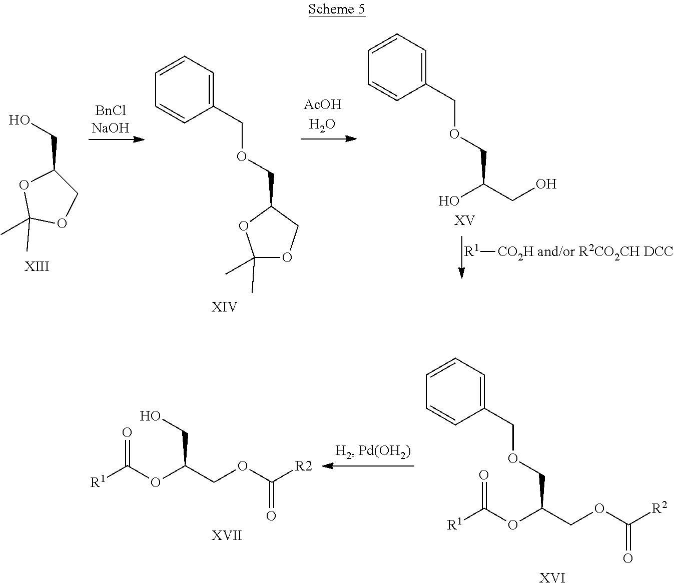

As illustrated in Scheme 8, a compound of Formula XVIII is converted to a benzyl ether giving a compound of Formula XIX. The ketal of a compound of Formula XIX is then cleaved giving a compound of Formula IX. One of ordinary skill in the art will recognize that alternate reagents and reaction conditions are useful for formation of a benzyl ether. One of ordinary skill in the art will also recognize that alternatives to the benzyl ether protecting group, and associated reaction conditions for their formation, are useful. Various appropriate alcohol protecting groups are broadly described in “Green's Protective Groups in Organic Synthesis”. Such benzyl ether alternatives include, but are not limited to, trimethylsilyl ethers, tert-butyl dimethylsilyl ethers, triisopropylsilyl ethers, tert-butyl diphenylsilyl ethers, acetates, benzoates, 4-nitrobenzoates, tert-butyl ethers, 4-methoxybenzyl ethers and the like. Similarly, one of ordinary skill in the art will recognize that alternative reaction conditions are useful for the cleavage of acetals and ketals. Such conditions are generally described in “Green's Protective Groups in Organic Synthesis”.

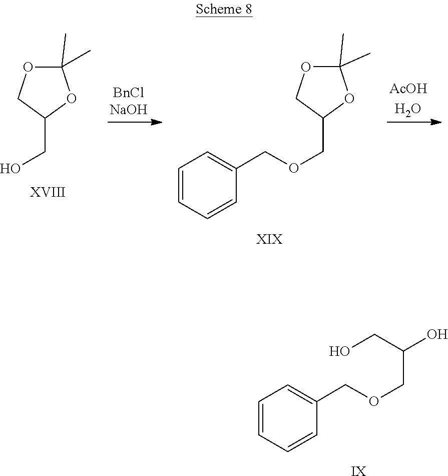

As illustrated in Scheme 9, a compound of Formula IX is converted to a compound of Formula XX. A compound of Formula XX is a bis-carbonate, a bis-ester or a bis-carbamate. On hydrogenation, the benzyl ether of compound XX is cleaved giving a compound of Formula XXI.

One of ordinary skill in the art will recognize that a bis-carbonate version of a compound of Formula XX can be prepared by reacting a compound of Formula IX with functional reagents that include, but are not limited to, chloroformates, pyrocarbonates and the like. One of ordinary skill in the art will recognize that carbonate formation can include use of bases such as, but not limited to, triethylamine, triisopropylamine, diisopropylethylamine, DBU, N-methylmorpholine, N-methylpyridine, N,N-dimethylpiperazine and the like. One of ordinary skill in the art will recognize that carbonate formation can include use of acyl transfer catalysts such as, but not limited to, DMAP, pyridine, 2-methylpyridine and the like. One of ordinary skill in the art will recognize that carbonate formation can include use of Lewis acid catalysts such as, but not limited to, zinc chloride, zinc acetate, zinc bromide, aluminum trichloride, titanium trichloride, titanium isopropoxide, boron trifluoride, tin chloride, alumina, silica gel and the like.

One of ordinary skill in the art will recognize that a bis-acetate version of a compound of Formula XX can be prepared by reacting a compound of Formula IX with functional reagents that include, but are not limited to, anhydrides, acid chlorides, acyl imidazoles, acyl succinimides and the like. Additionally, one of ordinary skill in the art will recognize that carboxylic acids in the presence of activating agents will produce similar results. Suitable activating agents include, but are not limited to, DCC, EDC, HBTU, BOP, PyBOP, carbonyl diimidazole, disuccinimidyl carbonate and the like.

One of ordinary skill in the art will recognize that a bis-carbamate version of a compound of Formula XX can be prepared by reacting a compound of Formula IX with functional reagents that include, but are not limited to, isocyanates and the like. One of ordinary skill in the art will recognize that bases can facilitate carbamate formation. Such bases include, but are not limited to, triethylamine, triisopropylamine, diisopropylethylamine, DBU, N-methylmorpholine, N-methylpyridine, N,N-dimethylpiperazine and the like. One of ordinary skill in the art will recognize that acyl transfer catalysts can facilitate carbamate formation. Such acyl transfer catalysts include, but are not limited to, DMAP, pyridine, 2-methylpyridine and the like. Additionally, one of ordinary skill in the art will recognize that introduction of Lewis acid catalysts may facilitate carbamate formation. Such Lewis acid catalysts include, but are not limited to, zinc chloride, zinc acetate, zinc bromide, aluminum trichloride, titanium trichloride, titanium isopropoxide, boron trifluoride, tin chloride, alumina, silica gel and the like.

One of ordinary skill in the art will also recognize that alternatives to the benzyl ether protecting group, and associated reaction conditions for their cleavage, are useful. Various appropriate alcohol protecting groups are broadly described in “Green's Protective Groups in Organic Synthesis”. Such benzyl ether alternatives include, but are not limited to, trimethylsilyl ethers, tert-butyl dimethylsilyl ethers, triisopropylsilyl ethers, tert-butyl diphenylsilyl ethers, acetates, benzoates, 4-nitrobenzoates, tert-butyl ethers, 4-methoxybenzyl ethers and the like.

One of ordinary skill in the art will recognize that, for Scheme 7, R⁵ and R⁶ may be replaced with R⁷ and R⁸, respectively. Furthermore, one of ordinary skill in the art will understand that, when R⁵ and R⁶ are replaced with R⁷ and R⁸, cleavage of protecting groups may be required. One of ordinary skill in the art will recognize that suitable protecting groups associated with R⁷ and R⁸ are dependent upon the functional groups for which said protecting groups may be required. Such protecting groups may be selected from the group consisting of but not limited to Ac, C₁-C₆ branched or unbranched alkyl, tert-Butyl, Benzyl, 4-Methoxybenzyl, Trityl, Benzoyl, para-nitrobenzoyl, MOM, BOM, Boc, Cbz, Fmoc or Si comprising the core of a silyl ether. Various appropriate protecting groups are broadly described in “Green's PROTECTIVE GROUPS IN ORGANIC SYNTHESIS”.

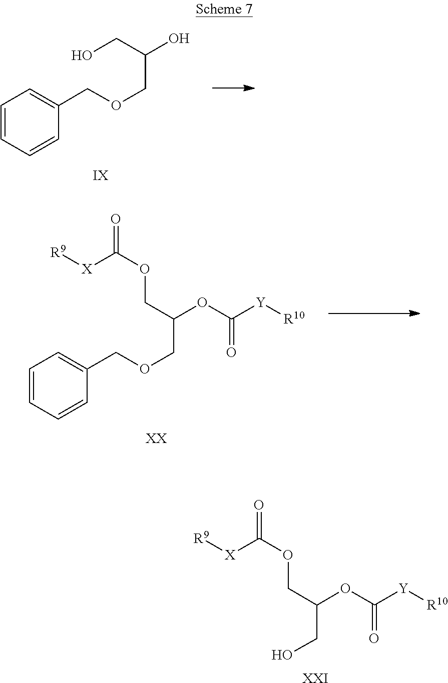

As illustrated in Scheme 8, a compound of Formula XVII couples to a compound of Formula XXI through a phosphodiester linkage. Subsequent optional protecting group cleavage and optional salt formation of the phosphodiester gives a compound of Formula XXII. One of ordinary skill in the art will recognize that a compound of Formula CVII and a compound of Formula XXI each contain primary hydroxyl groups. One of ordinary skill in the art will also recognize that formation of phosphodiesters between two different alcohols is achievable through use of a variety of phosphorus reagents and reaction conditions. Phosphorus reagents useful for the generation of phosphodiesters include, but are not limited to, POCl₃,

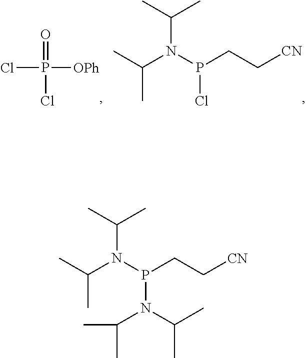

and the like. In some instances, the two alcohols are combined simultaneously with the phosphorus reagent. In some instances, the two hydroxyl groups are reacted with the phosphorus reagent in sequence. In some instances, the phosphodiester formation requires additional steps including, but not limited to, oxidation, deprotection or a combination thereof either executed as single additional steps or as multiple additional steps. One of ordinary skill in the art will recognize that bases can facilitate reaction with phosphorus reagents useful for phosphodiester formation. Such bases include, but are not limited to, triethylamine, triisopropylamine, diisopropylethylamine, DBU, N-methylmorpholine, N-methylpyridine, N,N-dimethylpiperazine and the like. One of ordinary skill in the art will recognize that acyl transfer catalysts can facilitate reaction with phosphorus reagents useful for phosphodiester formation. Such acyl transfer catalysts include, but are not limited to, DMAP, pyridine, 2-methylpyridine and the like. One of ordinary skill in the art will recognize that useful reagents for phosphodiester salt formation include, but are not limited to, DOWEX Na⁺ resin, sodium bicarbonate, sodium carbonate and the like.

One of ordinary skill in the art will recognize that, for Scheme 8, R⁵ and R⁶ may be replaced with R⁷ and R⁸, respectively. Furthermore, one of ordinary skill in the art will understand that, when R⁵ and R⁶ are replaced with R⁷ and R⁸, cleavage of protecting groups may be required. One of ordinary skill in the art will recognize that suitable protecting groups associated with R⁷ and R⁸ are dependent upon the functional groups for which said protecting groups may be required. Such protecting groups may be selected from the group consisting of but not limited to Ac, C₁-C₆ branched or unbranched alkyl, tert-Butyl, Benzyl, 4-Methoxybenzyl, Trityl, Benzoyl, para-nitrobenzoyl, MOM, BOM, Boc, Cbz, Fmoc or Si comprising the core of a silyl ether. Various appropriate protecting groups are broadly described in “Green's Protective Groups in Organic Synthesis”.

One of ordinary skill in the art will recognize that compounds of Formula XXII include compounds 1, 2, 3, 7, 8, 9, 10, 11, 12, 13, 14, 19, 20, 22, 23, 27, 28, 29, 31, 32, 33, 34, 35, 36, 37, 38, 39, 40, 41, 42, 43, 44, 45, 46, 47, 48, 49, 50 and 51.

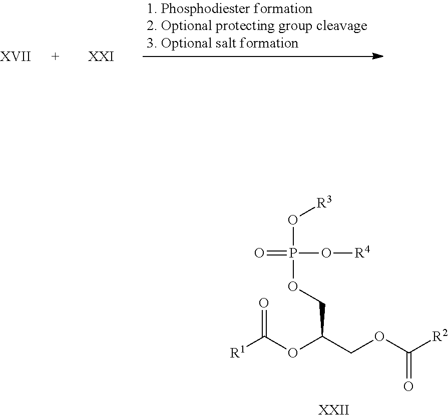

With reference to Schemes 1-8, one of ordinary skill in the art will recognize that, collectively, said schemes enable the preparation of various stereoisomers of a compound of Formula I and of Formula Ia. Furthermore, one of ordinary skill in the art will recognize that the various forms of the compounds of this invention include salt forms other than Na. With reference to different salt forms, compounds of Formula I and of Formula Ia, wherein R⁴ is as generally defined, can be converted from the OH form or from a given salt form into an alternate salt form. Reagents useful for such form interconversions include, but are not limited to, magnesium chloride, calcium chloride and the like. Furthermore, one of ordinary skill in the art will recognize that functional groups present as part of R⁵ and R⁶ may exist in salt forms appropriate for the nature of said functional groups. For example, when R⁵ and/or R⁶ contain a carboxylic acid unit, said carboxylic acid may be in the form of a pharmaceutically acceptable salt form comprising a salt counterion including, but not limited to, Li⁺, Na⁺, K⁺, Zn²⁺, Mg²⁺, Ca²⁺, Cs²⁺, ammonium, tetraalkylammonium and the like. Likewise, when R⁵ and/or R⁶ contain an amine unit, said amine may be in the form of a pharmaceutically acceptable comprising a pharmaceutically acceptable acid including, but not limited to, HCl, HBr, AcOH, MsOH, Fumararic acid, maleic acid and the like. One of ordinary skill in the art will recognize that, including R⁴ conversion, compounds of Formula XXII include compounds 1, 2, 3, 7, 8, 9, 10, 11, 12, 13, 14, 19, 20, 22, 23, 27, 28, 29, 31, 32, 33, 34, 35, 36, 37, 38, 39, 40, 41, 42, 43, 44, 45, 46, 47, 48, 49, 50 and 51.

It is contemplated that any embodiment discussed in this specification can be implemented with respect to any method, kit, reagent, or composition of the invention, and vice versa. Furthermore, compositions of the invention can be used to achieve methods of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

In executing the following exemplary synthetic protocols, the following relates to particulars relevant to equipment and analytical protocols. HPLC analyses were carried utilizing an XBridge C8 column (50×4.6 mm, 3.5μ) with the following method. Solvent A=25% ammonia in water, B=Acetonitrile; Flow Rate: 1 ml/min.

### Example 3. Preparation of Sodium (R)-2,3-bis(tetradecanoyloxy)propyl (2,3-diacetoxypropyl) Phosphate (Compound 1)

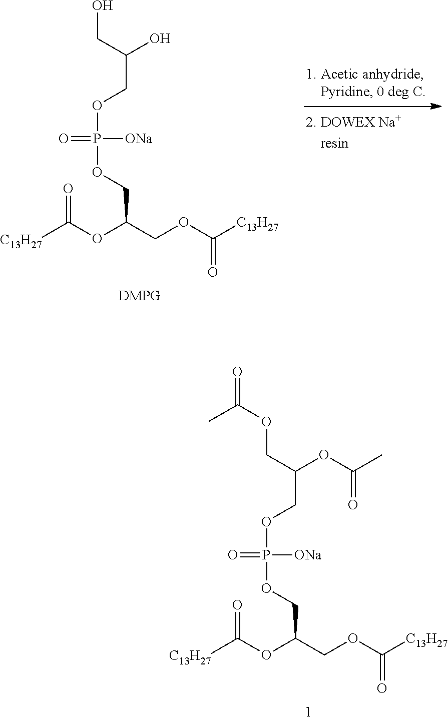

Acetic anhydride (3.5 ml, 36.3 mmol, 10 equiv) was added to a solution of DMPG sodium salt (2.5 g, 3.63 mmol) in dry pyridine (50 ml, 20 vol) at room temperature (25° C.) under nitrogen atmosphere. DMAP (88 mg, 0.725 mmol, 0.2 equiv) was added to the mixture and heated to 100° C. for 48 h. Upon completion of the reaction (as confirmed by TLC analysis, 20% MeOH in DCM, R_{f}˜0.6, identified by Phosphomolybdic acid stain), the solvent was evaporated, and the crude product was passed through column chromatography packed with neutral silica gel (230-400 mech). (Note: Silica gel was neutralized by washing with 10% ammonia in methanol). The product was eluted with dichloromethane containing 10% methanol to afford (R)-2,3-bis(tetradecanoyloxy)propyl (2,3-diacetoxypropyl) phosphate as its ammonium salt. The resultant ammonium salt was exchanged to Na salt by passing through a pad of Dowex® 50WX8 Na+ resin in dichloromethane containing 10% methanol. The product fractions were collected and concentrated. The product was dissolved in a mixture of acetonitrile and water (5 ml: 15 ml) and lyophilized to give the sodium salt of (R)-2,3-bis(tetradecanoyloxy)propyl (2,3-diacetoxypropyl) phosphate as light brown solid. Yield: 1.2 g (44%). ¹H-NMR (400 MHz, DMSO-d₆): δ_{H} 4.99-5.05 (m, 2H), 4.20-4.29 (m, 2H), 4.07 (m, 2H), 3.67 (m, 4H), 2.26 (t, J=4.49 Hz, 4H), 2.01 (s, 6H), 1.49 (m, 4H), 1.23 (m, 40H), and 0.85 (t, J=4.04 Hz, 6H) ppm. ¹³C-NMR (100 MHz, DMSO-d₆): δ_{H} 172.63, 172.45, 170.37, 170.08, 71.07, 70.99, 70.89, 63.85, 62.98, 62.81, 33.99, 33.83, 31.85, 29.68, 29.62, 29.56, 29.39, 29.35, 29.30, 29.07, 29.00, and 24.94 ppm. LCMS (ELSD): 749.1 (M−23).

### Example 4. Preparation of Sodium (R)-2, 3-bis (tetradecanoyloxy)propyl ((2,2-dimethyl-1,3-dioxolan-4-yl)methyl) Phosphate (Compound 12)

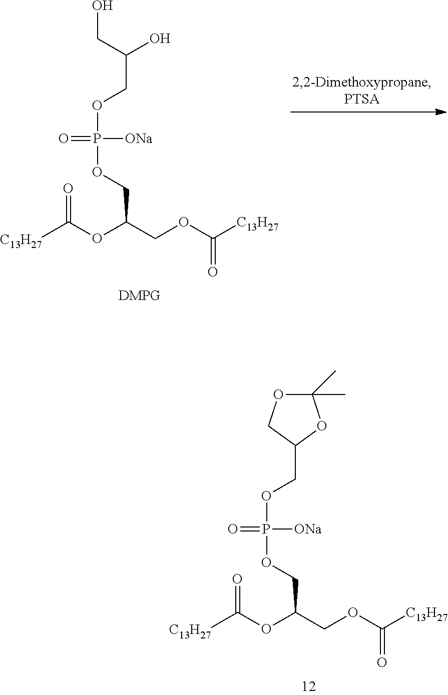

2,2-Dimethoxypropane (7.54 g, 72.5 mmol, 10 equiv) and p-Toluenesulfonic acid (72 mg, 0.378 mmol, 0.052 equiv) was added to a solution of DMPG sodium salt (5 g, 7.25 mmol) in toluene (200 ml, 40 vol). The mixture was heated to 140° C. for 16 h. The solvent was evaporated and the crude product (5.6 g) was taken for the next step without further purification.

### Example 5. Preparation of Sodium (R)-2, 3-bis (tetradecanoyloxy)propyl ((2-heptadecyl-1,3-dioxolan-4-yl)methyl) Phosphate (Compound 4)

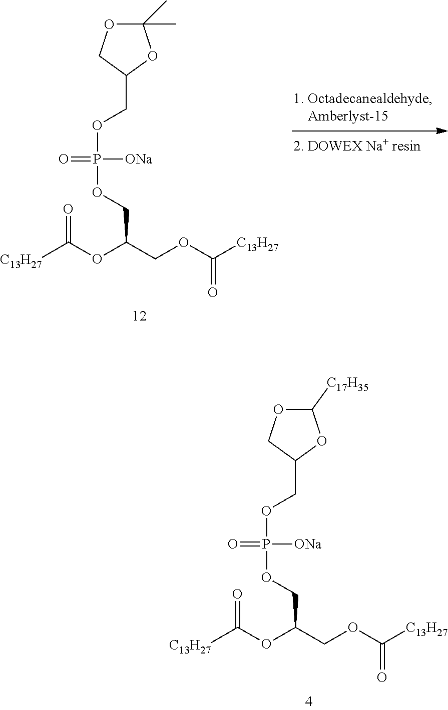

Octadecanaldehyde (4.55 g, 16.97 mmol, 3 equiv) was added to a solution of crude Compound 12 (4 g, 5.65 mmol) in 1,2-dichloroethane (80 ml, 20 vol) at room temperature (25° C.) followed by Amberlyst-15 (800 mg, 20 wt %). The mixture was stirred at 80° C. for 48 h. Upon completion of the reaction (as confirmed by TLC analysis, 15% MeOH in DCM, R_{f}˜0.4, identified by Phosphomolybdic acid stain), the reaction mixture was filtered and washed with aqueous NaHCO₃ solution (1×80 ml). The aqueous layer was extracted with DCM (3×50 ml) and the combined organic layers were dried over sodium sulphate. The organic layer was concentrated, and the crude product was passed through column chromatography packed with neutral silica gel (230-400 mech). (Note: Silica gel was neutralized by washing with 10% ammonia in methanol). The product was eluted with dichloromethane containing 10-12% methanol to afford (R)-2, 3-bis (tetradecanoyloxy)propyl ((2-heptadecyl-1,3-dioxolan-4-yl)methyl) phosphate as its ammonium salt (1.8 g). The (R)-2, 3-bis (tetradecanoyloxy)propyl ((2-heptadecyl-1,3-dioxolan-4-yl)methyl) phosphate was further purified by triturating with a mixture of DCM: MeOH (9 ml: 90 ml). The resultant solid was filtered and washed with methanol (1×10 ml). The ammonium salt was exchanged to Na salt by passing through a pad of Dowex® 50WX8 Na+ resin using 10% methanol in dichloromethane. The product fractions were collected and concentrated to give the sodium (R)-2, 3-bis (tetradecanoyloxy)propyl ((2-heptadecyl-1,3-dioxolan-4-yl)methyl) phosphate as off-white solid. Yield: 1.168 g (24.6%, 2 steps). ¹H-NMR (400 MHz, DMSO-d₆): δ_{H} 5.27 (m, 1H), 4.97-4.84 (m, 1H), 4.40-4.35 (m, 3H), 4.26-4.12 (m, 2H), 4.07 (m, 1H), 4.00-3.96 (m, 1H), 3.87 (m, 1H), 3.68 (m, 1H), 2.31 (t, J=7.40 Hz, 4H), 1.61 (m, 6H), 1.26 (m, 70H), and 0.89 (t, J=6.04 Hz, 9H) ppm. ¹³C-NMR (100 MHz, CDCl₃): δ_{H} 173.51, 173.40, 105.20, 104.61, 74.47, 70.55, 67.11, 66.52, 65.88, 63.94, 62.74, 34.27, 34.15, 34.08, 34.02, 31.94, 31.93, 29.83, 29.80, 29.77, 29.75, 29.73, 29.71, 29.68, 29.51, 29.46, 29.40, 29.37, 29.30, 29.29, 24.97, 24.88, 24.46, 24.10, 22.69, 14.16, and 14.15 ppm.

### Example 6. Preparation of Sodium (R)-2,3-bis(tetradecanoyloxy)propyl ((2-pentadecyl-1,3-dioxolan-4-yl)methyl) Phosphate (Compound 5)

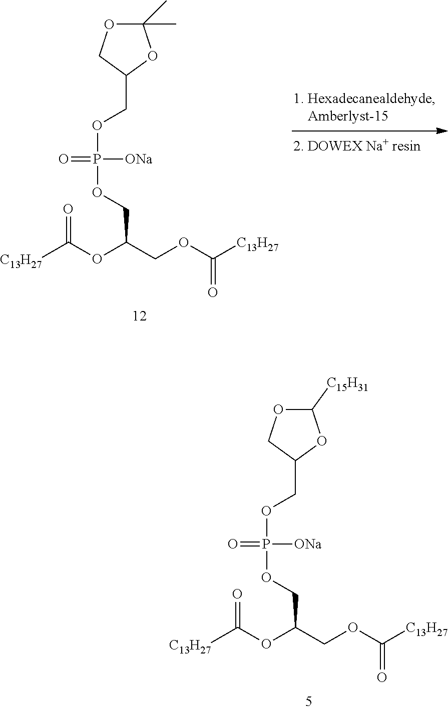

Hexadecanaldehyde (6.12 g, 25.4 mmol, 3 equiv) was added to a solution of crude Compound 12 (6 g, 8.42 mmol) in 1,2-dichloroethane (120 ml, 20 vol) at room temperature (25° C.). To this was added Amberlyst-15 (1.2 g, 20 wt %) and the mixture was stirred at 80° C. for 48 h. Upon completion of the reaction (as confirmed by TLC analysis, 15% MeOH in DCM, R_{f}˜0.4, identified by Phosphomolybdic acid stain), the reaction mixture was filtered and washed with aqueous sodium bicarbonate solution (1×100 ml). The aqueous layer was extracted with DCM (3×60 ml) and the combined organic layer as dried over anhydrous sodium sulphate. The organic layer was concentrated, and the crude product was passed through a bed of neutral silica gel (230-400 mech). (Note: Silica gel was neutralized by washing with 10% ammonia in methanol). The product was eluted with dichloromethane containing 10% methanol to afford (R)-2,3-bis(tetradecanoyloxy)propyl ((2-pentadecyl-1,3-dioxolan-4-yl)methyl) phosphate as its ammonium salt. The ammonium salt was exchanged to sodium salt by passing through a pad of Dowex® 50WX8 Na+ resin using 10% methanol in dichloromethane. The product fractions were collected and concentrated to give the sodium salt of (R)-2,3-bis(tetradecanoyloxy)propyl ((2-pentadecyl-1,3-dioxolan-4-yl)methyl) phosphate as off-white solid (1.30 g, 17.3% yield over 2 steps). ¹H-NMR (400 MHz, DMSO-d₆): δ_{H} 5.24 (m, 1H), 4.99-4.81 (2 t, J=4.4 Hz, 1H), 4.42 (m, 1H), 4.26-4.17 (m, 2H), 4.12-3.65 (m, 6H), 2.34-2.28 (m, 4H), 1.60 (m, 6H), 1.32 (m, 66H), and 0.90 (t, J=7.2 Hz, 9H) ppm. ¹³C-NMR (100 MHz, CDCl₃): δ_{H} 173.59, 105.19, 104.63, 74.6, 70.74, 67.22, 66.41, 65.71, 63.58, 62.83, 34.33, 34.16, 34.11, 34.05, 31.95, 29.81, 29.79, 29.72, 29.68, 29.51, 29.47, 29.41, 29.31, 24.98, 24.90, 24.49, 24.12, 22.70, and 14.09 ppm.

### Example 7. Preparation of Sodium (R)-2,3-bis(tetradecanoyloxy)propyl ((2-pentyl-1,3-dioxolan-4-yl)methyl) Phosphate (Compound 6)

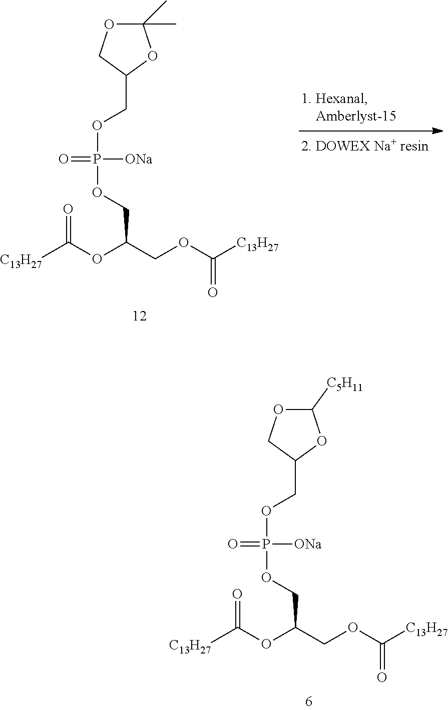

Hexanal (4.17 ml, 33.94 mmol, 6 equiv) was added to a solution of crude Compound 12 (4 g, 5.65 mmol) in DCM (80 ml, 20 vol) at room temperature (25° C.). To this was added Amberlyst-15 (800 mg, 20 wt %) and the mixture was stirred at room temperature for 16 h. Upon completion of the reaction (as confirmed by TLC analysis, 15% MeOH in DCM, Rf˜0.4, identified by Phosphomolybdic acid stain), the reaction mixture was filtered and washed with aqueous sodium bicarbonate solution (80 ml). The aqueous layer was extracted with DCM (3×50 ml) and the combined organic layer was dried over anhydrous sodium sulphate. The organic layer was concentrated under reduced pressure and the crude product was passed through as plug of neutral silica (230-400 mesh) eluting with dichloromethane containing 10% methanol to afford (R)-2,3-bis(tetradecanoyloxy)propyl ((2-pentyl-1,3-dioxolan-4-yl)methyl) phosphate as its ammonium salt. The resultant ammonium salt was exchanged to sodium salt by passing through a pad of Dowex® 50WX8 Na+ resin using 10% methanol in dichloromethane. The product fractions were collected and concentrated to give the sodium salt of (R)-2,3-bis(tetradecanoyloxy)propyl ((2-pentyl-1,3-dioxolan-4-yl)methyl) phosphate as light brown sticky solid. Yield: 1.77 g (41.74%). R_{f}=0.4 in 10:1.5/DCM:MeOH. ¹H-NMR (400 MHz, DMSO-d₆): δ_{H} 5.08 (m, 1H), 4.85-4.77 (m, 1H), 4.28 (m, 1H), 4.09-4.00 (m, 2H), 3.78-3.62 (m, 3H), 3.58-3.48 (m, 2H), 2.26 (t, J=5.24 Hz, 4H), 1.50 (m, 6H), 1.27 (m, 47H), and 0.85 (t, J=0.85 Hz, 9H) ppm. ¹³C-NMR (100 MHz, CDCl₃): δ_{H} 173.65, 173.57, 105.21, 104.61, 74.64, 74.57, 70.76, 70.69, 67.17, 67.07, 66.36, 65.61, 63.54, 62.84, 34.32, 34.11, 34.03, 33.91, 31.95, 31.87, 31.77, 29.78, 29.76, 29.71, 29.65, 29.49, 29.45, 29.40, 29.30, and 24.97 ppm.

### Example 8. Preparation of Sodium 2,3-bis(butyryloxy)propyl ((R)-2,3-bis(tetradecanoyloxy)propyl) Phosphate (Compound 7)

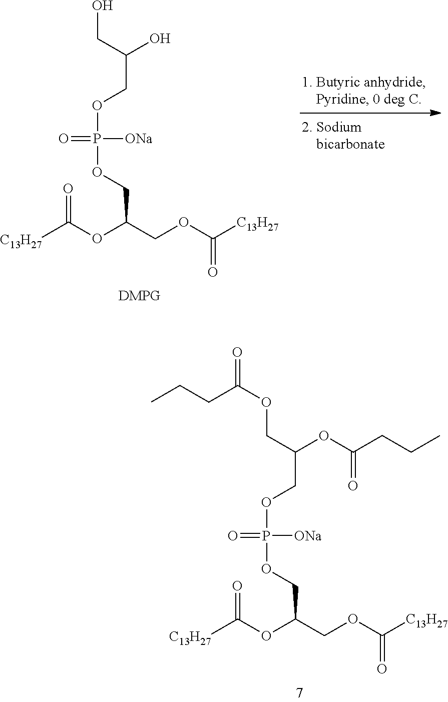

Butyric anhydride (8.96 g, 56.61 mmol, 13 equiv) was added to a solution of DMPG sodium salt (3.0 g, 4.35 mmol) in dry pyridine (60 ml, 20 vol) at room temperature (25° C.) under nitrogen atmosphere. DMAP (1.59 g, 13.07 mmol, 3.0 equiv) was added to the mixture in portions and the mixture was stirred at room temperature for 20 h. Upon completion of the reaction (as confirmed by TLC and LCMS analysis, 20% MeOH in DCM, R_{f}˜0.6, identified by Phosphomolybdic acid stain), the solvent was evaporated, and the crude product was passed through a plug of silica gel (230-400 mesh) eluting with dichloromethane containing 10% of methanol to afford the product as thick gum. This was diluted with ethyl acetate (20 vol) and washed with 1.5 N HCl (10 vol) followed by water. The organic layer was then stirred with aqueous NaHCO₃ solution (3 equiv in 5 vol of water) at room temperature for 30 min. The organic layer was separated, dried over Na₂SO₄ and concentrated under vacuum to get sodium 2,3-bis(butyryloxy)propyl ((R)-2,3-bis(tetradecanoyloxy)propyl) phosphate as a thick syrup (1.6 g, 44% yield). ¹H-NMR (400 MHz, CDCl₃): δ_{H} 5.23-5.25 (m, 2H), 4.40-4.43 (m, 2H), 4.18-4.24 (m, 2H), 3.93 (m, 4H), 2.28-2.33 (m, 8H), 1.58-1.68 (m, 8H), 1.27-1.33 (m, 40H), and 0.95-0.97 (m, 6H) ppm. ¹³C-NMR (100 MHz, CDCl₃): δ_{H} 173.57, 173.45, 70.72, 63.51, 62.70, 36.08, 35.91, 34.29, 34.08, 31.94, 29.75, 29.69, 29.65, 29.62, 29.45, 29.42, 29.39, 29.26, 29.24, 24.94, 24.87, 22.69, 18.31, 18.28, 14.10, 13.62 and 13.57 ppm.

### Example 9. Preparation of Sodium 2,3-bis((3-methylbutanoyl)oxy)propyl ((R)-2,3-bis(tetradecanoyloxy)propyl) Phosphate (Compound 8)

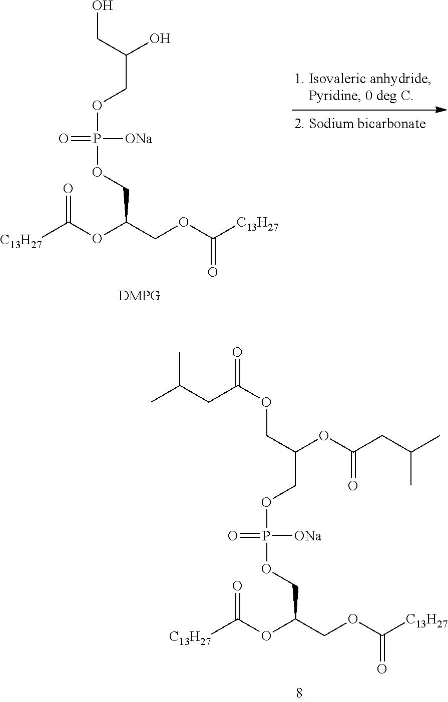

Isovaleric anhydride (10.55 g, 56.66 mmol, 13 equiv) was added to a solution of DMPG sodium salt (3.0 g, 4.35 mmol) in dry pyridine (60 ml, 20 vol) at room temperature (25° C.) under nitrogen atmosphere. To this was added DMAP (1.59 g, 13.07 mmol, 3.0 equiv) in portions and the mixture was stirred at room temperature for 20 h. Upon completion of the reaction (as confirmed by TLC and LCMS analysis, 20% MeOH in DCM, R_{f}˜0.6, identified by Phosphomolybdic acid stain), the solvent was evaporated and the crude product was passed through a plug of silica gel (230-400 mesh) eluting with dichloromethane containing 10% of methanol to afford the product as thick gum. This was diluted with ethyl acetate (20 vol) and washed with 1.5 N HCl (10 vol) followed by water. The organic layer was then stirred with aqueous NaHCO₃ solution (3 equiv in 5 vol of water) at room temperature for 30 min. The organic layer was separated, dried over Na₂SO₄ and concentrated under vacuum to get sodium 2,3-bis((3-methylbutanoyl)oxy)propyl ((R)-2,3-bis(tetradecanoyloxy)propyl) phosphate as a thick syrup (2.4 g, 64% yield). ¹H-NMR (400 MHz, CDCl₃): δ_{H} 5.24-5.29 (m, 2H), 4.40-4.44 (m, 2H), 4.18-4.22 (m, 2H), 3.95 (m, 4H), 2.19-2.34 (m, 8H), 2.06-2.12 (m, 2H), 1.58-1.61 (m, 4H), 1.27-1.33 (m, 40H), 0.96 (d, J=6.8 Hz, 12H) and 0.90 (t, J=7.2 Hz, 6H) ppm. ¹³C-NMR (100 MHz, CDCl₃): δ_{H} 173.51, 173.38, 172.80, 172.71, 70.69, 70.62, 63.59, 62.72, 43.27, 43.07, 34.27, 34.07, 31.92, 29.73, 29.67, 29.63, 29.60, 29.44, 29.41, 29.36, 29.25, 29.24, 25.50, 24.93, 24.86, 22.67, 22.34, 22.30, and 14.07 ppm. LCMS (ELSD): 833.5 (M−23). Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ. Rt (min): 5.74; Area %-98.84.

### Example 10. Preparation of Sodium 2,3-bis(isobutyryloxy)propyl ((R)-2,3-bis(tetradecanoyloxy)propyl) phosphate (Compound 9)

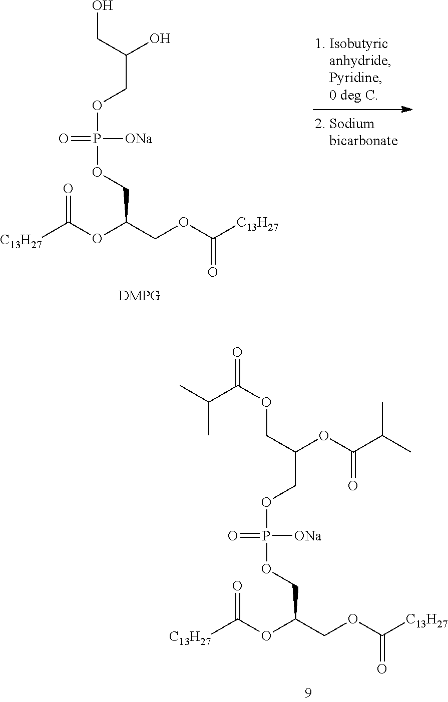

Isobutyric anhydride (10.55 g, 56.66 mmol, 13 equiv) was added to a solution of DMPG sodium salt (3.0 g, 4.35 mmol) in dry pyridine (60 ml, 20 vol) at room temperature (25° C.) under nitrogen atmosphere. DMAP (1.59 g, 13.07 mmol, 3.0 equiv) was added to this mixture in portions, and the mixture was stirred at room temperature for 20 h. Upon completion of the reaction (as confirmed by TLC and LCMS analysis, 20% MeOH in DCM, R_{f}˜0.6, identified by Phosphomolybdic acid stain), the solvent was evaporated and the crude product was passed through a plug of silica gel (230-400 mesh) eluting with dichloromethane containing 10% of methanol to afford the product as thick gum. This was diluted with ethyl acetate (20 vol) and washed with 1.5 N HCl (10 vol) followed by water. The organic layer was then stirred with aqueous NaHCO₃ solution (3 equiv in 5 vol of water) at room temperature for 30 min. The organic layer was separated, dried over Na₂SO₄ and concentrated under vacuum to get sodium 2,3-bis(isobutyryloxy)propyl ((R)-2,3-bis(tetradecanoyloxy)propyl) phosphate as a thick syrup (1.6 g, 44% yield). ¹H-NMR (400 MHz, CDCl₃): δ_{H} 5.24-5.27 (m, 2H), 4.39-4.44 (m, 2H), 4.17-4.23 (m, 2H), 3.94 (m, 4H), 2.52-2.60 (m, 2H), 2.28-2.33 (m, 4H), 1.58-1.61 (m, 4H), 1.31-1.33 (m, 40H), 1.32 (t, J=6.8 Hz, 12H) and 0.89 (t, J=7.2 Hz, 6H) ppm. ¹³C-NMR (100 MHz, CDCl₃): δ_{H} 176.74, 173.53, 173.41, 70.72, 63.50, 62.73, 34.28, 34.07, 33.93, 33.85, 31.92, 29.73, 29.68, 29.62, 29.60, 29.43, 29.39, 29.37, 29.24, 29.22, 24.92, 24.86, 22.68, 18.97, and 14.10 ppm.

### Example 11. Preparation of Sodium 2,3-bis((ethoxycarbonyl)oxy)propyl ((R)-2,3-bis (tetra decanoyloxy)propyl) Phosphate (Compound 2)

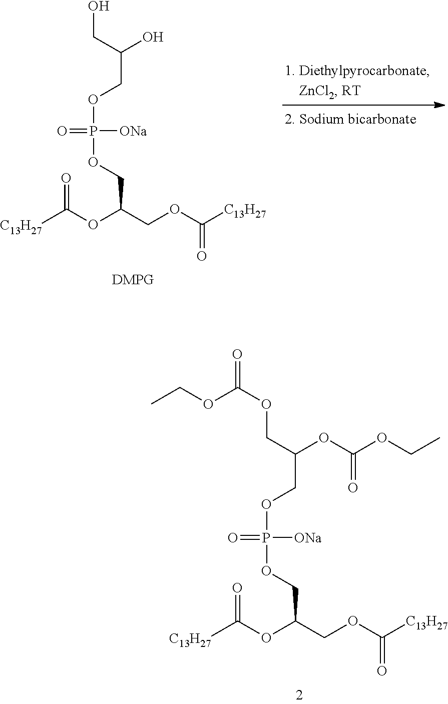

To a suspension of DMPG-Na (500.0 g, 0.7258 mol, 1.0 equiv) in toluene (15 vol) was added diethylpyrocarbonate (1176.5 g, 7.258 mol, 10 equiv) followed by anhydrous ZnCl₂ (128.61 g, 0.943 mol, 1.3 equiv) at room temperature under nitrogen. The mixture was stirred at 37-40° C. for 30 h. After completion of reaction, the reaction mixture was cooled to room temperature AND filtered through a thin bed of Celite®. The filtrate was concentrated under vacuum maintaining the bath temperature below 40° C. The sticky residue was dissolved in EtOAc (30 vol) and washed with water (5 vol×2). The organic layer was dried over Na₂SO₄ and concentrated under vacuum maintaining the bath temperature below 45° C. to get a sticky residue. The crude product (680 g) was purified by silica gel column chromatography (230-400 mesh) using 5-20% of MeOH in dichloromethane as gradient. The product fractions were concentrated to get 385 g of pure product. This was dissolved in a mixture of EtOAc and water (15:3 vol)) and cooled to ˜5° C. To this was added HCl solution (0.5 N, 2 equiv) and the mixture was stirred for 15-20 minutes at ˜5° C. The organic layer was separated and washed with 0.5 N HCl (3 vol×1) and water (5 vol×1). The organic layer was slowly basified with NaHCO₃ solution (4 equiv in 5 vol of water) at room temperature. The mixture was stirred for 2 h and the organic layer was separated, dried over Na₂SO₄ and concentrated to get sodium 2,3-bis((ethoxycarbonyl)oxy)propyl ((R)-2,3-bis(tetradecanoyloxy)propyl) phosphate as a thick syrup (300.0 g, 49% yield). ¹H-NMR (400 MHz, CDCl₃): δ_{H} 5.23-5.25 (m, 1H), 5.10-5.11 (m, 1H), 4.49-4.36 (m, 2H), 4.35-4.16 (m, 6H), 4.03-3.91 (m, 4H), 2.34-2.28 (m, 4H), 1.61-1.57 (m, 4H), 1.35-1.27 (m, 46H), and 0.89 (t, J=68 Hz, 6H) ppm. ¹³C-NMR (100 MHz, CDCl₃): δ_{H} 173.56, 173.50 154.97, 154.77, 154.73, 74.51, 74.44, 70.71, 70.64, 65.88, 64.48, 64.41, 64.23, 63.55, 63.20, 62.74, 34.25, 34.04, 31.93, 29.76, 29.74, 29.70, 29.66, 29.62, 29.46, 29.42, 29.39, 29.26, 29.23, 24.93, 24.86, 22.69, and 14.10 ppm.

### Example 12. Preparation of Magnesium-2,3-bis((ethoxycarbonyl)oxy)propyl((R)-2,3-bis(tetradecanoyloxy)propyl) Phosphate (Compound 10)

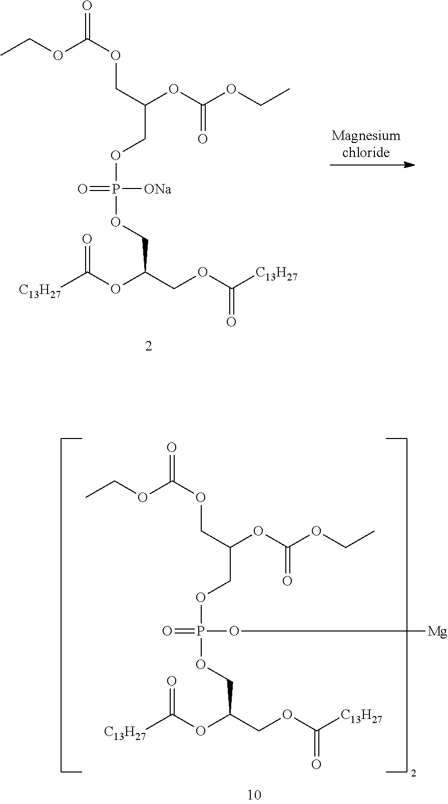

A solution of magnesium chloride (0.571 g, 6.00 mmol, 0.5 equiv) in water (10 vol) was added to sodium 2,3-bis((ethoxycarbonyl)oxy)propyl ((R)-2,3-bis(tetradecanoyloxy)propyl) phosphate (10 g, 12.00 mmol, 1.0 equiv) in ethanol (100 ml), and the mixture was stirred at room temperature for 14 h. The mixture was diluted with water (200 ml) and the precipitate was filtered, washed with water (100 ml) and dried under vacuum to afford magnesium-2,3-bis((ethoxycarbonyl)oxy)propyl((R)-2,3-bis(tetradecanoyloxy)propyl) phosphate as an off-white solid (8.0 g, 79% yield). ¹H-NMR (400 MHz, CDCl₃): δ_{H} 5.27 (m, 1H), 5.13 (m, 1H), 4.50-4.00 (m, 14H), 2.34-2.28 (m, 4H), 1.60-1.59 (m, 4H), 1.35 (m, 46H), and 0.89 (t, J=6.4 Hz, 6H) ppm. ¹³C-NMR (100 MHz, CDCl₃): δ_{H} 173.50, 173.31, 154.91, 154.60, 74.21, 70.31, 65.84, 64.35, 64.19, 63.96, 63.60, 62.70, 34.15, 34.00, 31.92, 29.75, 29.68, 29.66, 29.62, 29.48, 29.43, 29.37, 29.26, 29.22, 24.91, 22.67, 14.13 and 14.07 ppm.

### Example 13. Preparation of Calcium-2,3-bis((ethoxycarbonyl)oxy)propyl ((R)-2,3-bis(tetradecanoyloxy)propyl) Phosphate (Compound 11)

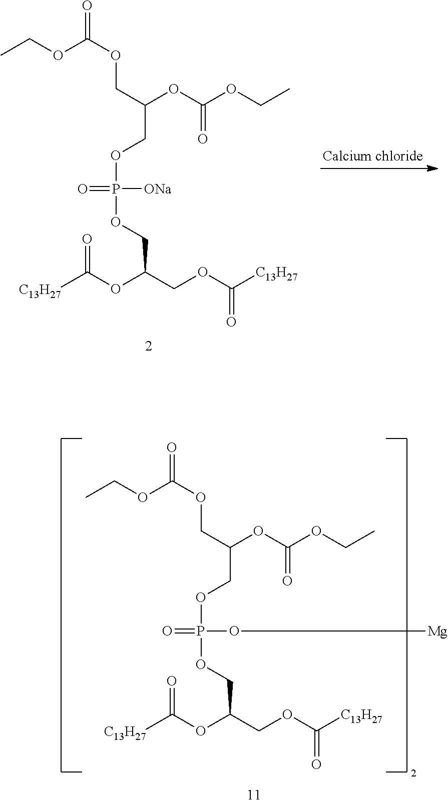

A solution of calcium chloride (0.666 g, 6.00 mmol, 0.5 equiv) in water (10 vol) was added to a solution of sodium 2,3-bis((ethoxycarbonyl)oxy)propyl ((R)-2,3-bis(tetradecanoyloxy)propyl) phosphate (10 g, 12.00 mmol, 1.0 equiv) in ethanol (100 ml) at room temperature and the mixture was stirred at room temperature for 14 h. The mixture was diluted with water (200 ml) and the precipitate was filtered, washed with water (100 ml) and dried under vacuum to afford calcium-2,3-bis((ethoxycarbonyl)oxy)propyl((R)-2,3-bis(tetradecanoyloxy)propyl) phosphate as an off white solid (8.2 g, 83% yield). ¹H-NMR (400 MHz, CDCl₃): δ_{H} 5.28 (m, 1H), 5.19 (m, 1H), 4.51-4.40 (m, 2H), 4.36-4.14 (m, 6H), 3.90-4.10 (m, 4H), 2.34-2.28 (m, 4H), 1.59 (m, 4H), 1.33-1.27 (m, 46H), and 0.89 (t, J=6.8 Hz, 6H) ppm. ¹³C-NMR: (100 MHz, CDCl₃): δ_{H} 173.54, 154.99, 74.44, 70.59, 68.70, 65.95, 64.49, 64.26, 63.92, 62.71, 34.17, 34.00, 31.91, 29.74, 29.72, 29.67, 29.61, 29.46, 29.42, 29.37, 29.25, 29.20, 24.88, 24.80, 22.66, 14.11 and 14.05 ppm.

### Example 14. Preparation of Sodium 2,3-bis((4-hydroxybutanoyl)oxy)propyl ((R)-2,3-bis(tetradecanoyloxy)propyl) Phosphate (Compound 31)

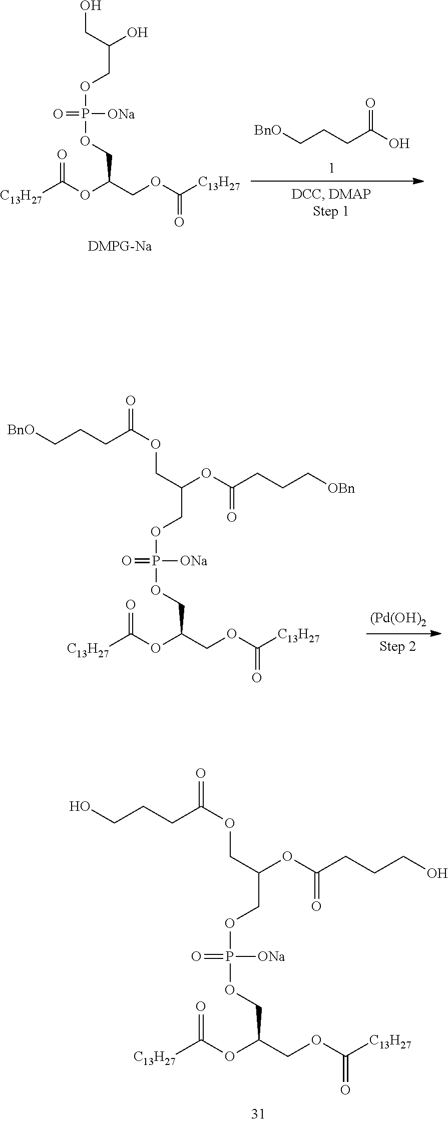

Step 1: Sodium 2,3-bis((4-(benzyloxy)butanoyl)oxy)propyl ((R)-2,3-bis(tetradecanoyloxy) propyl) phosphate.

To a stirred solution of 4-(benzyloxy)butanoic acid (3.38 g, 17.42 mmol, 3 equiv) in DCM (100 ml), dicyclohexylmethanediimine (3.59 g, 17.42 mmol, 3 equiv) was added at RT and the mixture was stirred for 30 min. N,N-dimethylpyridin-4-amine (0.355 g, 2.90 mmol, 0.5 equiv) and sodium (R)-2,3-bis(tetradecanoyloxy)propyl (2,3-dihydroxypropyl) phosphate (4.0 g, 5.81 mmol, 1 equiv) was added at RT. The reaction mixture was stirred at RT for 32 h, and was monitored by LCMS. Upon completion, reaction mixture was diluted with DCM (50 ml) and stirred for 10 min and filtered. The filtrate was washed with water (50 ml×1), 0.5N HCl (50 ml×1) and 10% NaHCO₃ (50 ml×1). Combined organic layer was dried over sodium sulfate, filtered, and concentrated to get 6.4 g of crude product. The crude product (6.4 g) was purified by column chromatography using basic 230-400 mesh silica gel (pre basified using ammonia). The product was eluted at 0-10% of methanol in DCM. Pure fractions were collected and concentrated to get sodium 2,3-bis((4-(benzyloxy)butanoyl)oxy)propyl ((R)-2,3-bis(tetradecanoyloxy)propyl) phosphate (3.5 g) as colourless syrup, which was dissolved in DCM (100 ml), washed with 0.5N HCl (50 ml×1), 10% NaHCO₃ solution (50 ml×2). Combined organic layer was dried over sodium sulphate, filtered and concentrated under reduced pressure to get sodium 2,3-bis((4-(benzyloxy)butanoyl)oxy)propyl ((R)-2,3-bis(tetradecanoyloxy)propyl) phosphate (3.1 g, 51% yield) as colourless syrup.

¹H NMR (400 MHz, DMSO-d6) δ (ppm)=7.36-7.26 (m, 10H), 5.09-5.03 (m, 2H), 4.43 (d, J=1.5 Hz, 4H), 4.32-4.22 (m, 2H), 4.07 (dd, J=6.6, 11.9 Hz, 2H), 3.76-3.65 (m, 4H), 3.43-3.38 (m, 4H), 2.41-2.30 (m, 4H), 2.29-2.20 (m, 4H), 1.84-1.73 (m, 4H), 1.55-1.43 (m, 4H), 1.22 (s, 40H), 0.90-0.80 (m, 6H). LCMS: Mol. formula: C₅₆H₉₀NaO₁₄P, formula weight: 1041.29, exact mass: 1018.61. observed mass: 1017.7 [M−1]⁻, RT=3.70 min, purity: 99.42%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: Sodium 2,3-bis((4-hydroxybutanoyl)oxy)propyl ((R)-2,3-bis(tetradecanoyloxy)propyl) phosphate.

To a stirred solution of sodium 2,3-bis((4-(benzyloxy)butanoyl)oxy)propyl ((R)-2,3-bis(tetradecanoyloxy)propyl) phosphate (1.4 g, 1.344 mmol, 1 equiv) in THF (30 ml) was added dihydroxypalladium, 20% dry basis (1.2 g, 8.54 mmol) at RT under nitrogen atmosphere. The reaction was stirred under 60 psi H₂ pressure for 16 h. The progress of the reaction was monitored by LCMS and TLC. Upon completion, the reaction mixture was filtered through celite bed, washed with THF (60 ml), and the filtrate was concentrated under reduced pressure at RT to get 1 g of product. Crude product was dissolved in DCM (20 ml), Amberlite IR120 Na resin (2 g) was added, stirred for 1.5 h and filtered. Filtrate was concentrated and further dried under vacuum to get sodium 2,3-bis((4-hydroxybutanoyl)oxy)propyl ((R)-2,3-bis(tetradecanoyloxy)propyl) phosphate (720 mg, 63.1% yield) as an off white solid.

¹H NMR (400 MHz, DMSO-d6) δ (ppm)=5.09-5.01 (m, 2H), 4.68 (td, J=5.3, 15.0 Hz, 2H), 4.32-4.19 (m, 2H), 4.16-4.03 (m, 2H), 3.79-3.64 (m, 4H), 3.41-3.36 (m, 4H), 2.38-2.21 (m, 8H), 1.76-1.56 (m, 4H), 1.55-1.46 (m, 4H), 1.24 (s, 40H), 0.91-0.80 (m, 6H). LCMS: Mol. formula: C₄₂H₇₈NaO₁₄P, formula weight: 861.04, exact mass: 838.52, observed mass: 837.4 [M−1]⁻, RT=2.99 min, purity: 99.28%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ. HPLC: RT=4.17 min; purity: 99.54%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

### Example 15. Preparation of (2R)-3-(((2,3-bis((4-aminobutanoyl)oxy)propoxy)(hydroxy)-phosphoryl)-oxy)propane-1,2-diyl Ditetradecanoate Dihydrochloride (Compound 32)

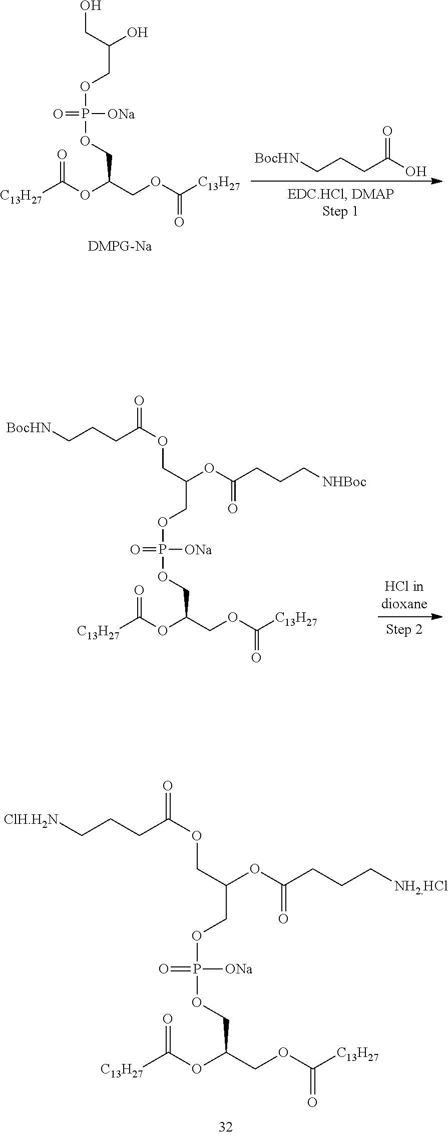

Step 1: Sodium 2,3-bis((4-((tert-butoxycarbonyl)amino)butanoyl)oxy)propyl ((R)-2,3-bis(tetradecanoyloxy)propyl) phosphate.

To a stirred solution of 4-((tert-butoxycarbonyl) amino) butanoic acid (4.43 g, 21.78 mmol, 3 equiv) in chloroform (75 ml) was added EDC·HCl (3.48 g, 18.15 mmol, 2.5 equiv) and 1H-benzo[d][1,2,3]triazol-1-ol (2.452 g, 18.15 mmol, 2.5 equiv) at RT. The reaction mixture was stirred at RT for 30 min. Sodium (R)-2,3-bis(tetradecanoyloxy)propyl (2,3-dihydroxypropyl) phosphate (5 g, 7.26 mmol, 1 equiv) and N, N-dimethylpyridin-4-amine (2.217 g, 18.15 mmol, 2.5 equiv) was added, and reaction mixture was refluxed for 16 h. The progress of the reaction was monitored by LCMS. After completion, the reaction mixture was diluted with DCM (100 ml), washed with water (50 ml×2), cold 1.5 N HCl (50 ml×2) and 10% NaHCO₃ solution (50 ml xl). The organic layer was dried over sodium sulfate, filtered and concentrated to get 7.5 g of crude product. The crude product (7.5 g) was purified by column chromatography using 230-400 mesh silica gel. The product was eluted at 0-5% of methanol in DCM. Pure fractions were collected and concentrated to get sodium 2,3-bis((4-((tert-butoxycarbonyl) amino) butanoyl) oxy) propyl ((R)-2,3-bis(tetradecanoyloxy)propyl) phosphate (3.3 g, 42.2% yield) as pale-yellow thick syrup.

¹H NMR (400 MHz, DMSO-d6) δ (ppm)=6.97-6.73 (m, 2H), 5.15-5.03 (m, 2H), 4.36-4.18 (m, 2H), 3.92 (br d, J=4.0 Hz, 4H), 2.98-2.89 (m, 4H), 2.36-2.20 (m, 6H), 1.67-1.57 (m, 4H), 1.57-1.45 (m, 4H), 1.37 (s, 18H), 1.24 (s, 40H), 0.89-0.82 (m, 6H). LCMS: Mol. formula: C₅₂H₉₆N₂NaO₁₆P, formula weight: 1059.30, exact mass: 1036.66, observed mass: 1035.6 [M−1]⁻, RT=3.44 min, purity: 99.14%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: (2R)-3-(((2,3-bis((4-aminobutanoyl)oxy)propoxy)(hydroxy)phosphoryl)oxy)propane-1,2-diylditetradecanoate dihydrochloride.

To a stirred solution of sodium 2,3-bis((4-((tert-butoxycarbonyl)amino)butanoyl)oxy)propyl ((R)-2,3-bis(tetradecanoyloxy)propyl) phosphate (0.7 g, 0.661 mmol) in 1,4-Dioxane (7 ml) was added hydrogen chloride (4.0 M HCl in 1,4-Dioxane, 7 ml, 28.0 mmol) at 10° C. The reaction mixture was stirred at 15-20° C. for 1 h 20 min. The progress of the reaction was monitored by LCMS. Upon completion, the reaction mixture was concentrated under reduced pressure at RT. The residue was co-evaporated with ethyl acetate (25 ml×1) to get (2R)-3-(((2,3-bis((3-aminopropanoyl)oxy)propoxy)(hydroxy)phosphoryl)oxy)-propane-1,2-diyl ditetradecanoate hydrochloride (360 mg, 76%) as an off white solid.

¹H NMR (400 MHz, DMSO-d6) δ (ppm)=8.19-7.87 (m, 6H), 5.17-5.07 (m, 2H), 4.33-4.24 (m, 2H), 4.20-4.03 (m, 2H), 4.03-3.93 (m, 2H), 3.92-3.84 (m, 2H), 2.82 (br d, J=6.3 Hz, 4H), 2.38-2.24 (m, 4H), 1.92-1.74 (m, 4H), 1.64-1.37 (m, 4H), 1.31-1.01 (m, 40H), 0.86 (t, J=6.7 Hz, 6H). LCMS: Mol. formula: C₄₂H₈₃Cl₂N₂O₁₂P, formula weight: 910.00, exact mass: 836.55. observed mass: 837.5 [M+1]⁺, RT=3.12 min, purity: 96.97%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ. HPLC: RT=6.61 min; purity: 95.49%. Method: Column: Xbridge C8(50λ4.6) mm, 3.5 μm, Mobile Phase A:0.1% TFA in water, Mobile Phase B: Acetonitrile, Flow rate:2.0 ml/min.

### Example 16. (2R)-3-(((2,3-bis((L-leucyl)oxy)propoxy)(hydroxy)phosphoryl)oxy)propane-1,2-diyl Ditetradecanoate Dihydrochloride (Compound 33)

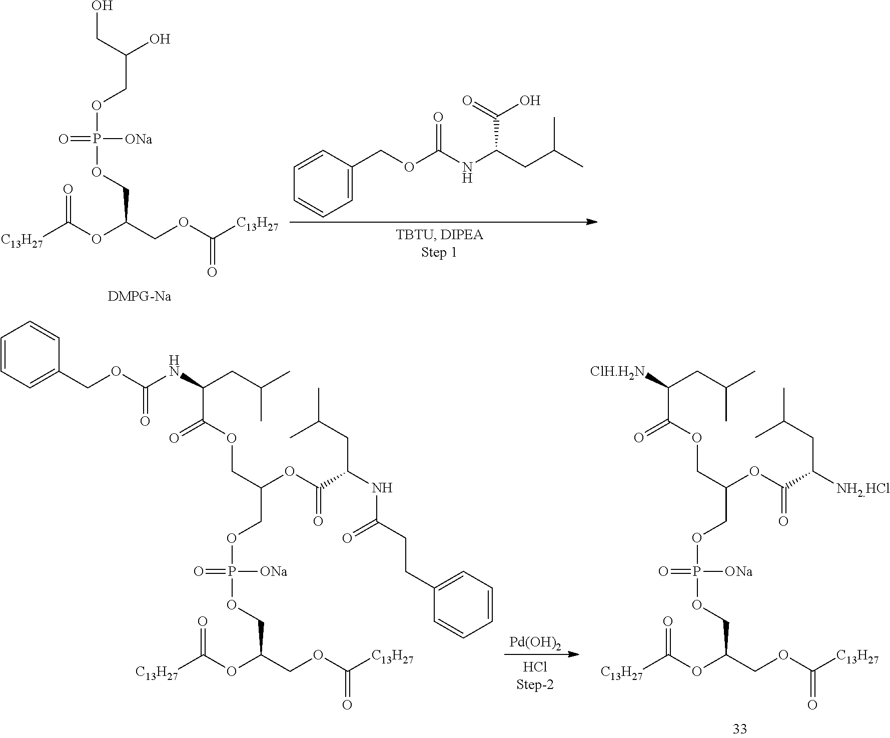

Step 1: Sodium 2,3-bis((((benzyloxy)carbonyl)-L-leucyl)oxy)propyl ((R)-2,3-bis(tetradecanoyloxy)propyl) phosphate.

To a stirred solution of ((benzyloxy)carbonyl)-L-leucine (4.81 g, 18.15 mmol), 2.5 equiv) in DCM (125 ml), TBTU (9.32 g, 29.0 mmol, 4 equiv) was added at RT and stirred for 30 min. Sodium (R)-2,3-bis(tetradecanoyloxy)propyl (2,3-dihydroxypropyl) phosphate (5.0 g, 7.26 mmol, 1 equiv), N-ethyl-N-isopropylpropan-2-amine (6.32 ml, 36.3 mmol, 5 equiv) were added at 5° C. and the reaction was stirred at RT for 18 h, monitored by LCMS. Upon completion, reaction mixture was diluted with DCM (75 ml) washed with water (50 ml×2), cold 1.5N HCl (50 ml×2), NaHCO₃ solution (50 ml×1). The combined organic layer was washed with brine, dried over sodium sulfate, filtered and concentrated under reduced pressure to give 7.8 g product. The crude product was purified by column chromatography using basic 230-400 mesh silica gel (pre basified using ammonia). The product was eluted at 0-10% of methanol in ethyl acetate. Pure fractions were collected and concentrated to get 3.6 g product. This product was dissolved in DCM (100 ml), washed with of 0.5N HCl (50 ml×1), 10% NaHCO₃ solution (50 ml×2). Combined organic layer was dried over sodium sulphate, filtered, and concentrated under reduced pressure to get sodium 2,3-bis((((benzyloxy)carbonyl)-L-leucyl)oxy)propyl ((R)-2,3-bis(tetradecanoyloxy)propyl) phosphate (3.1 g, 35.8% yield) as colorless thick syrup.

¹H NMR (400 MHz, DMSO-d6) δ (ppm)=7.81-7.68 (m, 2H), 7.41-7.28 (m, 10H), 5.11-4.97 (m, 6H), 4.30 (br d, J=11.8 Hz, 2H), 4.15-4.00 (m, 4H), 3.82-3.63 (m, 4H), 2.33-2.15 (m, 4H), 1.75-1.36 (m, 10H), 1.30-1.15 (m, 40H), 0.92-0.79 (m, 18H). LCMS: Mol. formula: C₆₂H₁₀₀N₂NaO₁₆P, formula weight: 1183.44, exact mass: 1160.69, observed mass: 1159.6 [M−1]⁻, RT=3.88 min, purity: 99.14%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: (2R)-3-(((2,3-bis((L-leucyl)oxy)propoxy)(hydroxy)phosphoryl)oxy)propane-1,2-diyl ditetradecanoate dihydrochloride.

To a stirred solution of sodium 2,3-bis((((benzyloxy)carbonyl)-L-leucyl)oxy)propyl ((R)-2,3-bis(tetradecanoyloxy)propyl) phosphate (0.500 g, 0.422 mmol, 1 equiv) in DCM (20 ml), dihydroxypalladium, 20% dry basis (0.250 g, 0.422 mmol) was added at RT under nitrogen atmosphere. The reaction was stirred under under hydrogen bladder pressure for 3 h. The progress of the reaction was monitored by LCMS and TLC. Upon completion, the reaction mixture was filtered through celite bed, washed with mixture of THF (25 ml) and DCM (25 ml). 4M HCl in 1,4 dioxane (3.5 eq) was added to the reaction mixture (filtrate) at 0° C. and stirred for 10 min at 0° C. The mixture was concentrated under reduced pressure at RT and dried to get (2R)-3-(((2,3-bis((L-leucyl)oxy)propoxy)(hydroxy)-phosphoryl)oxy)propane-1,2-diyl ditetradecanoate dihydrochloride (0.260 g, 66.5% yield) as grey solid.

¹H NMR (400 MHz, DMSO-d6) δ (ppm)=8.91-8.26 (m, 6H), 5.21-5.06 (m, 2H), 4.40 (br d, J=4.2 Hz, 2H), 4.28 (br d, J=11.7 Hz, 1H), 4.19-3.77 (m, 8H), 2.34-2.18 (m, 4H), 1.85-1.60 (m, 6H), 1.57-1.44 (m, 4H), 1.24 (s, 40H), 0.97-0.79 (m, 18H). LCMS: Mol. formula: C₄₆H₉₁Cl₂N₂O₁₂P, formula weight: 966.11, exact mass: 892.62, observed mass: 893.5 [M+1]⁺, RT=3.26 min, purity: 97.46%. Method info: Column: X-BRIDGE C8 (50*4.6) 3.5 μm, Mobile phase:A:0.1% TFA in H₂O B: 0.1% TFA in ACN, Flow Rate: 1.5 ml/min. HPLC: RT=7.13 min; purity: 98.95%. Method info: A:0.1% TFA in H2O, B:ACN, Flow Rate: 1.5 ml/min. COLUMN: XBridge C8(50×4.6) mm, 3.5 μm.

### Example 17. Sodium 2,3-bis((3-hydroxypropanoyl)oxy)propyl ((R)-2,3-bis(tetradecanoyl-oxy)propyl) Phosphate (Compound 34)

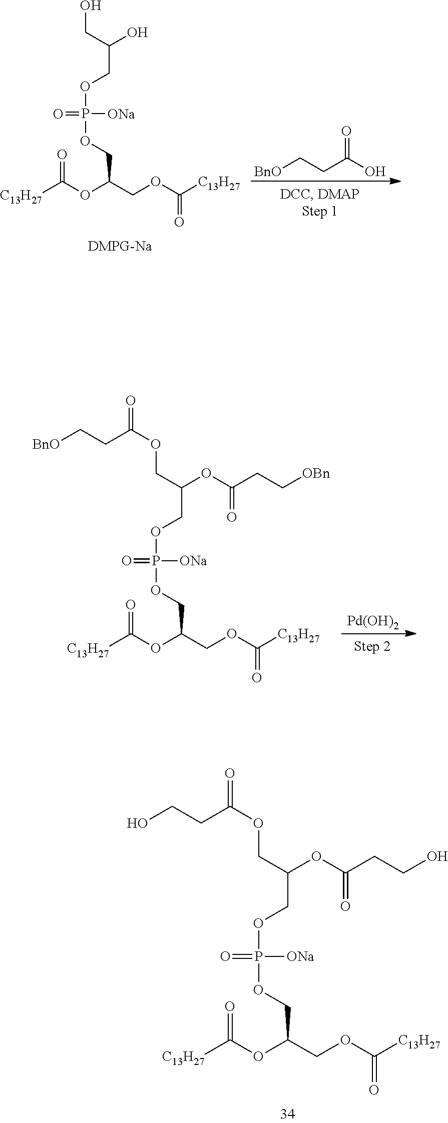

Step 1: Sodium 2,3-bis((3-(benzyloxy)propanoyl)oxy)propyl ((R)-2,3-bis(tetradecanoyloxy) propyl) phosphate.

To a stirred solution of 3-(benzyloxy)propanoic acid (3.92 g, 21.78 mmol, 3 equiv) in DCM (125 ml), dicyclohexylmethanediimine (4.49 g, 21.78 mmol, 3 equiv) was added at RT, and stirred for 30 min. N,N-dimethylpyridin-4-amine (0.443 g, 3.63 mmol, 0.5 equiv) and sodium (R)-2,3-bis(tetradecanoyloxy)propyl (2,3-dihydroxypropyl) phosphate (5.0 g, 7.26 mmol, 1 equiv) was added at RT. The reaction mixture was stirred at RT for 32 h, and was monitored by LCMS. Upon completion, reaction mixture was diluted with DCM (75 ml) and stirred for 10 min and filtered. The filtrate was washed with water (50 ml×1), 0.5N HCl (50 ml xl) and 10% NaHCO₃ (50 ml×1). Combined organic layer was dried over sodium sulfate, filtered, and concentrated to get 7.4 g of crude product. The crude product (7.4 g) was purified by column chromatography using basic 230-400 mesh silica gel (pre basified using ammonia). The product was eluted at 0-10% of methanol in DCM. Pure fractions were collected and concentrated to get 3.6 g of product as colorless syrup. The product was dissolved in DCM (100 ml), washed with 0.5N HCl (50 ml×1), 10% NaHCO₃ solution (50 ml×2). Combined organic layer was dried over sodium sulphate, filtered and concentrated under reduced pressure to get sodium 2,3-bis((3-(benzyloxy)propanoyl)oxy)propyl ((R)-2,3-bis(tetradecanoyloxy)propyl) phosphate (3 g, 40.65% yield) as colourless thick syrup.

¹H NMR (400 MHz, DMSO-d6) δ (ppm)=7.45-7.20 (m, 10H), 5.12-5.04 (m, 2H), 4.45 (d, J=3.3 Hz, 4H), 4.33-4.23 (m, 2H), 4.19-4.03 (m, 2H), 3.79-3.68 (m, 4H), 3.68-3.56 (m, 4H), 2.56 (td, J=6.3, 8.6 Hz, 4H), 2.27-2.20 (m, 4H), 1.53-1.43 (m, 4H), 1.22 (br s, 40H), 0.93-0.78 (m, 6H). LCMS: Mol. formula: C₅₄H₈₆NaO₁₄P, formula weight: 1013.23, exact mass: 990.58, observed mass: 991.6 [M+1]+, RT=3.67 min, purity: 99.76%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: Sodium 2,3-bis((3-hydroxypropanoyl)oxy)propyl ((R)-2,3-bis(tetradecanoyloxy)propyl) phosphate.

To a stirred solution of sodium 2,3-bis((3-(benzyloxy)propanoyl)oxy)propyl ((R)-2,3-bis(tetradecanoyloxy)propyl) phosphate (2.8 g, 2.76 mmol, 1 equiv) in THF (50 ml) was added dihydroxypalladium, 20% dry basis (2 g, 2.85 mmol) at RT under nitrogen atmosphere. The reaction was stirred under 60 psi H₂ pressure for 16 h. The progress of the reaction was monitored by LCMS and TLC. Upon completion, the reaction mixture was filtered through celite bed, washed with THF (100 ml). Filtrate was concentrated under reduced pressure at RT to get 1.85 g of 2,3-bis((3-hydroxypropanoyl)oxy)propyl ((R)-2,3-bis(tetradecanoyloxy)propyl) phosphate. The crude product was dissolved in DCM (20 ml), Amberlite IR120 Na resin (3.6 g) was added, the mixture was stirred for 1.5 h and filtered. Filtrate was concentrated and further dried under vacuum to get sodium 2,3-bis((3-hydroxypropanoyl)oxy)propyl ((R)-2,3-bis(tetradecanoyloxy)propyl) phosphate (1.5 g, 65.2% yield) as an off white solid.

¹H NMR (400 MHz, DMSO-d6) δ (ppm)=5.09-4.96 (m, 2H), 4.30-4.23 (m, 2H), 4.19-4.13 (m, 4H), 3.81-3.50 (m, 8H), 2.47-2.37 (m, 4H), 2.26 (br t, J=6.5 Hz, 4H), 1.63-1.37 (m, 4H), 1.24 (s, 40H), 0.85 (br t, J=6.5 Hz, 6H). LCMS: Mol. formula: C₄₀H₇₄NaO₁₄P, formula weight: 832.98, exact mass: 810.49, observed mass: 809.4 [M−1]⁻, RT=2.93 min, purity: 99.65%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ. HPLC: RT=4.15 min, purity: 99.59%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

### Example 18. (2R)-3-(((2,3-bis((3-aminopropanoyl)oxy)propoxy)(hydroxy)phosphoryl)-oxy)propane-1,2-diyl Ditetradecanoate Dihydrochloride (Compound 35)

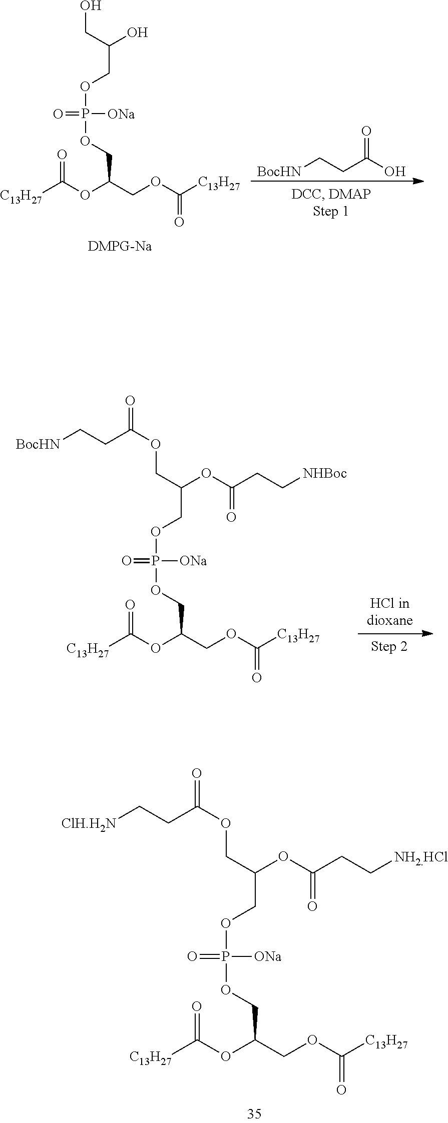

Step 1: Sodium 2,3-bis((3-((tert-butoxycarbonyl)amino)propanoyl)oxy)propyl ((R)-2,3-bis(tetradecanoyloxy)propyl) phosphate.

To a stirred solution of 3-((tert-butoxycarbonyl) amino)propanoic acid (10.30 g, 54.4 mmol, 2.5 equiv) in DCM (325 ml) was added dicyclohexylmethanediimine (13.48 g, 65.3 mmol, 3 equiv) at RT, and stirred for 30 min. N,N-dimethylpyridin-4-amine (1.330 g, 10.89 mmol, 0.5 equiv) and sodium (R)-2,3-bis(tetradecanoyloxy)propyl (2,3-dihydroxypropyl) phosphate (15.0 g, 21.78 mmol, 1 equiv) was added at RT. The reaction mixture was stirred at RT for 16 h, and was monitored by LCMS. Upon completion, the reaction mixture was diluted with DCM (100 ml) stirred for 10 min and filtered. The filtrate was washed with water (100 ml×1), 0.5N HCl (50 ml×1) and 10% NaHCO₃ (50 ml×1). Combined organic layer was dried over sodium sulfate, filtered, and concentrated to get 24.2 g of crude product. The crude product was purified by column chromatography using basic 230-400 mesh silica gel (pre basified using ammonia). The product was eluted at 0%-10% of methanol in ethyl acetate. Pure fractions were collected and concentrated to get pure 11.8 gm product. 11.8 gm product was dissolved in DCM (200 ml), washed with 0.5N HCl (50 ml×2), 10% NaHCO₃ solution (50 ml×2). Combined organic layer was dried over sodium sulphate, filtered and concentrated under reduced pressure to get sodium 2,3-bis((3-((tert-butoxycarbonyl)amino)propanoyl)oxy)propyl ((R)-2,3-bis(tetradecanoyloxy)propyl) phosphate (10.66 g, 47.35% yield) as pale yellow syrup.

¹H NMR (400 MHz, DMSO-d6) δ (ppm)=7.09-6.89 (m, 2H), 5.08-4.99 (m, 2H), 4.32-4.19 (m, 2H), 4.09 (dt, J=6.5, 12.8 Hz, 2H), 3.74 (br dd, J=6.0, 12.0 Hz, 4H), 3.22-3.08 (m, 4H), 2.42 (br t, J=6.8 Hz, 4H), 2.29-2.22 (m, 4H), 1.55-1.45 (m, 4H), 1.37 (s, 18H), 1.24 (s, 40H), 0.90-0.81 (m, 6H). LCMS: Mol. formula: C₅₀H₉₂N₂NaO₁₆P, formula weight: 1031.25, exact mass: 1008.63, observed mass: 1009.7 [M+1]⁺, RT=3.49 min, purity: 98.40%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: (2R)-3-(((2,3-bis((3-aminopropanoyl)oxy)propoxy)(hydroxy)phosphoryl)oxy)propane-1,2-diyl ditetradecanoate hydrochloride.

To a stirred solution of sodium 2,3-bis((3-((tert-butoxycarbonyl)amino)propanoyl)oxy)propyl ((R)-2,3-bis(tetradecanoyloxy)propyl) phosphate (6 g, 5.82 mmol) in DCM (60 ml) was added hydrogen chloride (4M in 1,4-Dioxane, 30 ml, 120 mmol) at 0° C. The reaction mixture was stirred at 10-15° C. for 2.5 h, and monitored by LCMS. Upon completion, the reaction mixture was concentrated under reduced pressure at RT. The residue was co-evaporated with ethyl acetate (75 ml) to and dried to give (2R)-3-(((2,3-bis((3-aminopropanoyl) oxy) propoxy) (hydroxy)phosphoryl)oxy)propane-1,2-diyl ditetradecanoate dihydrochloride (4.9 g, 99.62% yield) as off white solid.

¹H NMR (400 MHz, DMSO-d6) δ (ppm)=8.26-8.02 (m, 6H), 5.18-5.09 (m, 2H), 4.32-4.26 (m, 2H), 4.18-4.00 (m, 4H), 3.98-3.93 (m, 2H), 3.03 (br d, J=5.8 Hz, 4H), 2.74 (dt, J=3.2, 6.5 Hz, 4H), 2.33-2.22 (m, 4H), 1.60-1.43 (m, 4H), 1.24 (s, 40H), 0.91-0.80 (m, 6H). LCMS: Mol. formula: C₄₀H₇₈ClN₂O₁₂P, formula weight: 881.95, exact mass: 808.52. observed mass: 809.5 [M+1]⁺, RT=2.96 min, purity: 97.87%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ. HPLC: RT=6.60 min; purity: 99.98%. Column: Xbridge C8(50×4.6) mm, 3.5 μm, Mobile Phase A:0.1% TFA in water, Mobile Phase B: Acetonitrile, Flow rate:2.0 ml/min.

### Example 19. (2R)-3-(((2,3-bis((L-valyl)oxy)propoxy)(hydroxy)phosphoryl)oxy)propane-1,2-diyl Ditetradecanoate Dihydrochloride (Compound 36)

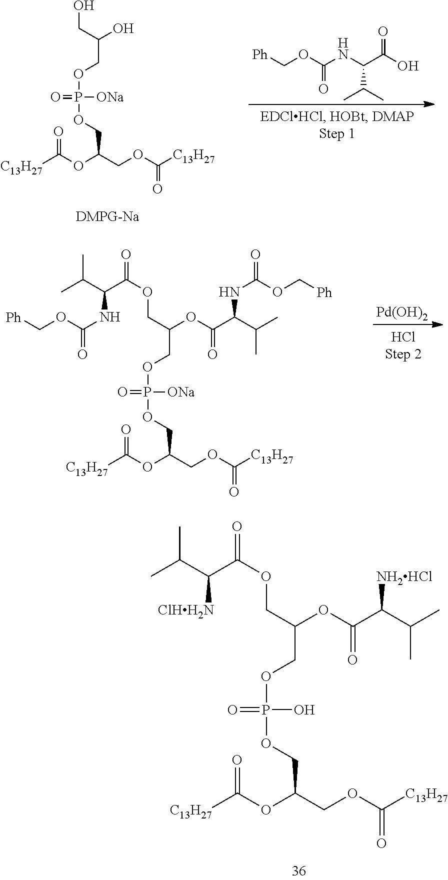

Step 1: Sodium 2,3-bis((((benzyloxy)carbonyl)-L-valyl)oxy)propyl ((R)-2,3-bis(tetradecanoyloxy) propyl) phosphate.

To a stirred solution of ((benzyloxy)carbonyl)-L-valine (4.56 g, 18.15 mmol, 2.5 equiv) in chloroform (125 ml) was added EDC·HCl (4.17 g, 21.78 mmol, 3 equiv) and 1H-benzo[d][1,2,3]triazol-1-ol (2.94 g, 21.78 mmol, 3 equiv) was added at RT, stirred for 30 min. Sodium (R)-2,3-bis(tetradecanoyloxy)propyl (2,3-dihydroxypropyl) phosphate (5.0 g, 7.26 mmol, 1 equiv) and N,N-dimethylpyridin-4-amine (2.66 g, 21.78 mmol, 3 equiv) were added at RT. The reaction mixture was refluxed for 16 h and monitored by LCMS. Upon completion, reaction mixture was diluted with DCM (75 ml) washed with water (50 ml×2), cold 1.5N HCl (50 ml×2), 10% NaHCO₃ solution (50 ml×1). The combined organic layer was washed with brine, dried over sodium sulfate, filtered, and concentrated under reduced pressure to get 8.1 g of crude product. The crude was purified by reverse phase chromatography using (C-18 column) using methanol and water. Pure fractions were collected, concentrated, and further extracted with DCM, washed with of 0.5N HCl (50 ml×1), 10% NaHCO₃ solution (50 ml×2). Combined organic layer was dried over sodium sulphate, filtered, and concentrated under reduced pressure to get sodium 2,3-bis((((benzyloxy)carbonyl)-L-valyl)oxy)propyl ((R)-2,3-bis(tetradecanoyloxy)propyl) phosphate (3.6 g, 43.5% yield) as colorless thick syrup.

¹H NMR (400 MHz, DMSO-d6) δ (ppm)=7.73-7.63 (m, 2H), 7.41-7.28 (m, 10H), 5.14-5.01 (m, 6H), 4.44-4.25 (m, 2H), 4.18-3.93 (m, 4H), 3.82-3.64 (m, 4H), 2.30-2.19 (m, 4H), 2.13-2.01 (m, 2H), 1.54-1.44 (m, 4H), 1.23 (s, 40H), 0.91-0.81 (m, 18H). LCMS: Mol. formula: C₆₀H₉₆N₂NaO₁₆P, formula weight: 1155.39, exact mass: 1132.66, observed mass: 1133.7 [M+1]⁺, RT=3.67 min, purity: 97.85%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: (2R)-3-(((2,3-bis((L-valyl)oxy)propoxy)(hydroxy)phosphoryl)oxy)propane-1,2-diyl ditetradecanoate dihydrochloride.

To a stirred solution of sodium 2,3-bis((((benzyloxy)carbonyl)-L-valyl)oxy)propyl ((R)-2,3-bis(tetradecanoyloxy)propyl) phosphate (0.500 g, 0.433 mmol, 1 equiv) in DCM (20 ml) was added dihydroxypalladium, 20% dry basis (0.250 g, 0.356 mmol) at RT under nitrogen atmosphere. The reaction was stirred at RT under hydrogen bladder pressure for 3 h. The progress of the reaction was monitored by LCMS and TLC. Upon completion, the reaction mixture was filtered through celite bed, washed with mixture of THF (50 ml) and DCM (50 ml). 4M HCl in 1,4 dioxane (3.5 eq) was added to the reaction mixture (filtrate) at 0° C., stirred for 10 min at 0° C. The mixture was concentrated under reduced pressure at RT and dried to get (2R)-3-(((2,3-bis((L-valyl)oxy)propoxy)(hydroxy)phosphoryl)oxy)propane-1,2-diyl ditetradecanoate dihydrochloride (0.330 g, 85% yield) as grey solid.

¹H NMR (400 MHz, DMSO-d6) δ (ppm)=8.85-8.59 (m, 6H), 5.29-5.07 (m, 2H), 4.46-4.38 (m, 2H), 4.32-4.24 (m, 1H), 4.17-3.95 (m, 4H), 3.92-3.75 (m, 4H), 2.33-2.13 (m, 6H), 1.50 (br d, J=6.5 Hz, 4H), 1.24 (s, 40H), 1.05-0.92 (m, 12H), 0.91-0.79 (m, 6H). LCMS: Mol. formula: C₄₄H₈₇Cl₂N₂O₁₂P, formula weight: 938.06, exact mass: 864.58, observed mass: 865.5 [M+1]⁺, RT=3.23 min, purity: 99.19%. Method info: Column: X-BRIDGE C8 (50*4.6) 3.5 μm, Mobile phase:A:0.1% TFA in H2O B: 0.1% TFA in ACN, Flow Rate: 1.5 ml/min. HPLC: RT=7.04 min; purity: 99.92%. Method info: A:0.1% TFA in H2O, B:ACN, Flow Rate: 1.5 ml/min COLUMN: XBridge C8(50×4.6) mm, 3.5 μm.

### Example 20. Sodium 2,3-bis((5-hydroxypentanoyl)oxy)propyl ((R)-2,3-bis(tetradecanoyloxy)propyl) Phosphate (Compound 37)

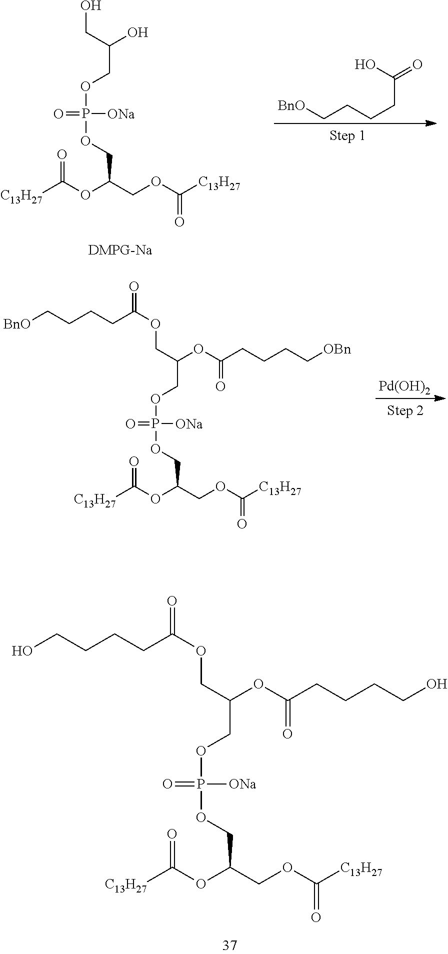

Step 1: Sodium 2,3-bis((5-(benzyloxy)pentanoyl)oxy)propyl ((R)-2,3-bis(tetradecanoyloxy) propyl) phosphate.

To a stirred solution of 5-(benzyloxy)pentanoic acid (4.16 g, 19.96 mmol), 2.5 equiv) in DCM (150 ml), dicyclohexylmethanediimine (4.94 g, 23.95 mmol, 3 equiv) was added at RT, and the mixture was stirred for 30 min. N,N-dimethylpyridin-4-amine (0.488 g, 3.99 mmol, 0.5 equiv) and sodium (R)-2,3-bis(tetradecanoyloxy)propyl (2,3-dihydroxypropyl) phosphate (5.5 g, 7.98 mmol, 1 equiv) was added at RT. The reaction mixture was stirred at RT for 24 h, and was monitored by LCMS. Upon completion, reaction mixture was diluted with DCM (75 ml) and stirred for 10 min and filtered. The filtrate was washed with water (50 ml×1), 0.5N HCl (50 ml xl) and 10% NaHCO₃ (50 ml×1). Combined organic layer was dried over sodium sulfate, filtered, and concentrated to get 8.2 g of crude product. The crude product (8.2 g) was purified by column chromatography using basic 230-400 mesh silica gel (pre basified using ammonia). The product was eluted at 0-10% of methanol in ethyl acetate. Pure fractions were collected and concentrated to get 3.8 g sodium 2,3-bis((5-(benzyloxy)pentanoyl)oxy)propyl ((R)-2,3-bis(tetradecanoyloxy)propyl) phosphate as colorless syrup. This product was dissolved in DCM (100 ml), washed with of 0.5N HCl (50 ml×1), 10% NaHCO₃ solution (50 ml×2). Combined organic layer was dried over sodium sulphate, filtered and concentrated under reduced pressure to get sodium 2,3-bis((5-(benzyloxy)pentanoyl)oxy)propyl ((R)-2,3-bis(tetradecanoyloxy)propyl) phosphate (3.5 g, 40.0% yield) as colorless thick syrup.

¹H NMR (400 MHz, DMSO-d6) δ (ppm)=7.37-7.24 (m, 10H), 5.09-5.02 (m, 2H), 4.42 (s, 4H), 4.33-4.23 (m, 2H), 4.13-4.02 (m, 2H), 3.75-3.62 (m, 4H), 3.44-3.36 (m, 4H), 2.35-2.19 (m, 8H), 1.67-1.40 (m, 12H), 1.22 (s, 40H), 0.90-0.80 (m, 6H). LCMS: Mol. formula: C₅₈H₉₄NaO₁₄P, formula weight: 1069.34, exact mass: 1046.65, observed mass: 1047.7 [M+1]+, RT=3.79 min, purity: 99.73%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

### Step 2: Sodium 2,3-bis((5-hydroxypentanoyl)oxy)propyl ((R)-2,3-bis(tetradecanoyloxy)propyl) phosphate

To a stirred solution of sodium 2,3-bis((5-(benzyloxy)pentanoyl)oxy)propyl ((R)-2,3-bis(tetradecanoyloxy)propyl) phosphate (1 g, 0.935 mmol, 1 equiv) in THF (20 ml) was added dihydroxypalladium, 20% dry basis (1 g, 7.12 mmol) at RT under nitrogen atmosphere. The reaction was stirred under 60 psi H2 pressure for 16 h. The progress of the reaction was monitored by LCMS and TLC. Upon completion, the reaction mixture was filtered through celite bed, washed with THF (50 ml). Filtrate was concentrated under reduced pressure at RT to get 0.750 g of sodium 2,3-bis((5-hydroxypentanoyl)oxy)propyl ((R)-2,3-bis(tetradecanoyloxy)propyl) phosphate. This product was further (0.750 g) was dissolved in DCM (10 ml), was added Amberlite IR120 Na resin (1.5 g). Reaction was stirred for 1.5 h and filtered. Filtrate was concentrated and further dried under vacuum to get sodium 2,3-bis((5-hydroxypentanoyl)oxy)propyl ((R)-2,3-bis(tetradecanoyloxy)propyl) phosphate (0.660 g, 79.4% yield) as an off white solid.

¹H NMR (400 MHz, DMSO-d6) δ (ppm)=5.05 (br s, 2H), 4.55 (br s, 2H), 4.33-4.18 (m, 2H), 4.09 (br dd, J=5.8, 11.8 Hz, 2H), 3.74-3.66 (m, 4H), 3.48-3.43 (m, 4H), 2.36-2.18 (m, 8H), 1.59-1.37 (m, 12H), 1.23 (s, 40H), 0.85 (br t, J=6.5 Hz, 6H). LCMS: Mol. formula: C₄₄H₈₂NaO₁₄P, formula weight: 889.09, exact mass: 866.55, observed mass: 865.5 [M−1]⁻, RT=2.94 min, purity: 99.78%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ. HPLC: RT=3.39 min; purity: 96.4%. Method: Mobile Phase A: 1 ml of 25% ammonia solution in 1000 ml of MilliQ Water (pH:9 with Acetic acid). Mobile Phase B: acetonitrile. How Rate: 1.0 ml/min. COLUMN: XBridge C8 (50×4.6) mm, 3.5μ.

### Example 21. (2R)-3-(((2,3-bis((5-aminopentanoyl)oxy)propoxy)(hydroxy)phosphoryl)oxy)-propane-1,2-diyl Ditetradecanoate Dihydrochloride (Compound 38)

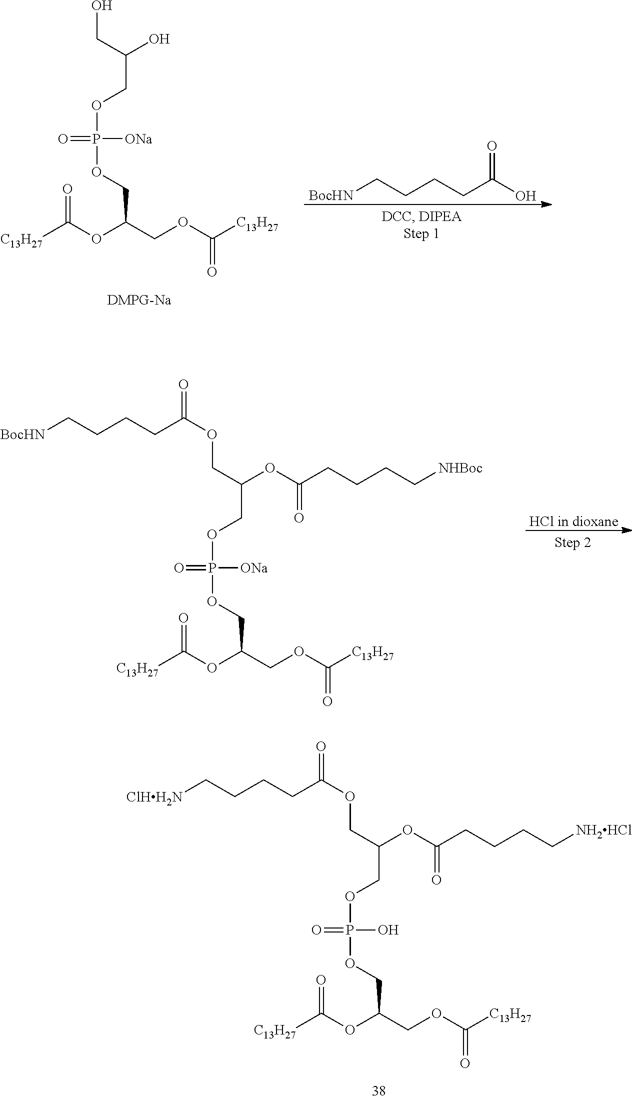

Step 1: Sodium 2,3-bis((5-((tert-butoxycarbonyl)amino)pentanoyl)oxy)propyl ((R)-2,3-bis(tetradecanoyloxy)propyl) phosphate.

To a stirred solution of 5-((tert-butoxycarbonyl)amino)pentanoic acid (3.78 g, 17.42 mmol, 3 equiv) in DCM (100 ml), dicyclohexylmethanediimine (4.19 g, 20.348 mmol, 3.5 equiv), N,N-dimethylpyridin-4-amine (0.177 g, 1.452 mmol, 0.25 equiv) and sodium (R)-2,3-bis(tetradecanoyloxy)propyl (2,3-dihydroxypropyl) phosphate (4.0 g, 5.81 mmol, 1 equiv) were added at RT. The reaction mixture was stirred at RT for 24 h, and was monitored by LCMS. Upon completion, reaction mixture was diluted with DCM (50 ml) and stirred for 10 min and filtered. The filtrate was washed with water (50 ml×1), 0.5N HCl (50 ml×1) and 10% NaHCO₃ (50 ml×1). Combined organic layer was dried over sodium sulfate, filtered, and concentrated to get 6.2 g of crude product. The crude product (6.2 g) was purified by column chromatography using silica gel (230-400 mesh silica gel), and the product was eluted with 0-8% of methanol in DCM. Pure fractions were collected and concentrated to get sodium 2,3-bis((5-((tert-butoxycarbonyl)amino)pentanoyl)oxy)propyl ((R)-2,3-bis(tetradecanoyloxy)propyl) phosphate (3.5 g, 56.4%) as pale yellow thick syrup.

¹H NMR (400 MHz, DMSO-d6) δ (ppm)=6.95-6.74 (m, 2H), 5.08-4.99 (m, 2H), 4.33-4.16 (m, 2H), 4.13-4.05 (m, 2H), 3.80-3.64 (m, 4H), 2.90 (q, J=6.4 Hz, 4H), 2.30-2.18 (m, 7H), 1.57-1.39 (m, 11H), 1.37 (s, 20H), 1.24 (s, 40H), 0.88-0.84 (m, 6H). LCMS: Mol. formula: C₅₄H₁₀nN₂NaO₁₆P, formula weight: 1087.36, exact mass: 1064.69, observed mass: 1063.7 [M−1]⁻, RT=3.51 min, purity: 99.56%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: (2R)-3-(((2,3-bis((5-aminopentanoyl)oxy)propoxy)(hydroxy)phosphoryl)oxy)propane-1,2-diyl ditetradecanoate dihydrochloride.

To a stirred solution of solution of 2,3-bis((5-((tert-butoxycarbonyl)amino)pentanoyl)oxy)propyl ((R)-2,3-bis(tetradecanoyloxy)propyl) phosphate (1.1 g, 1.033 mmol, 1 equiv) in 1,4-Dioxane (11 ml) was added hydrogen chloride (4.0 M HCl in 1,4-Dioxane, 11 ml, 44.0 mmol) at 10° C. The reaction mixture was stirred at 15-20° C. for 1 h 30 min. The progress of the reaction was monitored by LCMS. Upon completion, the reaction mixture was concentrated under reduced pressure at RT. The residue was co-evaporated with ethyl acetate (25 ml×1) to get (2R)-3-(((2,3-bis((5-aminopentanoyl)oxy)propoxy)(hydroxy)phosphoryl)oxy)propane-1,2-diyl ditetradecanoate dihydrochloride (760 mg, 76%) as an off white solid.

¹H NMR (400 MHz, DMSO-d6) δ (ppm)=8.16-8.02 (m, 6H), 5.16-5.06 (m, 2H), 4.33-4.19 (m, 2H), 4.17-4.09 (m, 2H), 4.04-3.96 (m, 1H), 3.92-3.73 (m, 4H), 2.75 (br s, 4H), 2.41-2.18 (m, 8H), 1.64-1.46 (m, 12H), 1.24 (s, 40H), 0.93-0.78 (m, 6H). LCMS: Mol. formula: C₄₄H₈₇Cl₂N₂O₁₂, formula weight: 938.06, exact mass: 864.58. observed mass: 865.5 [M+1]⁺, RT=3.15 min, purity: 97.29%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ. HPLC: RT=6.71 min; purity: 94.83%. Method: Column: Xbridge C8(50×4.6) mm, 3.5 μm, Mobile Phase A:0.1% TFA in water, Mobile Phase B: Acetonitrile, Flow rate:2.0 ml/min.

### Example 22. (3S,3'S)-4,4′-((3-((((R)-2,3-bis(tetradecanoyloxy)propoxy)(hydroxy)phosphoryl)oxy) propane-1,2-diyl)bis(oxy))bis(3-amino-4-oxobutanoic acid) Dihydrochloride (Compound 39)

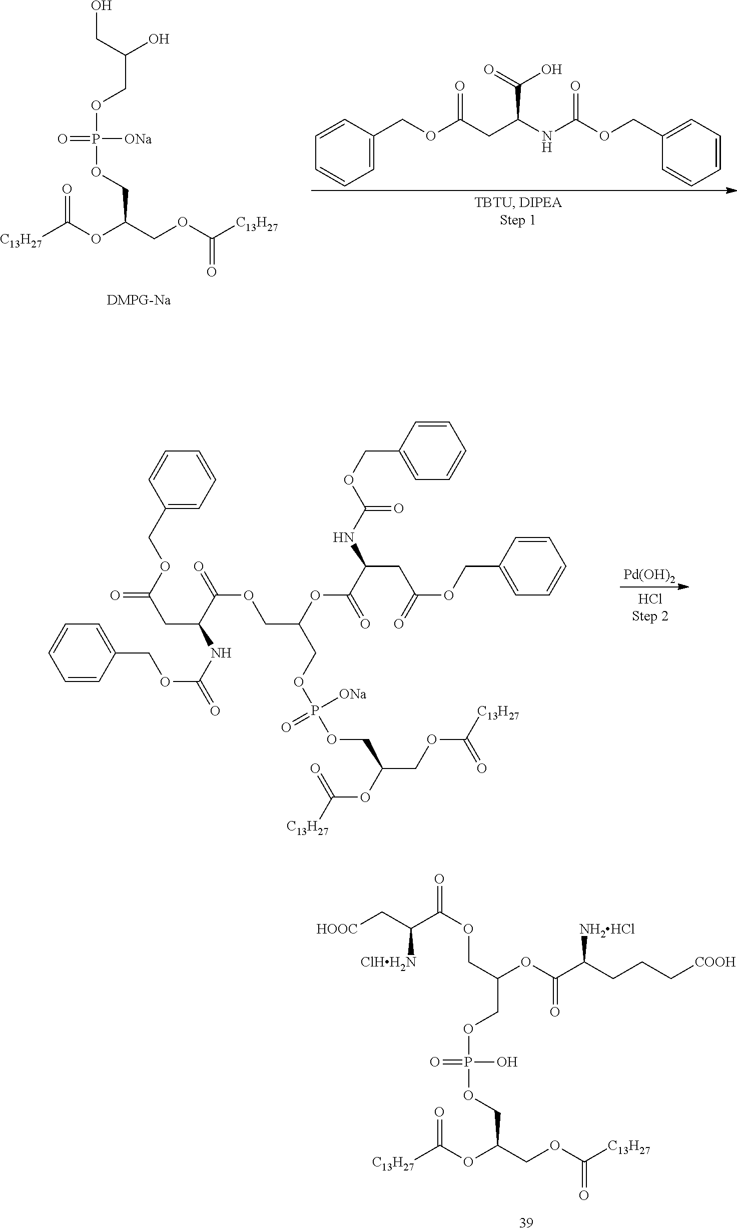

Step 1: Sodium 2,3-bis(((S)-4-(benzyloxy)-2-(((benzyloxy)carbonyl)amino)-4-oxobutanoyl)oxy)propyl ((R)-2,3-bis(tetradecanoyloxy)propyl) phosphate.

To a stirred solution of (R)-4-(benzyloxy)-2-(((benzyloxy)carbonyl)amino)-4-oxobutanoic acid (5.19 g, 14.52 mmol, 2.5 equiv) in DCM (100 ml), TBTU (7.46 g, 23.23 mmol, 4 equiv) was added at RT and stirred for 30 min. Sodium (R)-2,3-bis(tetradecanoyloxy)propyl (2,3-dihydroxypropyl) phosphate (4 g, 5.81 mmol, 1 equiv) and N-ethyl-N-isopropylpropan-2-amine (5.06 ml, 29.0 mmol, 5 equiv) was added at 5° C., stirred at RT for 20 h. Reaction progress was monitored by LCMS. Upon completion, reaction mixture was diluted with DCM (75 ml) washed with water (50 ml×2), cold 1.5N HCl (50 ml×2), 10% NaHCO₃ (50 ml xl). The combined organic layer was washed with brine, dried over sodium sulfate, filtered and concentrated under reduced pressure to get 7.1 g of crude product. The crude was purified by column chromatography using basic 230-400 mesh silica gel (pre basified using ammonia). The product was eluted at 0-10% of methanol in ethyl acetate. Pure fractions were collected and concentrated to get 3.4 g of product which was dissolved in DCM (100 ml), washed with 0.5N HCl (50 ml×1), 10% NaHCO₃ solution (50 ml×2). The combined organic layer was dried over sodium sulphate, filtered, and concentrated under reduced pressure to get sodium 2,3-bis(((S)-4-(benzyloxy)-2-(((benzyloxy)carbonyl)amino)-4-oxobutanoyl)oxy)propyl ((R)-2,3-bis(tetradecanoyloxy)propyl) phosphate (3.1 g, 38.8% yield) as colorless thick syrup.

¹H NMR (400 MHz, DMSO-d6) δ (ppm)=8.11-7.92 (m, 2H), 7.37-7.29 (m, 20H), 5.11-4.99 (m, 8H), 5.06 (br s, 2H), 4.63-4.44 (m, 2H), 4.34-4.01 (m, 4H), 3.71 (br dd, J=5.0, 10.0 Hz, 4H), 3.02-2.66 (m, 4H), 2.23 (br t, J=7.3 Hz, 4H), 1.47 (br s, 4H), 1.29-1.15 (m, 40H), 0.85 (t, J=6.8 Hz, 6H). LCMS: Mol. formula: C₇₂H₁₀₀N₂NaO₂₀P, formula weight: 1367.55, exact mass: 1344.67, observed mass: 1343.7 [M−1]⁻, RT=3.74 min, purity: 99.70%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: (3S,3'S)-4,4′-((3-((((R)-2,3-bis(tetradecanoyloxy)propoxy)(hydroxy)phosphoryl)oxy) propane-1,2-diyl)bis(oxy))bis(3-amino-4-oxobutanoic acid) dihydrochloride.

To a stirred solution of sodium 2,3-bis(((S)-4-(benzyloxy)-2-(((benzyloxy)carbonyl)amino)-4-oxobutanoyl)oxy)propyl ((R)-2,3-bis(tetradecanoyloxy)propyl) phosphate (1.5 g, 1.097 mmol, 1 equiv) in DCM (30 ml) was added dihydroxypalladium, 20% dry basis (0.750 g, 1.068 mmol) at RT under nitrogen atmosphere. The reaction was stirred at RT under hydrogen bladder pressure for 4 h and was monitored by LCMS and TLC. Upon completion, the reaction mixture was filtered through celite bed, washed with mixture of THF (50 ml) and DCM (50 ml). Filtrate was concentrated under reduced pressure to get 1.0 g product which was triturated with MTBE (20 ml×2), stirred for 15 min, filtered and dried to get 720 mg of product. This product was dissolved in DCM (10 vol) was added 4M HCl in 1,4 dioxane (4 equiv) at 0° C. and stirred for 30 min at 0° C. The mixture was concentrated under reduced pressure at RT and dried to get bis(tetradecanoyloxy)propoxy)(hydroxy)phosphoryl)oxy)propane-1,2-diyl)bis(oxy))bis(3-amino-4-oxobutanoic acid) dihydrochloride (0.450 g, 42.45% yield) as grey solid.

¹H NMR (400 MHz, DMSO-d6) δ (ppm)=9.20-8.24 (m, 6H), 5.20-5.07 (m, 2H), 4.48-4.25 (m, 5H), 4.16-3.94 (m, 4H), 3.86 (br d, J=5.5 Hz, 4H), 2.96 (br d, J=5.5 Hz, 4H), 2.36-2.18 (m, 4H), 1.55-1.45 (m, 4H), 1.24 (s, 40H), 0.90-0.81 (m, 6H). LCMS: Mol. formula: C₄₂H₇₉Cl₂N₂O₁₆P, formula weight: 969.97, exact mass: 896.50, observed mass: 897.4 [M+1]⁺, RT=3.12 min, purity: 98.80%. Method info: Column: X-BRIDGE C8 (50*4.6) 3.5 μm, Mobile phase:A:0.1% TFA in H2O B: 0.1% TFA in ACN, Flow Rate: 1.5 ml/min. HPLC: RT=6.68 min; purity: 99.92%. Method info: A:0.1% TFA in H2O, B:ACN, How Rate: 1.5 ml/min. COLUMN: XBridge C8(50×4.6) mm, 3.5 μm.

### Example 23. Sodium 2,3-bis((4-carboxybutanoyl)oxy)propyl ((R)-2,3-bis(tetradecanoyloxy)-propyl) Phosphate (Compound 40)

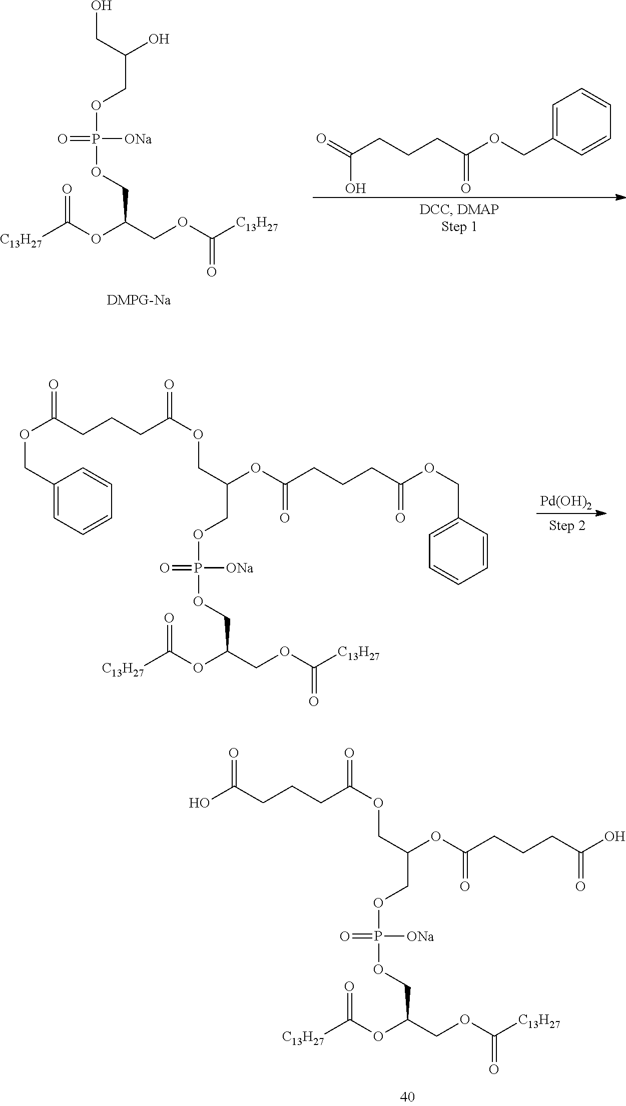

Step 1: Sodium 2,3-bis((5-(benzyloxy)-5-oxopentanoyl)oxy)propyl ((R)-2,3-bis(tetradecanoyl-oxy)propyl) phosphate.

To a stirred solution of 5-(benzyloxy)-5-oxopentanoic acid (4.03 g, 18.15 mmol, 2.5 equiv) in DCM (125 ml), dicyclohexylmethanediimine (4.49 g, 21.78 mmol, 3 equiv) was added at RT, and the mixture was stirred for 30 min. N, N-dimethylpyridin-4-amine (0.443 g, 3.63 mmol, 0.5 equiv) and sodium (R)-2,3-bis(tetradecanoyloxy)propyl (2,3-dihydroxypropyl) phosphate (5.0 g, 7.26 mmol, 1 equiv) was added at RT, and the reaction mixture was stirred at RT for 16 h. Reaction progress was monitored by LCMS. Upon completion, reaction mixture was diluted with DCM (75 ml) and stirred for 10 min and filtered. The filtrate was washed with water (50 ml×1), 0.5N HCl (50 ml xl) and 10% NaHCO₃ (50 ml×1). Combined organic layer was dried over sodium sulfate, filtered, and concentrated to get 8.0 g of crude product. The crude product (8.0 g) was purified by column chromatography using basic 230-400 mesh silica gel (pre basified using ammonia). The product was eluted at 0-10% of methanol in ethyl acetate. Pure fractions were collected and concentrated to get 4.0 μg sodium 2,3-bis((5-(benzyloxy)-5-oxopentanoyl)oxy)propyl ((R)-2,3-bis(tetradecanoyloxy)propyl) phosphate as colorless syrup. This product was dissolved in DCM (100 ml), washed with of 0.5N HCl (50 ml×1), 10% NaHCO₃ solution (50 ml×2). Combined organic layer was dried over sodium sulphate, filtered and concentrated under reduced pressure to get sodium 2,3-bis((5-(benzyloxy)-5-oxopentanoyl)oxy)propyl ((R)-2,3-bis(tetradecanoyloxy)propyl) phosphate (3.67 g, 45.9% yield) as colorless thick syrup.

¹H NMR (400 MHz, DMSO-d6) δ (ppm)=7.38-7.29 (m, 10H), 5.08 (s, 6H), 4.29 (br d, J=11.8 Hz, 2H), 4.14-4.04 (m, 2H), 3.72 (br s, 4H), 2.42-2.18 (m, 12H), 1.83-1.72 (m, 4H), 1.54-1.43 (m, 4H), 1.22 (s, 40H), 0.92-0.77 (m, 6H). LCMS: Mol. formula: C₅₈H₉₀NaOI₆P, formula weight: 1097.31, exact mass: 1074.60, observed mass: 1073.5 [M−1]⁻, RT=3.58 min, purity: 99.68%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5 μp.

Step 2: Sodium 2,3-bis((4-carboxybutanoyl)oxy)propyl ((R)-2,3-bis(tetradecanoyloxy)propyl) phosphate.

To a stirred solution of sodium 2,3-bis((5-(benzyloxy)-5-oxopentanoyl)oxy)propyl ((R)-2,3-bis(tetradecanoyloxy)propyl) phosphate (1.0 g, 0.911 mmol, 1 equiv) in DCM (20 ml) was added dihydroxypalladium, 20% dry basis (0.600 g, 4.27 mmol) at RT under nitrogen atmosphere. The reaction was stirred under hydrogen bladder pressure for 16 h, and was monitored by LCMS and TLC. Upon completion, the reaction mixture was filtered through celite bed, washed with THF (25 ml) and DCM (50 ml). Filtrate was concentrated under reduced pressure at RT to get 0.650 g of sodium 2,3-bis((4-carboxybutanoyl)oxy)propyl ((R)-2,3-bis(tetradecanoyloxy)propyl) phosphate. This product was dissolved in DCM (10 ml), Amberlite IR120 Na resin (3.5 g) was added, stirred for 1.5 h and filtered. Filtrate was concentrated and further dried under vacuum to get sodium 2,3-bis((4-carboxybutanoyl)oxy)propyl ((R)-2,3-bis(tetradecanoyloxy)propyl) phosphate (0.520 g, 62.2% yield) as an off white solid.

¹H NMR (400 MHz, DMSO-d6) δ (ppm)=12.93-12.12 (m, 2H), 5.08 (br s, 2H), 4.34-4.18 (m, 2H), 4.11 (br dd, J=6.5, 11.0 Hz, 2H), 3.85-3.70 (m, 4H), 2.40-2.18 (m, 12H), 1.87-1.60 (m, 4H), 1.56-1.43 (m, 4H), 1.23 (s, 40H), 0.85 (br t, J=6.5 Hz, 6H). LCMS: Mol. formula: C₄₄H₇₈NaOI₆P, formula weight: 917.06, exact mass: 894.51, observed mass: 917.5 [M+Na]+, RT=3.57 min, purity: 98.07%. Method info: Column: X-BRIDGE C8 (50*4.6) 3.5 μm. Mobile phase:A:0.1% TFA in H2O B: 0.1% TFA in CAN, How Rate: 1.5 ml/min. HPLC: RT=7.77 min; purity: 98.14%. Method info: A:0.1% TFA in H2O, B:ACN, How Rate: 1.5 ml/min COLUMN: XBridge C8(50×4.6) mm, 3.5 μm.

### Example 24. Sodium 2,3-bis((3-carboxypropanoyl)oxy)propyl ((R)-2,3-bis(tetradecanoyl-oxy)propyl) Phosphate (Compound 41)

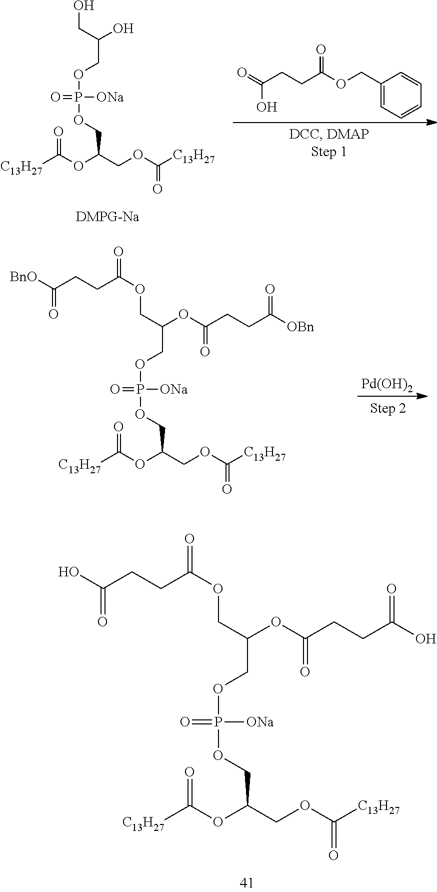

Step 1: Sodium 2,3-bis((4-(benzyloxy)-4-oxobutanoyl)oxy)propyl ((R)-2,3-bis(tetradecanoyloxy) propyl) phosphate.

To a stirred solution of 4-(benzyloxy)-4-oxobutanoic acid (3.78 g, 18.15 mmol, 2.5 equiv) in DCM (125 ml), dicyclohexylmethanediimine (4.49 g, 21.78 mmol, 3 equiv) was added at RT, and the mixture was stirred for 30 min. N, N-dimethylpyridin-4-amine (0.443 g, 3.63 mmol, 0.5 equiv) and sodium (R)-2,3-bis(tetradecanoyloxy)propyl (2,3-dihydroxypropyl) phosphate (5.0 g, 7.26 mmol, 1 equiv) was added at RT, and stirred at RT for 18 h. Reaction progress was monitored by LCMS. Upon completion, reaction mixture was diluted with DCM (75 ml) and stirred for 10 min and filtered. The filtrate was washed with water (50 ml×1), 0.5N HCl (50 ml xl) and 10% NaHCO₃ (50 ml×1). Combined organic layer was dried over sodium sulfate, filtered, and concentrated to get 6.8 g of crude product. The crude was purified by column chromatography using basic 230-400 mesh silica gel (pre basified using ammonia). The product was eluted at 0-10% of methanol in ethyl acetate. Pure fractions were collected and concentrated to get 3.4 g sodium 2,3-bis((4-(benzyloxy)-4-oxobutanoyl) oxy) propyl ((R)-2,3-bis(tetradecanoyloxy)propyl) phosphate as colorless syrup. This product was dissolved in DCM (100 ml), washed with of 0.5N HCl (50 ml×1), 10% NaHCO₃ solution (50 ml×2). Combined organic layer was dried over sodium sulphate, filtered, and concentrated under reduced pressure to get sodium 2,3-bis((4-(benzyloxy)-4-oxobutanoyl) oxy) propyl ((R)-2,3-bis(tetradecanoyloxy)propyl) phosphate (3.1 g, 40.4. % yield) as colorless thick syrup.

¹H NMR (400 MHz, DMSO-d6) δ (ppm)=7.41-7.26 (m, 10H), 5.08 (s, 6H), 4.31-4.19 (m, 2H), 4.17-4.05 (m, 2H), 3.79-3.66 (m, 4H), 2.64-2.54 (m, 8H), 2.29-2.18 (m, 4H), 1.53-1.44 (m, 4H), 1.22 (br s, 40H), 0.84 (br t, J=6.8 Hz, 6H). LCMS: Mol. formula: C₅₆H₈₆NaO₁₆P, formula weight: 1069.25, exact mass: 1046.57, observed mass: 1045.5 [M−1]⁻, RT=3.61 min, purity: 99.47%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: Sodium 2,3-bis((3-carboxypropanoyl)oxy)propyl ((R)-2,3-bis(tetradecanoyloxy)propyl) phosphate.

To a stirred solution of 2,3-bis((4-(benzyloxy)-4-oxobutanoyl)oxy)propyl ((R)-2,3-bis(tetradecanoyloxy)propyl) phosphate (1.0 g, 0.935 mmol, 1 equiv) in DCM (20 ml) was added dihydroxypalladium, 20% dry basis (0.600 g, 0.854 mmol) at RT under nitrogen atmosphere. The reaction was stirred under under hydrogen bladder pressure for 16 h. The progress of the reaction was monitored by LCMS and TLC. Upon completion, the reaction mixture was filtered through celite bed, washed with THF (25 ml) and DCM (50 ml). Filtrate was concentrated under reduced pressure at RT to get 0.750 g of sodium 2,3-bis((3-carboxypropanoyl)oxy)propyl ((R)-2,3-bis(tetradecanoyloxy)propyl) phosphate. This product was dissolved in DCM (10 ml), Amberlite IR120 Na resin (4.5 g) was added, stirred for 1.5 h and filtered. Filtrate was concentrated and further dried under vacuum to get sodium 2,3-bis((3-carboxypropanoyl)oxy)propyl ((R)-2,3-bis(tetradecanoyloxy)propyl) phosphate (0.600 g, 72.2% yield) as an off white solid.

¹H NMR (400 MHz, DMSO-d6) δ (ppm)=12.95-11.04 (m, 2H), 5.12-4.97 (m, 2H), 4.33-4.05 (m, 4H), 3.76 (br s, 4H), 2.64-2.54 (m, 12H), 1.57-1.41 (m, 4H), 1.23 (br s, 40H), 0.91-0.78 (m, 6H). LCMS: Mol. formula: C₄₂H₇₄NaO₁₆P, formula weight: 889.00, exact mass: 866.48, observed mass: 889.4 [M+Na]+, RT=3.60 min, purity: 98.80%. Method: Column: X-BRIDGE C8 (50*4.6) 3.5 m, Mobile phase:A:0.1% TFA in H2O B: 0.1% TFA in ACN, How Rate: 1.5 ml/min. HPLC: RT=7.80 min; purity: 98.46%. Method info: A:0.1% TFA in H2O, B:ACN, Flow Rate: 1.5 ml/min. COLUMN: XBridge C8(50×4.6) mm, 3.5 μm.

### Example 25. (4S,4'S)-5,5′-((3-((((R)-2,3-bis(tetradecanoyloxy)propoxy)(hydroxy)phosphoryl)oxy)propane-1,2-diyl)bis(oxy))bis(4-amino-5-oxopentanoic acid) Dihydrochloride (Compound 42)

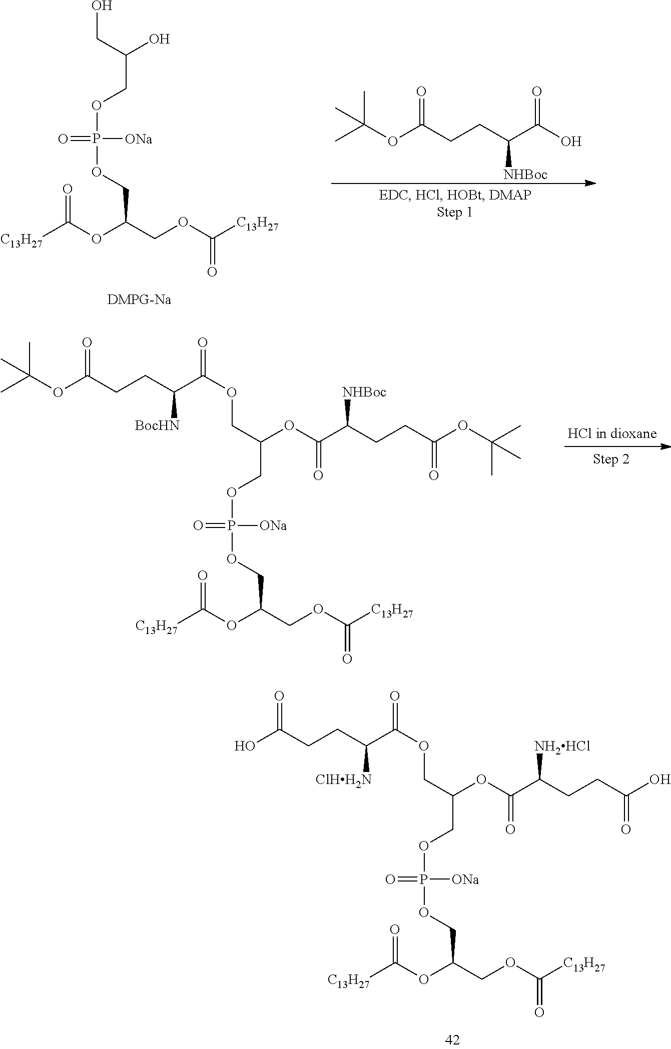

Step 1: Sodium 2,3-bis(((S)-5-(tert-butoxy)-2-((tert-butoxycarbonyl)amino)-5-oxopentanoyl) oxy)propyl ((R)-2,3-bis(tetradecanoyloxy)propyl) phosphate.

To a stirred solution of (S)-5-(tert-butoxy)-2-((tert-butoxycarbonyl)amino)-5-oxopentanoic acid (6.61 g, 21.78 mmol, 3.0 equiv) in chloroform (75 ml), EDC·HCl (3.48 g, 18.15 mmol, 2.5 equiv) and 1H-benzo[d][1,2,3]triazol-1-ol (2.452 g, 18.15 mmol, 2.5 equiv) were added at RT, stirred for 30 min. Sodium (R)-2,3-bis(tetradecanoyloxy)propyl (2,3-dihydroxypropyl) phosphate (5.0 g, 7.26 mmol, 1 equiv) and N,N-dimethylpyridin-4-amine (2.217 g, 18.15 mmol, 2.5 equiv) was added at RT and the reaction mixture was refluxed for 16 h. Added additional EDC·HCl (3.48 g, 18.15 mmol, 2.5 equiv), 1H-benzo[d][1,2,3]triazol-1-ol (2.452 g, 18.15 mmol, 2.5 equiv) and N,N-dimethylpyridin-4-amine (2.217 g, 18.15 mmol, 2.5 equiv) and reaction was refuxed for 2 h. Reaction progress was monitored by LCMS, upon completion, reaction mixture was diluted with DCM (75 ml) washed with water (50 ml×2), cold 1.5N HCl (50 ml×2), 10% NaHCO₃ solution (50 ml xl). The combined organic layer was washed with brine, dried over sodium sulfate, filtered, and concentrated under reduced pressure to get 8.1 g of crude product. This crude product was purified by column chromatography using 230-400 mesh silica gel. The product was eluted with 0-10% of methanol in DCM. Pure fractions were collected and concentrated to get sodium 2,3-bis(((S)-5-(tert-butoxy)-2-((tert-butoxycarbonyl)amino)-5-oxopentanoyl)oxy)propyl ((R)-2,3-bis(tetradecanoyloxy)propyl) phosphate (3.9 g, 42.6% yield) as colorless thick syrup.

¹H NMR (400 MHz, DMSO-d6) δ (ppm)=7.38-7.16 (m, 2H), 5.05 (br s, 2H), 4.39-4.21 (m, 2H), 4.17-3.93 (m, 4H), 3.79-3.57 (m, 4H), 2.33-2.16 (m, 10H), 2.00-1.93 (m, 2H), 1.82-1.65 (m, 4H), 1.38 (br d, J=4.0 Hz, 36H), 1.24 (s, 40H), 0.86-0.82 (m, 6H). LCMS: Mol. formula: C₆₂H₁₁₂N₂NaO₂OP, formula weight: 1259.54, exact mass: 1236.76, observed mass: 1235.8[M−1]⁻, RT=4.04 min, purity: 99.87%. Method: Mobile Phase A: 1 ml of 25% ammonia solution in 1000 ml of MilliQ Water (pH:9 with Acetic acid). Mobile Phase B: acetonitrile. Flow Rate: 1.0 ml/min. COLUMN: XBridge C8 (50×4.6) mm, 3.5μ.

Step 2: (4S,4'S)-5,5′-((3-((((R)-2,3-bis(tetradecanoyloxy)propoxy)(hydroxy)phosphoryl) oxy)propane-1,2-diyl)bis(oxy))bis(4-amino-5-oxopentanoic acid) dihydrochloride.

To a stirred solution of 2,3-bis(((S)-5-(tert-butoxy)-2-((tert-butoxycarbonyl) amino)-5-oxopentanoyl) oxy) propyl ((R)-2,3-bis(tetradecanoyloxy)propyl) phosphate (1 g, 0.809 mmol, 1 equiv) in 1,4-Dioxane (10 ml) was added hydrogen chloride (4.0 M in 1,4-Dioxane, 15 ml, 60.0 mmol) at 10° C. and the mixture was stirred at RT for 4 h. Reaction was monitored by LCMS, upon completion, was concentrated under reduced pressure at RT. The residue was co-evaporated with ethyl acetate (25 ml×1) and dried to get (4S,4'S)-5,5′-((3-((((R)-2,3-bis(tetradecanoyloxy)propoxy) (hydroxy)phosphoryl)oxy)propane-1,2-diyl)bis(oxy))bis(4-amino-5-oxopentanoic acid) dihydrochloride (0.680 g, 84.2% yield) as off-white solid.

¹H NMR (400 MHz, DMSO-d6) δ (ppm)=13.74-11.16 (m, 2H), 9.06-8.58 (m, 6H), 5.28-5.02 (m, 2H), 4.43-4.24 (m, 5H), 4.17-3.97 (m, 6H), 3.96-3.79 (m, 4H), 2.30-2.17 (m, 4H), 2.14-1.94 (m, 4H), 1.55-1.39 (m, 4H), 1.23 (s, 40H), 0.90-0.80 (m, 6H). LCMS: Mol. formula: C₄₄H₈₃Cl₂N₂O₁₆P, formula weight: 998.02, exact mass: 924.53, observed mass: 925.4 [M+1]⁺, RT=3.14 min, purity: 97.26%. Method info: Column: X-BRIDGE C8 (50*4.6) 3.5 μm, Mobile phase:A:0.1% TFA in H2O B: 0.1% TFA in ACN, Flow Rate: 1.5 ml/min. HPLC: Rt=6.64 min; purity: 97.2%. Method info: A:0.1% TFA in H2O, B:ACN, Flow Rate: 1.5 ml/min. COLUMN: XBridge C8 (50×4.6) mm, 3.5 μm.

### Example 26. (2R)-3-(((2,3-bis((3-(methylamino)propanoyl)oxy)propoxy)(hydroxy)-phosphoryl)oxy)propane-1,2-diyl Ditetradecanoate Dihydrochloride (Compound 43)

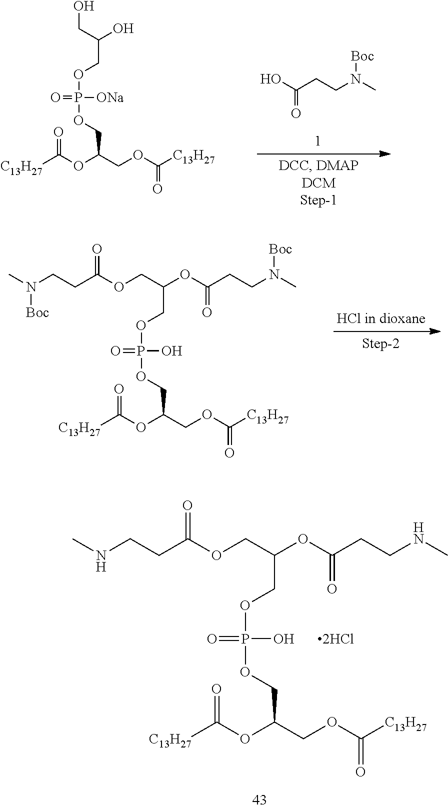

Step 1: (2R)-3-(((2,3-bis((3-((tert-butoxycarbonyl)(methyl)amino)propanoyl)oxy)-propoxy)(hydroxy)phosphoryl)oxy)propane-1,2-diyl ditetradecanoate.

To a stirred solution of 3-((tert-butoxycarbonyl)(methyl)amino)propanoic acid 1 (4.43 g, 21.78 mmol) in dichloromethane (100 mL) was added dicyclohexylmethanediimine (4.49 g, 21.78 mmol) at room temperature and the mixture was stirred for 30 min. N, N-dimethylpyridin-4-amine (0.443 g, 3.63 mmol) and sodium (R)-2,3-bis(tetradecanoyloxy)propyl(2,3-dihydroxypropyl) phosphate (5.0 g, 7.26 mmol) were added to the mixture at room temperature and stirred for overnight. Upon completion of the reaction (as monitored by LCMS), the reaction mixture was diluted with DCM (100 mL) and stirred for 10 min.

The solid was filtered off and the filtrate was washed with water (1×50 mL), HCl (0.5 N sol., 1×100 mL) and NaHCO₃ (10% soln., 1×100 mL). The combined organic layer was dried over sodium sulfate, filtered, and concentrated to afford 7.3 g of the crude product. The crude product was purified by silica gel (230-400 mesh) column chromatography. The product was eluted with 0-10% of methanol in DCM to afford (2R)-3-(((2,3-bis((3-((tert-butoxycarbonyl)-(methyl)amino)propanoyl)oxy)propoxy)(hydroxy)phosphoryl)oxy)propane-1,2-diyl ditetradeca-noate (4.2 g) as colourless syrup, which was dissolved in DCM (100 mL), washed with 0.5N HCl (1×50 mL) and 10% NaHCO₃ solution (2×50 mL). The combined organic layer was dried over sodium sulphate, filtered and concentrated under reduced pressure to get (2R)-3-(((2,3-bis((3-((tert-butoxycarbonyl)(methyl)amino)propanoyl)oxy)propoxy)(hydroxy)phosphoryl)oxy)-propane-1, 2-diyl ditetradecanoate 2 (3.0 g, 2.86 mmol, 39.4% yield) as colourless syrup.

¹H NMR (400 MHz, CDCl₃) δ (ppm): 5.24 (m, 2H), 4.42-4.37 (m, 2H), 4.21-4.13 (m, 2H), 3.96-3.95 (m, 4H), 3.51 (m, 4H), 2.88 (m, 5H), 2.65-2.59 (m, 5H), 2.36-2.28 (m, 4H), 1.62-1.60 (m, 4H), 1.46 (s, 18H), 1.32-1.27 (m, 40H), 0.90 (t, J=7.20 Hz, 6H).

Step 2: (2R)-3-(((2,3-bis((3-(methylamino)propanoyl)oxy)propoxy)(hydroxy)phosphoryl)oxy)propane-1,2-diyl ditetradecanoate dihydrochloride.

To a stirred solution of (2R)-3-(((2,3-bis((3-((tert-butoxycarbonyl)(methyl)amino)-propanoyl)oxy)propoxy)(hydroxy)phosphoryl)oxy)propane-1,2-diyl ditetradecanoate 2 (0.500 g, 0.482 mmol) in dichloromethane (5 mL) was added HCl (4M soln. in dioxane, 5.0 mL, 20 mmol) at 0° C. The reaction mixture was stirred at 10-15° C. for 3 h. The progress of the reaction was monitored by LCMS. Upon completion, the reaction mixture was concentrated under reduced pressure, and the residue was co-evaporated with ethyl acetate (2×10 mL) and n-hexane (1×15 mL). To the resultant residue, n-hexane (1×10 mL) was added. stirred for 5 min. and decanted. The resultant gum was dried under high vacuum to get (2R)-3-(((2,3-bis((3-(methylamino)propanoyl)oxy)propoxy)(hydroxy)phosphoryl)oxy)propane-1,2-diyl ditetradeca-noate dihydrochloride (0.340 g, 0.370 mmol, 77% yield) as off white solid.

¹H NMR (400 MHz, DMSO-d6) δ (ppm): 9.14 (m, 4H), 5.15-5.13 (m, 2H), 4.31-4.27 (m, 4H), 4.14-4.00 (m, 4H), 3.95-3.92 (m, 2H), 3.13-3.09 (m, 4H), 2.81 (t, J=7.20 Hz, 4H), 2.55 (m, 6H), 2.31-2.25 (m, 4H), 1.52-1.48 (m, 4H), 1.27 (m, 40H), 0.86 (t, J=6.80 Hz, 6H). LCMS: m/z: 837.4 [M+1]⁺, RT=2.58 min, purity: 99.42%. Method: Mobile Phase A: 0.1% Formic acid in water, Mobile Phase B: 0.05% Formic acid in ACN. How rate:0.8 mL/min. Column: BEH C18 (50×2.1) mm, 1.7μ. HPLC: RT=6.67 min; purity: 98.67%. Method: Column: Xbridge C₈ (50×4.6) mm, 3.5 m, Mobile Phase A:0.1% TFA in water, Mobile Phase B: Acetonitrile, Flow rate:2.0 mL/min.

### Example 27. (2R)-3-(((2,3-bis((3-(isopropylamino)propanoyl)oxy)propoxy)(hydroxy)-phosphoryl) oxy)propane-1,2-diyl Ditetradecanoate Dihyrochloride (Compound 44)

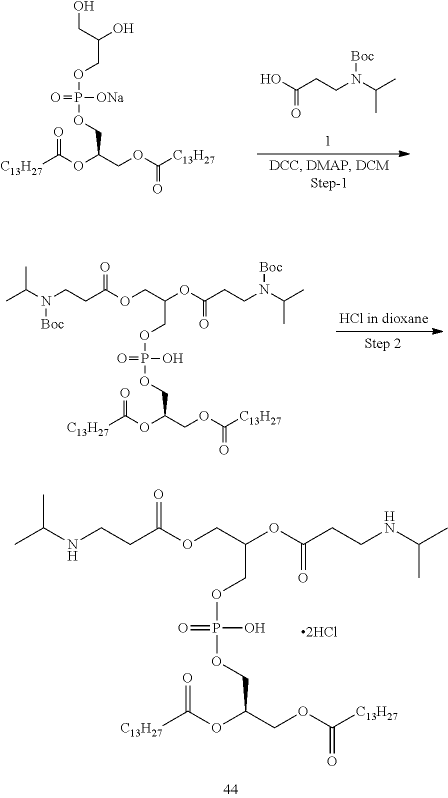

Step 1: (2R)-3-(((2,3-bis((3-((tert-butoxycarbonyl)(isopropyl)amino)propanoyl)oxy)-propoxy) (hydroxy)phosphoryl)oxy)propane-1,2-diyl ditetradecanoate.

To a stirred solution of 3-((tert-butoxycarbonyl)(isopropyl)amino)propanoic acid 1 (5.04 g, 21.78 mmol) in dichloromethane (100 mL) was added dicyclohexylmethanediimine (4.49 g, 21.78 mmol) at room temperature and the mixture was stirred for 30 min. N, N-dimethylpyridin-4-amine (0.443 g, 3.63 mmol) and sodium (R)-2,3-bis(tetradecanoyloxy)propyl(2,3-dihydroxypropyl) phosphate (5.0 g, 7.26 mmol) were added to the mixture at room temperature and stirred for overnight. Upon completion of the reaction (as monitored by LCMS), the reaction mixture was diluted with DCM (100 mL) and stirred for 10 min.

The solid was filtered off and the filtrate was washed with water (1×50 mL), HCl (0.5 N sol., 1×100 mL) and NaHCO₃ (10% soln., 1×100 mL). The combined organic layer was dried over sodium sulfate, filtered, and concentrated to afford 8.2 g of the crude product. The crude product was purified by silica gel (230-400 mesh) column chromatography. The product was eluted with 0-10% of methanol in DCM to afford (2R)-3-(((2,3-bis((3-((tert-butoxycarbonyl) (isopropyl)amino)propanoyl)oxy)propoxy)(hydroxy)phosphoryl)oxy)propane-1,2-diyl ditetra-decanoate (6.7 g) as colourless syrup, which was dissolved in DCM (100 mL), washed with 0.5N HCl (1×50 mL) and 10% NaHCO₃ solution (2×50 mL). The combined organic layer was dried over sodium sulphate, filtered and concentrated under reduced pressure to get (2R)-3-(((2,3-bis((3-((tert butoxycarbonyl)(isopropyl)amino)propanoyl)oxy)propoxy)(hydroxy)phosphoryl)oxy)prop-ane-1,2-diyl-ditetradecanoate 2 (5.1 g, 4.62 mmol, 63.6% yield) as colourless syrup.

¹H NMR (400 MHz, CD₃OD) δ (ppm): 5.25-5.24 (m, 2H), 4.48-4.44 (m, 2H), 4.25-4.18 (m, 4H), 4.04-3.99 (m, 4H), 3.41-3.37 (m, 4H), 2.65-2.59 (m, 4H), 2.38-2.32 (m, 4H), 1.63-1.60 (m, 4H), 1.49 (s, 18H), 1.31 (s, 41H), 1.18-1.16 (m, 12H), 0.92 (t, J=6.80 Hz, 6H). LCMS: m/z: 1091.4 [M−1]⁻, RT=5.38 min, purity: 99.82%. Method: Mobile Phase A: 1 mL of 25% ammonia solution in 1000 mL of MilliQ Water (pH:9 with Acetic acid). Mobile Phase B: acetonitrile. Flow rate:1.0 ml/min. COLUMN: XBridge C8 (50×4.6) mm, 3.5μ.

Step 2: (2R)-3-(((2,3-bis((3-(isopropylamino)propanoyl)oxy)propoxy)(hydroxy)-phosphoryl)oxy)propane-1,2-diyl ditetradecanoate dihydrochloride.

To a stirred solution of sodium(2R)-3-(((2,3-bis((3-((tert-butoxycarbonyl)(isopropyl)-amino)propanoyl)oxy)propoxy)(hydroxy)phosphoryl)oxy)propane-1,2-diyl-ditetradecanoate 2 (2.0 g, 1.829 mmol) in dichloromethane (20 mL) was added HCl (4M soln. in dioxane, 9.15 mL, 36.6 mmol) at 0° C. The reaction mixture was stirred at 10-15° C. for 3 h. The progress of the reaction was monitored by LCMS. Upon completion, the reaction mixture was concentrated under reduced pressure, and the residue was co-evaporated with ethyl acetate (2×25 mL) and n-hexane (1×25 mL). To the residue, n-hexane (2×15 mL) was added, stirred for 5 min. and decanted. The resultant gum was dried under high vacuum to get (2R)-3-(((2,3-bis((3-(isopropylamino)propa

noyl)oxy)propoxy)(hydroxy)phosphoryl)oxy)propane-1,2-diyl ditetradecanoate·2HCl P-014 (1.5 g, 1.537 mmol, 84% yield) as off white solid

¹H NMR (400 MHz, DMSO-d6) δ (ppm): 9.29-9.16 (m, 4H), 5.15-5.13 (m, 2H), 4.29-4.27 (m, 3H), 4.14-3.93 (m, 5H), 3.28-3.26 (m, 2H), 3.15-3.10 (m, 4H), 2.86-2.82 (m, 4H), 2.31-2.25 (m, 4H), 1.51-1.48 (m, 4H), 1.26-1.24 (m, 52H), 0.86 (t, J=6.80 Hz, 6H). LCMS: m/z: 893.4 [M+1]⁺, RT=2.61 min, purity: 99.38%. Method: Mobile Phase A: 0.1% Formic acid in water, Mobile Phase B: 0.05% Formic acid in ACN. How rate:0.8 mL/min. Column: BEH C18 (50×2.1) mm, 1.7μ. HPLC: RT=6.95 min; purity: 99.56%. Method: Column: Xbridge C8(50×4.6) mm, 3.5 μm, Mobile Phase A:0.1% TFA in water, Mobile Phase B: Acetonitrile, Flow rate: 2.0 mL/min.

### Example 28. (2R)-3-(((2,3-bis((3-(isobutylamino)propanoyl)oxy)propoxy)(hydroxy)-phosphoryl)oxy)propane-1,2-diyl Ditetradecanoate Dihydrochloride (Compound 45)

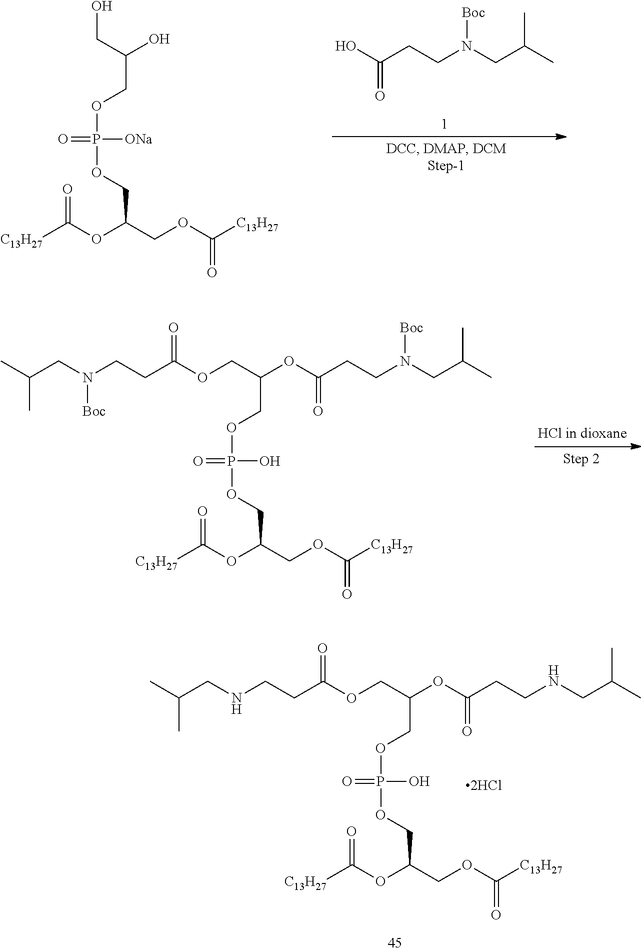

Step 1: (2R)-3-(((2,3-bis((3-((tert-butoxycarbonyl)(isobutyl)amino)propanoyl)oxy)-propoxy)(hydroxy)phosphoryl)oxy)propane-1,2-diyl ditetradecanoate.

To a stirred solution of 3-((tert-butoxycarbonyl)(isobutyl)amino)propanoic acid 1 (5.34 g, 21.78 mmol) in dichloromethane (100 mL) was added dicyclohexylmethanediimine (4.49 g, 21.78 mmol) at room temperature and the mixture was stirred for 30 min. N, N-dimethylpyridin-4-amine (0.443 g, 3.63 mmol) and sodium (R)-2,3-bis(tetradecanoyloxy)propyl(2,3-dihydroxypropyl) phosphate (5.0 g, 7.26 mmol) were added to the mixture at room temperature and stirred for overnight. Upon completion of the reaction (as monitored by LCMS), the reaction mixture was diluted with DCM (100 mL) and stirred for 10 min.

The solid was filtered off and the filtrate was washed with water (1×50 mL), HCl (0.5 N sol., 1×100 mL) and NaHCO₃ (10% soln., 1×100 mL). The combined organic layer was dried over sodium sulfate, filtered, and concentrated to afford (8.4 g) of the crude product. The crude product was purified by silica gel (230-400 mesh) column chromatography. The product was eluted with 0-10% of methanol in DCM to afford (2R)-3-(((2,3-bis((3-((tert-butoxycarbonyl) (isobutyl)amino)propanoyl)oxy)propoxy)(hydroxy)phosphoryl)oxy)propane-1,2-diyl ditetradecanoate (6.2 g) as colourless syrup, which was dissolved in DCM (100 mL), washed with 0.5 N HCl (1×50 mL), 10% NaHCO₃ solution (2×50 mL). The combined organic layer was dried over sodium sulphate, filtered and concentrated under reduced pressure to get (2R)-3-(((2,3-bis((3-((tert-butoxycarbonyl)(isobutyl)amino)propanoyl)oxy)propoxy)(hydroxy)phosphoryl)-oxy)propane-1,2-diyl ditetradecanoate 2 (3.8 g, 3.35 mmol, 46.2% yield) as colourless syrup.

¹H NMR (400 MHz, CD₃OD) δ (ppm): 5.51-5.51 (m, 2H), 4.88-4.44 (m, 2H), 4.23-4.21 (m, 2H), 4.03-4.03 (m, 4H), 3.51-3.48 (m, 4H), 3.10-3.07 (m, 4H), 2.67-2.61 (m, 4H), 2.32 (m, 4H), 1.95-1.92 (m, 2H), 1.64-1.60 (m, 4H), 1.48 (m, 18H), 1.32 (m, 40H), 0.94-0.90 (m, 18H). LCMS: m/z: 1119.4 [M−1]⁻, RT=5.65 min, purity: 99.49%. Method: Mobile Phase A: 1 mL of 25% ammonia solution in 1000 mL of MilliQ Water (pH:9 with Acetic acid). Mobile Phase B: acetonitrile. Flow rate:1.0 ml/min. COLUMN: XBridge C8 (50×4.6) mm, 3.5μ.

Step 2: (2R)-3-(((2,3-bis((3-(isobutylamino)propanoyl)oxy)propoxy)(hydroxy)phos-phoryl)oxy)propane-1,2-diyl ditetradecanoate dihydrochloride.

To a stirred solution of (2R)-3-(((2,3-bis((3-((tert-butoxycarbonyl) (isobutyl)-amino)propanoyl)oxy)propoxy)(hydroxy)phosphoryl)oxy)propane-1,2-diyl ditetradecanoate 2 (2.0 g, 1.783 mmol) in dichloromethane (20 mL) was added HCl (4M soln. in dioxane, 8.92 mL, 35.7 mmol) at 0° C. The reaction mixture was stirred at 10-15° C. for 3 h. The progress of the reaction was monitored by LCMS. Upon completion, the reaction mixture was concentrated under reduced pressure, and the residue was co-evaporated with ethyl acetate (2×25 mL) and n-hexane (1×25 mL). To the residue, n-hexane (2×15 mL) was added. stirred for 5 min. and decanted. The resultant gum was dried under high vacuum to get (2R)-3-(((2,3-bis((3-(isobutyl-amino)propanoyl)oxy)propoxy)(hydroxy)phosphoryl)oxy)propane-1,2-diyl ditetradecanoate dihydrochloride (1.38 g, 1.374 mmol, 77% yield) as off white solid.

¹H NMR (400 MHz, DMSO-d6) δ (ppm): 9.26-8.78 (m, 4H), 5.11 (m, 2H), 4.30-4.27 (m, 3H), 4.14-3.90 (m, 5H), 3.14-3.12 (m, 4H), 2.89-2.85 (m, 4H), 2.76-2.75 (m, 4H), 2.30-2.25 (m, 4H), 2.03-1.98 (m, 2H), 1.51-1.50 (m, 4H), 1.39-1.24 (m, 40H), 1.17-0.94 (m, 12H), 0.86 (t, J=6.80 Hz, 6H). LCMS: m/z: 921.4 [M+1]⁺, RT=2.62 min, purity: 99.17%.

Method: Mobile Phase A: 0.1% Formic acid in water, Mobile Phase B: 0.05% Formic acid in ACN. Flow rate:0.8 mL/min. COLUMN: BEH C18 (50×2.1) mm, 1.7μ. HPLC: RT=7.11 min; purity: 99.52%. Method: Column:Xbridge C8(50×4.6) mm, 3.5 μm, Mobile Phase A:0.1% TFA in water, Mobile Phase B: Acetonitrile, Flow rate:2.0 mL/min.

### Example 29. (2R)-3-(((2,3-bis((3-((cyclopropylmethyl)amino)propanoyl)oxy)-propoxy)(hydroxy)phosphoryl)oxy)propane-1,2-diyl Ditetradecanoate Dihydrochloride (Compound 46)

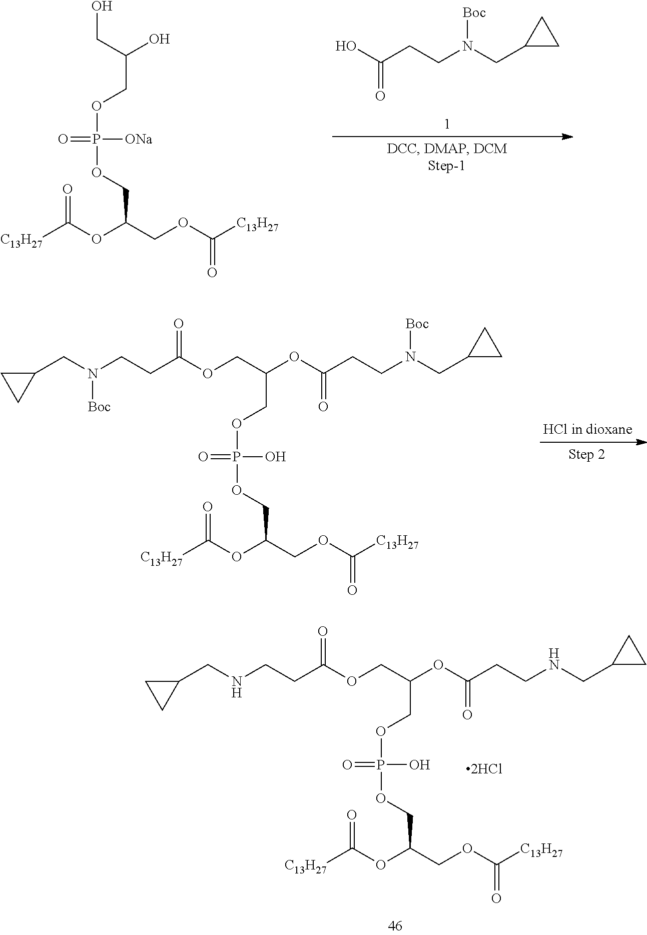

Step 1: (2R)-3-(((2,3-bis((3-((tertbutoxycarbonyl)(cyclopropylmethyl)amino)propanoyl)-oxy)propoxy)(hydroxy)phosphoryl)oxy)propane-1,2-diyl ditetradecanoate.

To a stirred solution of 3-((tert-butoxycarbonyl)(cyclopropylmethyl)amino)propanoic acid 1 (5.30 g, 21.78 mmol) in dichloromethane (100 mL) was added dicyclohexylmethanediimine (4.49 g, 21.78 mmol) at room temperature and the mixture was stirred for 30 min. N, N-dimethylpyridin-4-amine (0.443 g, 3.63 mmol) and sodium (R)-2,3-bis(tetradecanoyloxy)propyl(2,3-dihydroxypropyl)phosphate (5.0 g, 7.26 mmol) were added to the mixture at room temperature and stirred for overnight. Upon completion of the reaction (as monitored by LCMS), the reaction mixture was diluted with DCM (100 mL) and stirred for 10 min.

The solid was filtered off and the filtrate was washed with water (1×50 mL), HCl (0.5 N sol., 1×100 mL) and NaHCO₃ (10% soln., 1×100 mL). The combined organic layer was dried over sodium sulfate, filtered, and concentrated to afford (8.2 g) of the crude product. The crude product was purified by silica gel (230-400 mesh) column chromatography. The product was eluted with 0-10% of methanol in DCM. To afford (2R)-3-(((2,3-bis((3-((tert-butoxycarbonyl) (cyclopropylmethyl)amino)propanoyl)oxy)propoxy)(hydroxy)phosphoryl)oxy)propane-1,2-diyl ditetradecanoate (6.2 g) as colourless syrup, which was dissolved in DCM (100 mL), washed with 0.5N HCl (1×50 mL), 10% NaHCO₃ solution (2×50 mL). The combined organic layer was dried over sodium sulphate, filtered and concentrated under reduced pressure to get (2R)-3-(((2,3-bis((3-((tert-butoxycarbonyl)(cyclopropylmethyl)amino)propanoyl)oxy)propoxy)-(hydroxy)phosphoryl)oxy)propane-1,2-diyl ditetradecanoate 2 (4.6 g, 4.08 mmol, 56.1% yield) as colourless syrup.

¹H NMR (400 MHz, CD₃OD) δ (ppm): 5.31-5.23 (m, 2H), 4.52-4.49 (m, 2H), 4.28-4.18 (m, 2H), 4.02-4.01 (m, 4H), 3.59-3.55 (m, 4H), 3.18-3.15 (m, 4H), 2.67-2.65 (m, 4H), 2.34-2.32 (m, 4H), 1.63-1.62 (m, 4H), 1.49-1.48 (m, 18H), 1.31 (m, 40H), 1.01-0.92 (m, 2H), 0.90 (t, J=2.80 Hz, 6H), 0.54-0.51 (m, 4H). LCMS: m/z: 1115.4 [M−1]⁻, RT=5.39 min, purity: 99.36%. Method: Mobile Phase A: 1 mL of 25% ammonia solution in 1000 mL of MilliQ Water (pH:9 with Acetic acid). Mobile Phase B: acetonitrile. Flow rate:1.0 ml/min. COLUMN: XBridge C8 (50×4.6) mm, 3.5μ.

Step 2: (2R)-3-(((2,3-bis((3-((cyclopropylmethyl)amino)propanoyl)oxy)propoxy)(hy-droxy)phosphoryl)oxy)propane-1,2-diyl ditetradecanoate dihydrochloride.

To a stirred solution of (2R)-3-(((2,3-bis((3-((tertbutoxycarbonyl)(cyclopropylmethyl)-amino)propanoyl)oxy)propoxy)(hydroxy)phosphoryl)oxy)propane-1,2-diyl ditetradecanoate 2 (0.500 g, 0.447 mmol) in dichloromethane (5 mL) was added HCl (4M soln. in dioxane, 2.4 mL, 8.95 mmol) at 0° C. The reaction mixture was stirred at 10-15° C. for 3 h. The progress of the reaction was monitored by LCMS. Upon completion, the reaction mixture was concentrated under reduced pressure, and the residue was co-evaporated with ethyl acetate (2×10 mL) and n-hexane (1×10 mL). To the residue, n-hexane (1×10 mL) was added. stirred for 5 min. and decanted. The resultant gum was dried under high vacuum to get (2R)-3-(((2,3-bis((3-((cyclopropylmethyl)amino)propanoyl)oxy)propoxy)(hydroxy)phosphoryl)oxy)propane-1,2-diyl ditetradecanoate dihydrochloride (0.368 g, 0.368 mmol, 82% yield) as off white solid.

¹H NMR (400 MHz, DMSO-d6) δ (ppm): 9.40-8.94 (m, 4H), 5.14-5.12 (m, 2H), 4.30-4.27 (m, 3H), 4.14-4.00 (m, 4H), 3.95-3.92 (m, 2H), 3.17-3.12 (m, 4H), 2.86-2.81 (m, 8H), 2.30-2.25 (m, 4H), 1.51-1.50 (m, 4H), 1.24 (m, 40H), 1.11-1.06 (m, 2H), 0.86 (t, J=6.80 Hz, 6H), 0.57-0.55 (m, 4H), 0.39-0.37 (m, 4H). LCMS: m/z: 915.4 [M+1]⁺, RT=2.59 min, purity: 98.99%.

Method: Mobile Phase A: 0.1% Formic acid in water, Mobile Phase B: 0.05% Formic acid in ACN. Flow rate:0.8 mL/min. Column: BEH C18 (50×2.1) mm, 1.7μ. HPLC: RT=7.02 min; purity: 99.27%. Method: Column: XBridge C8(50×4.6) mm, 3.5 μm, Mobile Phase A: 0.1% TFA in water, Mobile Phase B: Acetonitrile, Flow rate: 2.0 mL/min.

### Example 30. (2R)-3-(((2,3-bis((3-(benzylamino)propanoyl)oxy)propoxy)(hydroxy)-phosphoryl)oxy)propane-1,2-diyl Ditetradecanoate Dihydrochloride (Compound 47)

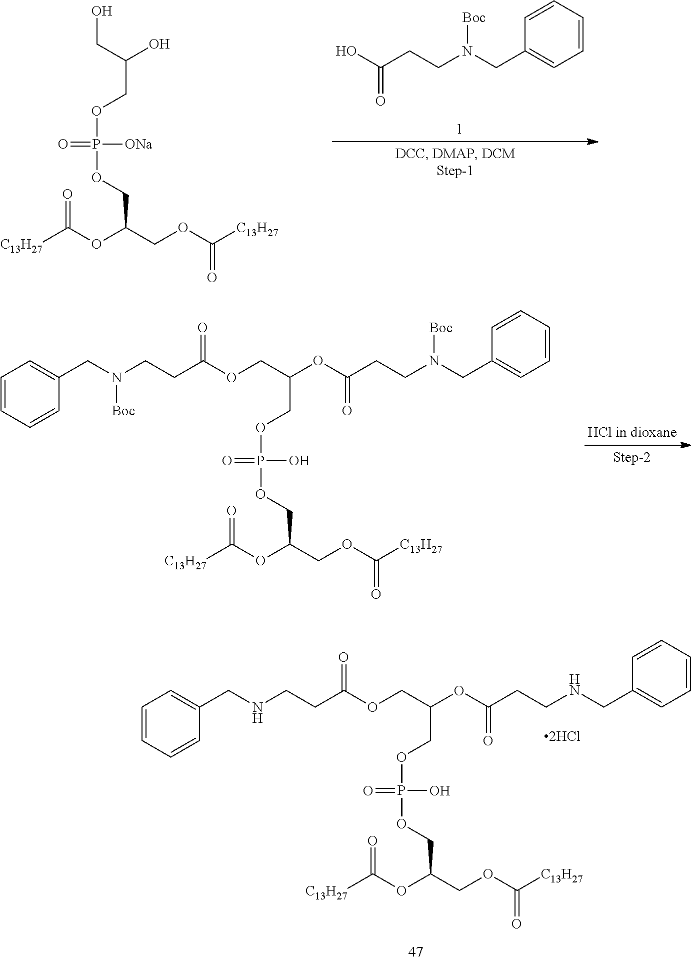

Step 1: (2R)-3-(((2,3-bis((3-(benzyl(tert-butoxycarbonyl)amino)propanoyl)oxy)propoxy)-(hydroxy)phosphoryl)oxy)propane-1,2-diyl ditetradecanoate.

To a stirred solution of 3-(benzyl(tert-butoxycarbonyl)amino)propanoic acid 1 (3.65 g, 13.07 mmol) in dichloromethane (80 mL) was added dicyclohexylmethanediimine (2.70 g, 13.07 mmol) at room temperature and the mixture was stirred for 30 min. N, N-dimethylpyridin-4-amine (0.266 g, 2.178 mmol) and sodium (R)-2,3-bis(tetradecanoyloxy)propyl(2,3-dihydroxypropyl) phosphate (3.0 g, 4.36 mmol) were added to the mixture and stirred at room temperature for overnight. Upon completion of the reaction (as confirmed by LCMS), the reaction mixture was diluted with dichloromethane (80 mL) and stirred for 10 min.

The solid was filtered off and the filtrate was washed with water (1×50 mL), HCl (0.5 N soln., 1×80 mL) and NaHCO₃ (10% soln., 1×80 mL). The combined organic layer was dried over sodium sulfate, filtered, and concentrated to get the crude product (6.5 g). The crude product was purified by silica gel (230-400 mesh) column chromatography. The product was eluted with 0-10% of methanol in DCM to afford (2R)-3-(((2,3-bis((3-(benzyl(tert-butoxycarbonyl)amino)propanoyl)oxy)propoxy)(hydroxy)phosphoryl)oxy)-propane-1,2-diyl-ditetradecanoate (4.8 g) as colourless syrup, which was dissolved in dichloromethane (100 mL), washed with HCl (0.5 N sol., 1×50 mL) and NaHCO₃ (10% soln., 2×50 mL). The combined organic layer was dried over sodium sulphate, filtered and concentrated under reduced pressure to get (2R)-3-(((2,3-bis((3-(benzyl(tert-butoxycarbonyl)amino)propanoyl)oxy)propoxy)(hydroxy)-phosphoryl)oxy)propane-1,2-diyl ditetradecanoate 2 (3.2 g, 2.66 mmol, 61.2% yield) as pale yellow gum.

¹H NMR (400 MHz, CD₃OD) δ (ppm): 7.35-7.24 (m, 10H), 5.22 (m, 2H), 4.49-4.40 (m, 4H), 4.22-4.17 (m, 2H), 4.00-3.98 (m, 6H), 3.53-3.50 (m, 4H), 2.59-2.58 (m, 4H), 2.35-2.30 (m, 4H), 1.61-1.60 (m, 4H), 1.59-1.44 (m, 18H), 1.34-1.30 (m, 41H), 0.92 (t, J=6.80 Hz, 6H). LCMS: m/z: 1187.3 [M−1]⁻, RT=5.39 min, purity: 99.57%. Method: Mobile Phase A: 1 mL of 25% ammonia solution in 1000 mL of MilliQ Water (pH:9 with Acetic acid). Mobile Phase B: acetonitrile. Flow rate:1.0 ml/min. Column: XBridge C₈ (50×4.6) mm, 3.5μ.

Step 2: (2R)-3-(((2,3-bis((3-(benzylamino)propanoyl)oxy)propoxy)(hydroxy)phos-phoryl)oxy)propane-1,2-diyl ditetradecanoate dihydrochloride

To a stirred solution of solution of (2R)-3-(((2,3-bis((3-(benzyl(tert-butoxycarbonyl)amino)propanoyl)oxy)propoxy)(hydroxy)phosphoryl)oxy)propane-1,2-diyl-ditetradecanoate 2 (2.0 g, 1.681 mmol) in dichloromethane (20 mL) was added HCl (4M in dioxane, 8.41 mL, 33.6 mmol) at 10° C. The reaction mixture was stirred at 15-20° C. for 2 h. Upon completion the reaction (as confirmed by LCMS), the reaction mixture was concentrated under reduced pressure at room temperature. The residue was co-evaporated with ethyl acetate (2×25 mL) and n-hexane (1×25 mL). To this residue, n-hexane (2×15 mL) was added, stirred for 5 min. and decanted. The resultant gum was dried under high vacuum to get (2R)-3-(((2,3-bis((3-(benzylamino)propanoyl)oxy)propoxy)(hydroxy)phosphoryl)oxy)propane-1,2-diylditetradecanoate dihydrochloride (1.52 g, 1.417 mmol, 84% yield) as a off white solid.

¹H NMR (400 MHz, DMSO-d6) δ (ppm): 9.63-9.63 (m, 4H), 7.60-7.57 (m, 4H), 7.43-7.40 (m, 6H), 5.16-5.11 (m, 2H), 4.31-4.25 (m, 3H), 4.15-4.01 (m, 7H), 4.00-3.92 (m, 2H), 3.11-3.17 (m, 4H), 2.90-2.85 (m, 4H), 2.30-2.24 (m, 4H), 1.49 (m, 4H), 1.23 (m, 40H), 0.85 (t, J=9.20 Hz, 6H). LCMS: m/z: 989.4.4 [M+1]⁺, RT=2.67 min, purity: 98.23%. Method: Mobile Phase A: 0.1% Formic acid in water, Mobile Phase B: 0.05% Formic acid in ACN. Flow rate:0.8 mL/min. COLUMN: BEH C18 (50×2.1) mm, 1.7μ. HPLC: RT=7.23 min; purity: 99.70%. Method: Column: Xbridge C₈ (50×4.6) mm, 3.5 m, Mobile Phase A: 0.1% TFA in water, Mobile Phase B: Acetonitrile, Flow rate: 2.0 mL/min.

### Example 31. (2R)-3-(((2,3-bis(((S)-3-aminobutanoyl)oxy)propoxy)(hydroxy)-phosphoryl)oxy)propane-1,2-diyl Ditetradecanoate Dihydrochloride (Compound 48)

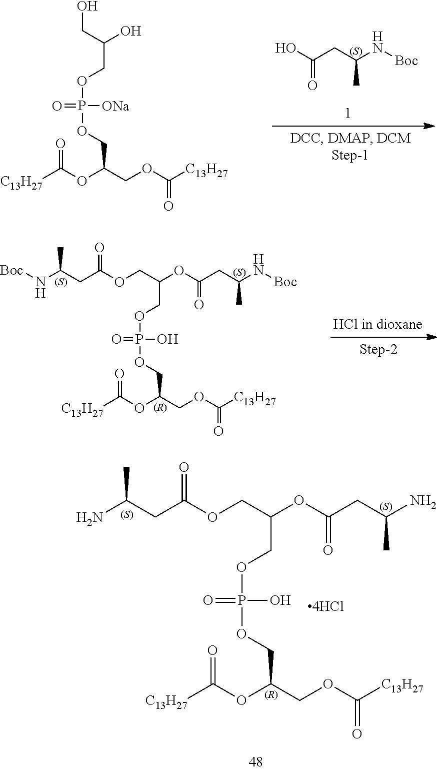

Step 1: (2R)-3-(((2,3-bis(((S)-3-((tert-butoxycarbonyl)amino)butanoyl)oxy)propoxy)-(hydroxy)phosphoryl)oxy)propane-1,2-diyl ditetradecanoate.

To a stirred solution of (S)-3-((tert-butoxycarbonyl)amino)butanoic acid 1 (3.98 g, 19.60 mmol) in dichloromethane (90 mL) was added dicyclohexylmethanediimine (4.04 g, 19.60 mmol) at room temperature and the mixture was stirred for 30 min. N, N-dimethylpyridin-4-amine (0.399 g, 3.27 mmol) and sodium (R)-2,3-bis(tetradecanoyloxy)propyl(2,3-dihydroxypropyl) phosphate (4.5 g, 6.53 mmol) were added to the mixture at room temperature and stirred for overnight. Upon completion of the reaction (as monitored by LCMS), the reaction mixture was diluted with DCM (100 mL) and stirred for 10 min.

The solid was filtered off and the filtrate was washed with water (1×50 mL), HCl (0.5 N sol., 1×100 mL) and NaHCO₃ (10% soln., 1×100 mL). The combined organic layer was dried over sodium sulfate, filtered, and concentrated to afford (7.3 g) of the crude product. The crude product was purified by silica gel (230-400 mesh) column chromatography. The product was eluted with 0-10% of methanol in DCM to afford (2R)-3-(((2,3-bis(((S)-3-((tert-butoxycarbonyl)amino)butanoyl)oxy)propoxy)(hydroxy)phosphoryl)oxy)propane-1,2-diyl di-tetradecanoate (5.8 g) as colourless syrup, which was dissolved in DCM (100 mL), washed with 0.5N HCl (1×50 mL), 10% NaHCO₃ solution (2×50 mL). The combined organic layer was dried over sodium sulphate, filtered and concentrated under reduced pressure to get (2R)-3-(((2,3-bis(((S)-3-((tert-butoxycarbonyl)amino)butanoyl)oxy)propoxy)(hydroxy)phosphoryl)oxy)-propane-1,2-diyl-ditetradecanoate 2 (3.8 g, 3.63 mmol, 55.5% yield) as colourless syrup.

¹H NMR (400 MHz, CD₃OD) δ (ppm): 6.85-6.83 (m, 2H), 5.03 (m, 2H), 4.30-4.26 (m, 2H), 4.20-4.03 (m, 2H), 3.81-3.68 (m, 6H), 2.50-2.46 (m, 2H), 2.34-2.26 (m, 6H), 1.51-1.49 (m, 4H), 1.37 (s, 18H), 1.24 (m, 40H), 1.06 (m, 6H), 0.91 (t, J=6.8 Hz, 6H). LCMS: m/z: 1035.5 [M−1]⁻, RT=4.96 min, purity: 98.95%. Method: Mobile Phase A: 1 mL of 25% ammonia solution in 1000 mL of MilliQ Water (pH:9 with Acetic acid). Mobile Phase B: acetonitrile. Flow rate:1.0 ml/min. COLUMN: XBridge C₈ (50×4.6) mm, 3.5μ.

Step 2: (2R)-3-(((2,3-bis(((S)-3-aminobutanoyl)oxy)propoxy)(hydroxy)phosphoryl)-oxy)propane-1,2-diyl ditetradecanoate dihydrochloride.

To a stirred solution of solution of (2R)-3-(((2,3-bis(((S)-3-((tert-butoxycarbonyl)-amino)butanoyl)oxy)propoxy)(hydroxy)phosphoryl)oxy)propane-1,2-diyl ditetradecanoate (0.500 g, 0.482 mmol) in DCM (5 ml) was added HCl (4M in dioxane, 2.410 ml, 9.64 mmol) at 10° C. The reaction mixture was stirred at 15-20° C. for 3 h. The progress of the reaction was monitored by LCMS. Upon completion, the reaction mixture was concentrated under reduced pressure, and the residue was co-evaporated with ethyl acetate (2×10 mL) and n-hexane (1×15 mL). To the residue, n-hexane (2×10 mL) was added. stirred for 5 min. and decanted. The resultant gum was dried under high vacuum to get (2R)-3-(((2,3-bis(((S)-3-aminobutanoyl)-oxy)propoxy)(hydroxy)phosphoryl)oxy)propane-1,2-diyl ditetradecanoate dihydrochloride (0.332 g, 0.334 mmol, 69.4% yield) as a off white solid.

¹H NMR (400 MHz, DMSO-d6) δ (ppm): 8.27-8.32 (m, 6H), 5.17-5.13 (m, 2H), 4.30-4.27 (m, 3H), 4.14-3.93 (m, 6H), 3.52-3.50 (m, 2H), 2.84-2.78 (m, 2H), 2.68-2.60 (m, 2H), 2.31-2.25 (m, 4H), 1.51-1.50 (m, 4H), 1.26-1.24 (m, 46H), 0.86 (t, J=6.80 Hz, 6H). LCMS: m/z: 836.9 [M+1]⁺, RT=2.65 min, purity: 99.68%. Method: Mobile Phase A: 0.1% Formic acid in water, Mobile Phase B: 0.05% Formic acid in ACN. Flow rate:0.8 mL/min. COLUMN: BEH C18 (50×2.1) mm, 1.7μ. HPLC: RT=6.69 min; purity: 99.25%. Method: Column:Xbridge C8(50×4.6) mm, 3.5 μm, Mobile Phase A:0.1% TFA in water, Mobile Phase B: Acetonitrile, Flow rate:2.0 mL/min.

### Example 32. (2R)-3-(((2,3-bis(((R)-3-aminobutanoyl)oxy)propoxy)(hydroxy)-phosphoryl)oxy)propane-1,2-diyl Ditetradecanoate Dihydrochloride (Compound 49)

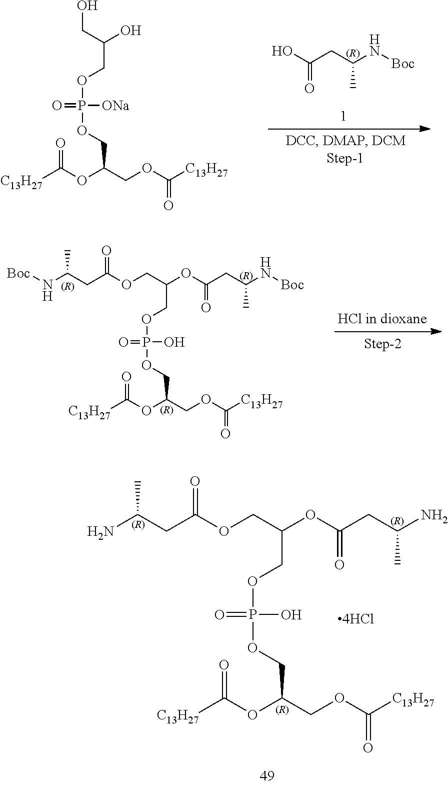

Step 1: (2R)-3-(((2,3-bis(((R)-3-((tert-butoxycarbonyl)amino)butanoyl)oxy)propoxy)-(hydroxy)phosphoryl)oxy)propane-1,2-diyl ditetradecanoate.

To a stirred solution of (R)-3-((tert-butoxycarbonyl)amino)butanoic acid 1 (3.98 g, 19.60 mmol) in dichloromethane (90 mL) was added dicyclohexylmethanediimine (4.04 g, 19.60 mmol) at room temperature and the mixture was stirred for 30 min. N, N-dimethylpyridin-4-amine (0.399 g, 3.27 mmol) and sodium (R)-2,3-bis(tetradecanoyloxy)propyl(2,3-dihydroxypropyl) phosphate (4.5 g, 6.53 mmol) were added to the mixture at room temperature and stirred for overnight. Upon completion of the reaction as monitored by LCMS, the reaction mixture was diluted with DCM (100 mL) and stirred for 10 min.

The solid was filtered off and the filtrate was washed with water (1×50 mL), HCl (0.5 N sol., 1×100 mL) and NaHCO₃ (10% soln., 1×100 mL). The combined organic layer was dried over sodium sulfate, filtered, and concentrated to afford the crude product (7.0 g). The crude product was purified by silica gel (230-400 mesh) column chromatography. The product was eluted with 0-10% of methanol in DCM. To afford (2R)-3-(((2,3-bis(((R)-3-((tert-butoxycarbonyl)amino)butanoyl)oxy)propoxy)(hydroxy)phosphoryl)oxy)propane-1,2-diyl-ditetradecanoate (5.6 g) as colourless syrup, which was dissolved in DCM (100 mL), washed with 0.5N HCl (1×50 mL), 10% NaHCO₃ solution (2×50 mL). The combined organic layer was dried over sodium sulphate, filtered and concentrated under reduced pressure to get (2R)-3-(((2,3-bis(((R)-3-((tert-butoxycarbonyl)amino)butanoyl)oxy)propoxy)(hydroxy)phosphoryl)oxy)pro-pane-1,2-diyl ditetradecanoate 2 (3.73 g, 3.60 mmol, 55.0% yield) as colourless syrup.

¹H NMR (400 MHz, CD₃OD) δ (ppm): 6.85-6.83 (m, 2H), 5.03 (m, 2H), 4.30-4.26 (m, 2H), 4.20-4.03 (m, 2H), 3.81-3.68 (m, 6H), 2.50-2.46 (m, 2H), 2.34-2.26 (m, 6H), 1.51-1.49 (m, 4H), 1.37 (s, 18H), 1.24 (m, 40H), 1.06 (m, 6H), 0.91 (t, J=6.8 Hz, 6H). LCMS: m/z: 1035.5 [M−1]⁻, RT=4.95 min, purity: 99.77%. Method: Mobile Phase A: 1 mL of 25% ammonia solution in 1000 mL of MilliQ Water (pH:9 with Acetic acid). Mobile Phase B: acetonitrile. Flow rate:1.0 ml/min. Column: XBridge C8 (50×4.6) mm, 3.5μ.

Step 2: ((2R)-3-(((2,3-bis(((R)-3-aminobutanoyl)oxy)propoxy)(hydroxy)phosphoryl)-oxy)propane-1,2-diyl ditetradecanoate dihydrochloride.

To a stirred solution of solution of (2R)-3-(((2,3-bis(((R)-3-((tert-butoxycarbonyl)-amino)butanoyl)oxy)propoxy)(hydroxy)phosphoryl)oxy)propane-1,2-diyl ditetradecanoate (0.500 g, 0.482 mmol) in DCM (5 ml) was added HCl (4M in dioxane, 2.410 ml, 9.64 mmol) at 10° C. The reaction mixture was stirred at 15-20° C. for 3 h. The progress of the reaction was monitored by LCMS. Upon completion, the reaction mixture was concentrated under reduced pressure, and the residue was co-evaporated with ethyl acetate (2×15 mL) and n-hexane (1×15 mL). To the residue, n-hexane (2×10 mL) was added. stirred for 5 min. and decanted. The resultant gum was dried under high vacuum to get (2R)-3-(((2,3-bis(((R)-3-aminobutanoyl)oxy)propoxy)(hydroxy) phosphoryl)oxy)propane-1,2-diyl ditetradecanoate dihydrochloride (0.320 g, 0.348 mmol, 72.2% yield) as a off white solid.

¹H NMR (400 MHz, DMSO-d6) δ (ppm)=8.31-8.27 (m, 6H), 5.16-5.14 (m, 2H), 4.30-4.25 (m, 3H), 4.14-3.94 (m, 6H), 3.52-3.48 (m, 2H), 2.83-2.77 (m, 2H), 2.66-2.60 (m, 2H), 2.30-2.25 (m, 4H), 1.51-1.50 (m, 4H), 1.24 (m, 46H), 0.85 (t, J=6.80 Hz, 6H), LCMS: m/z: 836.8 [M+1]⁺, RT=2.65 min, purity: 98.87%. Method: Mobile Phase A: 0.1% Formic acid in water, Mobile Phase B: 0.05% Formic acid in ACN. Flow rate:0.8 mL/min. COLUMN: BEH C18 (50×2.1) mm, 1.7μ. HPLC: RT=6.69 min; purity: 98.93%. Method: Column:Xbridge C8 (50×4.6) mm, 3.5 μm, Mobile Phase A: 0.1% TFA in water, Mobile Phase B: Acetonitrile, Flow rate: 2.0 mL/min.

### Example 33. 3-(((2,3-bis((3-amino-3-methylbutanoyl)oxy)propoxy)(hydroxy)-phosphoryl)-oxy)propane-1,2-diyl Ditetradecanoate Dihydrochloride (Compound 51)

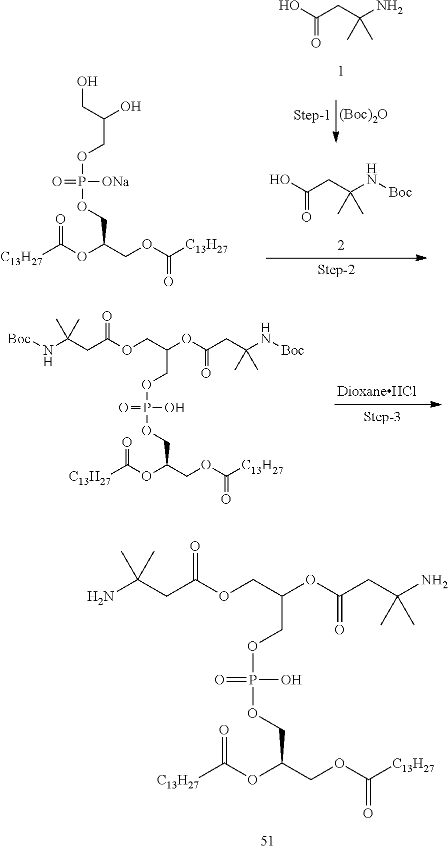

Step 1: 3-((tert-butoxycarbonyl)amino)-3-methylbutanoic acid.

To a stirred solution of 3-amino-3-methylbutanoic acid (2.7 g, 23.05 mmol) and NaOH (1M solution, 23.05 mL, 23.05 mmol) in 1,4-dioxane (30 mL) was added Boc-anhydride (6.88 mL, 30.0 mmol). The reaction mixture was stirred at room temperature for overnight. Upon completion of the reaction (as monitored by LCMS), the volatiles were removed under reduced pressure and crude was diluted with water (40 mL), washed with MTBE (20 mL). The aqueous layer was acidified (pH-4 to 5) with 1.5 N HCl and extracted with EtOAc (3×50 mL). The combined organic layer was dried over sodium sulfate, filtered and concentrated to afford 3-((tert-butoxycarbonyl)amino)-3-methylbutanoic acid 2 (2.0 g, 9.02 mmol, 39.1% yield) as a cololurless liquid.

¹H NMR (400 MHz, CDCl3) δ (ppm)=2.76 (m, 2H), 1.47 (s, 9H), 1.42 (s, 6H). LCMS: m/z: 216.0 [M+1]+, RT=1.60 min, purity: 99.62%.

Step 2: (2R)-3-(((2,3-bis((3-((tert-butoxycarbonyl)amino)-3-methylbutanoyl)oxy)pro-poxy)(hydroxy)phosphoryl)oxy)propane-1,2-diyl ditetradecanoate.

To a stirred solution of 3-((tert-butoxycarbonyl)amino)-3-methylbutanoic acid (2.082 g, 9.58 mmol) in dichloromethane (20 mL) was added dicyclohexylmethanediimine (1.797 g, 8.71 mmol) at room temperature and the mixture was stirred for 30 min. N, N-dimethylpyridin-4-amine (0.177 g, 1.452 mmol) and sodium (R)-2,3-bis(tetradecanoyloxy)propyl(2,3-dihydroxypropyl) phosphate (2.0 g, 2.90 mmol) were added to the mixture at room temperature and stirred for overnight. Upon completion of the reaction (as monitored by LCMS), the reaction mixture was diluted with DCM (50 mL) and stirred for 10 min.

The solid was filtered off and the filtrate was washed with water (1×30 mL), HCl (0.5 N sol., 1×50 mL) and NaHCO₃ (10% soln., 1×50 mL). The combined organic layer was dried over sodium sulfate, filtered, and concentrated to afford the crude product (4.0 g). The crude product was purified by silica gel (230-400 mesh) column chromatography. The product was eluted with 0-10% of methanol in DCM. To afford (2R)-3-(((2,3-bis((3-((tert-butoxycarbonyl)amino)-3-methylbutanoyl)oxy)propoxy)(hydroxy)phosphoryl)oxy)propane-1,2-diyl ditetradecanoate (1.6 g) as colourless syrup, which was dissolved in DCM (50 mL), washed with 0.5N HCl (30 mL×1), 10% NaHCO₃ solution (30 mL×2). The combined organic layer was dried over sodium sulphate, filtered and concentrated under reduced pressure to afford (2R)-3-(((2,3-bis((3-((tert-butoxycarbonyl)amino)-3-methylbutanoyl)oxy)propoxy)(hydroxy)phosphoryl)oxy)propane-1,2-diyl ditetradecanoate, 3 (1.2 g, 1.115 mmol, 38.4% yield) as a colourless syrup.

¹H NMR (400 MHz, CD₃OD) δ (ppm): 5.23 (m, 2H), 4.49-4.42 (m, 2H), 4.23-4.17 (m, 2H), 4.03-3.99 (m, 4H), 3.53-3.48 (m. 4H), 2.79-2.76 (m, 4H), 2.36-2.32 (m, 4H), 1.64-1.62 (m, 4H), 1.45 (s, 18H), 1.38-1.31 (m, 48H), 0.92 (t, J=7.20 Hz, 6H), LCMS: m/z: 1063.6 [M−1]⁻, RT=5.53 min, purity: 99.88%. Method: Mobile Phase A: 1 mL of 25% ammonia solution in 1000 mL of MilliQ Water (pH:9 with Acetic acid). Mobile Phase B: acetonitrile. Flow rate:1.0 ml/min. COLUMN: XBridge C8 (50×4.6) mm, 3.5μ.

Step 3: 3-(((2,3-bis((3-amino-3-methylbutanoyl)oxy)propoxy)(hydroxy)phosphoryl)-oxy)propane-1,2-diyl ditetradecanoate dihydrochloride.

To a stirred solution of solution of (2R)-3-(((2,3-bis((3-((tert-butoxycarbonyl)amino)-3-methylbutanoyl)oxy)propoxy)(hydroxy)phosphoryl)oxy)propane-1,2-diyl ditetradecanoate (0.700 g, 0.657 mmol) in DCM (7 ml) was added HCl (4M in dioxane, 3.29 ml, 13.14 mmol) at 10° C. The reaction mixture was stirred at 15-20° C. for 3 h and the progress of the reaction was monitored by LCMS. Upon completion, the reaction mixture was concentrated under reduced pressure, and the residue was co-evaporated with ethyl acetate (2×15 mL) and n-hexane (1×15 mL). To the residue, n-hexane (2×10 mL) was added. stirred for 5 min. and decanted. The resultant gum was dried under high vacuum to get (2R)-3-(((2,3-bis((3-amino-3-methylbutanoyl)oxy)propoxy)(hydroxy)phosphoryl)oxy)propane-1,2-diyl ditetradecanoate dihydrochloride (0.510 g, 0.504 mmol, 77% yield) as a off white solid.

¹H NMR (400 MHz, DMSO-d6) δ (ppm)=8.43-8.41 (m, 6H), 5.19-5.13 (m, 2H), 4.35-4.21 (m, 3H), 4.14-3.95 (m, 6H), 2.81-2.69 (m, 4H), 2.28-2.24 (m, 4H), 1.51-1.49 (m, 4H), 1.36 (s, 12H), 1.23 (m, 39H), 0.85 (t, J=6.80 Hz, 6H). LCMS: m/z: 865.6 [M+1]+, RT=2.63 min, purity: 95.92%. Method: Mobile Phase A: 0.1% Formic acid in water, Mobile Phase B: 0.05% Formic acid in ACN. Flow rate:0.8 mL/min. COLUMN: BEH C18 (50×2.1) mm, 1.7μ. HPLC: RT=6.79 min; purity: 99.11%. Method: Column:Xbridge C8(50×4.6) mm, 3.5 μm, Mobile Phase A:0.1% TFA in water, Mobile Phase B: Acetonitrile, Flow rate:2.0 mL/min.

### Example 34. Cardiac Response Testing

Efficacy evaluation of the compounds of the present invention involved ECG measurements of adult male Hartley guinea pigs wherein PR, QRS, QT, QTc, JT, RR were recorded. In typical experiments, subcutaneous Kaha TR50B bio potential telemeters were surgically implanted in adult male Hartley guinea pigs weighing 300 to 350 g at enrollment. One lead was sutured to the apex of the heart, while another was sutured to the side of the aorta. The animals were allowed to recover from surgery for 5 days prior to being returned to the testing colony. Following recovery, animals were subjected to two rounds of evaluation as follows:

In Round 1 of the testing, baseline ECG records were obtained for 5 minutes prior to exposing the animals to single oral doses of moxifloxacin (20 mg/kg), administered orally to 8 guinea pigs. ECG signals were acquired continuously for 6 hours post administration of moxifloxacin. The animals were then returned to their housing to washout the drug over 5 to 7 days.

In Round 2 of the testing of the 8 guinea pigs, baseline ECGs were obtained for 5 minutes to compare these baseline intervals with the intervals measured prior to the 1st exposure to moxifloxacin (above). The animals were administered a single oral dose of 2 mg/kg of test compound (selected from Compounds 1-12). Concomitantly, 6 animals were gavaged with the same batch of moxifloxacin (20 mg/kg). Another 2 animals were given moxifloxacin (20 mg/kg) only. The purpose of dosing these animals with moxifloxacin only was to verify whether a 2nd exposure to moxifloxacin would result in enhanced QT prolongation. ECGs were acquired continuously for 6 hours. The animals were then returned to their housing to washout the drug over 5 to 7 days.

ECG analysis over 5 minutes pre-dose and 6 hours post dose consisted in was automated based on pattern recognition algorithms. The analyzed data were binned into 5-minute segments. Intervals such as PR, QRS, QT, QTc, JT and RR were analyzed automatically using AD Instruments LabChart Pro v8. The accuracy of the measurements was verified manually using digital cursors by randomly selecting 3 to 5 segments at any given time postdose. There were no noted discrepancies between automated and manual intervals outside of arrhythmic episodes. The frequency of arrhythmia was quantified and expressed as “% of ECG time spent in abnormal sinus rhythm over entire duration of the recording”.

The table below list the protection observed by test compounds against Moxifloxacin-induced QTc prolongation.

| | Moxifloxacin | Test Compound | | | | | | | | | | | | | | | | | | | |
|---|---|---|---|---|---|---|---|---|---|---|---|---|---|---|---|---|---|---|---|---|---|
| | 40 mg/kg | 6 | | 4 | | 2 | | 1 | | 5 | | 11* | | 10* | | 7* | | 8* | | 9* | |
| | Protection indicated as percent (%) of Moxifloxacin effect | | | | | | | | | | | | | | | | | | | | |
| Protection at | n/a | 74 | | 95 | | 95 | | 118 | | 35 | | 83 | | 83 | | 83 | | 74 | | 91 | |
| 1 h post dose | | | | | | | | | | | | | | | | | | | | | |
| Protection at | n/a | 110 | | 88 | | 88 | | 122 | | 113 | | 94 | | 88 | | 100 | | 97 | | 94 | |
| 2 h post dose | | | | | | | | | | | | | | | | | | | | | |
| Protection at | n/a | 115 | | 22 | | 22 | | 100 | | 126 | | 91 | | 72 | | 119 | | 100 | | 100 | |
| 4 h post dose | | | | | | | | | | | | | | | | | | | | | |
| Protection at | n/a | 138 | | 90 | | 90 | | 116 | | 176 | | 116 | | 116 | | 147 | | 132 | | 116 | |
| 6 h post dose | | | | | | | | | | | | | | | | | | | | | |
| Time of | n/a | 2 | h | 2 | h | 2 | h | 2 | h | 2 | h | 2 | h | 2 | h | 2 | h | 2 | h | 2 | h |
| maximal | | | | | | | | | | | | | | | | | | | | | |
| protection | | | | | | | | | | | | | | | | | | | | | |
| Duration of | n/a | 6 | h | 4 | h | 4 | h | 6 | h | 6 | h | 6 | h | 6 | h | 6 | h | 6 | h | 6 | h |
| complete | | | | | | | | | | | | | | | | | | | | | |
| protection | | | | | | | | | | | | | | | | | | | | | |
| Onset of | n/a | 1 | h | 1 | h | 1 | h | 1 | h | 2 | h | 1 | h | 1 | h | 1 | h | 1 | h | 1 | h |
| protection | | | | | | | | | | | | | | | | | | | | | |
| Maximal | 29 ms | 3 | ms | 8 | ms | 4 | ms | 0 | ms | 0 | ms | 8 | ms | 2 | ms | 4 | ms | 2 | ms | 3 | ms |
| prolongation | | | | | | | | | | | | | | | | | | | | | |

*Data for these compounds are preliminary

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### REFERENCES

Bouwer, N. J.-D. (2020). Cardiac monitoring in HER2-positive patients on trastuzumab treatment: A review and implications for clinical practice. The Breast, 52, 33-44.
Cardinale, D. C. (2010). Anthracycline-Induced Cardiomyopathy. Journal of the American College of Cardiology, 55(3), 213-220.
Keefe, D. (2001). Anthracycline-induced cardiomyopathy. Seminars in oncology, 28(12), 2-7.
Lee, K. W. (2021). Cytoprotective Effect of Vitamin D on Doxorubicin-Induced Cardiac Toxicity in Triple Negative Breast Cancer. International Journal of Molecular Sciences, 22(14), 7439-7456.
Romond, E. P. (2005). Trastuzumab plus adjuvant chemotherapy for operable HER2-positive breast cancer. New England Journal of Medicine, 353, 1673-1684.
Sordilo, P. S. (2015). The Prolonged QT Interval: Role of Pro-inflammatory Cytokines, Reactive Oxygen Species and the Ceramide and Sphingosine-1 Phosphate Pathways. In vivo, 29(6), 619-636.

What is claimed is:

1. A compound of Formula I, 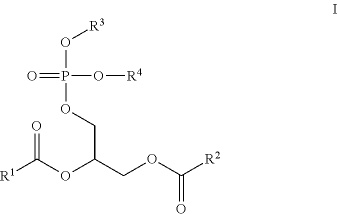
or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 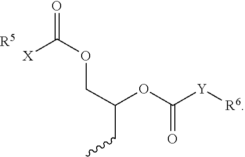
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic.

2. The compound of claim 1, represented by a compound of Formula IA, 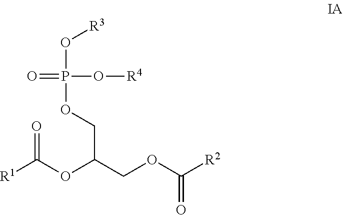
or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 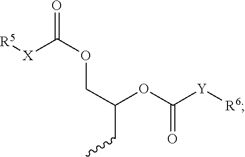
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, N(R⁷)₂ and COOH;
R⁶ is a C₁-C₁₀ branched or unbranched hydrocarbon optionally substituted with one or more groups selected from OH, N(R⁸)₂ and COOH;
Each R⁷ is independently H or a C₁-C₆ branched or unbranched alkyl group;
Each R⁸ is independently H or a C₁-C₆ branched or unbranched alkyl group;
X is a direct linkage;
Y is a direct linkage; and,
each stereogenic center is independently R, S or racemic, and
optionally, R⁴ is H, Li, Na, K, Mg, Ca, Zn, Cs, ammonium or tetraalkylammonium.

3. The compound of claim 1, wherein the compound is selected from at least one of:

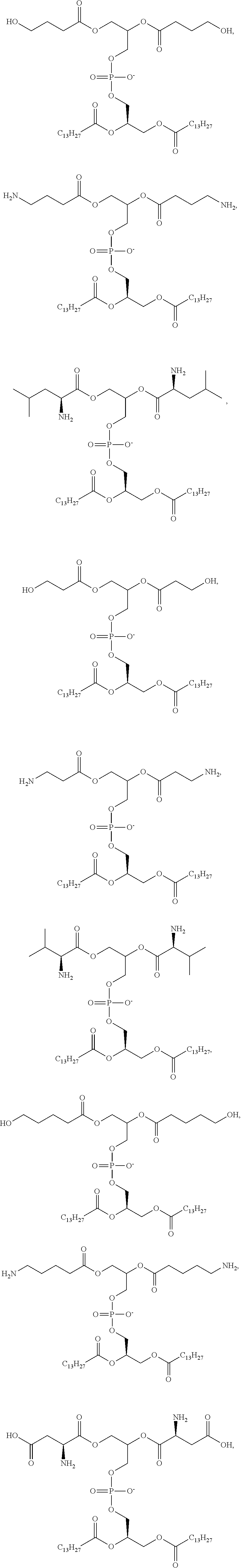

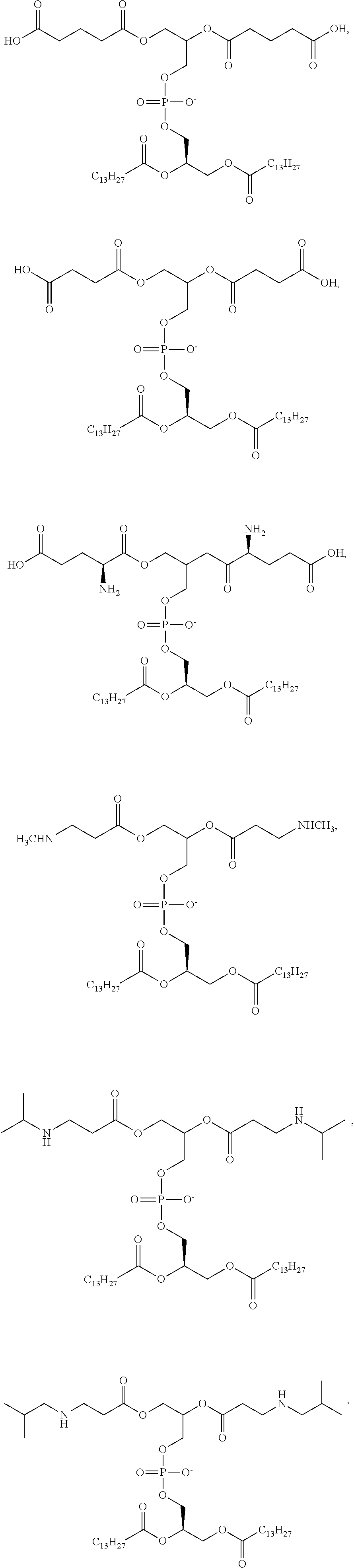

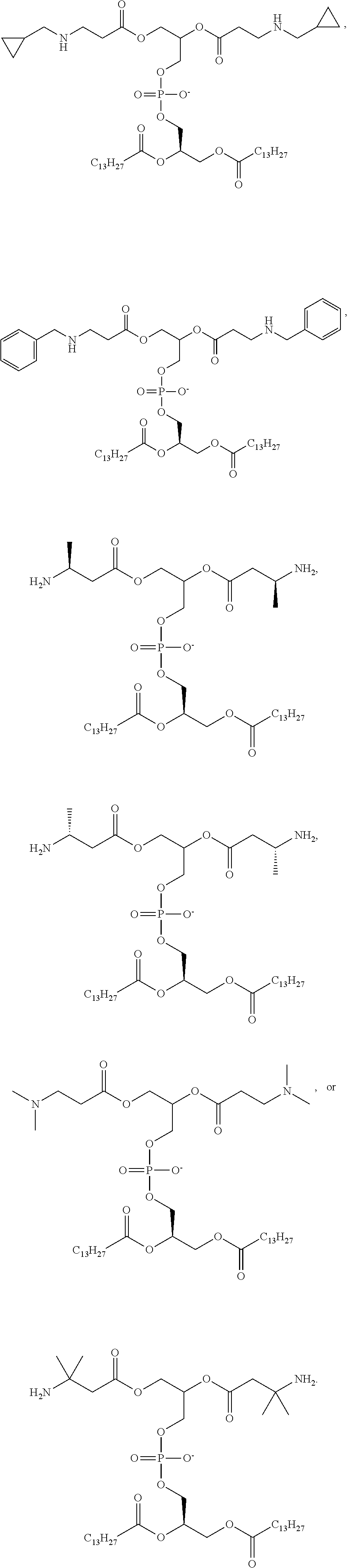

4. The compound of claim 1, wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt, wherein the compound is a single entity, a solvate, a hydrate, a crystal, an amorphous solid, a liquid or an oil.

5. A method of preparing a compound of Formula I 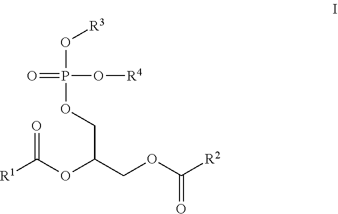
or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 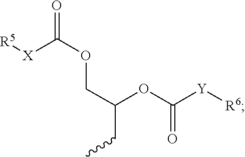
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic;
comprising the steps of:
converting the hydroxyl groups of a compound of Formula II to esters, carbonates, or carbamates 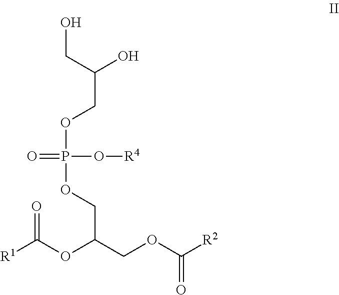
wherein, all substitutions are defined as above;
optionally converting a phosphorus-bound OH group to O—R⁴, wherein R⁴ is not H; and,
optionally removing one or more protecting groups; or
comprising the steps of:
linking a compound of Formula III with a compound of Formula IV through creation of a phosphate diester bridge 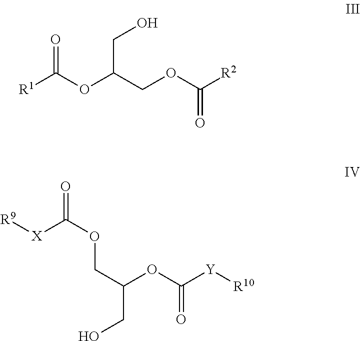
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
each R⁷ is independently H or a C₁-C₆ branched or unbranched alkyl group;
each R⁸ is independently H or a C₁-C₆ branched or unbranched alkyl group;
R⁹ is a C₁-C₁₀ branched or unbranched hydrocarbon substituted with one or more groups independently selected from OR¹¹, N(R⁷)R¹², N(R⁷)₂, SR¹, CN, COOR¹⁴, CONH₂, Cl, Br and I;
R¹⁰ is a C₁-C₁₀ branched or unbranched hydrocarbon substituted with one or more groups independently selected from OR¹¹, N(R⁸)R¹², N(R⁸)₂, SR¹³, CN, COOR¹⁴, CONH₂, Cl, Br and I;
each R¹¹ is independently H, Ac, Me, tert-Butyl, Benzyl, Trityl, Benzoyl, para-nitrobenzoyl, MOM, BOM or Si comprising the core of a silyl ether;
each R¹² is independently H, Me, Boc, Cbz, Fmoc, Benzyl, 4-Methoxybenzyl, tert-Butyl, or Trityl;
each R¹³ is independently H, Ac, Benzoyl, para-nitrobenzoyl or Trityl;
each R¹⁴ is independently H, C₁-C₆ branched or unbranched alkyl, Benzyl or 4-Methoxybenzyl;
X is a direct linkage, O or NH;
Y is a direct linkage, O or NH; and,
each stereogenic center is independently R, S or racemic;
optionally converting a phosphorus-bound OH group to O—R⁴, wherein R⁴ is not H; and,
optionally converting each OR¹¹, N(R⁷)R¹², N(R⁸)R¹², SR¹³ or COOR¹⁴ to OH, NHR⁷, NHR⁸, SH or COOH, respectively.

6. The method of claim 5, wherein the method is used for preparing a compound of Formula IA 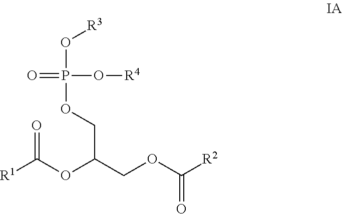
or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 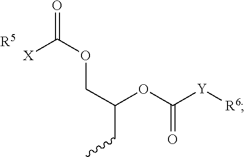
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, N(R⁷)₂ and COOH;
R⁶ is a C₁-C₁₀ branched or unbranched hydrocarbon optionally substituted with one or more groups selected from OH, N(R⁸)₂ and COOH;
each R⁷ is independently H or a C₁-C₆ branched or unbranched alkyl group;
each R⁸ is independently H or a C₁-C₆ branched or unbranched alkyl group;
X is a direct linkage;
Y is a direct linkage; and,
each stereogenic center is independently R, S or racemic;
comprising the steps of:
converting the hydroxyl groups of a compound of Formula II to esters, carbonates, or carbamates 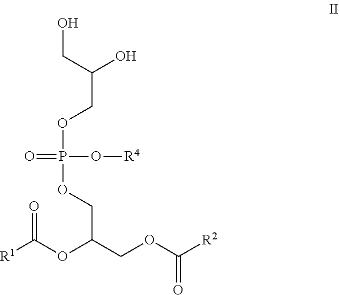
wherein, all substitutions are defined as above;
optionally converting a phosphorus-bound OH group to O—R⁴, wherein R⁴ is not H; and,
optionally removing one or more protecting groups; or
comprising the steps of:
linking a compound of Formula III with a compound of Formula IV through creation of a phosphate diester bridge 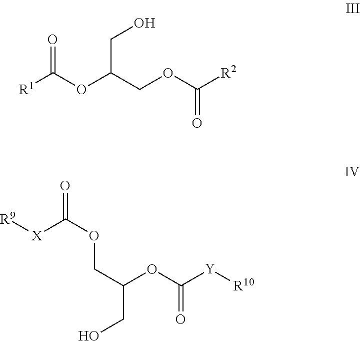
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
each R⁷ is independently H or a C₁-C₆ branched or unbranched alkyl group;
each R⁸ is independently H or a C₁-C₆ branched or unbranched alkyl group;
R⁹ is a C₁-C₁₀ branched or unbranched hydrocarbon substituted with one or more groups independently selected from OR¹¹, N(R⁷)R¹², N(R⁷)₂, and COOR¹⁴;
R¹⁰ is a C₁-C₁₀ branched or unbranched hydrocarbon substituted with one or more groups independently selected from OR¹¹, N(R⁸)R¹², N(R⁸)₂, and COOR¹⁴;
each R¹¹ is independently H, Ac, Me, tert-Butyl, Benzyl, Trityl, Benzoyl, para-nitrobenzoyl, MOM, BOM or Si comprising the core of a silyl ether;
each R¹² is independently H, Me, Boc, Cbz, Fmoc, Benzyl, 4-Methoxybenzyl, tert-Butyl, or Trityl;
each R¹⁴ is independently H, C₁-C₆ branched or unbranched alkyl, Benzyl or 4-Methoxybenzyl;
X is a direct linkage, O or NH;
Y is a direct linkage, O or NH; and,
each stereogenic center is independently R, S or racemic;
optionally converting a phosphorus-bound OH group to O—R⁴, wherein R⁴ is not H; and,
optionally converting each OR¹¹, N(R⁷)R¹², N(R⁸)R¹², or COOR¹⁴ to OH, NHR⁷, NHR⁸ or COOH, respectively.

7. The method of claim 5, wherein the method comprises the steps of converting the hydroxyl groups of a compound of Formula II to esters, carbonates, or carbamates 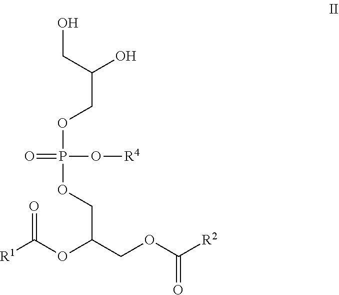
wherein, all substitutions are defined as above;
optionally converting a phosphorus-bound OH group to O—R⁴, wherein R⁴ is not H; and,
optionally removing one or more protecting groups.

8. The method of claim 5, wherein the method comprises the steps of linking a compound of Formula III with a compound of Formula IV through creation of a phosphate diester bridge 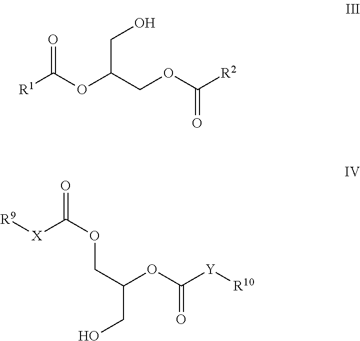
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
each R⁷ is independently H or a C₁-C₆ branched or unbranched alkyl group;
each R⁸ is independently H or a C₁-C₆ branched or unbranched alkyl group;
R⁹ is a C₁-C₁₀ branched or unbranched hydrocarbon substituted with one or more groups independently selected from OR¹¹, N(R⁷)R¹², N(R⁷)₂, SR¹³, CN, COOR¹⁴, CONH₂, Cl, Br and I;
R¹⁰ is a C₁-C₁₀ branched or unbranched hydrocarbon substituted with one or more groups independently selected from OR¹¹, N(R⁸)R¹², N(R⁸)₂, SR¹³, CN, COOR¹⁴, CONH₂, Cl, Br and I;
each R¹¹ is independently H, Ac, Me, tert-Butyl, Benzyl, Trityl, Benzoyl, para-nitrobenzoyl, MOM, BOM or Si comprising the core of a silyl ether;
each R¹² is independently H, Me, Boc, Cbz, Fmoc, Benzyl, 4-Methoxybenzyl, tert-Butyl, or Trityl;
each R¹³ is independently H, Ac, Benzoyl, para-nitrobenzoyl or Trityl;
each R¹⁴ is independently H, C₁-C₆ branched or unbranched alkyl, Benzyl or 4-Methoxybenzyl;
X is a direct linkage, O or NH;
Y is a direct linkage, O or NH; and,
each stereogenic center is independently R, S or racemic;
optionally converting a phosphorus-bound OH group to O—R⁴, wherein R⁴ is not H; and,
optionally converting each OR¹¹, N(R⁸)R¹², N(R⁸)R¹², SR¹³ or COOR¹⁴ to OH, NHR⁷, NHR⁸, SH or COOH, respectively, and optionally R⁴ is H, Li, Na, K, Mg, Ca, Zn, Cs, ammonium or tetraalkylammonium.

9. The method of claim 5, wherein the compound is selected from at least one of:

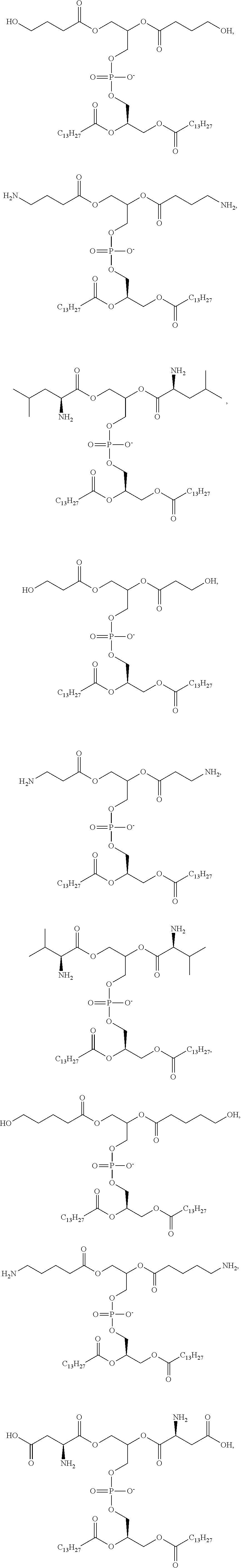

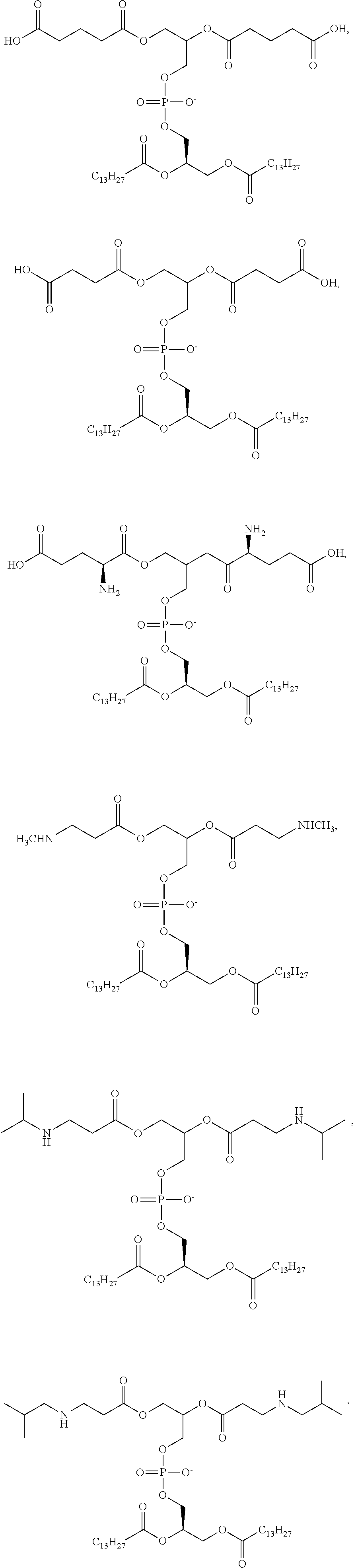

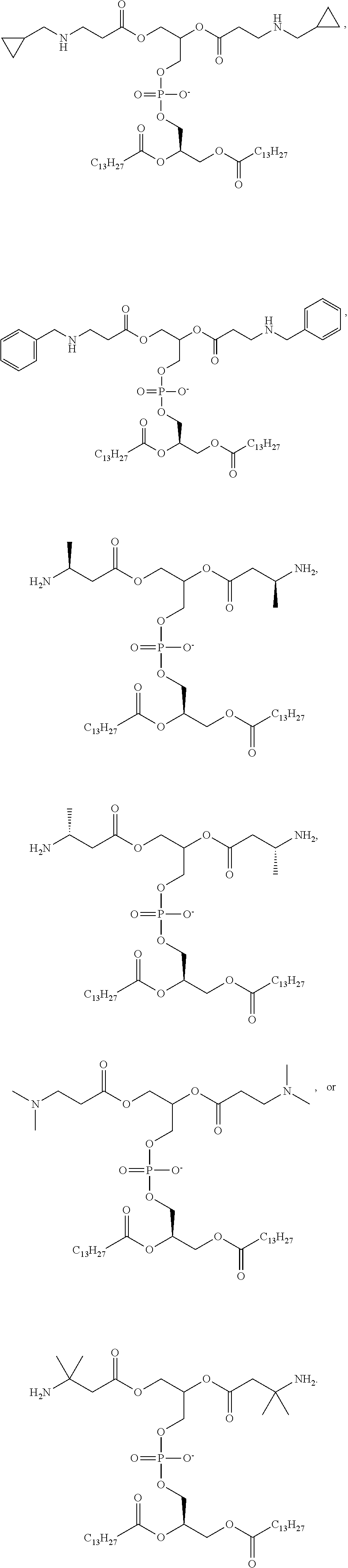

10. The method of claim 5, wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt, and optionally, the method produces compounds that individually exist as a single entity, a solvate, a hydrate, a crystal, an amorphous solid, a liquid, or an oil.

11. A pharmaceutical composition comprising a compound of Formula I and a pharmaceutically acceptable diluent or carrier 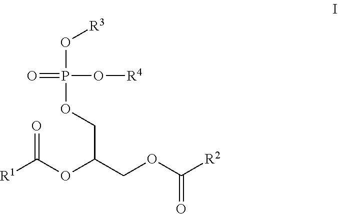
or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 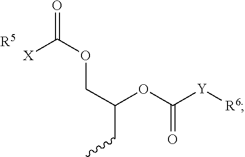
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic, and
optionally, R⁴ of the compound of Formula I is H, Li, Na, K, Mg, Ca, Zn, Cs, ammonium or tetraalkylammonium.

12. The pharmaceutical composition of claim 11, wherein the compound of Formula I is selected from one or more of:

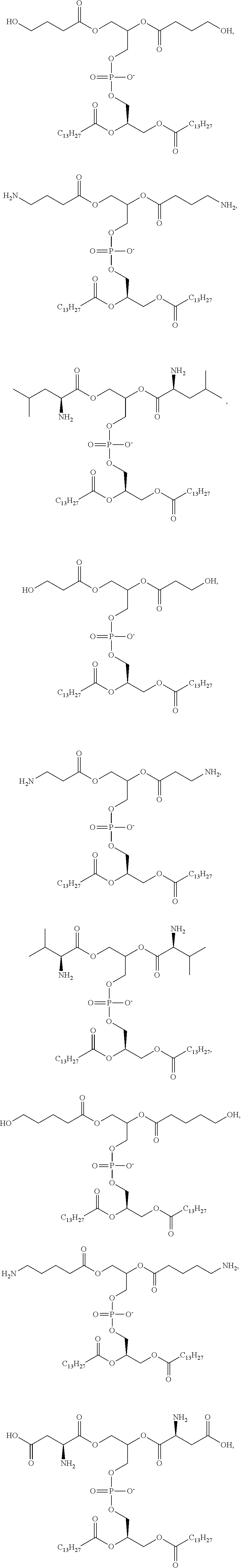

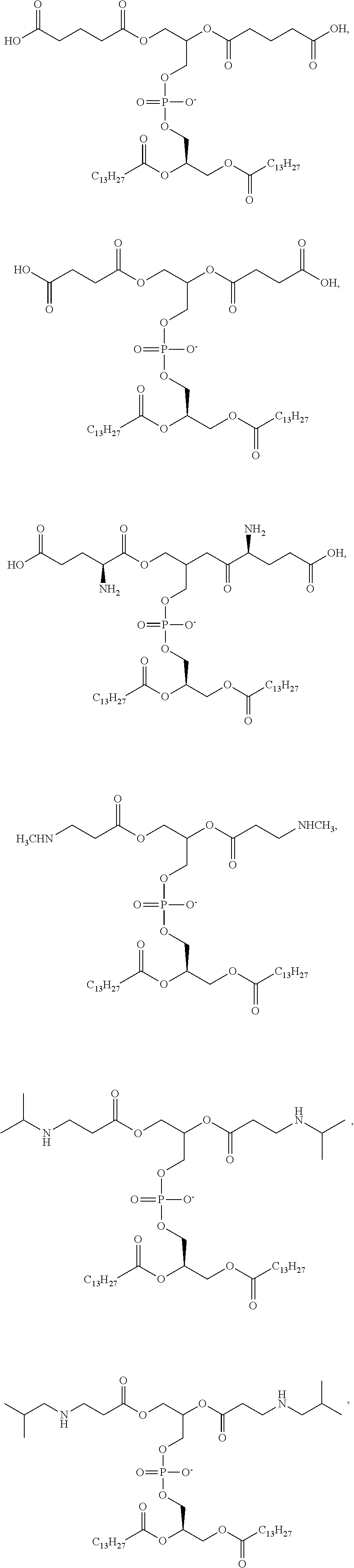

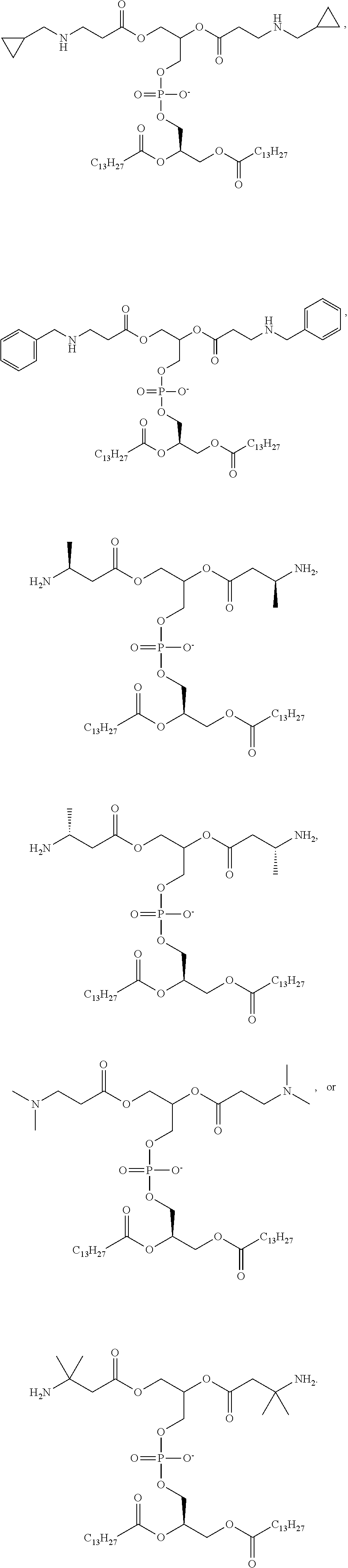

13. The pharmaceutical composition of claim 11, wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt, and optionally the compound of Formula I exists as a single entity, a solvate, a hydrate, a crystal, an amorphous solid, a liquid or an oil.

14. The pharmaceutical composition of claim 11, wherein the pharmaceutical composition further comprises one or more agents that induce a cardiopathy as a side effect.

15. The pharmaceutical composition of claim 14, wherein one or more agents that induces a cardiopathy as a side effect is selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone.

16. The pharmaceutical composition of claim 11, wherein the compound of Formula I is at least one of: exists as a single entity, a solvate, a hydrate, a crystal, an amorphous solid, a liquid or an oil; reduces or eliminates one or more of a cardiac channelopathy or a condition resulting from the irregularity or alteration in the cardiac pattern caused by the active agent used to treat a disease; is administered in an amount per unit dose of between about 1 mg and about 1 gram; or is formulated for oral, sublingual, transdermal, suppository, intrathecal, enteral, parenteral, intravenous, intraperitoneal, cutaneous, subcutaneous, topical, pulmonary, rectal, vaginal, or intramuscular administration, and optionally, the compound of Formula I is formulated for oral administration as a tablet, capsule, caplet, pill, powder, troche, lozenge, slurry, liquid solution, suspension, emulsion, elixir or oral thin film (OTF), a solid form, a solution, a suspension, or a soft gel form; or optionally, the solid form further comprises one or more excipients, binders, anti-adherents, coatings, disintegrants, fillers, flavors, dyes, colors, glidants, lubricants, preservatives, sorbents, sweeteners, derivatives thereof, or combinations thereof.

17. A method of reducing or eliminating one or more of a cardiac channelopathy, cardiac muscle damage, or a condition resulting from the irregularity or alteration in the cardiac pattern, in a human or animal subject, comprising the step of administering to the human or animal subject one or more of a compound of Formula I 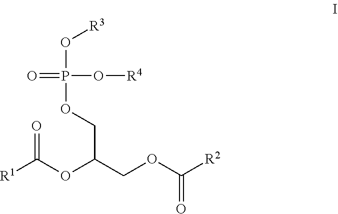
or a pharmaceutically acceptable salt thereof
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 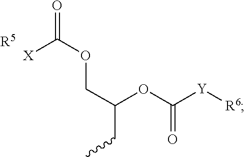
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic, and
optionally the R⁴ of the compound of Formula I is H, Li, Na, K, Mg, Ca, Zn, Cs, ammonium or tetraalkylammonium.

18. The method of claim 17, wherein the compound of Formula I at least one of: exists as a single entity, a solvate, a hydrate, a crystal, an amorphous solid, a liquid or an oil; reduces or eliminates one or more of a cardiac channelopathy or a condition resulting from the irregularity or alteration in the cardiac pattern caused by the active agent used to treat a disease; is administered in an amount per unit dose of between about 1 mg and about 1 gram; or is formulated for oral, sublingual, transdermal, suppository, intrathecal, enteral, parenteral, intravenous, intraperitoneal, cutaneous, subcutaneous, topical, pulmonary, rectal, vaginal, or intramuscular administration, and optionally, the compound of Formula I is formulated for oral administration as a tablet, capsule, caplet, pill, powder, troche, lozenge, slurry, liquid solution, suspension, emulsion, elixir or oral thin film (OTF), a solid form, a solution, a suspension, or a soft gel form; or optionally, the solid form further comprises one or more excipients, binders, anti-adherents, coatings, disintegrants, fillers, flavors, dyes, colors, glidants, lubricants, preservatives, sorbents, sweeteners, derivatives thereof, or combinations thereof.

19. The method of claim 17, wherein the compound of Formula I is co-administered with one or more agents that induce a cardiopathy as a side effect.

20. The method of claim 19, wherein the one or more active agent that induce a cardiopathy as a side effect are selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone.

21. The method of claim 17, wherein the compound of Formula I reduces or eliminates cardiopathies, such as QT prolongation, cardiac muscle damage, or AV block that is drug-induced or caused by a disease or condition.

22. The method of claim 17, wherein the compound is:

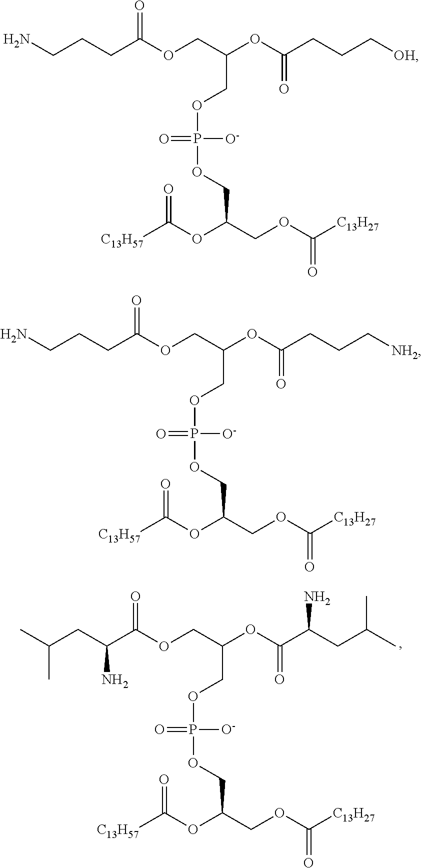

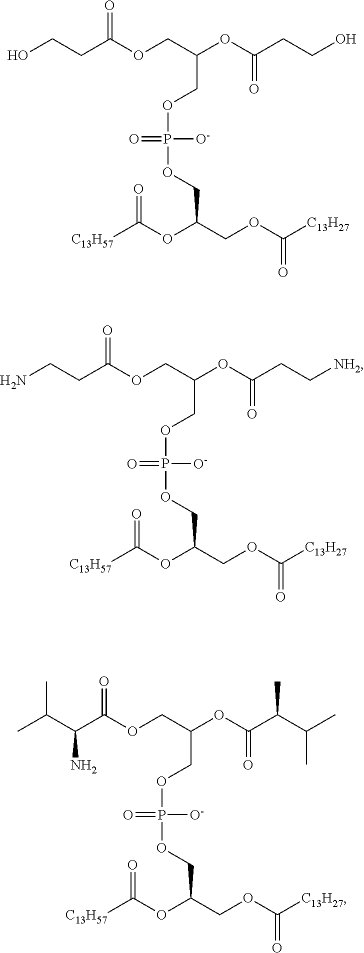

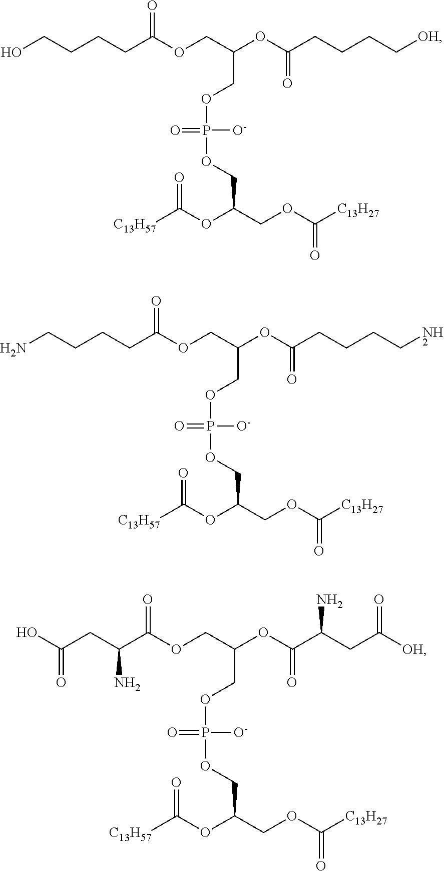

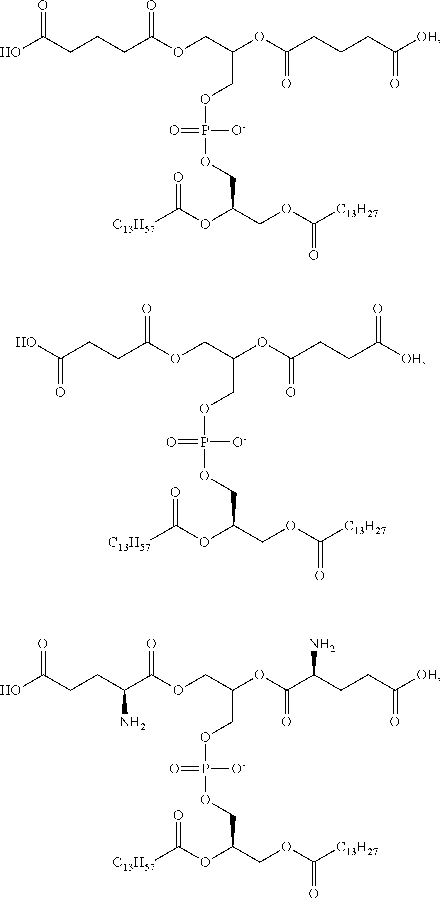

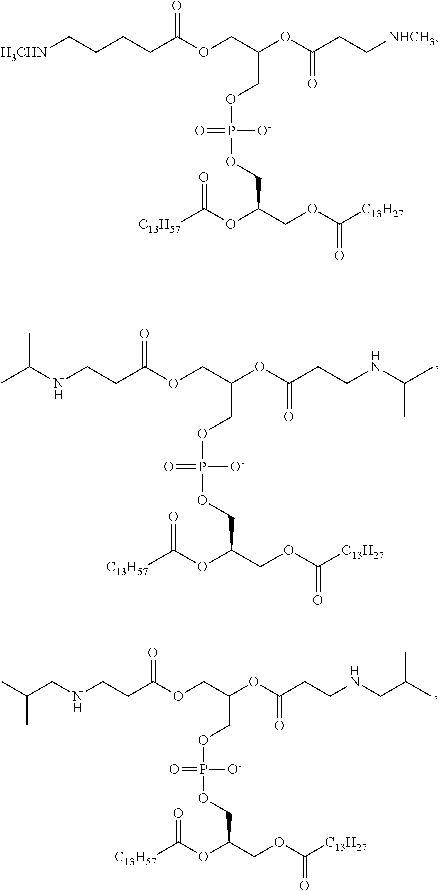

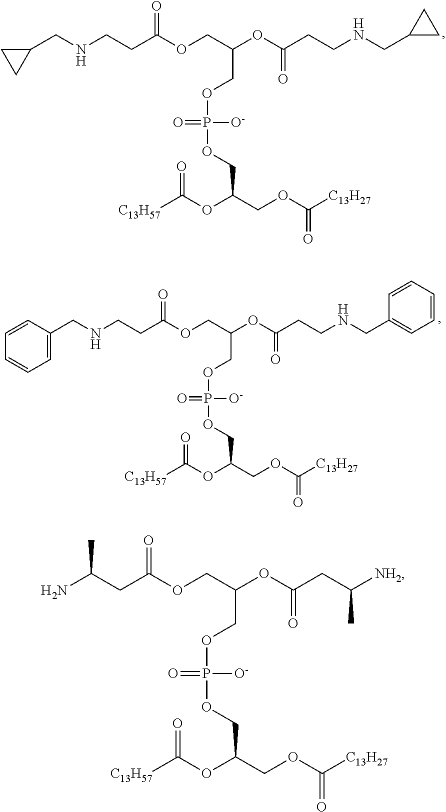

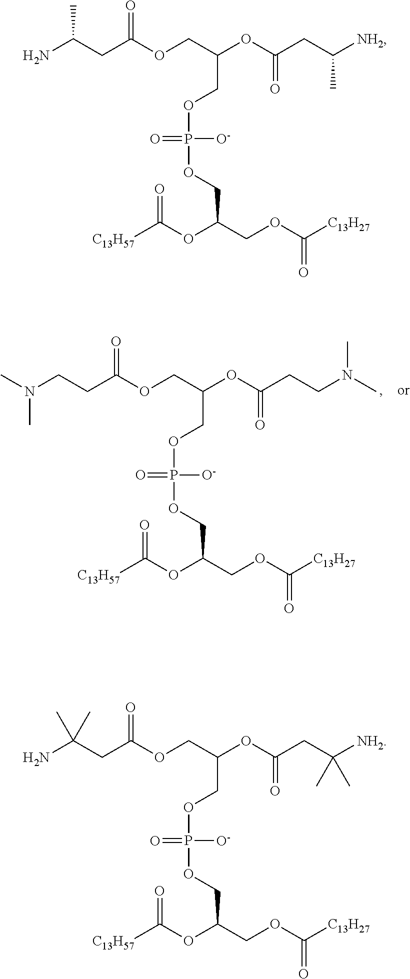

23. The method of claim 17, wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt, wherein the compound is a single entity, a solvate, a hydrate, a crystal, an amorphous solid, a liquid or an oil.

24. A method of reducing or eliminating a cardiotoxic or cardiopathic effect of one or more active agents comprising:
administering to a subject in need of treatment for a disease or disorder one or more one or more active agents that are cardiotoxic; and
providing a combination therapy with an effective amount of one or more lipids that reduce or eliminate the cardiotoxic effect of the one or more active agents, wherein the lipid has formula: 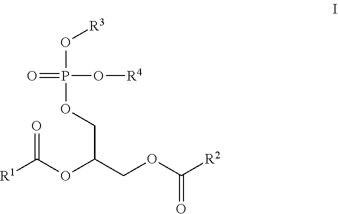
or a pharmaceutically acceptable salt thereof wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 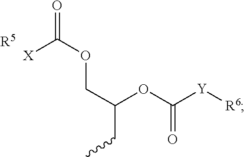
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic, and
wherein the reduction in cardiotoxicity is at least 25, 30, 40, 50, 60, 70, 75, 80, 90, 95, or 100% when compared to a treatment without the lipid, and optionally, wherein R⁴ is H, Li, Na, K, Mg, Ca, Zn, Cs, ammonium or tetraalkylammonium.

25. The method of claim 24, wherein the cardiotoxicity or cardiopathicity is selected from at least one of: minimal left ventricular dilation, contractile dysfunction, moderate valve regurgitation, a decline in left ventricular ejection fraction (LVEF), cardiac hypertrophy, reduced cardiac contractility, reduced cardiac output, pressure and volume overload hypertrophy, myocardial dysfunction, cardiac remodeling, post-myocardial infarction heart failure, or cardiopathy.

26. The method of claim 24, wherein the one or more active agents and the lipid are administered concurrently or the one or more active agents and the lipid are formulated for oral, sublingual, transdermal, suppository, intrathecal, enteral, parenteral, intravenous, intraperitoneal, cutaneous, subcutaneous, topical, pulmonary, rectal, vaginal, or intramuscular administration, or wherein the one or more lipids, the one or more active agents, or both, are infused over 3 hours.

27. The method of claim 24, wherein one or more agents that induces a cardiotoxic or cardiopathic effect is selected from at least one of: Adrenaline, Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Bretylium, Buprenorphine, Capecitabine, Chloral hydrate Clomipramine, Chloroquine, Chlorpromazine, Ciprofloxacin, Cisapride, Citalopram, Clarithromycin, Clomipramine, Clozapine, Cocaine, Crizotinib, Curcumin, Cyclobenzaprine, Cyclosporin, Dabrafenib, Dasatinib, Degarelix, Desipramine, Desvenlafaxine, Dexmedetomidine, Dexmethylphenidate, Dextroamphetamine, Dihydroartemisinin and Piperaquine, Diphenhydramine, Disopyramide, Dobutamine, Dofetilide, Dolasetron, Domperidone, Donepezil, Dopamine, Doxepin, Dronedarone, Droperidol, Ephedrine, Epinephrine, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Frusemide, Furosemide, Galantamine, Gatifloxacin, Gemifloxacin, Granisetron, Halofantrine, Haloperidol, Hydrochlorothiazide, Hydroxychloroquine, Hydroxyzine, Ibutilide, Iloperidone, Imipramine, Indapamide, Isoproterenol, Isradipine, Itraconazole, Ivabradine, Ketoconazole, Lapatinib, Leuprolide, Levalbuterol, Levofloxacin, Levomepromazine, Levomethadyl, Lisdexamfetamine, Lithium, Loperamide, Maprotiline, Mefloquine, Melipramine, Mesoridazine, Metaproterenol, Methadone, Methamphetamine, Methylphenidate, Metoclopramide, Mexiletine, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Octreotide, Ofloxacin, Olanzapine, Ondansetron, Orphenadrine, Oxaliplatin, Oxycodone, Oxytocin, Paliperidone, Papaverine HCl, Paroxetine, Pasireotide, Pazopanib, Pazopanib, Pentamidine, Perflutren lipid microspheres, Perphenazine, Phentermine, Phenylephrine, Phenylpropanolamine, Pimozide, Posaconazole, Probucol, Procainamide, Promethazine, Propafenone, Propofol, Propoxyphene, Protriptyline, Pseudoephedrine, Quetiapine, Quinidine, Quinine, Quinine sulfate, Ranolazine, Rilpivirine, Risperidone, Ritodrine, Ritonavir, Ritonavir and Lopinavir, Roxithromycin, Salbutamol, Salmeterol, Saquinavir, Sertindole, Sertraline, Sevoflurane, Sibutramine, Solifenacin, Sorafenib, Sotalol, Sparfloxacin, Spiramycin, Sulpiride, Sunitinib, Tacrolimus, Tamoxifen, Telaprevir, Telavancin, Telithromycin, Terbutaline, Terfenadine, Tetrabenazine, Thioridazine, Thiothixene, Tizanidine, Tizanidinev, Tolterodine, Toremifene, Torsemide, Trazodone, Trimethoprim and Sulfamethoxazole, Trimethoprim-Sulfa, Trimipramine, Vandetanib, Vardenafil, Vemurafenib, Venlafaxine, Voriconazole, Vorinostat, Ziprasidone, or Ziprasidone.

28. The method of claim 27, wherein a pharmaceutical composition comprising at least one of:
the one or more lipids further comprises one or more excipients, binders, anti-adherents, coatings, disintegrants, fillers, flavors, dyes, colors, glidants, lubricants, preservatives, sorbents, sweeteners, derivatives thereof, or combinations thereof;
a compound of Formula I in an amount per unit dose of about 0.1, 0.5, 1, 2, 3, 4, 5, 6, 7, 8, 9, 10, 15, 20, 24, 30, 40, 50, 60, 75, 80, 90, 100, 125, 150, 175, 200, 225, 250, 275, 300, 325, 350, 375, 400, 450, 500, 550, 600, 650, 700, 750, 800, 850, 900, 950, 1,000, 2,000, 3,000, 4,000, 5,000, 6,000, 7,000, 8,000, 9,000, or 10,000 milligrams per unit dose;
a formulation for oral, sublingual, transdermal, suppository, intrathecal, enteral, parenteral, intravenous, intraperitoneal, cutaneous, subcutaneous, topical, pulmonary, rectal, vaginal, or intramuscular administration;
a formulation for oral administration is a tablet, capsule, caplet, pill, powder, troche, lozenge, slurry, liquid solution, suspension, emulsion, elixir or oral thin film (OTF); or
the formulation is a solid form, a solution, a suspension, or a soft gel form.

29. The method of claim 24, wherein the compound of Formula I is selected from one or more of:

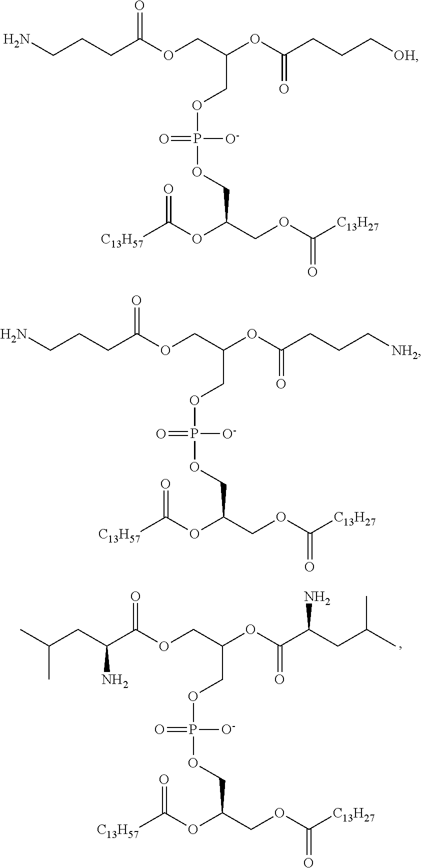

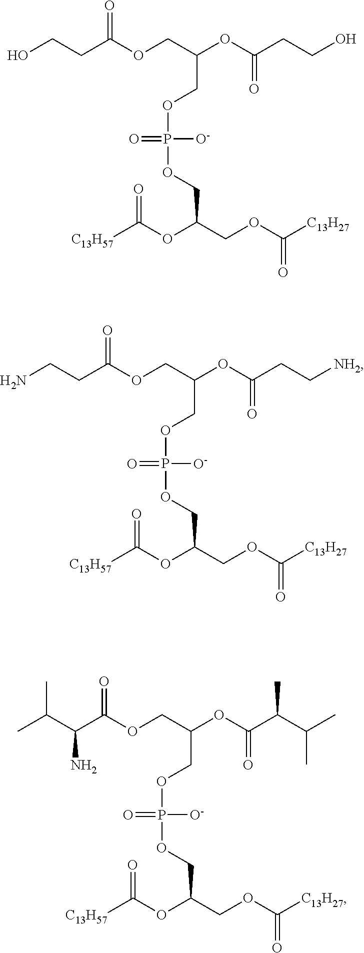

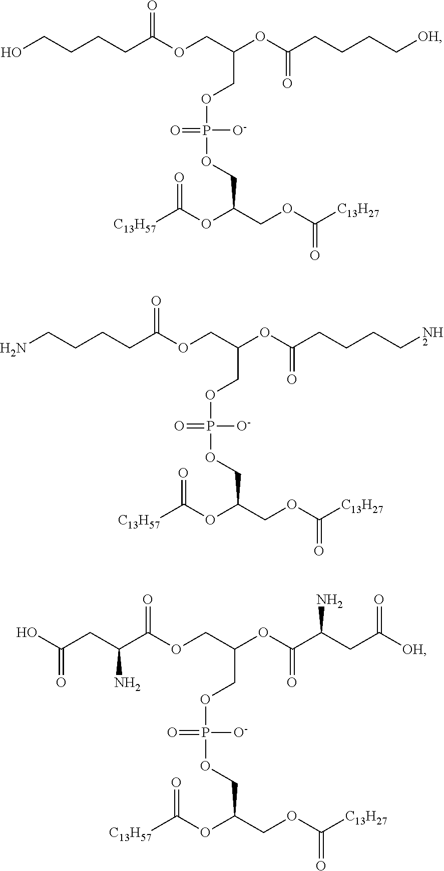

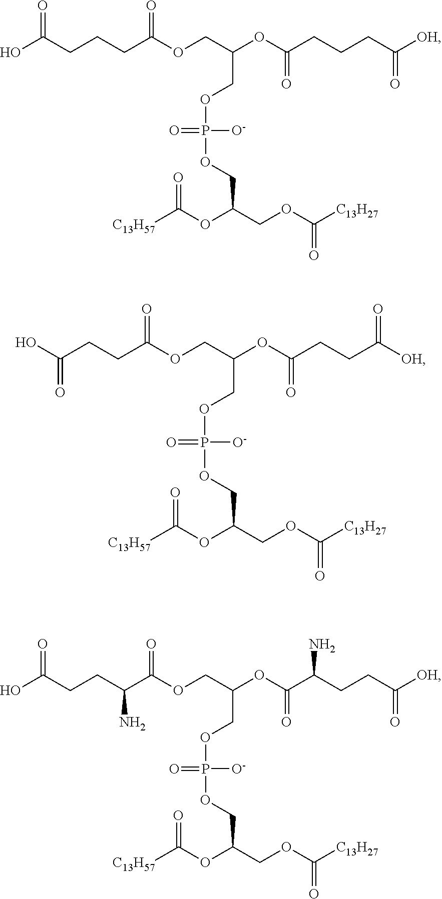

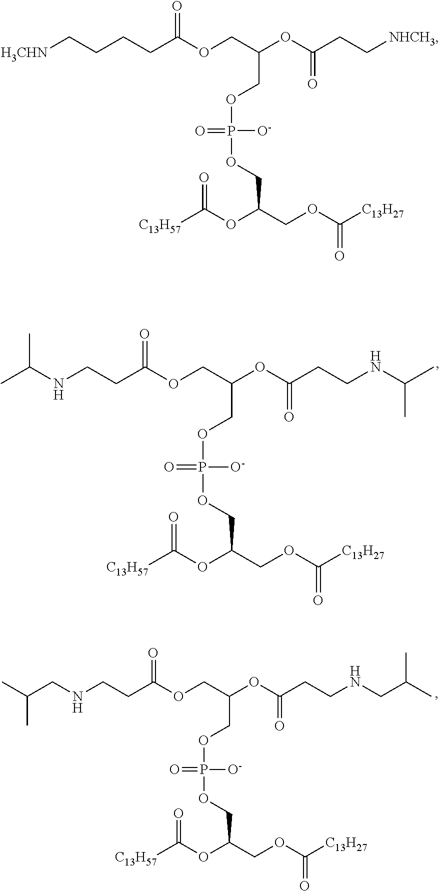

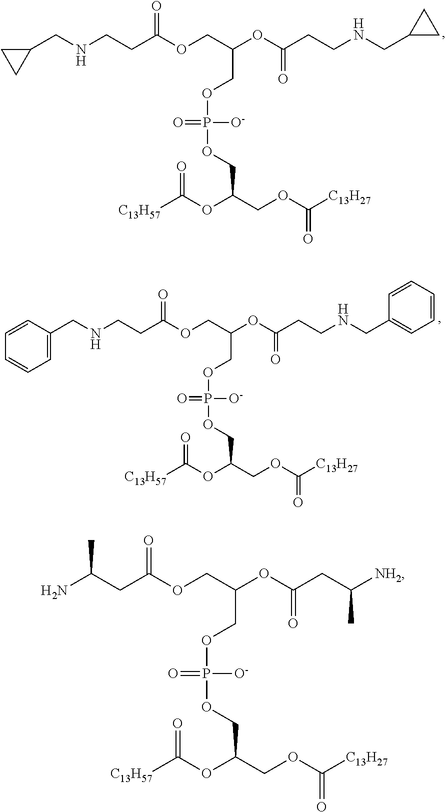

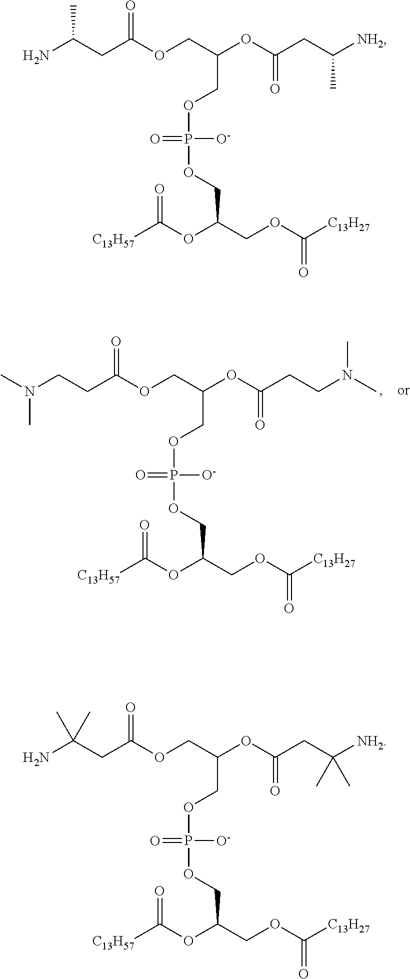

30. The method of claim 24, wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt, wherein the compound is a single entity, a solvate, a hydrate, a crystal, an amorphous solid, a liquid or an oil.

31. A method of reducing or eliminating a cardiotoxic effect of one or more antiproliferative agents comprising:
administering to a subject in need of treatment for a proliferative disorder one or more antiproliferative agents that are cardiotoxic; and providing a combination therapy with an effective amount of one or more lipids that reduce or eliminate the cardiotoxic effect of one or more antiproliferative agents, wherein the lipid has formula: 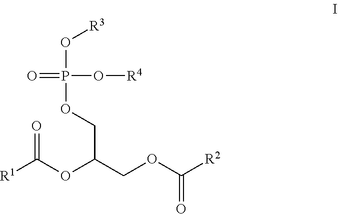
or a pharmaceutically acceptable salt thereof wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 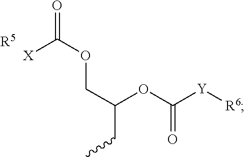
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NHAc, N(R⁷)₂, SH, CN, COOH, CONH₂, Cl, Br and I;
R⁶ is a C₁-C₁₀ branched or unbranched hydrocarbon optionally substituted with one or more groups selected from OH, OAc, OMe, NHAc, N(R⁸)₂, SH, CN, COOH, CONH₂, Cl, Br and I;
each R⁷ is independently H or a C₁-C₆ branched or unbranched alkyl group;
each R⁸ is independently H or a C₁-C₆ branched or unbranched alkyl group;
X is a direct linkage, O or NH;
Y is a direct linkage, O or NH; and,
each stereogenic center is independently R, S or racemic, and optionally, wherein R⁴ is H, Li, Na, K, Mg, Ca, Zn, Cs, ammonium or tetraalkylammonium; and wherein the reduction in cardiotoxicity is at least 25, 30, 40, 50, 60, 70, 75, 80, 90, 95, or 100% when compared to a treatment without the lipid.

32. The method of claim 31, wherein the cardiotoxicity is selected from at least one of: minimal left ventricular dilation, contractile dysfunction, moderate valve regurgitation, a decline in left ventricular ejection fraction (LVEF), cardiac hypertrophy, reduced cardiac contractility, reduced cardiac output, pressure and volume overload hypertrophy, myocardial dysfunction, cardiac remodeling, post-myocardial infarction heart failure, or cardiopathy; the one or more antiproliferative agents and the lipid are administered concurrently; the one or more antiproliferative agents and the lipid administered orally, or intravenously; the one or more lipids, the one or more antiproliferative agents, or both, are infused over 3 hours; or the one or more antiproliferative agents that induce a cardiopathy as a side effect are selected from at least one of: Bosutinib, Crizotinib, Dabrafenib, Dasatinib, Doxorubicin Lapatinib, Nilotinib, Sorafenib, Sunitinib, Vandetanib, or Vemurafenib.

33. The method of claim 31, wherein the compound of Formula I is selected from one or more of:

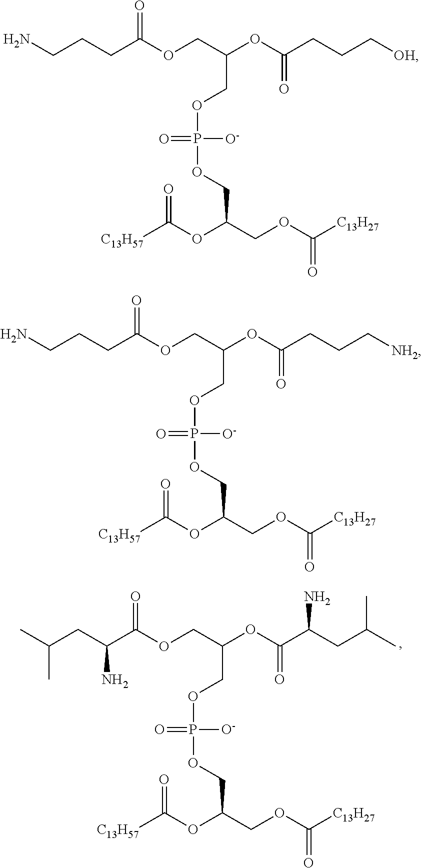

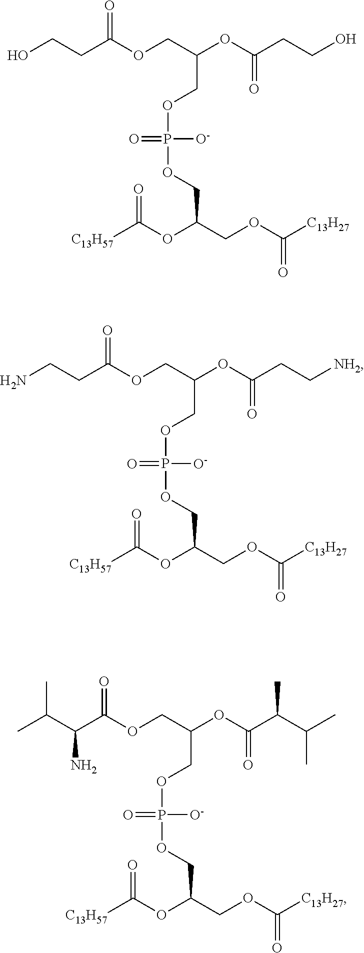

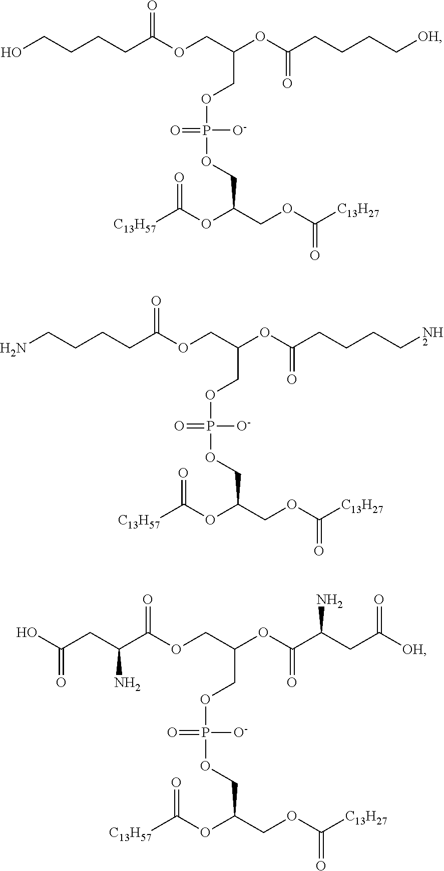

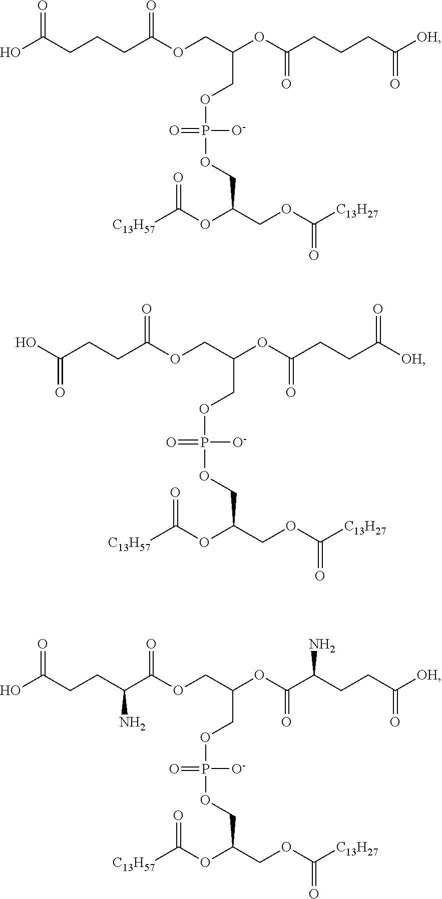

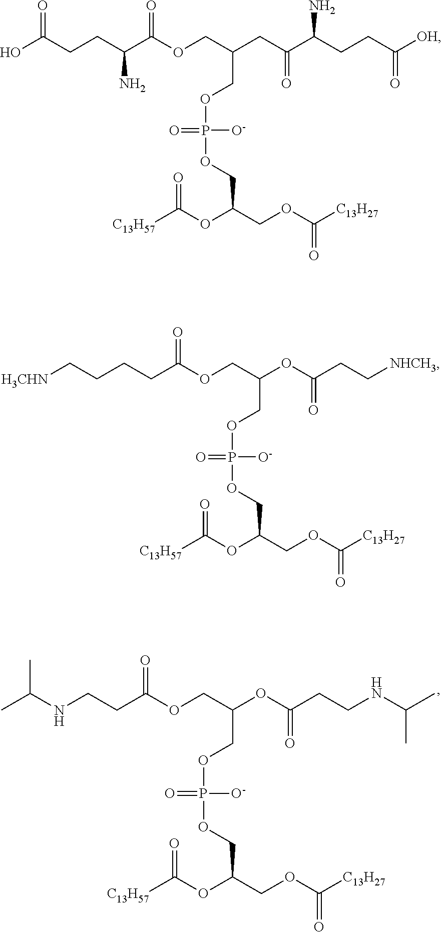

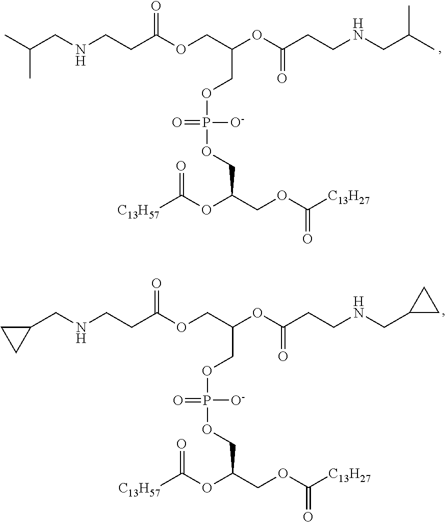

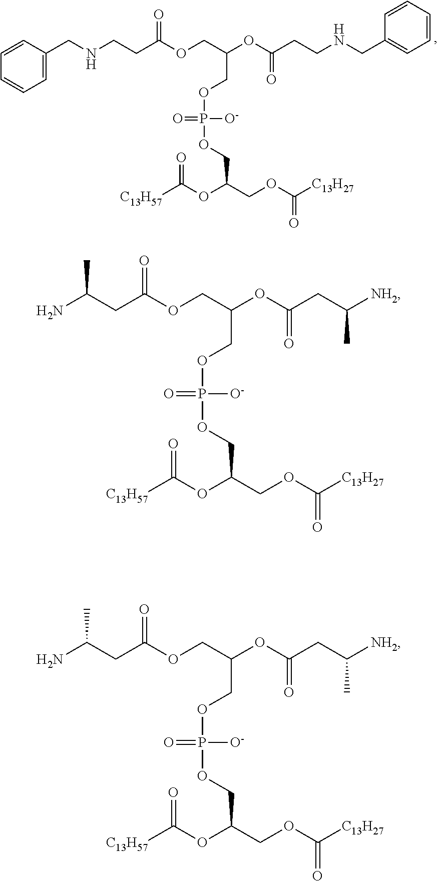

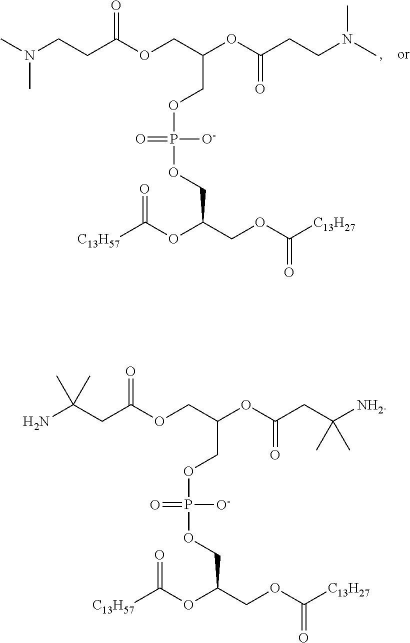

34. The method of claim 31, wherein any oxygen anion O⁻ of any compound is paired with H, Li, Na, K, Mg, Ca, Zn, Cs, ammonium or tetraalkylammonium; and wherein, any NH₂ or COOH of any compound is optionally in the form of a pharmaceutically acceptable salt, wherein the compound is a single entity, a solvate, a hydrate, a crystal, an amorphous solid, a liquid or an oil.
